(19) United States
(12) Patent Application Publication
(10) Pub. No.: US 2026/0002165 A1
(43) Pub. Date: Jan. 1, 2026

(54) TARGETING CELLS IN STRESSED GROWING CONDITIONS

(71) Applicant: SNIPR BIOME ApS, Copenhagen (DK)

(72) Inventors: Szabolcs SEMSEY, Copenhagen (DK); Jakob KRAUSE HAABER, Copenhagen (DK); Melissa Kviesgaard ERIKSEN, Copenhagen (DK)

(21) Appl. No.: 19/318,174

(22) Filed: Sep. 3, 2025

Related U.S. Application Data
(63) Continuation of application No. PCT/EP2024/055870, filed on Mar. 6, 2024.

(30) Foreign Application Priority Data
Mar. 6, 2023 (GB) 2303242.8
Mar. 8, 2023 (GB) 2303409.3

(51) Int. Cl. C12N 15/70; A01N 63/60; A01P 1/00; C12N 9/22; C12N 15/11; C12N 15/86

(52) U.S. Cl. CPC C12N 15/70; A01N 63/60; A01P 1/00; C12N 9/224; C12N 15/11; C12N 15/86; C12N 2310/20; C12N 2795/00032; C12N 2795/00043

(57) ABSTRACT

The present invention provides method of killing first cells (for example pathogenic cells) which are slow-growing under stress conditions (for example, stationary phase cells, stressed cells, persister cells, biofilm cells and dormant cells) by providing a vector encoding a toxic agent or component thereof under the control of a stress-phase active (SPA) promoter. The invention also provides methods of expressing proteins of interest (POIs) in such slow-growing cells under stress conditions, which POIs may be therapeutic molecules and reporter genes for example, by providing a vector encoding a POI under the control of a SPA promoter. There are provided methods of treatment using such vectors.

### CROSS REFERENCE TO RELATED APPLICATIONS

This application is a continuation of International Application No. PCT/EP2024/055870, filed internationally on Mar. 6, 2024, which claims priority to Great Britain Application No. 2303242.8, filed on Mar. 6, 2023, and Great Britain Application No. 2303409.3, filed on Mar. 8, 2023, the disclosures of each of which are hereby incorporated herein by reference in their entirety.

### REFERENCE TO AN ELECTRONIC SEQUENCE LISTING

The content of the electronic sequence listing (786212002301seglist.xml; Size: 10,491 bytes; and Date of Creation: Sep. 2, 2025) is herein incorporated by reference in its entirety.

### BACKGROUND TO INVENTION

Prokaryotes, such as bacteria and archaea, but also some yeast may reproduce by binary fission. In this process, the single cell divides into two identical daughter cells. The time required to complete this process is the generation time (doubling time). If the doubling time remains constant, the cell culture is in exponential growth.

Bacterial growth in batch culture can be modelled with four different phases: lag phase, exponential or log phase, stationary phase, and death phase. During lag phase, bacteria adapt themselves to growth conditions. It is the period where the individual bacteria are maturing and not yet able to divide. During this phase of the bacterial growth cycle, synthesis of RNA, enzymes, and other molecules occurs. The exponential phase (sometimes called the log phase or the logarithmic phase) is a period characterised by cell doubling. The number of new bacteria appearing per unit time is proportional to the present population. If growth is not limited, doubling will continue at a constant rate, so both the number of cells and the rate of population increase doubles with each consecutive time period. For this type of exponential growth, plotting the natural logarithm of cell number against time produces a straight line. In this regime, the generation time (doubling time) of cells is calculated as

Generation time=[(t₁−t₀)*log(2)]/[log(OD600ₜ₁)−log(OD600ₜ₀)] Equation 1:
where t₀ is the time at the first measurement, t₁ is time at the second measurement, OD600ₜ₀ is the optical density of the culture at the first measurement, OD600ₜ₁ is the optical density of the culture at the second measurement, and log is logarithm of any base.

The measurement of an exponential bacterial growth curve can be achieved by cell counting, colony counting, determining the turbidity of bacterial cultures, or alternatively by molecular biological methods such as qPCR or calculation of protein concentration.

The actual rate of this growth depends upon the growth conditions, which affect the frequency of cell division events and the probability of both daughter cells surviving. The theoretical minimum of the doubling time depends on the bacterial strain and is determined by the cell genome. The doubling time can be approximated in the laboratory, by optimizing the physical and chemical environment of cells. The environmental variables include temperature, pH, osmolarity, oxygen concentration, concentration and types of nutrient sources (e.g. carbon, nitrogen), meso- and microelements. The growth conditions and growth nutrients used when bacterial growth is obtained under optimised lab conditions is referred to as standard conditions. For example, when an E. coli strain, such as the K12 reference strain MG1655, is cultivated in LB (lysogeny broth) medium at 37° C. and in oxygenated conditions (Erlenmeyer flask filled to maximum 10% of volume and incubated with orbital shaking of 250 rpm), the bacteria can grow with a generation time of just 20 minutes during the exponential growth phase.

Environmental stress can be an external factor that has an adverse effect on the physiological welfare of cells, such as bacterial cells. Many bacteria can adapt to such stress conditions by altering gene expression. However, severe stress can substantially interfere with bacterial physiology, resulting in a state characterised by reduced growth (e.g. a doubling time lower compared to growth under standard conditions, such as those described in examples 1, 2 and 3 herein), or halted growth.

### SUMMARY OF THE INVENTION

When bacterial cells are not actively growing and dividing, most antibiotic compounds are not efficacious. For example, it is well known that slow growing cells (i.e. those displaying reduced growth (e.g. a doubling time lower compared to growth under standard conditions), or halted growth) inhabiting a biofilm or being in a persister state are tolerant to antibiotics, even though the same cells are susceptible to the antibiotics when growing exponentially in the lab. The inventors have advantageously realised means to decouple the killing potential of cytotoxic agents, and the expression of therapeutics and other products of interest, from the growth state of the target cells. In particular, the use of targeted nucleases (e.g. Cas nucleases) are useful in this respect, since the nuclease, if expressed in slow growing cells (e.g. in stationary phase cells, dormant cells, stressed cells and/or biofilm cells), will cut target nucleic acid (e.g. chromosomal DNA) of such cells, irrespective of the metabolic state. When the environment subsequently changes to favourable growth conditions where antibiotic-treated cells would normally resume growth, the agent (e.g. nuclease)-treated cells are dead and cannot resume growth.

Equally, expression of certain therapeutic molecules within such slow growing cells can be beneficial to provide a consistent supply of said therapeutics within a biofilm or similar.

To obtain actively expressed cytotoxic agents (e.g. using CRISPR/Cas systems), or actively expressed therapeutic and other proteins of interest (POIs) in the slow growing cells, we exploit promoter DNA sequences that are active under the corresponding environmental conditions in the target cells to advantageously drive expression of such agents and proteins.

The present invention therefore provides the following:

### In a First Configuration:

There is provided a method of killing first cells comprised by a cell population, the method comprising introducing a nucleic acid vector into the first cells,
wherein the vector comprises a nucleotide sequence that encodes (i) an agent that is toxic to the first cells, or (ii) a component of the toxic agent,
wherein the nucleotide sequence of the vector is under the control of a stress-phase active (SPA) promoter for expression of the agent or component thereof, and the sequence is expressed in the first cells to produce the toxic agent or component thereof, whereby the first cells are killed,
and wherein the SPA promoter shows at least the same promoter activity in in vitro stress conditions compared to standard conditions for reference cells which are the same composition as the first cells, and wherein
   (a) the standard conditions are a set of in vitro growing conditions which include growth nutrients and growing conditions, and in which the addition of further growth nutrients and change of growing conditions does not further improve the growth rate of the reference cells; and
   (b) in the stress conditions,
      (I) one or more of the growth nutrients of (a) has been altered; or
      (II) one or more of the growing conditions of (a) has been altered; or
      (III) one or more stress-inducing parameter(s) has been added;
   such that the growth rate of the reference cells is reduced, but otherwise the stress conditions are identical to the standard conditions of (a).

### In a Second Configuration:

There is provided a nucleic acid vector comprising a nucleotide sequence that encodes (i) an agent that is toxic to first cells comprised within a cell population, or (ii) encodes a component of the toxic agent,
wherein the nucleotide sequence of the vector is under the control of a stress-phase active (SPA) promoter for expression of the agent or component thereof,
and the sequence is expressed in the first cells to produce the toxic agent or component thereof, whereby the first cells are killed,
and wherein the SPA promoter shows at least the same promoter activity in in vitro stress conditions compared to standard conditions for reference cells which are the same composition as the first cells, and wherein
   (a) the standard conditions are a set of in vitro growing conditions which include growth nutrients and growing conditions, and in which the addition of further growth nutrients and change of growing conditions does not further improve the growth rate of the reference cells; and
   (b) in the stress conditions,
      (I) one or more of the growth nutrients of (a) has been altered; or
      (II) one or more of the growing conditions of (a) has been altered; or
      (III) one or more stress-inducing parameter(s) has been added;
   such that the growth rate of the reference cells is reduced, but otherwise the stress conditions are identical to the standard conditions of (a).

### In a Third Configuration

There is provided a method of producing a nucleic acid vector comprising a nucleotide sequence that encodes (i) an agent that is toxic to first cells comprised by a cell population or (ii) encodes a component of the toxic agent,
wherein the method comprises combining the nucleotide sequence and a stress-phase active (SPA) promoter together in a deoxyribonucleic acid such that the nucleotide sequence is placed under the control of the SPA promoter whereby a vector is produced, and wherein the SPA promoter shows at least the same promoter activity in in vitro stress conditions compared to standard conditions for reference cells which are the same composition as the first cells, and wherein
   (a) the standard conditions are a set of in vitro growing conditions which include growth nutrients and growing conditions, and in which the addition of further growth nutrients and change of growing conditions does not further improve the growth rate of the reference cells; and
   (b) in the stress conditions,
      (I) one or more of the growth nutrients of (a) has been altered; or
      (II) one or more of the growing conditions of (a) has been altered; or
      (III) one or more stress-inducing parameter(s) has been added;
   such that the growth rate of the reference cells is reduced, but otherwise the stress conditions are identical to the standard conditions of (a); and
optionally formulating the vector in a pharmaceutical composition comprising a diluent, excipient or carrier.

### In a Fourth Configuration

There is provided a method of expressing a product of interest (POI) in first cells comprised by a cell population comprising introducing a nucleic acid vector into the first cells,
wherein the vector comprises a nucleotide sequence of interest (NOI) that encodes the POI,
wherein the NOI is under the control of a stress-phase active (SPA) promoter, wherein the SPA promoter shows at least the same promoter activity in in vitro stress conditions compared to standard conditions for reference cells which are the same composition as the first cells, and wherein
   (a) the standard conditions are a set of in vitro growing conditions which include growth nutrients and growing conditions, and in which the addition of further growth nutrients and change of growing conditions does not further improve the growth rate of the reference cells; and
   (b) in the stress conditions,
      (I) one or more of the growth nutrients of (a) has been altered; or
      (II) one or more of the growing conditions of (a) has been altered; or
      (III) one or more stress-inducing parameter(s) has been added;
   such that the growth rate of the reference cells is reduced, but otherwise the stress conditions are identical to the standard conditions of (a),
and wherein the NOI and the SPA promoter are not associated in nature.

### In a Fifth Configuration

There is provided a nucleic acid vector for expressing a product of interest (POI) in first cells comprised by a cell population, wherein the vector comprises a nucleotide sequence of interest (NOI) that encodes the POI, wherein the NOI is under the control of a stress-phase active (SPA) promoter which shows at least the same promoter activity in in vitro stress conditions compared to standard conditions for reference cells which are the same composition as the first cells, and wherein
(a) the standard conditions are a set of in vitro growing conditions which include growth nutrients and growing conditions, and in which the addition of further growth nutrients and change of growing conditions does not further improve the growth rate of the reference cells; and
(b) in the stress conditions,
   (I) one or more of the growth nutrients of (a) has been altered; or
   (II) one or more of the growing conditions of (a) has been altered; or
   (III) one or more stress-inducing parameter(s) has been added;
such that the growth rate of the reference cells is reduced, but otherwise the stress conditions are identical to the standard conditions of (a),
and wherein the NOI and SPA promoter are not associated together in nature.

### In a Sixth Configuration

There is provided a method of producing a nucleic acid vector for expressing a product of interest (POI) in first cells comprised by a cell population, wherein the vector comprises a nucleotide sequence of interest (NOI) that encodes the POI,
wherein the method comprises combining the NOI and a stress-phase active (SPA) promoter together in a deoxyribonucleic acid such that the NOI is placed under the control of the SPA promoter whereby a vector is produced, and wherein the SPA promoter shows at least the same promoter activity in in vitro stress conditions compared to standard conditions for reference cells which are the same composition as the first cells, and wherein
   (a) the standard conditions are a set of in vitro growing conditions which include growth nutrients and growing conditions, and in which the addition of further growth nutrients and change of growing conditions does not further improve the growth rate of the reference cells; and
   (b) in the stress conditions,
      (I) one or more of the growth nutrients of (a) has been altered; or
      (II) one or more of the growing conditions of (a) has been altered; or
      (III) one or more stress-inducing parameter(s) has been added;
   such that the growth rate of the reference cells is reduced, but otherwise the stress conditions are identical to the standard conditions of (a),
and wherein the NOI and SPA promoter are not associated in nature; and
optionally formulating the vector in a pharmaceutical composition comprising a diluent, excipient or carrier.

### In a Seventh Configuration

There is provided a method of killing first cells comprised by a cell population, wherein
(c) the first cells comprise an alarmone (optionally one or both of alarmones ppGpp and pppGpp); and/or
(d) the first cells are selected from one or more of stationary phase cells, stressed cells, persister cells, biofilm cells and dormant cells;
the method comprising introducing a nucleic acid vector into the first cells, wherein the vector comprises a nucleotide sequence that encodes (i) an agent that is toxic to the first cells or (ii) encodes a component of the toxic agent,
wherein the nucleotide sequence is under the control of a stress-phase active (SPA) promoter in the vector for expression of the agent or component thereof and the sequence is expressed in the first cells to produce the toxic agent or component thereof, whereby the first cells are killed,
wherein the SPA promoter is a promoter that is active for transcription in the first cells.

### In an Eighth Configuration

There is provided a nucleic acid vector comprising a nucleotide sequence that encodes (i) an agent that is toxic to first cells comprised by a cell population or (ii) encodes a component of the toxic agent, wherein the nucleotide sequence is under the control of a stress-phase active (SPA) promoter in the vector, wherein
(c) the first cells comprise an alarmone (optionally one or both of alarmones ppGpp and pppGpp); and/or
(d) the first cells are selected from one or more of stationary phase cells, stressed cells, persister cells, biofilm cells and dormant cells;
and wherein the SPA promoter is a promoter that is active for transcription in the first cells.

### In a Ninth Configuration

There is provided a method of producing a nucleic acid vector comprising a nucleotide sequence a nucleotide sequence that encodes (i) an agent that is toxic to first cells comprised by a cell population or (ii) encodes a component of the toxic agent,
wherein the nucleotide sequence is under the control of a stress-phase active (SPA) promoter in the vector, wherein
   (c) the first cells comprise an alarmone (optionally one or both of alarmones ppGpp and pppGpp); and/or
   (d) the first cells are selected from one or more of stationary phase cells, stressed cells, persister cells, biofilm cells and dormant cells,
and wherein the method comprises combining the nucleotide sequence and a promoter together in a deoxyribonucleic acid such that the nucleotide sequence is placed under the control of the promoter whereby a vector is produced,
and wherein the SPA promoter is a promoter that is active for transcription in the first cells, and
optionally formulating the vector in a pharmaceutical composition comprising a diluent, excipient or carrier.

### In a Tenth Configuration

There is provided a method of expressing a product of interest (POI) in first cells comprised within a cell population comprising introducing a nucleic acid vector into the first cells,
wherein the vector comprises a nucleotide sequence of interest (NOI) that encodes the POI,
wherein the NOI is under the control of a stress-phase active (SPA) promoter in the vector, and wherein
   (c) the first cells comprise an alarmone (optionally one or both of alarmones ppGpp and pppGpp); and/or
   (d) the first cells are selected from one or more of stationary phase cells, stressed cells, persister cells, biofilm cells and dormant cells,
wherein the NOI and the SPA promoter are not associated together in nature,
and wherein the SPA promoter is a promoter that is active for transcription in the first cells.

### In an Eleventh Configuration

There is provided a nucleic acid vector for expressing a product of interest (POI) in in first cells comprised within a cell population, wherein the vector comprises a nucleotide sequence of interest (NOI) that encodes the POI,
wherein the NOI is under the control of a stress-phase active (SPA) promoter in the vector,
wherein
   (c) the first cells comprise an alarmone (optionally one or both of alarmones ppGpp and pppGpp); and/or
   (d) the first cells are selected from one or more of stationary phase cells, stressed cells, persister cells, biofilm cells and dormant cells,
wherein the NOI and the SPA promoter are not associated together in nature,
and wherein the SPA promoter is a promoter that is active for transcription in the first cells.

### In a Twelfth Configuration

There is provided a method of producing a nucleic acid vector for expressing a product of interest (POI) in in first cells comprised within a cell population, wherein the vector comprises a nucleotide sequence of interest (NOI) that encodes the POI,
wherein the NOI is under the control of a stress-phase active (SPA) promoter which is active in the first cells, wherein
   (c) the first cells comprise an alarmone (optionally one or both of alarmones ppGpp and pppGpp); and/or
   (d) the first cells are selected from one or more of stationary phase cells, stressed cells, persister cells, biofilm cells and dormant cells,
wherein the method comprises combining the nucleotide sequence and a promoter together in a deoxyribonucleic acid such that the nucleotide sequence is placed under the control of the promoter whereby a vector is produced,
wherein the NOI and the SPA promoter are not associated together in nature,
and wherein the SPA promoter is a promoter that is active for transcription in the first cells, and
optionally formulating the vector in a pharmaceutical composition comprising a diluent, excipient or carrier.

### In a Thirteenth Configuration

There is provided a nucleic acid vector for use in a method of treating or preventing a disease or condition in a patient that is mediated by the first cells (optionally wherein the first cells are pathogenic cells), wherein the treating or preventing comprises administering a vector as defined herein to the patient and killing the first cells.

### In a Fourteenth Configuration

There is provided a nucleic acid vector for use in a method of treating or preventing a disease or condition in a patient, wherein the method comprises administering a vector expressing an NOI as defined herein to the patient and delivering the POI to the first cells.

### In a Fifteenth Configuration

There is provided a nucleic acid vector for use in a method to treat or prevent a disease or condition mediated in a patient (e.g. a human or animal subject) by the first cells, or to treat a pathogenic infection of the first cells in the patient.

### BRIEF DESCRIPTION OF THE FIGURES

FIG. 1: Packaging the CRISPR-Guided Vectors (CGVs) into transducing particles. Bacterial cells used carry a defective P2 prophage on their chromosome. The defective prophage has all the genes required for the production and assembly of the phage structure, for specific packaging of the CGV into the phage head, and for host cell lysis (helper functions). Expression of these genes was enabled by the activator (P4 delta) carried by Plasmid 1. The different CGVs used in the experiments had the same overall structure and differed only in the promoter sequence upstream of the Cas genes.

FIG. 2: Killing of cells by CGVs in (A) standard growth conditions (fast growth) and in (B) stress conditions (slow growth). Stress was induced by the addition of serine hydroxamate (seryl-tRNA synthetase inhibitor) 2 hours before transduction by CGVs (0 time). Growth and survival of the cells was followed by measuring the optical density of the cultures. All the promoter-carrying CGVs inhibited cell growth more than the CGV without a promoter (used as a reference, =100%).

FIG. 3: Transcription units used in the CRISPR armed phages. The cas genes and the arrays were inserted into the phage genome as separate transcription units at two different locations. Both units were transcribed from two promoters, the non-regulated 0.23100 promoter and the stress-responsive bolA promoter.

FIG. 4: The effect of CRISPR armed phages on cells in (A) standard conditions (fast growth) and in (B) stress condition (slow growth). Stress was induced by introducing a temperature sensitive mutation in the ValS gene. In these cells, the stringent response (general stress response) is induced by limiting the charging of valyl-tRNAs. Wild type (08) or CRISPR armed (08.3) phages were added to exponentially growing cells at the time indicated by arrows. The number of phages added (1E8 or 5E8) was higher than the number of cells, allowing phage infection of the vast majority of cells. Growth and survival of the cells was followed by measuring the optical density of the cultures.

FIG. 5: Categories of genes that are preferentially upregulated in stationary phase.

### DETAILED DESCRIPTION

Environmental stress can be an external factor that has an adverse effect on the physiological welfare of cells, such as bacterial cells. Many bacteria can adapt to such stress conditions by altering gene expression. However, severe stress can substantially interfere with bacterial physiology, resulting in a state characterised by reduced growth (e.g. a doubling time lower compared to growth under standard conditions, such as those described in examples 1, 2 and 3 herein), or halted growth.

The reasons for termination of growth may be either exhaustion of an essential growth nutrient or accumulation of toxic products, inhibitory products or other stress-inducing parameters as described elsewhere herein. If an inhibitory product is produced by the cell, and accumulates in the medium, the growth rate will slow down (depending on inhibitor production) and at a certain level of inhibitor concentration, growth will stop. Ethanol production by yeast is an example of a fermentation in which the product is inhibitory to growth.

Examples of such slow/non-growing cells (herein referred to as “slow growing cells”) are:
1. Stationary phase cells: Stationary phase is for example the stage when growth ceases but cells remain metabolically active. The rate of formation of new cells is similar to the rate of cell death. Stationary phase cells typically form upon exhaustion of nutrients from the environment. In the stationary phase, elongated cells may become spherical and smaller with a rigid cell envelope, the cell wall is highly cross-linked, membrane fluidity reduces, and cells activate the stringent response mechanism in order to survive the stress. The activation of this mechanism allows the bacteria to reprogram the gene expression pattern to adapt to different stresses. In E. coli and several other bacterial species, the two key components of the bacterial stringent response are ppGpp and pppGpp. As a consequence, the cells divert their resources away from growth toward synthesizing amino acids so as to promote survival until nutrient conditions improve.

During the late stationary phase (sometimes referred to as long-term stationary phase), several remarkable adaptations take place. On continued starvation, one of the survival strategies includes bacteria entering a viable but non-culturable state (VBNC). In this state, bacteria remain metabolically active, but fail to form colonies on bacteriological media. Several bacteria including Rhodococcus biphenylivorans, Escherichia coli, Agrobacterium tumefaciens, Helicobacter pylori, Lactococcus lactis many Vibrio species, and Pseudomonas species have been shown to enter the VBNC state. The VBNC state poses a serious health risk as the dormant bacterial species could remain undetected in culturable conditions, though having the ability to cause infections. A variety of stresses is said to lead to the manifestation of VBNC state. Prolonged starvation also results in Growth Advantage in Stationary Phase (GASP) phenotype. The GASP phenomenon is a result of mutations in the RpoS allele which confers a gainful ability to continue growing during starvation conditions, thus replacing the parental population. These mutations allow the mutants to effectively scavenge the nutrients released by dead cells. A number of Gram-positive bacteria such as Listeria monocytogenes, Staphylococcus aureus, Enterococcus faecalis, and Bacillus globigii and Gram-negative bacteria including Campylobacter, Geotacter, Vibrio, E. coli, Pseudomonas, etc., have been found to enter the GASP state. The term “constant activity stationary phase” (CASP) describes the phenomenon of constant rate of protein synthesis observed in non-growing bacteria that have undergone over more than 60 h of starvation. On studying the protein production at this stage, it has been found that both the protein synthesis machinery including ribosomes, RNA polymerases, etc., and resources such as amino acids, nucleotides, etc., remain constant at CASP. Finally, constant promoter activity was observed in this experiment for up to 10 h of starvation. Another interesting phenomenon experienced by bacterial population in stationary phase is the “stationary phase contact-dependent inhibition” (SCDI). It requires physical contact between the evolved and original bacteria. In this process, it was observed that the evolved strains either killed or inhibited the growth of bacteria that they were derived from. The inhibiting ability of these strains is attributed to mutations within a single gene involved in glycogen synthesis pathway: gIgC (encoding ADP-glucose pyrophosphorylase). Astonishingly, all evolved strains overproduced glycogen which seemed to be necessary for SCDI to occur.
2. Dormant cells: Dormant cells may persist in a population (persister cells); dormant cells are non-dividing or slowly dividing cells that typically transiently survive treatment by antimicrobials. These cells may be multidrug tolerant; they are not multidrug resistant genetically, their multidrug tolerance is due to their physiological state.

Persister cells are multidrug tolerant cells present in bacterial populations studied to date. Persisters are not mutants, but rather phenotypic variants of the wild-type that upon reinoculation produce a culture with similar levels of tolerance. The number of persisters in an E. coli population remains constant throughout early-exponential phase, with a marked increase as cells enter late-exponential and early-stationary phases. Maintaining cells in exponential growth using repeated dilutions in fresh media, similar to growth in a chemostat, results in a complete loss of persisters. This lack of persistence demonstrates that these cells are not at a particular stage in the cell cycle and are not produced in response to antibiotics. In a study employing a microfluidic device to monitor cell growth, persisters were shown to be rare non-growing cells that pre-exist in a population. Persisters are responsible for multidrug tolerance of biofilms which account for the majority of infectious diseases in the developed world. The second messenger (p)ppGpp plays a key role in persister formation in E. coli populations
3. Stressed cells: These are cells where specific gene expression patterns are activated to enable cell survival in adverse and/or fluctuating conditions in the cells' immediate surroundings.
4. Biofilm cells: Biofilms are a collective of one or more types of microorganisms that grow on a surface. The metabolic state of the bacterial cells in such biofilms is heterogeneous but characterised by a large fraction of cells in stationary, dormant or stressed conditions.

Cells in exponential, stationary, and long-term stationary phases have different fates. As a consequence of starvation, many bacteria including the genera Bacillus and Clostridium form resistant spores helping them withstand the harsh surrounding environment. Non-optimal growth conditions also lead to the formation of biofilm in many bacterial species. Physiologically, biofilm bacteria are similar to stationary phase bacteria. One key transition is the formation of persisters induced during stationary phase, in biofilms, and also as a consequence of a general stress response. The formation of these bacterial persisters is understood to be the reason behind relapsing infections and is a major cause of drug resistance.

The inventors have advantageously realised means to decouple the killing potential of cytotoxic agents, and the expression of therapeutics and other products of interest, from the growth state of the target cells. In particular, the use of targeted nucleases (e.g. Cas nucleases) are useful in this respect, since the nuclease, if expressed in slow growing cells (e.g. in stationary phase cells, dormant cells, stressed cells and/or biofilm cells), will cut target nucleic acid (e.g. chromosomal DNA) of such cells irrespective of the metabolic state. When the environment subsequently changes to favourable growth conditions where antibiotic-treated cells would normally resume growth, the agent (e.g. nuclease)-treated cells are dead and cannot resume growth.

To obtain actively expressed cytotoxicity (e.g. using CRISPR/Cas systems) in the slow growing cells, we exploit promoter DNA sequences that are active under the corresponding environmental conditions in the target cells to advantageously drive expression of cytotoxic agents, therapeutics and other products of interest.

Being able to express products of interest (POIs) in these slow growing cells may also be beneficial because microorganisms growing outside laboratory conditions, including in the human body (or in a human microbiome) often are exposed to stress conditions imposed by the environment. Expression of therapeutic molecules, or beneficial metabolites under such conditions may improve efficacy and consistency in production in a local environment. For example, biofilm-like growth associated with the gut epithelium may be an advantageous niche for expression of POIs to excerpt their effect on receptors on the gut epithelium or translocate into the body to create systemic exposure.

### Definitions

“Cells under stressed conditions”, “stressed cells”, “slow-growing cells” and the like is used to refer to either (i) stationary phase cells, stressed cells, persister cells, biofilm cells and dormant cells; and/or (ii) cells which express or produce an alarmone; and/or (iii) cells which upregulate expression of genes associated with a stress response. These cells exhibit reduced or severely reduced growth, or are in a dormant state.

A “microbiome,” as used herein, refers to the totality of microbes in a particular environment (e.g. in/on an organism, in a marine environment (e.g. ocean), and/or in a terrestrial environment (e.g. soil)). In some embodiments, a microbiome may refer to the totality of microbes that reside, or are stably maintained, for example, on the surface and in deep layers of the skin, in the saliva and oral mucosa, in the conjunctiva, and in the gastrointestinal tracts of an organism. The microbiome may exist within any of the organs described elsewhere herein.

As used herein, “phagemid” refers to a bacteriophage-derived vector containing the replication origin of a plasmid and the packaging site of a bacteriophage. Examples of phagemids that may be used in accordance with the present disclosure include, without limitation, M13-derived phagemids containing the β origin for filamentous bacteriophage packaging such as, for example, pBluescript II SK (+/−) and KS (+/−) phagemids, pBC SK and KS phagemids, pADL and P1-based phagemids (see, e.g. Westwater C A et al., Microbiology 148, 943-50 (2002); Kittleson J T et al., ACS Synthetic Biology 1, 583-89 (2012); Mead D A et al., Biotechnology 10, 85-102 (1988)). Other phagemids may be used and, for example, can be made to work with packaging systems from natural, engineered or evolved bacteriophage.

### Methods and Vectors Containing SPA Promoters Whose Activity is Experimentally Confirmed

Targeting cells which are in a state of reduced growth or are not actively growing (slow-growing cells) can be particularly difficult, as many proteins are not expressed and much cellular machinery is downregulated.

The inventors have surprisingly discovered that some promoters are as active, if not more active when cells are growing under stressed conditions and how to effectively test for this particular activity for any given promoter, and that their activity can be harnessed to drive expression of cytotoxic agents, therapeutics and other products of interest.

Thus, there is provided the methods and vectors described in the first to sixth configurations hereinabove.

In any of the first to sixth configurations (or in any of the related configurations, embodiments or other disclosure herein), where it is said that “the SPA promoter shows at least the same promoter activity . . . ”, it is meant that, if and when tested, the SPA promoter is capable of showing such activity, rather than the SPA promoter is actively tested for the required activity.

In a particular embodiment, the first cells are selected from microorganism cells, bacterial cells, archaeal cells or fungal cells (e.g. yeast cells). The first cells may be bacterial cells. The first cells can be of the same strain or species. The first cells can be bacterial cells of the same strain. The first cells can be bacterial cells of the same species.

In a particular embodiment, the SPA promoter shows a statistically significant increase in promoter activity in in vitro stress conditions compared to standard conditions. The increase may be at least a 2-fold increase. In one embodiment, the increase in promoter activity is measured comparing total mRNA levels to mRNA levels transcribed from the SPA promoter in the in vitro stress conditions and in the standard conditions to determine the increase.

In a particular embodiment, the growth rate of the reference cells is determined by measuring the doubling time of the reference cells. The growth rate in the stress conditions may be statistically significantly slower than in the standard conditions, for example less than 50% of the doubling time of the reference cells in the standard conditions.

In a particular embodiment, the stress conditions are caused by an absence of a growth nutrient. In another embodiment the stress conditions are caused by a change in the growth conditions. In one embodiment, the stress conditions are caused by a change in the growth conditions or an absence of a growth nutrient, but not by the addition of a stress-inducing parameter.

In a particular embodiment, the toxic agent (or component thereof) or POI comprises one or more crRNA(s) or one or more RNA(s) (such as a guide RNA or crRNA), and at least one Cas (e.g. a Cas) nuclease that is capable of recognising and modifying (e.g. cutting) at least one target sequence (e.g. protospacer sequence) comprised by the first cells, wherein the or each crRNA or gRNA is operable in the first cells with a cognate Cas to guide the Cas to a target sequence (e.g. protospacer sequence) comprised by the first cells, wherein the Cas modifies (e.g. cuts) the target sequence (e.g. protospacer sequence) and the first cells are killed.

In another embodiment, the vector encodes a component of the toxic agent or an NOI, which encodes one or more (e.g. 1 to 5, such as 1 to 3) guided nuclease(s) or one or more (e.g. 1 to 5, such as 1 to 3) RNA(s) (such as a guide RNA or crRNA) for targeting a sequence comprised by the genome of the first cells.

Thus, a certain embodiment of the first configuration provides a method of killing first cells which are bacterial cells or archaeal cells of a first strain or species comprised by a cell population, the method comprising introducing a nucleic acid vector into the first cells,
wherein the vector comprises a nucleotide sequence that encodes (i) an agent that is toxic to the first cells, or (ii) a component of the toxic agent,
wherein the nucleotide sequence of the vector is under the control of a stress-phase active (SPA) promoter for expression of the agent or component thereof, and the sequence is expressed in the first cells to produce the toxic agent or component thereof, whereby the first cells are killed,
and wherein the SPA promoter shows a statistically significant increase (e.g. at least 2-fold increase) in promoter activity (e.g. as measured by comparing total mRNA levels to mRNA levels transcribed from the SPA promoter) in in vitro stress conditions compared to standard conditions for reference cells which are the same composition as the first cells, and wherein
   (a) the standard conditions are a set of in vitro growing conditions which include growth nutrients and growing conditions, and in which the addition of further growth nutrients and change of growing conditions does not further improve the growth rate of the reference cells; and
   (b) in the stress conditions,
      (I) one or more of the growth nutrients of (a) has been altered; or
      (II) one or more of the growing conditions of (a) has been altered; and optionally
      (III) one or more stress-inducing parameter(s) has been added;
   such that the growth rate of the reference cells is reduced by at least 50%, but otherwise the stress conditions are identical to the standard conditions of (a),
and wherein the growth rate is measured by determining the doubling time of the reference cells.

There is also provided an embodiment of the second configuration which is a vector having the features described in the paragraph above, and an embodiment of the third configuration which is a method of making such a vector.

A certain embodiment of the fourth configuration provides a method of expressing a product of interest (POI) in first cells which are bacterial cells or archaeal cells of a first strain or species wherein the first cells are comprised by a cell population, the method comprising introducing a nucleic acid vector into the first cells,
wherein the vector comprises a nucleotide sequence of interest (NOI) that encodes the POI,
wherein the NOI is under the control of a stress-phase active (SPA) promoter, wherein the SPA promoter shows a statistically significant increase (e.g. at least 2-fold increase) in promoter activity (e.g. as measured by comparing total mRNA levels to mRNA levels transcribed from the SPA promoter) in in vitro stress conditions compared to standard conditions for reference cells which are the same composition as the first cells, and wherein
   (a) the standard conditions are a set of in vitro growing conditions which include growth nutrients and growing conditions, and in which the addition of further growth nutrients and change of growing conditions does not further improve the growth rate of the reference cells; and
   (b) in the stress conditions,
      (I) one or more of the growth nutrients of (a) has been altered; or
      (II) one or more of the growing conditions of (a) has been altered; and optionally
      (III) one or more stress-inducing parameter(s) has been added;
   such that the growth rate of the reference cells is reduced by at least 50%, but otherwise the stress conditions are identical to the standard conditions of (a),
wherein the growth rate is measured by determining the doubling time of the reference cells, and wherein the NOI and the SPA promoter are not associated in nature.

There is also provided an embodiment of the fifth configuration which is a vector having the features described in the paragraph above, and an embodiment of the sixth configuration which is method of making such a vector.

In a particular embodiment, the promoter is selected from an E. coli hyaA, bolA, rpoH, yiaG or relB promoter (and optionally the first cells are E. coli cells) or an orthologue or homologue promoter from a different species (e.g. a Firmicutes, Bacilli, Bacillales, Staphylococcaceae or Staphylococcus species). In another embodiment, the promoter comprises a sequence selected from the sequences of SEQ ID Nos: 1 to 10 (in particular, any of SEQ ID Nos:1 to 4, or comprises all of SEQ ID Nos: 5 to 10).

Thus, there is provided a method of killing first cells comprised by a cell population, the method comprising introducing a nucleic acid vector into the first cells,
wherein the vector comprises a nucleotide sequence that encodes (i) an agent that is toxic to the first cells, or (ii) a component of the toxic agent,
wherein the nucleotide sequence of the vector is under the control of a stress-phase active (SPA) promoter selected from an E, coli hyaA, bolA, rpoH, yiaG or relB promoter (and optionally the first cells are E. coli cells) or an orthologue or homologue promoter from a different species (e.g. a Firmicutes, Bacilli, Bacillales, Staphylococcaceae or Staphylococcus species),
and wherein the nucleotide sequence and the SPA promoter are not associated in nature.

There is also provided is provided a nucleic acid vector comprising a nucleotide sequence that encodes (i) an agent that is toxic to first cells comprised within a cell population, or (ii) encodes a component of the toxic agent,
wherein the nucleotide sequence of the vector is under the control of a stress-phase active (SPA) promoter selected from an E. coli hyaA, bolA, rpoH, yiaG or relB promoter (and optionally the first cells are E. coli cells) or an orthologue or homologue promoter from a different species (e.g. a Firmicutes, Bacilli, Bacillales, Staphylococcaceae or Staphylococcus species),
and wherein the nucleotide sequence and the SPA promoter are not associated in nature.

There is also provided a method of producing a nucleic acid vector comprising a nucleotide sequence that encodes (i) an agent that is toxic to first cells comprised by a cell population or (ii) encodes a component of the toxic agent,
wherein the method comprises combining the nucleotide sequence and a stress-phase active (SPA) promoter together in a deoxyribonucleic acid such that the nucleotide sequence is placed under the control of the SPA promoter whereby a vector is produced, and wherein the SPA promoter is selected from an E. coli hyaA, bog rpoH, yiaG or relB promoter (and optionally the first cells are E. coli cells) or an orthologue or homologue promoter from a different species (e.g. a Firmicutes, Bacilli, Bacillales, Staphylococcaceae or Staphylococcus species),
and wherein the nucleotide sequence and the SPA promoter are not associated in nature, and
optionally formulating the vector in a pharmaceutical composition comprising a diluent, excipient or carrier.

There is also provided a method of expressing a product of interest (POI) in first cells comprised within a cell population, wherein the method comprises introducing a nucleic acid vector into the first cells,
wherein the vector comprises a nucleotide sequence of interest (NOI) that encodes the POI, wherein the NOI is under the control of a stress-phase active (SPA) promoter, wherein the SPA promoter selected from an E. coli hyaA, bolA, rpoH, yiaG or relB promoter (and optionally the first cells are E. coli cells) or an orthologue or homologue promoter from a different species (e.g. a Firmicutes, Bacilli, Bacillales, Staphylococcaceae or Staphylococcus species), and wherein the NOI and the SPA promoter are not associated in nature.

There is also provided a nucleic acid vector for expressing a product of interest (POI) in first cells comprised by a cell population, wherein the vector comprises a nucleotide sequence of interest (NOI) that encodes the POI, wherein the NOI is under the control of a stress-phase active (SPA) promoter selected from an E. coli hyaA, bolA, rpoH, yiaG or relB promoter (and optionally the first cells are E. coli cells) or an orthologue or homologue promoter from a different species (e.g. a Firmicutes, Bacilli, Bacillales, Staphylococcaceae or Staphylococcus species),
and wherein the NOI and the SPA promoter are not associated in nature.

There is also provided a method of producing a nucleic acid vector for expressing a product of interest (POI) in first cells comprised by a cell population, wherein the vector comprises a nucleotide sequence of interest (NOI) that encodes the POI,
wherein the method comprises combining the NOI and a stress-phase active (SPA) promoter together in a deoxyribonucleic acid such that the NOI is placed under the control of the SPA promoter whereby a vector is produced, and wherein the SPA promoter is selected from an E. coli hyaA, bolA, rpoH, yiaG or relB promoter (and optionally the first cells are E. coli cells) or an orthologue or homologue promoter from a different species (e.g. a Firmicutes, Bacilli, Bacillales, Staphylococcaceae or Staphylococcus species),
and wherein the NOI and the SPA promoter are not associated in nature,
and optionally formulating the vector in a pharmaceutical composition comprising a diluent, excipient or carrier.

### Toxic Agents, Components Thereof and POIs

In any configuration, embodiment or part of this disclosure, the POI may be an RNA or a protein. In any configuration, embodiment or part of this disclosure, the toxic agent, or component thereof may be an RNA or a protein.

In any configuration, embodiment or part of this disclosure which relates to a method of killing first cells using a toxic agent or component thereof, instead of killing first cells, the growth of the first cells may be reduced. In any configuration, embodiment or part of this disclosure which relates to a method of killing first cells using a toxic agent or component thereof, instead of killing first cells, the growth of the first cells may be halted.

The nucleotide sequences encoded by the vector or the NOIs may be synthetic sequences. The nucleotide sequences encoded by the vector or the NOIs may comprise several components, which are not found together in nature. The nucleotide sequences encoded by the vector or the NOIs may comprise several components, one of which is a synthetic sequence.

The nucleotide sequences encoded by the vector (e.g. the toxic agent(s) or component(s) thereof) may be sequences which are not associated with the SPA promoter in nature.

The vector may be delivered to cells which are under stress conditions and the vector delivers a toxic agent (or component thereof) or a POI which is a toxic agent (or component thereof), which may be any toxic agent (or component thereof) described herein. Such a toxic agent, or component thereof, may be designed to kill (or reduce the number of) the first cells (to which the vector is delivered). In this embodiment, the killing may be selective for the first cells. Alternatively, the toxic agent, or component thereof, may be designed to kill (or reduce the number of) second cells which are in the vicinity of the first cells (to which the vector is delivered). In this embodiment, the killing may be selective for the second cells. In one embodiment, the toxic agent, or component thereof, can be designed to kill both the first cells (to which the vector is delivered) and also second cells which are in the vicinity of the first cells. In one embodiment, the toxic agent may have general antimicrobial activity. By “in the vicinity”, it is meant that the second cells are located sufficiently nearby to the first cells such that when the first cell secretes the toxic agent, the toxic agent, or component thereof, can have a killing effect on the second cells. The second cells may be cells within the cell population which comprises the first cells. The second cells may be adjacent (e.g. immediately adjacent) to the first cells. The second cells may be contained with the same biofilm as the first cells. The second cells may be contained within the same microbiome of the same organ (as described herein) with a patient.

Thus, the toxic agent or component thereof, or the NOI may comprise a sequence that encodes a nuclease, such as a guided nuclease (optionally a Cas nuclease, TALEN nuclease, zinc finger nuclease or meganuclease), that is operable in the first cells to cut a target nucleotide sequence comprised by the first cells, whereby the first cells are killed.

The toxic agent or component thereof, or the NOT may comprise a sequence that encodes a nuclease, such as a guided nuclease (optionally a Cas nuclease, TALEN nuclease, zinc finger nuclease or meganuclease), that is operable in second cells comprised within the population, wherein the second cells are different to the first cells, to cut a target nucleotide sequence comprised by the second cells, whereby the second cells are killed.

The nucleotide sequence may encode component(s) of the toxic agent (or the POI is a component of a toxic agent) and the component(s) is/are operable with one or more further component(s) of the agent in the cell to kill the cell (which may be the first and/or second cells according to the various methods and vectors described herein). The component(s) of the toxic agent operate(s) with other component(s) of the agent to kill the cells. In this embodiment, the other component(s) may be part of an operon and under the transcriptional control of the SPA promoter. Alternatively, the other component(s) may be part of the vector, and under the control of a different promoter (e.g. a second SPA promoter, or a constitutive or inducible promoter). The other component(s) may be endogenous to the cell to be killed (i.e. the first or second cells), and for example may be endogenous CRISPR/Cas systems.

In one embodiment, the NOI or nucleotide sequence encodes component(s) of a toxic agent. The component(s) of the toxic agent may be one or more guided nuclease(s) for targeting a sequence comprised by the genome of the first cells. There may be one to three guided nucleases. There may be one or two guided nucleases. There may be one guided nuclease. There may be two guided nucleases. There may be three guided nucleases. The component of the toxic agent may be one or more RNA(s) (such as a guide RNA or crRNA) for targeting a sequence comprised by the genome of the first cells. There may be one to three RNA(s). There may be one or two RNA(s). There may be one RNA. There may be two RNAs. There may be three RNAs, the RNA(s) may be different to each other, and each may guide the guided nuclease to a different target sequence comprised by the genome of the first cells. Each target sequence may be a protospacer sequence. Each protospacer sequence may be comprised by a chromosomal sequence. The first cells may be bacterial cells.

In one embodiment, the NOT or nucleotide sequence encodes a toxic agent that is a CRISPR/Cas system that is capable of recognising and cutting at least one target sequence (e.g. protospacer sequence) comprised by the first cells, whereby the first cells are killed. The target sequence (e.g. protospacer sequence) may be comprised by a chromosomal sequence. The first cells may be bacterial cells.

In a particular embodiment, the toxic agent may comprise:
one or more crRNA(s) or one or more RNA(s) (such as a guide RNA or crRNA), and
at least one Cas (e.g. a Cas) nuclease that is capable of recognising and modifying (e.g. cutting) at least one target sequence (e.g. protospacer sequence) comprised by the first cells,

wherein the or each crRNA or gRNA is operable in the first cells with a cognate Cas to guide the Cas to a target sequence (e.g. protospacer sequence) comprised by the first cells, wherein the Cas modifies (e.g. cuts) the target sequence (e.g. protospacer sequence) and the first cells are killed. Where there is more than one crRNA or gRNA, each crRNA or gRNA recognises a different protospacer sequences comprised by the first cells.

Where the NOI or nucleotide sequence encodes component(s) of a toxic agent which is one or more RNA(s) (such as a guide RNA or crRNA), the vector may further encode a Cas that is operable in the first cells with a or the crRNA(s) or a or the RNA(s) (such as a guide RNA or crRNA) to guide the Cas to a target sequence (e.g. protospacer sequence) comprised by the first cells, wherein the Cas modifies (e.g. cuts) the target sequence (e.g. protospacer sequence) and the cells are killed.

Where the NOI or nucleotide sequence encodes component(s) of a toxic agent which is one or more RNA(s) (such as a guide RNA or crRNA), the vector may further encode a Cas nuclease that is operable in the first cells with a or the crRNA(s) or a or the RNA(s) (such as a guide RNA or crRNA) to guide the Cas to a target sequence (e.g. protospacer sequence) comprised by the first cells, wherein the Cas modifies (e.g. cuts) the target sequence (e.g. protospacer sequence) and the cells are killed.

Where the NOI or nucleotide sequence encodes component(s) of a toxic agent which is one or more RNA(s) (such as a guide RNA or crRNA), the vector may further encode a Cas nuclease selected from a Cas9, Cas3, Cas12 or Cas13, that is operable in the first cells with a or the crRNA(s) or a or the RNA(s) (such as a guide RNA or crRNA) to guide the Cas to a target sequence (e.g. protospacer sequence) comprised by the first cells, wherein the Cas modifies (e.g. cuts) the target sequence (e.g. protospacer sequence) and the cells are killed.

The NOI or nucleotide sequence may encode a toxic agent which is
one or more RNA(s) (such as a guide RNA or crRNA), and
a Cas that is operable in the first cells with a or the crRNA(s) or a or the RNA(s) (such as a guide RNA or crRNA) to guide the Cas to a target sequence (e.g. protospacer sequence) comprised by the first cells, wherein the Cas modifies (e.g. cuts) the target sequence (e.g. protospacer sequence) and the cells are killed.

The NOI or nucleotide sequence may encode a toxic agent which is
one or more RNA(s) (such as a guide RNA or crRNA), and
a Cas nuclease that is operable in the first cells with a or the crRNA(s) or a or the RNA(s) (such as a guide RNA or crRNA) to guide the Cas to a target sequence (e.g. protospacer sequence) comprised by the first cells, wherein the Cas modifies (e.g. cuts) the target sequence (e.g. protospacer sequence) and the cells are killed.

The NOI or nucleotide sequence may encode a toxic agent which is
one or more RNA(s) (such as a guide RNA or crRNA), and
a Cas nuclease selected from a Cas9, Cas3, Cas12 or Cas13, that is operable in the first cells with a or the crRNA(s) or a or the RNA(s) (such as a guide RNA or crRNA) to guide the Cas to a target sequence (e.g. protospacer sequence) comprised by the first cells, wherein the Cas modifies (e.g. cuts) the target sequence (e.g. protospacer sequence) and the cells are killed.

The toxic agent may be a toxin which is an antibiotic (e.g. any of the antibiotics described herein). The toxic agent may be a toxin which is an antifungal (e.g. any of the antifungals described herein). The toxic agent may be a toxin which is an antimicrobial (e.g. any of the antimicrobials described herein). The toxic agent may be a toxin which is an antiviral (e.g. any of the antivirals described herein). In these embodiments, the toxin may kill the first cells. Alternatively, the toxin may kill second cells which are different to the first cells (e.g. cells which are adjacent to, or in the vicinity of, or in the same microbiome as the first cells). The toxic agent may kill both the first cells and the second cells.

The toxic agent may be a bacteriocin, for example any of the bacteriocins described herein. The toxic agent may be a cecropin, for example any of the cecropins described herein. The toxic agent may be a moricin, for example any of the moricins described herein. The toxic agent may be a cupiennin, for example any of the cupiennins described herein. The toxic agent may be a oxyopinin, for example any of the oxyopinins described herein. The toxic agent may be a magainin, for example any of the magainins described herein. The toxic agent may be a dermaseptin, for example any of the dermaseptins described herein. The toxic agent may be a cathelicidin, for example any of the cathelicidin described herein. The toxic agent may be a protegrin, for example any of the protegrins described herein. The toxic agent may be hydramacin-1. The toxic agent may be papiliocin. The toxic agent may be poneratoxin. The toxic agent may be mastoparan. The toxic agent may be melittin. The toxic agent may be spinigerin. In these embodiments, the toxin may kill the first cells. Alternatively, the toxin may kill second cells which are different to the first cells (e.g. cells which are adjacent to, or in the vicinity of, or in the same microbiome as the first cells). The toxic agent may kill both the first cells and the second cells.

The toxic agent may be a cytostatic agent. The toxic agent may be a cytotoxic agent.

Instead of killing (or reducing growth), the methods disclosed herein may modify the genome (chromosome or plasmid) of the first cells, for example, wherein a new nucleotide sequence is introduced into the genome, a nucleotide sequence is deleted from the genome or transcription of one or more sequences of the genome is activated or inhibited. In this embodiment, the nucleotide sequence or NOI encodes a guided nuclease (e.g. a Cas, meganuclease, TALEN or zinc finger nuclease) that is guided to a target sequence comprised by the genomes of the first cells whereby the target sequence is modified, or the nucleotide sequence encodes a guiding RNA (e.g. a gRNA) or a crRNA that guides such a nuclease to modify the target site. In one embodiment, the modification may enhance the growth of the first cells, for example by deletion of, or inhibition of transcription of, a gene which expresses an inhibitory growth molecule, or by activating expression of a gene which enhances the growth of the first cells.

In some embodiments, e.g. where the NOI or nucleotide sequence encodes component(s) of a toxic agent which is one or more RNA(s) (such as a guide RNA or crRNA), the Cas nuclease may be provided by an endogenous Type I, II, III, IV or V CRISPR/Cas system of the first cells. A particular endogenous CRISPR/Cas system of the first cells is a Type II CRISPR/Cas system.

In some embodiments, e.g. where the NOI or nucleotide encodes a Cas, the tracrRNA sequence or DNA sequence expressing a tracrRNA sequence may be endogenous to the first cells. The Cas may be a Type II Cas.

In any embodiment, the target sequence of the nuclease is comprised by an antibiotic resistance gene, virulence gene or essential gene of the first cells.

The RNA(s) may comprise a sequence R1-S1-R1′ for expression and production of a crRNA (e.g. a single guide RNA), (i) wherein R1 is a first CRISPR repeat. R1′ is a second CRISPR repeat, and R1 or R1′ is optional; and (ii) S1 is a first CRISPR spacer that comprises or consists of a nucleotide sequence that is 95% or more identical to said target sequence. R1 and R1′ may be at least 95% identical respectively to the first and second repeat sequences of a CRISPR array of the first cells. R1 and R1′ may be at least 95% (e.g. 96%, 97%, 98%, 99% or 100%) identical respectively to the first (5′-most) and second (the repeat immediately 3′ of the first repeat) repeat sequences of a CRISPR array of the first cells. R1 and R1′ may be functional with a Type II Cas9 nuclease (e.g. a S. thermophilus, S. pyogenes or S. aureus Cas9) to modify the target in the first cells. R1 and R1′ may be functional with a Type I Cas3 to modify the target in the first cells.

In some embodiments, it may be advantageous, rather than to kill (or reduce the growth of) the cells which are under stress conditions (such as stationary phase cells, stressed cells, persister cells, biofilm cells and dormant cells), it may be desirable to deliver a molecule which has a beneficial effect in the first cells or in the vicinity of the first cells, such as in a microbiome.

In one embodiment, the POI is a selected from a therapeutic molecule, a molecule that is beneficial to the local environment of the cell (such as a beneficial metabolite that is not present, or present in too low a concentration), and a reporter molecule.

In one embodiment, the POI is a therapeutic molecule.

The therapeutic molecule may be an antibody therapy. In particular, the therapeutic molecule is an antibody fragment. The therapeutic molecule may comprise an antibody (or in particular an antibody fragment) comprising the binding domains of any of the specific antibody molecules described herein. The therapeutic molecule may comprise an antibody fragment which binds to any of the targets of any of the antibody molecules described herein.

The antibody therapy may be an immune checkpoint inhibitor antibody or fragment thereof. The antibody or fragment thereof may be selected from an anti-PD-L1, anti-PD-1, anti-CTLA4, anti-TIM3, anti-TNFa superfamily member (such as an anti-TNFa, TNFR1 or BAFF), anti-IL6R, anti-IL-4Ra, or anti-PCSK9.

Therapeutic molecules include therapeutic proteins. Therapeutic proteins may be used to, for example, replace a protein that is deficient or abnormal, augment an existing biological pathway, provide a novel function or activity, interfere with a molecule or organism, and/or deliver other compounds or proteins, such as a radionuclide, cytotoxic drug, or effector proteins. Therapeutic molecules include, without limitation, antibodies or fragments thereof, antibody-based drugs, Fc fusion proteins, anticoagulants, blood factors, bone morphogenetic proteins, engineered protein scaffolds, enzymes, growth factors, hormones, interferons, interleukins, and thrombolytics. Other examples include those that bind non-covalently to target (e.g. monoclonal antibodies or fragments thereof), those that affect covalent bonds (e.g. enzymes), and those that exert activity without specific interactions (e.g. serum albumin).

Therapeutic molecules include recombinant therapeutic proteins, used to treat, for example, cancers, immune disorders, infections and/or other diseases.

In some embodiments, the therapeutic protein is Etanercept, Bevacizumab, Rituximab, Adalimumab, Infliximab, Trastuzumab, Insulin glargine, Epoetin alfa, Pegfilgrastim, Ranibizumab, Darbepoetin alfa, Interferon beta-1α, Insulin aspart, Rhu insulin, Octocog alfa, Insulin lispro, Cetuximab, Peginterferon alfa-2a, Interferon beta-1b, Eptacog alfa, Insulin aspart, OnabotulinumtoxinA, Epoetin beta, Rec antihemophilic factor, Filgrastin, Insulin detemir, Natalizumab, Insulin (humulin) or Palivizumab.

Examples of antibodies, antibody fragments, and/or Fc fusion proteins include, without limitation, Abagovomab, Abciximab, Actoxumab, Adalimumab, Adecatumumab, Afelimomab, Afutuzumab, Alacizumab pegol, ALD, Alemtuzumab, Alirocumab, Altumomab pentetate, Amatuximab, Anatumomab mafenatox, Anifrolumab, Anrukinzumab, Apolizumab, Arcitumomab, Aselizumab, Atinumab, Atlizumab (or tocilizumab), Atorolimumab, Bapineuzumab, Basiliximab, Bavituximab, Bectumomab, Belimumab, Benralizumab, Bertilimumab, Besilesomab, Bevacizumab, Bezlotoxumab, Biciromab, Bimagrumab, Bivatuzumab mertansine, Blinatumomab, Blosozumab, Brentuximab vedotin, Briakinumab, Brodalumab, Canakinumab, Cantuzumab mertansine, Cantuzumab ravtansine, Caplacizumab, Capromab pendetide, Carlumab, Catumaxomab, Cedelizumab, Certolizumab, pegol, Cetuximab, Citatuzumab bogatox, Cixutumumab, Clazakizumab, Clenoliximab, Clivatuzumab tetraxetan, Conatumumab, Concizumab, Crenezumab, Dacetuzumab, Daclizumab, Dalotuzumab, Daratumumab, Demcizumab, Denosumab, Detumomab, Dorlimomab aritox, Drozitumab, Duligotumab, Dupilumab, Dusigitumab, Ecromeximab, Eculizumab, Edobacomab, Edrecolomab, Efalizumab, Efungumab, Eldelumab, Elotuzumab, Elsilimomab, Enavatuzumab, Enlimomab pegol, Enokizumab, Enoticumab, Ensituximab, Epitumomab cituxetan, Epratuzumab, Erlizumab, Ertumaxomab, Etaracizumab, Etrolizumab, Evolocumab, Exbivirumab, Fanolesomab, Faralimomab, Farletuzumab, Fasinumab, FBTA, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizu mab, Ibritumomab tiuxetan, Icrucumab, Igovomab, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lambrolizumab, Lampalizumab, Lebrikizumab, Lemalesomab, Lerdelimumab, Lexatumumab, Libivirumab, Ligelizumab, Lintuzumab, Lirilumab, Lodelcizumab, Lorvotuzumab mertansine, Lucatumumab, Lumiliximab, Mapatumumab, Margetuximab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD3, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Ocaratuzumab, Ocrelizumab, Odulimomab, Ofatumumab, Olaratumab, Olokizumab, Omalizumab, Onartuzumab, Oportuzumab monatox, Oregovomab, Orticumab, Otelixizumab, Oxelumab, Ozanezumab, Ozoralizumab, Pagibaximab, Palivizumab, Panitumumab, Panobacumab, Parsatuzumab, Pascolizumab, Pateclizumab, Patritumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, PRO, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Teneliximab, Teplizumab, Teprotumumab, TGN, Ticilinumab (or tremelimumab), Tildrakizumab, Tigatuzumab, TNX-, Tocilizumab (or atlizumab), Toralizumab, Tositumomab, Tovetumab, Tralokinumab, Trastuzumab, TRBS, Tregalizumab, Tremelimumab, Tucotuzumab celmoleukin, Tuvirumab, Ublituximab, Urelumab, Urtoxazumab, Ustekinumab, Vantictumab, Vapaliximab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab and Zolimomab aritox.

Examples of Fc fusion proteins include, without limitation, Etanercept, Alefacept, Abatacept, Rilonacept, Romiplostim, Belatacept and Aflibercept.

Examples of anticoagulants and/or blood factors include, without limitation, Protein C, Protein S, and antithrombin, Factors I-VIII, prothrombinase, prothrombin, thrombin von Willebrand Factor (vWF), fibrinogen, fibrin and fibrinopeptides.

Examples of bone morphogenetic proteins (BMPs) include, without limitation, BMP1-BMP7, BMP8a, BMP8b, BMP10, and BMP15.

Examples of enzymes include, without limitation, any of the enzymes assigned an Enzyme Commission Number (EC) number (e.g. EC1-EC6) by the International Union of Biochemistry and Molecular Biology (IUBMB) (Webb, Edwin C. Enzyme nomenclature 1992: recommendations of the Nomenclature Committee of the International Union of Biochemistry and Molecular Biology on the nomenclature and classification of enzymes. San Diego: Published for the International Union of Biochemistry and Molecular Biology by Academic Press. ISBN 0-12-227164-5 (1992), incorporated herein by reference). Other examples include: styrene monooxygenase (StyAB), toluene dioxygenase (TODC1C2AB), luciferase and lactase. In some embodiments, the enzyme is toluene dioxygenase. In some embodiments, the enzyme is styrene monoxygenase.

Examples of growth factors include, without limitation, Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha (TGF-α), Transforming growth factor beta (TGF-β), Tumor necrosis factor-alpha (TNF-a), Vascular endothelial growth factor (VEGF), placental growth factor (PIGF), Foetal Bovine Somatotrophin (FBS) and IL-1-IL7.

Examples of peptide hormones include, without limitation, Amylin (or Islet Amyloid Polypeptide), Antimullerian hormone (or Müllerian inhibiting factor or hormone), Adiponectin, Adrenocorticotropic hormone (or corticotropin), Angiotensinogen and angiotensin, Antidiuretic hormone (or vasopressin, arginine vasopressin), Atrial-natriuretic peptide (or atriopeptin), Brain natriuretic peptide, Calcitonin, Cholecystokinin, Corticotropin-releasing hormone, Enkephalin, Endothelin, Erythropoietin, Follicle-stimulating hormone, Galanin, Gastrin, Ghrelin, Glucagon, Gonadotropin-releasing hormone, Growth hormone-releasing hormone, Human chorionic gonadotropin, Human placental lactogen, Growth hormone, Inhibin, Insulin, Insulin-like growth factor (or somatomedin), Leptin, Lipotropin, Luteinizing hormone, Melanocyte stimulating hormone, Motilin, Orexin, Oxytocin, Pancreatic polypeptide, Parathyroid hormone, Prolactin, Prolactin releasing hormone, Relaxin, Renin, Secretin, Somatosta tin, Thrombopoietin, Thyroid-stimulating hormone (or thyrotropin), and Thyrotropin-releasing hormone.

Examples of interferons (IFNs) include, without limitation, IFN-α, IFN-β, IFN-ω and IFN-γ.

Examples of interleukins include, without limitation, interleukin 1-17. The interleukin may be Interleukin-4, Interleukin-6, Interleukin-10, Interleukin-11 or Interleukin-13.

Other examples of therapeutic proteins include, without limitation, Insulin (blood glucose regulator), Pramlintide acetate (glucose control), Growth hormone GH (growth failure), Pegvisoman (growth hormone receptor antagonist), Mecasermin (IGF1, growth failure), Factor VIII (coagulation factor), Factor IX (coagulation factor, Protein C concentrate (anti-coagulation), al-proteinase inhibitor (anti-trypsin inhibitor), Erythropoietin (stimulates erythropoiesis), Filgrastim (granulocyte colony-stimulating factor, G-CSF; stimulates neutrophil proliferation), Sargramostim 36, 37 (granulocytemacrophage colony-stimulating factor, GM-CSF), Oprelvekin (interleukin-11, IL-11), Human follicle-stimulating hormone (FSH), Human chorionic gonadotropin (HCG), Lutropin-α (human luteinizing hormone), Interleukin 2 (IL-2), Interleukin-1 Receptor Agonist, Denileukin diftitox (fusion of IL-2 and Diphtheria toxin), Interferon alfacon 1 (consensus interferon), Interferon-α2a (IFN-α2a), Interferon-α2b (IFN-α2b), Interferon-αn3 (IFN-αn3), Interferon-β1a (rIFN-β), Interferon-βIb (rIFN-β), Interferon-γIb (IFNγ), Salmon calcitonin (32-amino acid linear polypeptide hormone), Teriparatide (part of human parathyroid hormone 1-34 residues), Exenatide (Incretin mimetic with actions similar to glucagon-like peptide 1), Octreotide (octapeptide that mimics natural somatostatin), Dibotermin-α (recombinant human bone morphogenic protein 2), Recombinant human bone morphogenic protein 7, Histrelin acetate (gonadotropin-releasing hormone; GnRH), Palifermin (Keratinocyte growth factor, KGF), Becaplermin (platelet-derived growth factor, PDGF), Nesiritide (recombinant human B-type natriuretic peptide), Lepirudin (recombinant variant of hirudin, another variant is Bivalirudin), Anakinra (interleukin 1 (IL-1) receptor antagonist), Enfuviritide (an HIV-1 gp41-derived peptide), β-Glucocerebrosidase (hydrolyzes to glucose and ceramide), Alglucosidase-α (degrades glycogen), Laronidase (digests glycosaminoglycans within lysosomes), Idursulfase (cleaves β-sulfate preventing GAGs accumulation), Galsulfase (cleave terminal sulphage from GAGs), Agalsidase-β (human α-galactosidase A, hydrolyzes glycosphingolipids), Lactase (digest lactose), Pancreatic enzymes (lipase, amylase, protease; digest food), Adenosine deaminase (metabolizes adenosine), Tissue plasminogen activator (tPA, serine protease involved in the breakdown of blood clots), Factor VIIa (serine protease, causes blood to clot), Drotrecogin-α (serine protease, human activated protein C), Trypsin (serine protease, hydrolyzes proteins), Botulinum toxin type A (protease, inactivates SNAP-25 which is involved in synaptic vesicle fusion), Botulinum toxin type B (protease that inactivates SNAP-25 which is involved in synaptic vesicle fusion), Collagenase (endopeptidase, digest native collagen), Human deoxyribonuclease I (endonuclease, DNase I, cleaves DNA), Hyaluronidase (hydrolyzes hyaluronan), Papain (cysteine protease, hydrolyzes proteins), L-Asparaginase (catalyzes the conversion of L-asparagine to aspartic acid and ammonia), Rasburicase (urate oxidase, catalyzes the conversion of uric acid to allantoin), Streptokinase (Anistreplase is anisoylated plasminogen streptokinase activator complex (APSAC)), and Antithrombin III (serine protease inhibitor).

Other therapeutic POIs are described elsewhere herein.

In one embodiment, the POI is a molecule that is beneficial to the local environment of the cell. The POI may be a beneficial metabolite that is not present or present in too low a concentration in the local environment of the cell. In these embodiments, the first cells may be comprised by a patient, and the POI is beneficial to the patient when expressed in the local environment of the first cells. Particularly, the patient is a human patient. The POI may be insulin (related to benefits in diabetes). The POI may be GLP-1 (related to benefits in obesity). The POI may be an indole derivate, such as indole-3-acetic acid (IAA), indole-3-propionic acid (IPA), etc.; or a short chain fatty acids (SCFA), such as butyrate, acetate or propionate; or a bacterial sphingolipid. These molecules improve the intestinal barrier and prevent “leaky gut”. The POI may be an interleukin (e.g. any of the interleukins described elsewhere herein). The POI may be a molecule that produces an anti-inflammatory effect. The POI may be a tissue growth factors (e.g. any of the tissue growth factors described herein). Tissue growth factors are useful for healing epithelial barriers.

In one embodiment, the POI is a reporter molecule. Reporter genes encoding the reporter molecules disclosed herein may also be used to assay the activity of a potential SPA promoter according to the methods described elsewhere herein. Such reporter genes may encode any of the reporter molecules described. A reporter molecule refers to a protein molecule that can be used to measure gene expression and generally produce a measurable signal such as fluorescence, luminescence or colour. The presence of a reporter in a cell or organism is readily observed. For example, fluorescent proteins (e.g. green fluorescent protein (GFP)) cause a cell to fluoresce when excited with light of a particular wavelength, luciferases cause a cell to catalyse a reaction that produces light, and enzymes such as β-galactosidase convert a substrate to a coloured product.

The skilled person will be aware of different ways to measure or quantify a reporter molecule, depending on the particular reporter molecule, such as microscopy, flow cytometry and plate readers.

Fluorescent proteins may be used for visualizing or quantifying gene product expression. Fluorescence can be readily quantified using a microscope, plate reader or flow cytometer equipped to excite the fluorescent protein with the appropriate wavelength of light. Several different fluorescent proteins are available, including GFP, RFP, YFP, ECFP, mCherrry, mOrange, mBanana and the like.

Luciferases may also be used for visualizing or quantifying gene product expression, particularly for measuring low levels of gene expression, as cells tend to have little to no background luminescence in the absence of a luciferase. Luminescence can be readily quantified using a plate reader or luminescence counter. Examples of genes encoding luciferases for that may be used include, without limitation, dmMyD88-linker-Rluc, dmMyD88-linker-Rluc-linker-PEST191, luxAB, NanoLuc, Renilla reniformis luciferase, and firefly luciferase (from Photinus pyralis).

Enzymes that produce coloured substrates (“colorimetric enzymes”) may also be used for visualizing or quantifying gene product expression. Enzymatic products may be quantified using spectrophotometers or other instruments that can take absorbance measurements including plate readers. Like luciferases, enzymes such as β-galactosidase can be used for measuring low levels of gene expression because they tend to amplify low signals. Examples of genes encoding colorimetric enzymes that may be used in accordance with the present disclosure include, without limitation, lacZ alpha fragment, lacZ (encoding beta-galactosidase, full-length), and xylE.

In any configuration, embodiment or part of this disclosure the NOI, or the nucleotide may encode, or the vector may further encode, one or more phage structural proteins (such as one to 10 structural proteins). The POI may encode one to 5 phage structural proteins (such as 2 to 5 structural proteins). The phage structural protein(s) may be one or more spike proteins. The phage structural protein(s) may be one or more base-plate proteins. The phage structural protein(s) may be one or more phage capsid proteins. The phage structural protein(s) may be one or more phage tail fibre proteins. The POI may encode any combination of spike protein(s), base-plate protein(s), phage capsid protein(s) and phage tail fibre protein(s). Where the vector further encodes one or more phage structural proteins, the nucleotide encoding said structural proteins may be under the control of an SPA promoter (which may be the same or different SPA promoter to the SPA promoter regulating the expression of the toxic agent, component thereof, or POI). Where one or more phage structural proteins are under the control of an SPA promoter, this will allows the phage to replicate in first cells under stress conditions, which may aid in infectivity. Alternatively, where the vector further encodes one or more phage structural proteins, the nucleotide encoding said structural proteins may be under the control of an inducible promoter or a constitutive promoter.

### Growth Nutrients and Growth Conditions

These embodiments are applicable to all parts of the disclosure where the SPA is tested for promoter activity in standard conditions and stress conditions.

It is well-known that all cells require certain minimum nutrients to ensure optimal growth of the cells. There are many complete media available to the skilled person and resources to determine optimal conditions for growing different species and strains of cells, such as microorganism cells, bacterial cells, archaeal cells or fungal cells (e.g. yeast cells), in particular bacterial cells and archaeal cells, for example, bacterial cells. Conditions for standard conditions tend to be relatively consistent between bacteria with the exception of extremophiles. Bacteria have optimal growth conditions under which they thrive, but once outside of those conditions the stress can result in either reduced or stalled growth, dormancy (such as formation spores), or death.

Particularly, under standard conditions, the growth nutrients are of a composition and concentration, and the growth conditions are such that together they provide optimal conditions for the reference cells to grow (e.g. in the exponential phase), such as optimal laboratory conditions (as can readily be determined by the skilled person). Optimal conditions are those which are not substantially improved by changing the growth nutrient composition or concentrations or by changing the growth conditions. The skilled addressee understands what is meant by the term of art “optimal conditions” for growth.

According to any embodiment of the first to sixth configurations, instead of part (a) reading “(a) the standard conditions are a set of in vitro growing conditions which include growth nutrient and growing conditions, and in which the addition of further growth nutrient and change of growing conditions does not further improve the growth rate of the reference cells, part (a) reads “(a) the standard conditions are a set of in vitro growing conditions which include growth nutrients and growing conditions, under which the reference cells are growing under optimal laboratory conditions”.

In other embodiments of the first to sixth configurations, instead of part (a) reading “(a) the standard conditions are a set of in vitro growing conditions which include growth nutrients and growing conditions, and in which the addition of further growth nutrients and change of growing conditions does not further improve the growth rate of the reference cells”, part (a) reads “(a) the standard conditions are a set of in vitro growing conditions which include growth nutrients and growing conditions, under which the reference cells are growing such that a doubling time can be achieved and measured”. Most cells, when growing well, do so sufficiently quickly that it is possible to measure and calculate a doubling time. The measured doubling time under standard conditions will depend on the species and strain of the reference cells, and will be readily apparent to the skilled person. Representative doubling times under standard conditions are shown for certain strains in Table 1 and in the Examples hereinbelow. The measured doubling time under standard conditions may be less than 2 hours. The measured doubling time under standard conditions may be less than 1.5 hours. The measured doubling time under standard conditions may be less than 1 hour. The measured doubling time under standard conditions may be less than 45 minutes. The measured doubling time under standard conditions may be less than 30 minutes. The measured doubling time under standard conditions may be less than 20 minutes. The measured doubling time under standard conditions may be less than 15 minutes. The doubling time may be measured according to any of the methods of measuring doubling time described herein.

In other embodiments of the first to sixth configurations, instead of part (a) reading “(a) the standard conditions area set of in vitro growing conditions which include growth nutrients and growing conditions, and in which the addition of further growth nutrients and change of growing conditions does not further improve the growth rate of the reference cell”, part (a) reads “(a) the standard conditions are a set of in vitro growing conditions which include growth nutrients and growing conditions, under which the reference cells are growing in the exponential phase”. As described elsewhere herein, cells have certain characteristics which are readily determined by one of skill in the art to identify if they are in the exponential phase or not.

In other embodiments of the first to sixth configurations, instead of part (a) reading “(a) the standard conditions are a set of in vitro growing conditions which include growth nutrients and growing conditions, and in which the addition of further growth nutrients and change of growing conditions does not further improve the growth rate of the reference cell”, part (a) reads “(a) the standard conditions are a set of in vitro growing conditions which include growth nutrients and growing conditions, under which the reference cells are growing and are not cells which are selected from stationary phase cells, stressed cells, persister cells, biofilm cells and dormant cell”. As described elsewhere herein, cells have certain characteristics which are readily determined by one of skill in the art to identify if they are stationary phase cells, stressed cells, persister cells, biofilm cells or dormant cells or not.

Complete media composition and growth conditions under standard conditions are often recommended upon purchase of the reference cells, and provide excellent, if not optimal laboratory growing conditions for the reference cells: thus, in one embodiment, the growth nutrients and growth conditions under standard conditions are according to the supplier's recommendations for the reference cells. In one embodiment, the growth nutrients under standard conditions are comprised by a complete media. Sometimes this is referred to as in the art as an “enriched culture medium” see Bonnet et al., “Bacterial culture through selective and non-selective conditions: the evolution of culture media in clinical microbiology”, New Microbes New Infect., 2020 March; 34: 100622, published online 2019 Nov. 30. doi: 10.1016/j.nmni.2019.100622, which is incorporated herein by reference. Complete media may be solid (e.g. agar) or liquid, as required. In one embodiment, the complete media under standard conditions is selected from lysogeny broth (LB), tryptic soy broth (TSB), brain-heart infusion (BHI), Nutrient broth, and Gifu anaerobic broth (GAM), and their agar counterparts. In another embodiment, the reference cells, growth nutrients and growth conditions under standard conditions are as provided for in Table 1 below.

Alternatively, the MediaDB database (see https://mediadb.systemsbiology.netl) is one source of information where the skilled person is able to input a certain species or strain of bacteria and obtain a description of growth nutrients for standard conditions which have been tested to provide excellent growing nutrients for that species or strain. However, there may be situations where no such generally-accepted growth nutrients for standard conditions are available.

The growth nutrients and/or growth conditions under standard conditions may be composed to mimic the naturally occurring growth nutrients found in a human or animal microbiome. The growth nutrients and/or growth conditions under standard conditions may mimic the naturally occurring growth conditions and/or growth conditions found in a microbiome comprised within an organ. The microbiome may be selected from a gut, a lung, a skin, and a blood microbiome, in particular a human microbiome. The first cells may be bacterial or archaeal cells, e.g. bacterial cells. The growth nutrients and/or growth conditions under standard conditions may mimic the naturally occurring growth conditions and/or growth conditions found in a microbiome selected from a gut, skin, blood, lung, nasal, oral, vaginal, penile, urinary tract, bladder, urethra, kidney, liver or heart microbiome, in particular of a human. The growth nutrients and/or growth conditions under standard conditions may mimic the naturally occurring growth nutrients and/or growth conditions of the first cells in their native environment.

The growth nutrients and/or growth conditions under standard conditions for the reference cells may be determined according to public databases and textbooks.

TABLE 1
Particular growth nutrients and growth conditions for selected bacteria

| | Medium | | | | Growth |
|---|---|---|---|---|---|
| | | Concentration | | | rate |
| Species | Compound | (mM) | Conditions | | (1/h) |
| Acinetobacter | Ammonium chloride | 20.6 | pH: | 7.4 | 0.95 |
| baumannii ATCC | Calcium chloride | 1.02 | Temperature (° C.): | 37 | |
| 19606 | anhydrous | | | | |
| | EDDHA | 0.2 | | | |
| | Iron(III) chloride | 0.2 | | | |
| | Magnesium chloride | 0.492 | | | |
| | Potassium chloride | 49.6 | | | |
| | Sodium chloride | 85.6 | | | |
| | Sodium sulfate | 1 | | | |
| | Tromethamine | 99.9 | | | |
| Acinetobacter | Ammonium sulfate | 75,677 | pH: | 6.8 | 0.5373 |
| baylyi ADP1 | Calcium chloride | 0.603712 | Temperature (° C.): | 30 | |
| | anhydrous | | | | |
| | Cobalt nitrate | 0.000852146 | | | |
| | Cupric sulfate | 0.0015698 | | | |
| | Dibasic sodium phosphate | 50 | | | |
| | EDTA | 0.008554 | | | |
| | Ferrous sulfate | 0.03114 | | | |
| | Magnesium sulfate | 48,186 | | | |
| | Manganese sulfate | 0.009111 | | | |
| | Molybdic acid ammonium | 0.00016183 | | | |
| | salt tetrahydrate | | | | |
| | Nitrilotriacetic acid | 1,151 | | | |
| | Potassium dibasic | 50 | | | |
| | phosphate | | | | |
| | Sodium borate | 0.0004641 | | | |
| | Succinate | 10 | | | |
| | Zinc sulfate | 0.038082 | | | |
| Clostridium | 4-Aminobenzoate | 0.00729 | pH: | 7 | 0.233676 |
| acetobutylicum | Ammonium phosphate | 45.43 | Temperature (° C.): | 34 | |
| P262 | Biotin | 0.00020465 | Anaerobic | | |
| | Calcium carbonate | 199,826 | | | |
| | Calcium chloride | 0.0720819 | | | |
| | anhydrous | | | | |
| | Cysteine-HCl | 2,847 | | | |
| | D-Glucose | 333,037 | | | |
| | Ferrous sulfate | 0.03596 | | | |
| | Magnesium sulfate | 0.877863 | | | |
| | Manganese sulfate | 0.04483 | | | |
| | Potassium dibasic | 502,296 | | | |
| | phosphate | | | | |
| | Potassium dihydrogen | 0.293932 | | | |
| | phosphate | | | | |
| | Sodium chloride | 136,893 | | | |
| | Sodium citrate | 476,152 | | | |
| | Sodium molybdate | 0.04133 | | | |
| | Thiamine HCl | 0.00296 | | | |
| | Zinc sulfate | 0.017389 | | | |
| Escherichia coli | Ammonium chloride | 186,947 | pH: | 7 | 0.33472 |
| BL21 | Calcium chloride | 0.1 | Temperature (° C.): | 37 | |
| | anhydrous | | | | |
| | Dibasic sodium phosphate | 47,743 | | | |
| | Glycerol | 21,718 | | | |
| | Magnesium sulfate | 2 | | | |
| | Potassium dihydrogen | 22,045 | | | |
| | phosphate | | | | |
| | Sodium chloride | 85,557 | | | |
| Escherichia coli | Ammonium chloride | 93,474 | pH: | 7 | 0.8304 |
| BW25113 | Ammonium sulfate | 18,919 | Temperature (° C.): | 37 | |
| | Calcium chloride | 0.01163 | | | |
| | anhydrous | | | | |
| | Cobalt chloride | 0.00227 | | | |
| | Cupric sulfate | 0.0019223 | | | |
| | Iron(III) chloride | 0.18535 | | | |
| | Magnesium sulfate | 3 | | | |
| | Manganese sulfate | 0.001775 | | | |
| | Potassium dibasic | 419,058 | | | |
| | phosphate | | | | |
| | Sodium EDTA | 0.18828 | | | |
| | Sodium dihydrogen | 30,005 | | | |
| | phosphate | | | | |
| | Sodium sulfate | 140,449 | | | |
| | Zinc sulfate | 0.001878 | | | |
| | beta-D-Glucose | 55,506 | | | |
| Klebsiella | Ammonium chloride | 19 | Temperature (° C.): | 37 | 1,084 |
| pneumoniae | Calcium chloride | 0.1 | | | |
| | anhydrous | | | | |
| | Dibasic sodium phosphate | 49 | | | |
| | Glucose | 11 | | | |
| | Magnesium sulfate | 2 | | | |
| | Potassium dihydrogen | 22 | | | |
| | phosphate | | | | |
| | Sodium chloride | 8.6 | | | |
| Lactobacillus | 4-Aminobenzoate | 0.07292 | pH: | 5 | 0.6 |
| plantarum | Adenine | 0.074 | Temperature (° C.): | 37 | |
| wcfs1 | Ammonium citrate | 265,269 | | | |
| | Ascorbate | 283,897 | | | |
| | Biotin | 0.01023 | | | |
| | Calcium pantothenate | 0.00456 | | | |
| | Cysteine-HCl | 0.82477 | | | |
| | D-Glucose | 555,062 | | | |
| | Folate | 0.00227 | | | |
| | Glycine | 233,116 | | | |
| | Guanine | 0.06617 | | | |
| | Inosine | 0.01864 | | | |
| | L-Alanine | 269,391 | | | |
| | L-Arginine | 0.71757 | | | |
| | L-Aspartate | 315,552 | | | |
| | L-Glutamate | 339,836 | | | |
| | L-Histidine | 0.96681 | | | |
| | L-Isoleucine | 160,098 | | | |
| | L-Leucine | 362,125 | | | |
| | L-Lysine | 300,978 | | | |
| | L-Methionine | 0.83775 | | | |
| | L-Phenylalanine | 166,475 | | | |
| | L-Proline | 586,293 | | | |
| | L-Serine | 323,532 | | | |
| | L-Threonine | 188,885 | | | |
| | L-Tryptophan | 0.24482 | | | |
| | L-Tyrosine | 137,977 | | | |
| | L-Valine | 277,422 | | | |
| | Lipoate | 0.00485 | | | |
| | Nicotinate | 0.00812 | | | |
| | Orotate | 0.03203 | | | |
| | Potassium dibasic | 574,053 | | | |
| | phosphate | | | | |
| | Potassium dihydrogen | 367,415 | | | |
| | phosphate | | | | |
| | Pyridoxamine HCl | 0.02443 | | | |
| | Pyridoxine HCl | 0.00973 | | | |
| | Riboflavin | 0.00266 | | | |
| | Sodium acetate | 121,901 | | | |
| | Thiamine HCl | 0.00332 | | | |
| | Thymidine | 0.02064 | | | |
| | Uracil | 0.08922 | | | |
| | Xanthine | 0.06574 | | | |
| Lactococcus | Alanine | 2,694 | pH: | 6.6 | 0.78 |
| lactis MG1363 | Ammonium sulfate | 1,362 | Temperature (° C.): | 30 | |
| | Arginine | 0.6889 | Microaerobic | | |
| | Asparagine | 2,573 | | | |
| | Biotin | 0.04093 | | | |
| | Calcium pantothenate | 0.002099 | | | |
| | Cysteine | 1,403 | | | |
| | DL-Serine | 3,235 | | | |
| | DL-Tyrosine | 1,601 | | | |
| | DL-methionine | 0.8042 | | | |
| | Glucose | 55.51 | | | |
| | Glutamine | 3.49 | | | |
| | Glycine | 2,265 | | | |
| | Histidine | 0.7089 | | | |
| | Isoleucine | 1,525 | | | |
| | Leucine | 3,583 | | | |
| | Lysine-HCl | 1,916 | | | |
| | Magnesium chloride | 0.9838 | | | |
| | Manganese chloride | 9.93E−02 | | | |
| | Nicotinate | 0.008123 | | | |
| | Phenylalanine | 1,695 | | | |
| | Potassium dibasic | 42.92 | | | |
| | phosphate | | | | |
| | Potassium dihydrogen | 66.13 | | | |
| | phosphate | | | | |
| | Proline | 5,906 | | | |
| | Pyridoxamine | 0.02973 | | | |
| | Sodium acetate | 9,996 | | | |
| | Thiamine | 0.003769 | | | |
| | Threonine | 1,931 | | | |
| | Tryptophan | 0.2448 | | | |
| | Valine | 2,817 | | | |
| Mycobacterium | ACES | 54.9 | pH: | 6.9 | 0.0472 |
| tuberculosis | Biotin | 0.000409 | Temperature (° C.): | 37 | |
| H37Rv | Calcium chloride | 0.00374 | Aerobic | | |
| | anhydrous | | | | |
| | Cobalt chloride | 0.00202 | | | |
| | Cupric sulfate | 0.0001 | | | |
| | Ferrous sulfate | 0.367 | | | |
| | Glycerol | 27.4 | | | |
| | Glycine | 1.33 | | | |
| | L-Alanine | 1.12 | | | |
| | L-Arginine | 0.574 | | | |
| | L-Asparagine | 15.1 | | | |
| | L-Aspartate | 0.751 | | | |
| | L-Glutamate | 0.68 | | | |
| | L-Isoleucine | 0.762 | | | |
| | L-Leucine | 0.762 | | | |
| | L-Phenylalanine | 0.605 | | | |
| | L-Serine | 0.952 | | | |
| | Magnesium sulfate | 0.8869 | | | |
| | Manganese chloride | 0.000101 | | | |
| | Potassium dihydrogen | 1.63 | | | |
| | phosphate | | | | |
| | Potassium hydroxide | 1 | | | |
| | Pyruvate | 9.09 | | | |
| | Sodium bicarbonate | 0.5 | | | |
| | Tween 80 | 1.53 | | | |
| | Zinc sulfate | 0.00141 | | | |
| Porphyromonas | Alanine | 11,224 | Temperature (° C.): | 37 | 0.067 |
| gingivalis W83 | Aspartate | 0.7513 | Anaerobic | | |
| | Calcium chloride | 0.9011 | | | |
| | anhydrous | | | | |
| | Cysteine-HCl | 0.4758 | | | |
| | Hemin | 0.007669 | | | |
| | L-Glutamate | 0.6797 | | | |
| | Magnesium sulfate | 28,401 | | | |
| | Menadione | 0.002904 | | | |
| | Phenylalanine | 0.6054 | | | |
| | Potassium dibasic | 11,481 | | | |
| | phosphate | | | | |
| | Potassium dihydrogen | 73,483 | | | |
| | phosphate | | | | |
| | Sodium chloride | 17,112 | | | |
| Pseudomonas | Ammonium chloride | 187 | Temperature (° C.): | 30 | 0.56 |
| putida KT2440 | Boric acid | 0.013 | | | |
| | Cobalt chloride | 0.0039 | | | |
| | Cupric chloride | 0.00015 | | | |
| | D-Glucose | 17 | | | |
| | Magnesium chloride | 0.00038 | | | |
| | Magnesium sulfate | 4.3 | | | |
| | Nickel chloride | 0.00021 | | | |
| | Potassium dibasic | 220 | | | |
| | phosphate | | | | |
| | Sodium chloride | 86 | | | |
| | Sodium dihydrogen | 194 | | | |
| | phosphate | | | | |
| | Sodium molybdate | 0.00031 | | | |
| | Zinc Chloride | 0.00092 | | | |
| Salmonella | Ammonium chloride | 140,211 | Temperature (° C.): | 37 | 0.612 |
| enterica | Calcium chloride | 0.0901024 | | | |
| Typhimurium | anhydrous | | | | |
| LT2 | D-Glucose | 111,012 | | | |
| | Ferrous sulfate | 0.0017982 | | | |
| | L-Methionine | 0.26807 | | | |
| | Magnesium chloride | 0.983719 | | | |
| | Potassium chloride | 134,136 | | | |
| | Potassium dibasic | 487,944 | | | |
| | phosphate | | | | |
| | Potassium dihydrogen | 308,628 | | | |
| | phosphate | | | | |
| | Sodium sulfate | 140,805 | | | |
| | Thymine | 0.031717 | | | |
| Staphylococcus | Ammonium sulfate | 30.3 | Temperature (° C.): | 37 | 0.55 |
| aureus N315 | Biotin | 0.000409 | | | |
| | Calcium chloride | 0.15 | | | |
| | anhydrous | | | | |
| | D-Glucose | 27.8 | | | |
| | D-Glutamate | 1.71 | | | |
| | Ferrous sulfate | 0.0216 | | | |
| | L-Arginine | 0.718 | | | |
| | L-Cysteine | 0.66 | | | |
| | L-Leucine | 1.14 | | | |
| | L-Phenylalanine | 0.908 | | | |
| | L-Proline | 1.74 | | | |
| | L-Threonine | 1.26 | | | |
| | L-Valine | 1.28 | | | |
| | Magnesium sulfate | 5.28 | | | |
| | Manganese sulfate | 0.0368 | | | |
| | Nicotinate | 0.0162 | | | |
| | Pantothenate | 0.0074 | | | |
| | Potassium chloride | 40.2 | | | |
| | Potassium dihydrogen | 1.03 | | | |
| | phosphate | | | | |
| | Sodium chloride | 163 | | | |
| | Thiamine | 0.00665 | | | |
| | Tromethamine | 100 | | | |
| Streptococcus | 4-Aminobenzoate | 0.07292 | pH: | 6.7 | 2.2 |
| thermophilus | Adenine | 0.074 | Temperature (° C.): | 37 | |
| ST11 | Alanine | 2,694 | | | |
| | Ammonium citrate | 28.69 | | | |
| | Arginine | 0.7463 | | | |
| | Ascorbate | 2,839 | | | |
| | Asparagine | 2,649 | | | |
| | Aspartate | 3,456 | | | |
| | Biotin | 0.04093 | | | |
| | Calcium chloride | 0.3401 | | | |
| | anhydrous | | | | |
| | Calcium pantothenate | 0.002099 | | | |
| | Cobalt chloride | 0.01051 | | | |
| | Cupric sulfate | 0.0004005 | | | |
| | Cysteine | 2,063 | | | |
| | DL-Serine | 3,235 | | | |
| | DL-Tyrosine | 1,601 | | | |
| | DL-methionine | 0.8713 | | | |
| | Ferrous chloride | 0.2515 | | | |
| | Folate | 0.002266 | | | |
| | Glutamine | 2,668 | | | |
| | Glycine | 2,398 | | | |
| | Guanine | 0.06617 | | | |
| | Histidine | 0.9667 | | | |
| | Inosine | 0.01864 | | | |
| | Isoleucine | 1,677 | | | |
| | L-Glutamate | 2,719 | | | |
| | Lactose | 29.21 | | | |
| | Leucine | 3,659 | | | |
| | Lipoate | 0.01212 | | | |
| | Lysine | 3.01 | | | |
| | Magnesium chloride | 0.9838 | | | |
| | Manganese sulfate | 0.1657 | | | |
| | Nicotinate | 0.008123 | | | |
| | Orotate | 0.03203 | | | |
| | Phenylalanine | 1,695 | | | |
| | Potassium dibasic | 14.31 | | | |
| | phosphate | | | | |
| | Potassium dihydrogen | 22.04 | | | |
| | phosphate | | | | |
| | Proline | 5,906 | | | |
| | Pyridoxamine HCl | 0.02443 | | | |
| | Pyridoxine HCl | 0.009725 | | | |
| | Riboflavin | 0.002657 | | | |
| | Sodium acetate | 12.19 | | | |
| | Thiamine HCl | 0.002965 | | | |
| | Threonine | 1,931 | | | |
| | Thymidine | 0.0221 | | | |
| | Tryptophan | 0.2448 | | | |
| | Uracil | 0.08921 | | | |
| | Valine | 2,817 | | | |
| | Vitamin B12 | 0.0007378 | | | |
| | Xanthine | 0.06574 | | | |
| | Zinc sulfate | 0.1739 | | | |
| Pseudomonas | Ammonium sulfate | 1.51 | Temperature (° C.): | 37 | 1,048 |
| aeruginosa | Boric acid | 8.09E−02 | | | |
| PA01 | Calcium chloride | 0.01 | | | |
| | anhydrous | | | | |
| | Calcium sulfate | 0.001469 | | | |
| | Cobaltous sulfate | 3.56E−02 | | | |
| | Cupric sulfate | 0.0001253 | | | |
| | Dibasic sodium phosphate | 3.37 | | | |
| | Ferrous sulfate | 0.0007194 | | | |
| | Iron(III) chloride | 0.001 | | | |
| | Magnesium chloride | 5 | | | |
| | Manganese sulfate | 0.0001325 | | | |
| | Potassium dihydrogen | 2.2 | | | |
| | phosphate | | | | |
| | Sodium chloride | 179 | | | |
| | Sodium citrate | 0.1 | | | |
| | Sodium molybdate | 4.86E−02 | | | |
| | Zinc sulfate | 6.96E−02 | | | |
| Yersinia pestis | Alanine | 4.5 | pH: | 7.5 | 0.257277 |
| EV76 | Ammonium chloride | 10 | Temperature (° C.): | 37 | |
| | Biotin | 0.002 | | | |
| | Calcium chloride | 2.5 | | | |
| | anhydrous | | | | |
| | Calcium pantothenate | 0.004 | | | |
| | Citrate | 10 | | | |
| | Ferrous chloride | 0.1 | | | |
| | Glycine | 2.6 | | | |
| | HEPES | 25 | | | |
| | L-Arginine | 1 | | | |
| | L-Cysteine | 1 | | | |
| | L-Glutamate | 81.1 | | | |
| | L-Isoleucine | 3.8 | | | |
| | L-Leucine | 2 | | | |
| | L-Lysine | 1.1 | | | |
| | L-Methionine | 1.6 | | | |
| | L-Phenylalanine | 2.4 | | | |
| | L-Proline | 7 | | | |
| | L-Threonine | 1.4 | | | |
| | L-Tryptophan | 0.1 | | | |
| | L-Tyrosine | 1.1 | | | |
| | L-Valine | 6.8 | | | |
| | Magnesium chloride | 2.5 | | | |
| | Manganese chloride | 0.01 | | | |
| | Potassium Gluconate | 10 | | | |
| | Potassium dibasic | 2.5 | | | |
| | phosphate | | | | |
| | Thiamine | 0.003 | | | |

Determining excellent growth conditions for any given cell type is with the skill of one in the art. Starting from the information available on the native growing conditions for the reference cells and the information available from public resources such as databases (as discussed above), the literature and supplier's recommendations, a skilled person can devise a set of growth nutrients and growth conditions to test with the reference cells to determine the standard conditions.

In one embodiment, the growth rate of the cells is tested, e.g. by observing the doubling time (as described elsewhere herein). Each nutrient and condition can then be varied, according to the scientific method, and it can be determined if the growth rate can be further improved. Once changing the parameters does not result in any further improvement of the growth rate (e.g. doubling time), the standard conditions have been identified.

Thus, in embodiments where the SPA promoter is tested for activity, the growth nutrients under standard conditions comprise:
i. water;
ii. mineral salts, optionally selected from one or more of phosphate, sulphate, magnesium and calcium (e.g. all of phosphate, sulphate, magnesium and calcium);
iii. a source of nitrogen, optionally selected from ammonia, urea, amino acids, protein acid hydrolysate (e.g. casamino acids, soy protein acid hydrolysate), and enzymatically digested proteins (e.g. soy peptone, casein peptone, casein tryptone);
iv. a source of carbon, optionally an organic carbon source, such as a source selected from amino acids, sugars, starches and alcohols (e.g. both sugars and starch);
v. a source of iron (e.g. iron salts, such as iron sulphate or iron chloride)
vi. a source of vitamins that are required for growth of the reference cells; and/or
vii. any other component which is essential for the growth of the reference cells (including, but not limited to growth factors, purine and pyrimidine bases, light sources).

In one embodiment, the growth nutrients under standard conditions comprise at least one of each of i. to v. above.

In one embodiment, under standard conditions, the source of nitrogen is ammonia.

In embodiments where the SPA promoter is tested for activity, the growth conditions under standard conditions may comprise:
I. a specific temperature;
II. a specific pH; and
III. the oxygen levels.

Particularly where the reference cells natively grow within a human body, the reference cells may be grown at a temperature between 35 and 44° C. under standard conditions. Alternatively, the reference cells may be grown at a temperature between 35 and 40° C., e.g. between 36.5 and 37.5° C., e.g. at approximately 37.0° C. under standard conditions.

The reference cells under standard conditions may be grown at a pH between 1.0 and 4.0, in particular where the reference cells natively grow within the human stomach. The reference cells under standard conditions may be grown at a pH between 3.5 and 7.5 (e.g. a mean pH of approximately 6.6), in particular where the reference cells natively grow within the human small intestine. The reference cells under standard conditions may be grown at a pH between 5.0 and 7.5, in particular where the reference cells natively grow within the human large intestine. The pH within the colon can be between 6.5 and 8.5. Thus, reference cells which grow naturally within the colon may be grown under standard conditions at a pH between 6.5 and 8.5.

The pH under standard conditions may be between 3.0 and 4.0. The pH under standard conditions may be between 4.0 and 5.0. The pH under standard conditions may be between 5.0 and 6.0. The pH under standard conditions may be between 6.0 and 7.0. The pH under standard conditions may be between 7.0 and 8.0.

The reference cells under standard conditions may be grown at a pH between 6.5 and 7.5, e.g. approximately 7.0, in particular where the reference cells natively grow in or on the human skin, brain, lungs, heart, liver, testis, bone, cochlea, spleen, kidneys, or muscle.

For other pHs found within organs and bodily fluids in humans, see Gaohua, et al., 2021, “Crosstalk of physiological pH and chemical pKa under the umbrella of physiologically based pharmacokinetic modeling of drug absorption, distribution, metabolism, excretion, and toxicity”, Expert Opinion on Drug Metabolism & Toxicology, 17:9, 1103-1124, DOI: 10.1080/17425255.2021.1951223, incorporated herein by reference.

Where the reference cells are not anaerobes, the reference cells under standard conditions may be aerated by aeration, e.g. shaking or bubbling. In one embodiment, the reference cells under standard conditions may be aerated by shaking. In one embodiment, the shaking is carried out at 150-250 rpm (e.g. approximately 200 rpm) in an orbital shaker, optionally wherein the reference cells occupy no more than 10% of the volume of the container in which they are held, e.g. wherein the container is an Erlenmeyer flask.

Where the reference cells are anaerobes, the reference cells under standard conditions may be grown under anaerobic conditions.

### Stress Conditions and Stress-Inducing Parameters

These embodiments are applicable to all parts of the disclosure where the SPA is tested for promoter activity in standard conditions and stress conditions.

It is well known that for any given cell (such as microorganism cells, bacterial cells, archaeal cells or fungal cells (e.g. yeast cells), in particular bacterial cells), there may be certain conditions or substances which will induce a stress response and thus slow or halt their growth. It may be any change in the growth conditions (such as a sub-optimal temperature or pH) or lack of availability of a certain type of growth nutrient. These stressed cells grow much more slowly than under optimal conditions (such as the standard conditions described herein), and may be classed as stationary phase cells, stressed cells, persister cells, biofilm cells or dormant cells. When stressed, they may release certain markers of stress, as described herein.

External factors that induce a stress-response in the reference cells are termed “stress-inducing parameters” herein. For example, the parameter(s) are parameter(s) that are found in stress conditions to which the first cells are exposed in or on humans or animals. For example, the parameter(s) are parameter(s) that are found in stress conditions to which the first cells are exposed in an environment, e.g. a plant, soil or aqueous (e.g. waterway or water) environment. For example, the parameter(s) are parameter(s) that are found in stress conditions to which the first cells are exposed in a microbiome, e.g. a microbiome of a human or animal, such as a gut, skin, blood, lung, nasal, oral, vaginal, penile, urinary tract, bladder, urethra, kidney, liver or heart microbiome, in particular the gut, the lungs, the skin, and the blood, and in particular in a human.

A stress-inducing parameter may be selected from the:
A. presence of toxin (e.g. antibiotic, antifungal, antimicrobials or antiviral);
B. presence of a metabolite (e.g. a bacteriocin, a cecropin, a moricin, a cupiennin, an oxyopinin, a magainin, a dermaseptin or a cathelicidin);
C. presence of second cells which are different to the reference cells (e.g. bacteria, fungi);
D. presence of phages that are capable of infecting the reference cells (e.g. a tail phage or a filamentous phage);
E. presence of free radicals which are capable of inducing DNA damage in the reference cells; and
F. presence of oxygen, where the reference cells grow under anaerobic conditions.

The stress-inducing parameter is present at a level, concentration or amount which is sufficient to slow or halt the growth of the reference cells. The presence of the stress-inducing parameter may cause the reference cells to become stationary phase cells, stressed cells, persister cells, biofilm cells and/or dormant cells. A skilled person is able to determine, using common general knowledge which stress-inducing parameter is likely to act against the type of reference cell that is being tested. For example, AMP moricin (an antimicrobial peptide metabolite from insects) has a high activity against Gram-positive and Gram-negative but is less active against yeasts. Using resources at their disposal a skilled person is able to determine and test stress-inducing parameters for their effect on the growth of reference cells.

The stress-inducing parameter may be a toxin. Examples of toxins include, but are not limited to antibiotics, antifungals, antimicrobials and/or antiviral agents.

The toxin may be an antibiotic. Antibiotics can be divided into several classes. Antibiotics disclosed herein may function as any of a stress-inducing parameter, a toxic agent and/or a POI. An antibiotic can be an aminoglycoside, e.g. selected from amikacin, liposomal amikacin, gentamicin, plazomicin and tobramycin. An antibiotic can be a β-lactam inhibitor, e.g. selected from ceftolozane and cilastatin. An antibiotic can be a β-lactamase inhibitor, e.g. selected from avibactam, clavulanate, clavulanic acid, salbactam, tazobactam, relebactam and vaborbactam. An antibiotic can be a carbapenem, e.g. selected from doripenem, ertapenem, imipenem, and meropenem. An antibiotic can be a cephalosporin, e.g. selected from cefaclor, cefadroxil, cefazolin, cefdinir, cefditoren, cefepime, cefiderocol, cefixime, cefotaxime, cefotetan, cefoxitin, cefpodoxime, cefprozil, ceftaroline, ceftazidime, ceftibuten, ceftizoxime, ceftriaxone, cefuroxime and cephalexin. An antibiotic can be a fluoroquinolone, e.g. selected from ciprofloxacin, delafloxacin, gemifloxacin, levofloxacin and moxifloxacin. An antibiotic can be a folate pathway inhibitor, e.g. selected from sulfisoxazole, sulfamethoxazole and trimethoprim. An antibiotic can be a Fosfomycin, e.g. fosfomycin. An antibiotic can be a glycopeptide, e.g. selected from dalbavancin, oritavancin, telavancin and vancomycin. An antibiotic can be a glycocycline, e.g. tigecycline. An antibiotic can be a ketolide, e.g. telithromycine, An antibiotic can be a lincosamide, e.g. clindamycin. An antibiotic can be a lipopeptide, e.g. daptomycin. An antibiotic can be a macrocyclic compound, e.g. fidaxomicin. An antibiotic can be a macrolide, e.g. selected from azithromycin, clarithromycin and erythromycin. An antibiotic can be a monobactam, e.g. aztreonam. An antibiotic can be a nitrofuran, e.g. nitrofurantoin. An antibiotic can be a nitroimidazole, e.g. selected from metronidazole and tinidazole. An antibiotic can be a nucleoside analogue, e.g. selected from molnupiravir and remdesivir. An antibiotic can be an oxazolidinone, e.g. selected from linezolid and tedizolid. An antibiotic can be penicillin, e.g. selected from amoxicillin, ampicillin, dicloxacillin, nafcillin, oxacillin, penicillin G, penicillin V, piperacillin and ticarcillin. An antibiotic can be a phenicol, e.g. chloramphenicol. An antibiotic can be a polyene, e.g. selected from amphotericin B, liposomal amphotericin B and amphotericin B lipid complex. An antibiotic can be a polymerase acidic endonuclease inhibitor, e.g. baloxavir marboxil. An antibiotic can be a polymyxin, e.g. selected from colistimethate, colistin and polymyxin B. An antibiotic can be a pleuromutilin, e.g. lefamulin. An antibiotic can be a protease inhibitor, e.g. nirmatrelvir. An antibiotic can be rifampin. An antibiotic can be a streprogramin, e.g. selected from quinupristin and dalfopristin. An antibiotic can be a tetracycline, e.g. selected from eravacycline, minocycline, omadacycline and tetracycline. An antibiotic can be any combination of the foregoing (and the NOI or nucleotide sequence can encode more than one antibiotic to deliver a combination to the first cells).

The toxin may be an antifungal. Antifungals can be divided into several classes. An antifungal can be an azole, e.g. selected from fluconazole, isavuconazonium, itraconazole, posaconazole and voriconazole. An antifungal can be an echinocandin, e.g. selected from anidulafungin, caspofungin and micafungin.

The toxin may be an antimicrobial peptide or protein. Antimicrobial peptides and proteins can be divided into several classes. Antimicrobial peptides and proteins disclosed herein may function as any of a stress-inducing parameter, a toxic agent and/or a POI. An antimicrobial peptide and protein can be a defensin. The defensin can be a trans-defensin. The trans-defensin can be an α-defensin, e.g. selected from neutrophil defensin 1, neutrophil defensin 3, neutrophil defensin 4, defensin α1B, defensin 4 or defensin 5, human neutrophil peptide 1, human neutrophil peptide 2, human neutrophil peptide 3, human neutrophil peptide 4, human α-defensin 5 and human α-defensin 6. The trans-defensin can be a β-defensin, e.g. selected from β-defensin 1, α-defensin 2, β-defensin 3, β-defensin 103, β-defensin 106A, β-defensin 1068, β-defensin 107, β-defensin 110, β-defensin 136, human β-defensin 1, human β-defensin 2, human β-defensin 3, human β-defensin 4, human β-defensin 5 and human β-defensin 6. The trans-defensin can be an β-defensin. The defensin can be a cis-defensin, e.g. selected from selected from plectasin, NaD1, lucifensin and MGD-1. The antimicrobial peptide or protein may be one which is expressed from a gene module that encode instructions for cell death, e.g. selected from selected from pemI-pemK genes of plasmid R100, the phd-doc genes of phage PI, the ccdA-ccdB genes of plasmid F, mazE-mazF (or chpAI-chpAK), sof-gef, kicA-kicB, relB-relE, chpBI-chpBK and gef.

Human defensins are described in detail in Fruitwala et al., “Multifaceted immune functions of human defensins and underlying mechanisms”, Semin Cell Dev Biol. 2019 April; 88: 163-172, published online 2018 Mar. 13. doi: 10.1016/j.semcdb.2018.02.023, which is incorporated herein by reference. Other defensins are described in Gerdol et al, “Functional Insights From the Evolutionary Diversification of Big Defensins”, Front. Immunol., 30 Apr. 2020, Sec. Microbial Immunology, Volume 11-2020, https://doi.org/10.3389/finmu.2020.00758; Xu & Lu, “Defensins: A Double-Edged Sword in Host Immunity”, Front. Immunol., 7 May 2020, Sec. Microbial Immunology, volume 11-2020, https://doi.org/10.3389/fimmu.2020.00764; Ganz, “Defensins: antimicrobial peptides of innate immunity”, Nature Reviews Immunology, volume 3, pages 710-720 (2003); each of which is incorporated herein by reference.

The toxin may be an antiviral. Antivirals can be divided into several classes. An antiviral can be an M2 ion channel inhibitor, e.g. selected from amantadine and rimantadine. An antiviral can be a neuraminidase inhibitor, e.g. selected from oseltamivir, peramivir and zanamivir.

The stress-inducing parameter may be a metabolite. Metabolites may be divided in to several classes. Examples of metabolites include, but are not limited to bacteriocins, cecropins, moricins, and other metabolites described herein.

The metabolite may be a bacteriocin. The bacteriocin may be selected from bactofencin, laterosporulin, laterosporuli10, bacteriocin (such as bacteriocin AS-48), pentocin (such as pentocin JL-1), entianin, klebicin, enterocin (such as enterocin DD28 or enterocins DD93). The bacteriocin may be any of the bacteriocins listed in Table 1 or Table 2 of Benitez-Chao et al, “Bacteriocins: An Overview of Antimicrobial, Toxicity, and Biosafety Assessment by in vivo Models”, Frontiers in Microbiology, 15 Apr. 2021, Sec. Antimicrobials, Resistance and Chemotherapy Volume 12-2021, https://doi.org/10.3389/fmicb.2021.63069, which is incorporated herein by reference.

The metabolite may be a cecropin, for example selected from cecropin A, cecropin B, CECD, papiliocin, and cecropin P1.

The metabolite may be a moricin, for example selected from moricin, AMP moricin, Px-Mor, Bm-moricin A1 and Bm-moricin2.

The metabolite may be a cupiennin, for example selected from cupiennin 1a, cupiennin 1b, cupiennin 1c, cupiennin 1d, CSTX-3, CSTX-4, CSTX-5, and CSTX-6.

The metabolite may be an oxyopinin, for example selected from oxyopinin 1, oxyopinin 2a, oxyopinin 2b, oxyopinin 2c, oxyopinin 2d and oxyopinin 4a.

The metabolite may be a magainin, for example selected from magainin 1 and magainin 2.

The metabolite may be a dermaseptin, for example selected from DRS 01, DRS A3-A5, DRS B1-B9, DRS C3, DRS CA-1, DRS D12, DRS D14, DRS DA2-DA4, DRS DI1-DI5, DRS DU-1, DRS H1-H6, DRS H8-H10, DRS H12, DRS H13, DRS H15, DRS L1, DRS LI1, DRS PH, DRS PS3, DRS PS4, DRS S1-S8, DRS S9-S13 and DRS TA1.

The metabolite may be a cathelicidin, for example selected from LL-37, RL-37, mCRAMP, rCRAMP, CAP11, eCATH-1, eCATH-2 and eCATH-3, PMAP37, PMAP36 and PMAP23, PR39, PF-1, PF-2, BMAP27, BMAP 28, BMAP34, Bac5, Bac7, indolicidin, bactenecin-1, cathelicidin-4, myeloid cathelicidin, SMAP29, SMAP34, OaBac5, OaBac6, OaBac7.5, OaBac11, BAC5, BAC7.5, MAP34A, MAP346, MAP28, ChBac3.4, CATHL1, 2/CMAP27, Cathelicidin-B1, rtCath1, rtCath2, HFIAP-1, HFIAP-2, HFIAP-3 and Cathelicidin-1.

The metabolite may be a protegrin, for example selected from PG1, PG2, PG3, PG4 and PG5.

The metabolite may be hydramacin-1. The metabolite may be papiliocin. The metabolite may be poneratoxin. The metabolite may be mastoparan. The metabolite may be melittin. The metabolite may be spinigerin.

The stress-inducing parameter may be the presence of second cells which are different to the reference cells.

When the first cells are growing in the presence of other cells, these other cells can secrete molecules which can cause stress to nearby cells (such as the first cells), or even kill them. Thus, a stress-inducing parameter includes secretions from nearby cells which create stress. Any stress-inducing agent described herein, such as a toxin, a metabolite and/or a phage as described herein could be secreted and cause a slowing or halting of the first cells. Thus, in the in vitro test, which seeks to replicate these natural stress scenarios, the addition of any such stress-inducing parameter which results in a slowing or a halt in the growth of the reference cells can be the difference between the standard conditions to the stress conditions.

Equally, when the first cells are growing in the presence of other cells, these other cells can deplete nutrients from the environment, which can cause stress to nearby cells (such as the first cells), or even kill them. Thus, a stress-inducing parameter includes mimicking the effect of nearby cells in reducing nutrients, which creates stress for the first cells. Any nutrient described herein (such as the nutrients described in i. to vi. herein) can be depleted and cause a slowing or halting of the first cells. For example, the second cells could produce sideophores, which scavenges iron from the environment, and deprives the first cells/reference cells of iron, inducing a nutrient stress due to the lack of iron (see, for example, Schiessl et al., “Magnitude and Mechanism of Siderophore-Mediated Competition at Low Iron Solubility in the Pseudomonas aeruginosa Pyochelin System”, Front. Microbiol., 10 Oct. 2017, Sec. Evolutionary and Genomic Microbiology, Volume 8, https://doi.org/10.3389/fmicb.2017.01964, incorporated herein by reference). Another example is wherein the second cells produce a bacteriocin, as described in Frydenlund Michelsen et al., “Staphylococcus aureus Alters Growth Activity, Autolysis, and Antibiotic Tolerance in a Human Host-Adapted Pseudomonas aeruginosa Lineage”, J Bacteriol., 2014 November; 196(22): 3903-3911, doi: 10.1128/JB.02006-14, incorporated herein by reference. Thus, in the in vitro test, which seeks to replicate these natural stress scenarios, the reduction in a nutrient which results in a slowing or a halt in the growth of the reference cells can be the difference between the standard conditions to the stress conditions.

The stress-inducing parameter may the presence of phages that are capable of infecting the reference cells.

The phage may be a tail phage.

The tail phage may be a Faecalibacterium tail phage. Examples of Faecalibacterium tail phages include Faecalibacterium phage FP_Brigit, Faecalibacterium phage FP_Epona, Faecalibacterium phage FP_Lagaffe, Faecalibacterium phage FP_Lugh, Faecalibacterium phage FP_Mushu, Faecalibacterium phage FP_Oengus, Faecalibacterium phage FP_Taranis and Faecalibacterium phage FP_Toutatis.

The tail phage may be a Bacteroides tail phage. Examples of Bacteroides tail phages include Bacteroides phage crAss002, Bacteroides phage crAss001, Bacteroides phage DAC15 and Azobacteroides phage ProJPt-Bpl.

The tail phage may be a Bifidobacterium tail phage. Examples of Bifidobacterium tail phages include Bifidobacterium phage BadAargau2 and Bifidobacterium phage BadAztec1.

The tail phage may be a Clostridium tail phage. Examples of Clostridium tail phages include Clostridium phage CpV1, Clostridium phage CPD2, Clostridium phage CPS1, Clostridium phage phi24R, Clostridium phage phiCP7R, Clostridium phage CPS2, Clostridium phage phiCPV4, Clostridium phage phiZP2, Clostridium phage susfortuna, Clostridium phage phiCD505, Clostridium phage CDKM9, Clostridium phage CDKM15, Clostridium phage phiMMP02, Clostridium phage phiCD111, Clostridium phage phiCD146, Clostridium phage phiCD38-2, Clostridium phage phi CD119, Clostridium phage phiCDHM19, Clostridium phage phiCD506, Clostridium phage phiCD481-1, Clostridium phage phiCDHM11, Clostridium phage phiCDHM13, Clostridium phage phiCDHM14, Clostridium phage phiMMP04, Clostridium phage CDMH1, Clostridium phage phiMMP01, and Clostridium phage phiMMP03.

The tail phage may be a Enterococcus tail phage. Examples of Enterococcus tail phages include Enterococcus phage ECP3, Enterococcus phage EF24C, Enterococcus phage phiEF24C-P2, Enterococcus phage EFLK1, Enterococcus phage EFDG1, Enterococcus phage EFP01, Enterococcus phage EfV12-phi1, Enterococcus phage AE4_17, Enterococcus phage vB_EfaP_Ef6.2, Enterococcus phage vB_EfaP_Ef6.3, Enterococcus phage vB_EfaP_Ef7.2, Enterococcus phage vB_EfaP_Ef7.3, Enterococcus phage vB_EfaP_Ef7.4, Enterococcus phage vB_Efae230P-4, Enterococcus phage vB_EfaP_Efmus1, Enterococcus phage vB_EfaP_Efmus3, Enterococcus phage vB_EfaP_Efmus4, Enterococcus phage Idefix, Enterococcus phage vB_EfaP_IME195, Enterococcus phage vB_EfaP_IME199, Enterococcus phage vB_EfaP_Zip, Enterococcus phage 9183, Enterococcus phage nattely, Enterococcus phage vipetofem, Enterococcus phage vB_EfaS_140, Enterococcus phage vB_EfaS_AL2, Enterococcus phage vB_EfaS_AL3, Enterococcus phage AUEF3, Enterococcus phage Ec-ZZ2, Enterococcus phage IME_EF3, Enterococcus phage IME-EF4, Enterococcus phage EfaCPT1, Enterococcus phage vB_EfaS_IME196, Enterococcus phage LY0322, Enterococcus phage PMBT2, Enterococcus phage SANTOR1, Enterococcus phage phiSHEF2, Enterococcus phage phiSHEF4, Enterococcus phage phiSHEF5, Enterococcus phage phiFL1A, Enterococcus phage phiFL2A, Enterococcus phage phiFL3A, Enterococcus phage BC611, Enterococcus phage IME-EF1 and Enterococcus phage SAP6.

The tail phage may be a Klebsiella tail phage. Examples of Klebsiella tail phages include Klebsiella phage 0507-KN2-1, Klebsiella phage 13, Klebsiella phage 1513, Klebsiella phage 2044-307w, Klebsiella phage 3LV2017, Klebsiella phage 4LV2017, Klebsiella phage AltoGao, Klebsiella phage F19, Klebsiella phage GH-K3, Klebsiella phage GML-KpCol1, Klebsiella phage Henu1, Klebsiella phage JD001, Klebsiella phage JD18, Klebsiella phage JY917, Klebsiella phage K11, Klebsiella phage K5, Klebsiella phage K5-2, Klebsiella phage K5-4, Klebsiella phage K64-1, Klebsiella phage KL, Klebsiella phage KLPN1, Klebsiella phage KN1-1, Klebsiella phage KN3-1, Klebsiella phage KN4-1, Klebsiella phage KNP2, Klebsiella phage KOX1, Klebsiella phage KP-Rio/2015, Klebsiella phage KP15, Klebsiella phage KP179, Klebsiella phage KP1801, Klebsiella phage Kp2, Klebsiella phage KP27, Klebsiella phage KP32, Klebsiella phage KP32_isolate 192, Klebsiella phage KP32_isolate 194, Klebsiella phage KP32_isolate 195, Klebsiella phage KP32_isolate 196, Klebsiella phage KP34, Klebsiella phage KP36, Klebsiella phage KP8, Klebsiella phage KpCHEMY26, Klebsiella phage KpKT21phi1, Klebsiella phage KPN N137, Klebsiella phage KPN N141, Klebsiella phage KpS8, Klebsiella phage kpssk3, Klebsiella phage KPV15, Klebsiella phage KpV41, Klebsiella phage KpV475, Klebsiella phage KpV71, Klebsiella phage KPV811, Klebsiella phage LASTA, Klebsiella phage Magnus, Klebsiella phage Marfa, Klebsiella phage Matisse, Klebsiella phage May, Klebsiella phage Menlow, Klebsiella phage MezzoGao, Klebsiella phage Mineola, Klebsiella phage myPSH1235, Klebsiella phage NJS1, Klebsiella phage NTUH-K2044-K1-1, Klebsiella phage Pharr, Klebsiella phage phiB01E, Klebsiella phage PhiKpNIH-2, Klebsiella phage phiKpS2, Klebsiella phage PKO111, Klebsiella phage PKP126, Klebsiella phage Pylas, Klebsiella phage Seifer, Klebsiella phage SH-Kp 152234, Klebsiella phage SH-Kp 152410, Klebsiella phage Shelby, Klebsiella phage Sin4, Klebsiella phage Skenny, Klebsiella phage Soft, Klebsiella phage SopranoGao, Klebsiella phage ST13-OXA48phi12.1, Klebsiella phage ST147-VIM1phi7.1, Klebsiella phage ST15-OXA48phi14.1, Klebsiella phage ST16-OXA48phi5.4, Klebsiella phage ST437-OXA245phi4.1, Klebsiella phage ST437-OXA245phi4.2, Klebsiella phage ST512-KPC3phi13.2, Klebsiella phage Sugarland, Klebsiella phage Sushi, Klebsiella phage Sweeny, Klebsiella phage TAH8, Klebsiella phage TSK1, Klebsiella phage UPM 2146, Klebsiella phage vB_KIeM_RaK2, Klebsiella phage vB_Kp1, Klebsiella phage vB_Kpn_F48, Klebsiella phage vB_Kpn_IME260, Klebsiella phage vB_KpnM_BIS47, Klebsiella phage vB_KpnM_KB57, Klebsiella phage vB_KpnM_KpS110, Klebsiella phage vB_KpnM_KpV477, Klebsiella phage vB_KpnM_KpV52, Klebsiella phage vB_KpnM_KpV79, Klebsiella phage vB_KpnP_BIS33, Klebsiella phage vB_KpnP_IL33, Klebsiella phage vB_KpnP_IME205, Klebsiella phage vB_KpnP_KpV289, Klebsiella phage vB_KpnP_KpV48, Klebsiella phage vB_KpnP_KpV74, Klebsiella phage vB_KpnP_KpV763, Klebsiella phage vB_KpnP_KpV766, Klebsiella phage vB_KpnP_KpV767, Klebsiella phage vB_KpnP_P184, Klebsiella phage vB_KpnP_PRA33, Klebsiella phage vB_KpnP_SU503, Klebsiella phage vB_KpnP_SU552A, Klebsiella phage vB_KpnS_15-38_KLPPOU149, Klebsiella phage vB_KpnS_Alina, Klebsiella phage vB_KpnS_Call, Klebsiella phage vB_KpnS_Domnhall, Klebsiella phage vB_KpnS_FZ10, Klebsiella phage vB_KpnS_IME279, Klebsiella phage vB_KpnS_IMGroot, Klebsiella phage vB_KpnS_KpV522, Klebsiella phage vB_KpnS_SegesCirculi, Klebsiella phage vB_KpP_FBKp27, Klebsiella phage YMC16/01/N133_KPN_BP, Klebsiella phage ZCKP1, Klebsiella phage_vB_KpnP_IME321 and Klebsiella pneumoniae strain E16KP0115 plasmid unnamed.

The tail phage may be a Fusobacterium tail phage. An example of a Fusobacterium tail phage is Fusobacterium phage FNU1.

The tail phage may be a Lactobacillus tail phage. Examples of Lactobacillus tail phages include Lactobacillus phage 3-521, Lactobacillus phage 521B, Lactobacillus phage A2, Lactobacillus phage ATCC 8014-B1, Lactobacillus phage ATCC 8014-B2, Lactobacillus phage Bacchae, Lactobacillus phage BH1, Lactobacillus phage Bromius, Lactobacillus phage c2, Lactobacillus phage c5, Lactobacillus phage CL1, Lactobacillus phage CL2, Lactobacillus phage iA2, Lactobacillus phage Iacchus, Lactobacillus phage iLp1308, Lactobacillus phage iLp84, Lactobacillus phage J-1, Lactobacillus phage Lb, Lactobacillus phage Lb338-1, Lactobacillus phage LBR48, Lactobacillus phage Lc-Nu, Lactobacillus phage Ld17, Lactobacillus phage Ld25A, Lactobacillus phage Ld3, Lactobacillus phage LdI1, Lactobacillus phage Lenus, Lactobacillus phage LF1, Lactobacillus phage LfeInf, Lactobacillus phage U, Lactobacillus phage LL-Ku, Lactobacillus phage LP65, Lactobacillus phage Lpa804, Lactobacillus phage LpeD, Lactobacillus phage Lrm1, Lactobacillus phage Maenad, Lactobacillus phage Nyseid, Lactobacillus phage P1, Lactobacillus phage PhiAT3, Lactobacillus phage phiJL-1, Lactobacillus phage phiLdb, Lactobacillus phage PL-1, Lactobacillus phage PLE2, Lactobacillus phage PLE3, Lactobacillus phage SA-C12, Lactobacillus phage SAC12B, Lactobacillus phage Satyr, Lactobacillus phage Semele, Lactobacillus phage T25 and Lactobacillus phage ViSo-2018a.

The tail phage may be a Escherichia tail phage. Examples of Escherichia tail phages include Escherichia coli O157 typing phage 3, Escherichia coli strain 13P477T plasmid p13P477T-2, Escherichia phage 121Q, Escherichia phage 13a, Escherichia phage 172-1, Escherichia phage 186, Escherichia phage 1H12, Escherichia phage 285P, Escherichia phage 2B8, Escherichia phage 2G7b, Escherichia phage 2H10, Escherichia phage 4A7, Escherichia phage 4MG, Escherichia phage 500465-1, Escherichia phage 500465-2, Escherichia phage 503458, Escherichia phage 520873, Escherichia phage 64795_ec1, Escherichia phage 933W, Escherichia phage aalborv, Escherichia phage AAPEc6, Escherichia phage aaroes, Escherichia phage ADB-2, Escherichia phage alia, Escherichia phage alpha3, Escherichia phage anhysbys, Escherichia phage AnYang, Escherichia phage APCEc01, Escherichia phage APECc02, Escherichia phage AR1, Escherichia phage Arg0145, Escherichia phage atuna, Escherichia phage Av-05, Escherichia phage BA14, Escherichia phage BF23, Escherichia phage Bp4, Escherichia phage Bp7, Escherichia phage C1, Escherichia phage C119, Escherichia phage C130_2, Escherichia phage C5, Escherichia phage CAjan, Escherichia phage CBA120, Escherichia phage CF2, Escherichia phage chee24, Escherichia phage CICC 80001, Escherichia phage damhaus, Escherichia phage DE3, Escherichia phage DT371/2, Escherichia phage DT37C, Escherichia phage DR, Escherichia phage E21, Escherichia phage e4/1c, Escherichia phage Ebrios, Escherichia phage EC1-UPM, Escherichia phage EC121, Escherichia phage EC6, Escherichia phage EC6098, Escherichia phage ECA2, Escherichia phage ECBP1, Escherichia phage ECBP2, Escherichia phage ECBP5, Escherichia phage ECD7, Escherichia phage ECML-117, Escherichia phage ECML-134, Escherichia phage ECML-4, Escherichia phage EcNP1, Escherichia phage Eco_BIFF, Escherichia phage EC04, Escherichia phage EcoDS1, Escherichia phage ECP1, Escherichia phage EcS1, Escherichia phage EG1, Escherichia phage egaa, Escherichia phage EK99P-1, Escherichia phage EP75, Escherichia phage EPS7, Escherichia phage ESC013, Escherichia phage ESC05, Escherichia phage ESSI2_ev015, Escherichia phage ESSI2_ev040, Escherichia phage ESSI2_ev129, Escherichia phage ESSI2_ev239, Escherichia phage ev017, Escherichia phage ev099, Escherichia phage ev207, Escherichia phage ev243, Escherichia phage F2, Escherichia phage FEC14, Escherichia phage FEC19, Escherichia phage FFH2, Escherichia phage flopper, Escherichia phage fp01, Escherichia phage FV3, Escherichia phage G4, Escherichia phage GA2A, Escherichia phage Gostya9, Escherichia phage grams, Escherichia phage H8, Escherichia phage haarsle, Escherichia phage Henu7, Escherichia phage Henu8, Escherichia phage hemi, Escherichia phage HK022, Escherichia phage HK106, Escherichia phage HK446, Escherichia phage HK542, Escherichia phage HK544, Escherichia phage HK578, Escherichia phage HK629, Escherichia phage HK630, Escherichia phage HK633, Escherichia phage HK75, Escherichia phage HK97, Escherichia phage HP3, Escherichia phage HX01, Escherichia phage HY01, Escherichia phage HY02, Escherichia phage HY03, Escherichia phage HZ2R8, Escherichia phage HZP2, Escherichia phage 122, Escherichia phage ID2 Moscow/ID/2001, Escherichia phage ID21, Escherichia phage ID32, Escherichia phage ID52, Escherichia phage ID62, Escherichia phage If1, Escherichia phage IME08, Escherichia phage ime09, Escherichia phage IME11, Escherichia phage IMM-002, Escherichia phage 38-65, Escherichia phage JES2013, Escherichia phage JH2, Escherichia phage JK06, Escherichia phage JL1, Escherichia phage JLK-2012, Escherichia phage JMPW1, Escherichia phage JMPW2, Escherichia phage JS10, Escherichia phage JS98, Escherichia phage JSE, Escherichia phage K1-5, Escherichia phage K1F, Escherichia phage K1G, Escherichia phage K1H, Escherichia phage Klind1, Escherichia phage Klind2, Escherichia phage K30, Escherichia phage KBNP1711, Escherichia phage KIT03, Escherichia phage Lambda, Escherichia phage LL11, Escherichia phage LL2, Escherichia phage LL5, Escherichia phage LM33_P1, Escherichia phage Lw1, Escherichia phage Lyzl2581Vzw, Escherichia phage M13, Escherichia phage Mangalitsa, Escherichia phage mEp234, Escherichia phage mEpX1, Escherichia phage mEpX2, Escherichia phage Min27, Escherichia phage Minorna, Escherichia phage MLF4, Escherichia phage Mt1B1_P17, Escherichia phage Mu, Escherichia phage Murica, Escherichia phage mutPK1A2, Escherichia phage muut, Escherichia phage MX01, Escherichia phage N15, Escherichia phage N30, Escherichia phage N4, Escherichia phage NC-A, Escherichia phage NC28, Escherichia phage NC29, Escherichia phage NC35, Escherichia phage nepoznato, Escherichia phage nieznany, Escherichia phage NJ01, Escherichia phage OSYSP, Escherichia phage p000v, Escherichia phage P1, Escherichia phage P2, Escherichia phage P2_12H1, Escherichia phage P2_2H4, Escherichia phage P2_4A7b, Escherichia phage P2_482, Escherichia phage P2_4C9, Escherichia phage P2_4E6b, Escherichia phage P483, Escherichia phage P694, Escherichia phage P88, Escherichia phage P88_4811, Escherichia phage PA2, Escherichia phage PA28, Escherichia phage PBECO4, Escherichia phage PE3-1, Escherichia phage PE37, Escherichia phage PGN590, Escherichia phage PGT2, Escherichia phage phAPEC8, Escherichia phage PhaxI, Escherichia phage Phil, Escherichia phage phi191, Escherichia phage phi92, Escherichia phage phiAPCEc03, Escherichia phage phiC120, Escherichia phage phiE142, Escherichia phage phiEB49, Escherichia phage phiEco32, Escherichia phage phiEcoM-GJ1, Escherichia phage phiK, Escherichia phage phiKP26, Escherichia phage phiKT, Escherichia phage phiLLS, Escherichia phage phiSUSP1, Escherichia phage phiSUSP2, Escherichia phage phiV10, Escherichia phage phi174, Escherichia phage phT4A, Escherichia phage Pollock, Escherichia phage PP01, Escherichia phage pro147, Escherichia phage pro483, Escherichia phage PTXU04, Escherichia phage Qbeta, Escherichia phage QL01, Escherichia phage RB14, Escherichia phage RB16, Escherichia phage RB3, Escherichia phage RB32, Escherichia phage RB43, Escherichia phage RB49, Escherichia phage RB69, Escherichia phage RCS47, Escherichia phage Ro45lw, Escherichia phage Rogue1, Escherichia phage Rtp, Escherichia phage saus132, Escherichia phage SECphi27, Escherichia phage Seurat, Escherichia phage SF, Escherichia phage SH2026Stx1, Escherichia phage Skarpretter, Escherichia phage slur02, Escherichia phage slur03, Escherichia phage slur04, Escherichia phage slur07, Escherichia phage slur09, Escherichia phage slur16, Escherichia phage Sortsne, Escherichia phage SP15, Escherichia phage SRT7, Escherichia phage SRT8, Escherichia phage SSL-2009a, Escherichia phage St-1, Escherichia phage ST31, Escherichia phage ST32, Escherichia phage Stx2 II, Escherichia phage T1, Escherichia phage T2, Escherichia phage T4, Escherichia phage T5, Escherichia phage T7, Escherichia phage teqdroes, Escherichia phage teqhad, Escherichia phage teqskov, Escherichia phage tiwna, Escherichia phage TL-2011c, Escherichia phage Tis, Escherichia phage tonijn, Escherichia phage tonn, Escherichia phage tonnikala, Escherichia phage tuntematon, Escherichia phage tunus, Escherichia phage UFV-AREG1, Escherichia phage ukendt, Escherichia phage V18, Escherichia phage V5, Escherichia phage vB_Eco_mar001J1, Escherichia phage vB_Eco_mar003J3, Escherichia phage vB_Eco_mar004NP2, Escherichia phage vB_Eco_SLUR29, Escherichia phage vB_Ec_swan01, Escherichia phage vB_EcoM_005, Escherichia phage vB_EcoM_112, Escherichia phage vB_EcoM_ACG-C40, Escherichia phage vB_EcoM_Alf5, Escherichia phage vB_EcoM_AYO145A, Escherichia phage vB_EcoM_DalCa, Escherichia phage vB_EcoM_EC01230-10, Escherichia phage vB_EcoM_ECOO78, Escherichia phage vB_EcoM_F1, Escherichia phage vB_EcoM_G4498, Escherichia phage vB_EcoM_G4507, Escherichia phage vB_EcoM_G50, Escherichia phage vB_EcoM_G8, Escherichia phage vB_EcoM_G9062, Escherichia phage vB_EcoM_Goslar, Escherichia phage vB_EcoM_IME537, Escherichia phage vB_EcoMJS09, Escherichia phage vB_EcoM_KAW1E185, Escherichia phage vB_EcoM_KWBSE43-6, Escherichia phage vB_EcoM_Lutter, Escherichia phage vB_EcoM_NBG2, Escherichia phage vB_EcoM_0E5505, Escherichia phage vB_EcoM_Ozark, Escherichia phage vB_EcoM_PhAPEC2, Escherichia phage vB_EcoM_PHB05, Escherichia phage vB_EcoM_Schickermooser, Escherichia phage vB_EcoM_VR20, Escherichia phage vB_EcoM_VR25, Escherichia phage vB_EcoM_VR26, Escherichia phage vB_EcoM_VR7, Escherichia phage vB_EcoM_WFC, Escherichia phage vB_EcoM_WFH, Escherichia phage vB_EcoM-12474III, Escherichia phage vB_EcoM-4HA13, Escherichia phage vB_EcoM-ep3, Escherichia phage vB_EcoM-fFiEco06, Escherichia phage vB_EcoM-G28, Escherichia phage vB_EcoM-Ro121c4YLVW, Escherichia phage vB_EcoM-UFV13, Escherichia phage vB_EcoM-VpaE1, Escherichia phage vb_EcoM-VRS, Escherichia phage vB_EcoP_24B, Escherichia phage vB_EcoP_ACG-C91, Escherichia phage vB_EcoP_B, Escherichia phage vB_EcoP_C, Escherichia phage vB_EcoP_F, Escherichia phage vB_EcoP_G7C, Escherichia phage vB_EcoP_K, Escherichia phage vB_EcoP_PhAPECS, Escherichia phage vB_EcoP_PhAPEC7, Escherichia phage vB_EcoP_S523, Escherichia phage vB_EcoP_SU10, Escherichia phage vB_EcoS Sa1791w, Escherichia phage vB_EcoS_ACG-M12, Escherichia phage vB_EcoS_AHP42, Escherichia phage vB_EcoS_AHS24, Escherichia phage vB_EcoS_AKFV33, Escherichia phage vB_EcoS_AKS96, Escherichia phage vB_Ecos_CEB_EC3a, Escherichia phage vB_EcoS_ESC041, Escherichia phage vB_EcoS_FFH_1, Escherichia phage vB_EcoS_G29-2, Escherichia phage vB_EcoS_HdH2, Escherichia phage vB_EcoS_IME347, Escherichia phage vB_EcoS_IME542, Escherichia phage vB_EcoS_NBD2, Escherichia phage vB_EcoS_PHB17, Escherichia phage vB_EcoS_SH2, Escherichia phage vB_EcoS_swi2, Escherichia phage vB_EcoS_W011D, Escherichia phage vB_EcoS-122101, Escherichia phage vB_EcoS-95, Escherichia phage vB_EcoS-DELF2, Escherichia phage vB_EcoS-Golestan, Escherichia phage vB_EcoS-1ME253, Escherichia phage vB_vPM_PD06, Escherichia phage vB_vPM_PD112, Escherichia phage VEc3, Escherichia phage VEc33, Escherichia phage VEcB, Escherichia phage vojen, Escherichia phage WA45, Escherichia phage WG01, Escherichia phage Wphi, Escherichia phage wV7, Escherichia phage wV8, Escherichia phage YD-2008.s, Escherichia phage YUEEL01, Escherichia phage YZ1, Escherichia phage ZG49, Escherichia phage_Lidtsur, Escherichia phage_Vec13, Escherichia typing phage 1 and Escherichia virus KFS-EC.

The tail phage may be a Streptococcus tail phage. Examples of Streptococcus tail phages include Streptococcus phage 01205, Streptococcus phage 2972, Streptococcus phage 7201, Streptococcus phage 858, Streptococcus phage Abc2, Streptococcus phage ALQ13.2, Streptococcus phage C1, Streptococcus phage Cp-7, Streptococcus phage Cp1, Streptococcus phage DT1, Streptococcus phage Sfi11, Streptococcus phage Sfi19, Streptococcus phage Sfi21 and Streptococcus phage SP-QS1.

The tail phage may be a Providencia tail phage. Examples of Providencia tail phages include Providencia phage Kokobel1, Providencia phage PSTCR5, Providencia phage Redjac, Providencia phage vB_PreS_PR1, Providencia phage vB_PreS-PibeRecoleta, Providencia phage vB_PreS-Stilesk and Providencia phage vB_PstP_PS3.

The tail phage may be a Salmonella tail phage. Examples of Salmonella tail phages include

The tail phage may be a Helicobacter tail phage. Examples of Heliobacter tail phages include Helicobacter phage 1961P, Helicobacter phage KHP30 and Helicobacter phage KHP40.

The tail phage may be a Shella tail phage. Examples of Shigella tail phages include Shigella phage 2019SD1, Shigella phage 75/02 Stx, Shigella phage CM8, Shigella phage DS8, Shigella phage EP23, Shigella phage JK16, Shigella phage JK45, Shigella phage KNP5, Shigella phage MK-13, Shigella phage phi25-307, Shigella phage phiSboM-AG3, Shigella phage POCJ13, Shigella phage pSb-1, Shigella phage pSf-1, Shigella phage pSf-2, Shigella phage pSs-1, Shigella phage Sd1, Shigella phage Sf11 SMD-2017, Shigella phage Sf12, Shigella phage Sf13, Shigella phage Sf14, Shigella phage Sf17, Shigella phage Sf21, Shigella phage Sf22, Shigella phage Sf23, Shigella phage Sf24, Shigella phage Sf6, Shigella phage Sfin-1, Shigella phage Sfin-3, Shigella phage SfMu, Shigella phage SFN6B, Shigella phage SFPH2, Shigella phage SH6, Shigella phage SH7, Shigella phage SHBML-50-1, Shigella phage SHBML-50-1, Shigella phage Shf125875, Shigella phage Shfl1, Shigella phage Shfi2, Shigella phage SHFML-11, Shigella phage SHFML-26, Shigella phage SHSML-45, Shigella phage SHSML-52-1, Shigella phage SP18, Shigella phage Ss-VASD, Shigella phage SSP1, Shigella phage vB_SfiS-ISF001, Shigella phage vB_ShiP_A7, Shigella phage vB_SsoS_008, Shigella phage vB_SsoS-ISF002 and Shigella phage_Buco.

The tail phage may be a Pseudomonas tail phage. Examples of Pseudomonas tail phages include Pseudomonas aeruginosa PS75 adTyT-supercont1.7, Pseudomonas phage 119X, Pseudomonas phage 14-1, Pseudomonas phage 17A, Pseudomonas phage 22PfluR64PP, Pseudomonas phage 73, Pseudomonas phage Achelous, Pseudomonas phage Alpheus, Pseudomonas phage Andromeda, Pseudomonas phage antinowhere, Pseudomonas phage B3, Pseudomonas phage Bf7, Pseudomonas phage Bjorn, Pseudomonas phage BrSP1, Pseudomonas phage crassa, Pseudomonas phage D3, Pseudomonas phage D3112, Pseudomonas phage datas, Pseudomonas phage DL60, Pseudomonas phage DL62, Pseudomonas phage DL68, Pseudomonas phage DMS3, Pseudomonas phage Dobby, Pseudomonas phage EL, Pseudomonas phage Epa14, Pseudomonas phage Epa5, Pseudomonas phage EPa61, Pseudomonas phage Epa7, Pseudomonas phage F_HA0480sp/Pa1651, Pseudomonas phage F116, Pseudomonas phage F8, Pseudomonas phage gh-1, Pseudomonas phage H66, Pseudomonas phage Henninger, Pseudomonas phage inbricus, Pseudomonas phage JBD67, Pseudomonas phage JD18, Pseudomonas phage JG004, Pseudomonas phage JG024, Pseudomonas phage KNP, Pseudomonas phage KPP10, Pseudomonas phage KPP12, Pseudomonas phage KPP21, Pseudomonas phage KPP25, Pseudomonas phage Lana, Pseudomonas phage LBL3, Pseudomonas phage LIT1, Pseudomonas phage Littiefix, Pseudomonas phage LKA1, Pseudomonas phage LKD16, Pseudomonas phage LKO4, Pseudomonas phage LMA2, Pseudomonas phage LPB1, Pseudomonas phage LUZ19, Pseudomonas phage LUZ24, Pseudomonas phage LUZ7, Pseudomonas phage M6, Pseudomonas phage MP1412, Pseudomonas phage MP22, Pseudomonas phage MP29, Pseudomonas phage MP38, Pseudomonas phage MPK6, Pseudomonas phage MPK7Pseudomonas phage Nerthus, Pseudomonas phage NH-4, Pseudomonas phage nickie, Pseudomonas phage Njord, Pseudomonas phage Noxifer, Pseudomonas phage NP1, Pseudomonas phage NV1, Pseudomonas phage PA01, Pseudomonas phage PA1/KOR/2010, Pseudomonas phage PA10, Pseudomonas phage PA11, Pseudomonas phage PA26, Pseudomonas phage PA5, Pseudomonas phage PA7, Pseudomonas phage PA8P1, Pseudomonas phage PaBG, Pseudomonas phage PAE1, Pseudomonas phage PaGU11, Pseudomonas phage PAK_P1, Pseudomonas phage PAK_P2, Pseudomonas phage PAK_P3, Pseudomonas phage PAK_P4, Pseudomonas phage PaMx11, Pseudomonas phage PaMx25, Pseudomonas phage PaMx28, Pseudomonas phage PaMx41, Pseudomonas phage PaMx74, Pseudomonas phage PaP1, Pseudomonas phage PaP3, Pseudomonas phage PaP4, Pseudomonas phage PAXYB1, Pseudomonas phage PB1, Pseudomonas phage Persinger, Pseudomonas phage Pf-10, Pseudomonas phage Pf1, Pseudomonas phage Pf1 ERZ-2017, Pseudomonas phage pf16, Pseudomonas phage Pf3, Pseudomonas phage Pf3, Pseudomonas phage pf8_ST274-AUS411, Pseudomonas phage PFP1, Pseudomonas phage phCDa, Pseudomonas phage phi-2, Pseudomonas phage Phi-S1, Pseudomonas phage phi12, Pseudomonas phage phi13, Pseudomonas phage phi15, Pseudomonas phage phi2954, Pseudomonas phage phi3, Pseudomonas phage phi6, Pseudomonas phage phi8, Pseudomonas phage PhiCHU, Pseudomonas phage phiCTX, Pseudomonas phage phiIBB-PAA2, Pseudomonas phage phiIBB-PF7A, Pseudomonas phage phikF77, Pseudomonas phage phiKMV, Pseudomonas phage phiKTN6, Pseudomonas phage phiKZ, Pseudomonas phage phiMK, Pseudomonas phage phiNFS, Pseudomonas phage phiNN, Pseudomonas phage phiNV3, Pseudomonas phage phiPMW, Pseudomonas phage phiPsa17, Pseudomonas phage phiPSA2, Pseudomonas phage phiPsa374, Pseudomonas phage PhiR18, Pseudomonas phage phiYY, Pseudomonas phage PMBT14, Pseudomonas phage PMBT3, Pseudomonas phage PMG1, Pseudomonas phage PollyC, Pseudomonas phage PP7, Pseudomonas phage PPPL-1, Pseudomonas phage PPpW-4, Pseudomonas phage PRR1, Pseudomonas phage PspYZU01, Pseudomonas phage PspYZU05, Pseudomonas phage PspYZU08, Pseudomonas phage PT2, Pseudomonas phage PT5, Pseudomonas phage R12, Pseudomonas phage R26, Pseudomonas phage RLP, Pseudomonas phage sh12, Pseudomonas phage SL1, Pseudomonas phage SL2, Pseudomonas phage SM1, Pseudomonas phage SN, Pseudomonas phage tabemarius, Pseudomonas phage tf, Pseudomonas phage T., Pseudomonas phage UFV-P2, Pseudomonas phage uligo, Pseudomonas phage UNO-SLW1, Pseudomonas phage vB_Pae_BR319a, Pseudomonas phage vB_Pae_PS44, Pseudomonas phage vB_Pae-Kakheti25, Pseudomonas phage vB_Pae-SS2019XI, Pseudomonas phage vB_PaeM_C1-14_Ab28, Pseudomonas phage vB_PaeM_C2-10_Ab02, Pseudomonas phage vB_PaeM_C2-10_Abl, Pseudomonas phage vB_PaeM_CEB_DP1, Pseudomonas phage vB_PaeM_E215, Pseudomonas phage vB_PaeM_E217, Pseudomonas phage vB_PaeM_G1, Pseudomonas phage vB_PaeM_LS1, Pseudomonas phage vB_PaeM_MAG1, Pseudomonas phage vB_PaeM_PA01_Ab03, Pseudomonas phage vB_PaeM_PS24, Pseudomonas phage vB_PaeM_SCUT-S1, Pseudomonas phage vB_PaeM_SCUT-SI, Pseudomonas phage vB_PaeM_USP_1, Pseudomonas phage vB_PaeP_130_113, Pseudomonas phage vB_PaeP_C2-10_Ab09, Pseudomonas phage vB_PaeP_C2-10_Ab22, Pseudomonas phage vB_PaeP_PA01_1-15pyo, Pseudomonas phage vB_PaeP_PA01_Ab05, Pseudomonas phage vB_PaeP_PPA-ABTNL, Pseudomonas phage vB_PaeS_PA01_Ab18, Pseudomonas phage vB_PaeS_PA01_Ab19, Pseudomonas phage vB_PaeS_PM105, Pseudomonas phage vB_PaeS_SCH_Ab26, Pseudomonas phage vB_PsyM_KIL1, Pseudomonas phage vB_PsyM_KIL4, Pseudomonas phage vB_PsyP_3MF5, Pseudomonas phage VCM, Pseudomonas phage VSW-3, Pseudomonas phage WRT, Pseudomonas phage YMC11/06/C171_PPU_BP, Pseudomonas phage YuA, Pseudomonas phage ZC01, Pseudomonas phage ZC03, Pseudomonas phage ZC08, Pseudomonas phage Zigelbrucke, Pseudomonas phage Zuri, Pseudomonas sp. S4_EA_1b YA0848_38 and Pseudomonas virus Pa193.

The tail phage may be a Staphylococcus tail phage. Examples of Staphylococcus tail phages include Staphylococcus phage 187, Staphylococcus phage 23MRA, Staphylococcus phage 2638A, Staphylococcus phage 29, Staphylococcus phage 3 A-2017, Staphylococcus phage 37, Staphylococcus phage 3A, Staphylococcus phage 3MRA, Staphylococcus phage 42e, Staphylococcus phage 47, Staphylococcus phage 52A, Staphylococcus phage 53, Staphylococcus phage 55, Staphylococcus phage 676Z, Staphylococcus phage 69, Staphylococcus phage 6ec, Staphylococcus phage 71, Staphylococcus phage 77, Staphylococcus phage 80, Staphylococcus phage 80alpha, Staphylococcus phage 85, Staphylococcus phage 88, Staphylococcus phage 92, Staphylococcus phage 96, Staphylococcus phage A3R, Staphylococcus phage A5W, Staphylococcus phage Andhra, Staphylococcus phage B122, Staphylococcus phage B166, Staphylococcus phage B236, Staphylococcus phage Baq_Sau1, Staphylococcus phage BP39, Staphylococcus phage CNPH82, Staphylococcus phage CNPx, Staphylococcus phage CSA13, Staphylococcus phage EW, Staphylococcus phage Fi200W, Staphylococcus phage G1, Staphylococcus phage G15, Staphylococcus phage GRCS, Staphylococcus phage Henu2, Staphylococcus phage HSA84, Staphylococcus phage IME-SA1, Staphylococcus phage IME-SA118, Staphylococcus phage IME-SA119, Staphylococcus phage IME-SA2, Staphylococcus phage IME1348_01, Staphylococcus phage IME1361_01, Staphylococcus phage ISP, Staphylococcus phage JD007, Staphylococcus phage JS01, Staphylococcus phage K, Staphylococcus phage LH1, Staphylococcus phage LSA2366, Staphylococcus phage MCE-2014, Staphylococcus phage MSA6, Staphylococcus phage P108, Staphylococcus phage P1105, Staphylococcus phage P240, Staphylococcus phage P282, Staphylococcus phage P4W, Staphylococcus phage P630, Staphylococcus phage P954, Staphylococcus phage Pabna, Staphylococcus phage PH15, Staphylococcus phage phi 11, Staphylococcus phage phi 12, Staphylococcus phage phi 13, Staphylococcus phage phi2958PVL, Staphylococcus phage phi44AHJD, Staphylococcus phage phi7247PVL, Staphylococcus phage phiBU01, Staphylococcus phage phiETA, Staphylococcus phage phiETA2, Staphylococcus phage phiETA3, Staphylococcus phage phiIBB-SEP1, Staphylococcus phage phiIPLA-RODI, Staphylococcus phage phiJB, Staphylococcus phage phiMR11, Staphylococcus phage phiMR25, Staphylococcus phage phiNM1, Staphylococcus phage phiNM2, Staphylococcus phage phiNM3, Staphylococcus phage phiNM4, Staphylococcus phage phiPVL-CN125, Staphylococcus phage phiPVL108, Staphylococcus phage phiSA_BS1, Staphylococcus phage phiSA_BS2, Staphylococcus phage phiSA12, Staphylococcus phage phiSa2wa_st1, Staphylococcus phage phiSa2wa_st121mssa, Staphylococcus phage phiSa2wa_st22, Staphylococcus phage phiSa2wa_st30, Staphylococcus phage phiSa2wa_st5, Staphylococcus phage phiSa2wa_st78, Staphylococcus phage phiSauS-IPLA35, Staphylococcus phage phiSLT, Staphylococcus phage phiSP44-1, Staphylococcus phage Portland, Staphylococcus phage Portland, Staphylococcus phage PSa3, Staphylococcus phage PVL, Staphylococcus phage ROSA, Staphylococcus phage S24-1 DNA, Staphylococcus phage S25-3, Staphylococcus phage S25-4, Staphylococcus phage SA1014ruMSSAST7, Staphylococcus phage SA11, Staphylococcus phage SA13, Staphylococcus phage SA137ruMSSAST121PVL, Staphylococcus phage SA345ruMSSAST8, Staphylococcus phage SA46-CL1, Staphylococcus phage SAS, Staphylococcus phage SA7, Staphylococcus phage SA75, Staphylococcus phage SA780ruMSSAST101, Staphylococcus phage SA97, Staphylococcus phage SAP-2, Staphylococcus phage SAP-26, Staphylococcus phage SAP11, Staphylococcus phage SAP33, Staphylococcus phage Sb-1, Staphylococcus phage SCH1, Staphylococcus phage Sebago, Staphylococcus phage SH-St 15644, Staphylococcus phage SLPW, Staphylococcus phage SN8, Staphylococcus phage SP120, Staphylococcus phage SP197, Staphylococcus phage SP276, Staphylococcus phage SP5, Staphylococcus phage SpT152, Staphylococcus phage St 134, Staphylococcus phage StaphiN, Staphylococcus phage Stau2, Staphylococcus phage StauST398-1, Staphylococcus phage StauST398-2, Staphylococcus phage StauST398-3, Staphylococcus phage StauST398-4, Staphylococcus phage StauST398-5, Staphylococcus phage Team1, Staphylococcus phage TEM126, Staphylococcus phage tp310-1, Staphylococcus phage tp310-2, Staphylococcus phage Twort, Staphylococcus phage UPMK_2, Staphylococcus phage vB_SauM_Remus, Staphylococcus phage vB_SauM_Romulus, Staphylococcus phage vB_SauP_phiAGO1.3, Staphylococcus phage vB_SauS_fPfSau02, Staphylococcus phage vB_SauS_JS02, Staphylococcus phage vB_SauS_phi2, Staphylococcus phage vB_SauS-phiIPLA88, Staphylococcus phage vB_SauS-SAP27, Staphylococcus phage vB_SepiS-phiIPLA5, Staphylococcus phage vB_SepiS-phiIPLA7, Staphylococcus phage vB_SepM_phiIPLA-CiC, Staphylococcus phage vB_SepS_SEP9, Staphylococcus phage vB_SpsM_WIS42, Staphylococcus phage vB_SpsS_QT1, Staphylococcus phage vB_SscM-1, Staphylococcus phage X2 and Staphylococcus phage YMC/09/04/R1988.

The tail phage may be a Clostridioides tail phage. Examples of Clostridioides tail phages include Clostridioides phage phiC2 and Clostridioides phage phiCD27.

The tail phage may be a Acinetobacter tail phage. Examples of Acinetobacter tail phages include Acinetobacter phage 133, Acinetobacter phage AB1, Acinetobacter phage AB3, Acinetobacter phage Abp1, Acinetobacter phage AbP2, Acinetobacter phage AbTZA1, Acinetobacter phage Acj61, Acinetobacter phage Acj9, Acinetobacter phage AM101, Acinetobacter phage AP205, Acinetobacter phage AP22, Acinetobacter phage Fri1, Acinetobacter phage IME-200, Acinetobacter phage IMEAB3, Acinetobacter phage KARL-1, Acinetobacter phage Loki, Acinetobacter phage LZ35, Acinetobacter phage Petty, Acinetobacter phage phiAB1, Acinetobacter phage phiAb6, Acinetobacter phage Presley, Acinetobacter phage SH-Ab 15519, Acinetobacter phage SWH-Ab-1, Acinetobacter phage SWH-Ab-3, Acinetobacter phage vB_AbaM_Apostate, Acinetobacter phage vB_AbaM_B09_Aci01-1, Acinetobacter phage vB_AbaM_B09_Aci02-2, Acinetobacter phage vB_AbaM_B09_Aci05, Acinetobacter phage vB_AbaM_Berthold, Acinetobacter phage vB_AbaM_Kimel, Acinetobacter phage vB_AbaM_Konradin, Acinetobacter phage vB_AbaM_Lazarus, Acinetobacter phage vB_AbaM_ME3, Acinetobacter phage vB_AbaM_PhT2, Acinetobacter phage vB_AbaM-IME-AB2, Acinetobacter phage vB_AbaP_Acibel007, Acinetobacter phage vB_AbaP_AS11, Acinetobacter phage vB_AbaP_AS12, Acinetobacter phage vB_AbaP_B1, Acinetobacter phage vB_AbaP_1B3, Acinetobacter phage vB_AbaP_B5, Acinetobacter phage vB_AbaP_D2, Acinetobacter phage vB_AbaP_PD-6A3, Acinetobacter phage vB_AbaP_PD-AB9, Acinetobacter phage vB_ApiM_fHyAci03, Acinetobacter phage vB_ApiP_P1, Acinetobacter phage vB_ApiP_P2, Acinetobacter phage VB_ApiP_XC38, Acinetobacter phage WCHABP1, Acinetobacter phage WCHABP12, Acinetobacter phage WCHABP5, Acinetobacter phage YMC-13-01-C62, Acinetobacter phage YMC/09/02/B1251, Acinetobacter phage YMC11/11/R3177, Acinetobacter phage ZZ1, Acinetobacter phage_AbKT21phiIII, Acinetobacter phage_vB_AbaP_46-62_Aci07 and Acinetobacter phage_vB_AbaP_B09_Aci08.

Any of the tail phages described herein may be used to deliver the vector to the first cells. Where a tail phage is used to deliver the vector to the first cells, the first cells are of a species that is capable of being infected by the phage.

The phage may be a filamentous phage.

The filamentous phage may be a Vibrio filamentous phage. Examples of Vibrio filamentous phages include Vibrio phage CTXphi, Vibrio phage pre-CTX, Vibrio phage KSF1, Vibrio phage fs1, Vibrio phage ND1-fs1, Vibrio phage VEJ, Vibrio phage VGJ, Vibrio phage VP24-2_Ke, Vibrio phage VSK, Vibrio phage VSKK, Vibrio phage fs2, Vibrio phage VFJ, Vibrio phage VAIR, Vibrio phage VALG_phi6, Vibrio phage VfO3K6, Vibrio phage VfO4K68, Vibrio phage VCY, Vibrio phage VALG_phi8, Vibrio phage Vf12, Vibrio phage Vf33 and Vibrio phage XacF13.

The filamentous phage may be a Xanthomonas filamentous phage. Examples of Xanthomonas filamentous phages include Xanthomonas phage Cf1c, Xanthomonas phage XacF1, Xanthomonas phage Cf2, Xanthomonas phage phi Lf2, Xanthomonas phage phiLf UK, Xanthomonas phage phiXv2, Xanthomonas phage Xf109 and Xanthomonas phage Xf409.

The filamentous phage may be a Ralstonia filamentous phage. Examples of Ralstonia filamentous phages include Ralstonia phage Rs551, Ralstonia phage RS603, Ralstonia phage RSIBR3, Ralstonia phage RSM1, Ralstonia phage RSM3, Ralstonia phage RSMSuper, Ralstonia phage PE226, Ralstonia phage p12J, Ralstonia phage RS611, Ralstonia phage RSBg, Ralstonia phage RSS-TH1, Ralstonia phage RSS0 and Ralstonia phage RSS1.

The filamentous phage may be an Escherichia filamentous phage. Examples of Escherichia filamentous phages include Escherichia phage If1, Escherichia phage fd and Escherichia phage 122.

The filamentous phage may be an Enterobacteria filamentous phage. Examples of Enterobacteria filamentous phages include Enterobacteria phage f1 and Enterobacteria phage M13.

The filamentous phage may be an Erwinia filamentous phage. An example of an Erwinia filamentous phage includes Erwinia phage PEar6.

The filamentous phage may be a Pseudomonas filamentous phage. Examples of Pseudomonas filamentous phages include Pseudomonas phage Pf1, Pseudomonas phage pf8_ST274-AUS411 and Pseudomonas phage Pf3

The filamentous phage may be a Salmonella filamentous phage. An example of an Salmonella filamentous phage includes Salmonella phage IKe.

The filamentous phage may be a Stenotrophomonas filamentous phage. Examples of Stenotrophomonas filamentous phages include Stenotrophomonas phage PSH1, Stenotrophomonas phage SMA6, Stenotrophomonas phage phi SHP2, Stenotrophomonas phage SMA9 and Stenotrophomonas phage SMA7.

Any of the filamentous phages described herein may be used to deliver the vector to the first cells. Where a filamentous phage is used to deliver the vector to the first cells, the first cells are of a species that is capable of being infected by the phage.

The stress-inducing parameter may be the presence of free radicals. The level of free radicals can be increased by the addition of certain molecules, for example paraquat, menadione, hydrogen peroxide, transition metals (iron, copper, and chromium) in concentrations which will be readily apparent to the skilled person.

When the reference cells are anaerobes, the stress-inducing parameter may be the presence of oxygen, for example achieved by shaking to aerate the reference cell culture. The conditions for shaking may be any disclosed herein.

The stress-inducing parameter may also be a change in pH from a pH that the reference cells grow well in, to one which induces stress. Many cells grow in neutral to acidic conditions, so for many reference cells an alkaline pH would induce stress. Equally, many cells grow in acidic conditions, (e.g. between 2.0 and 3.0). For these cells, stress could be induced at pHs above ˜4.0. For cells which grow at a neutral pH (e.g. between 6.5 and 7.5), stress could be induced at a pH lower than ˜5.5 or at a pH higher than ˜8.5. The change in pH can be outside any of the ranges given for the native environments as described elsewhere herein.

The stress-inducing parameter may be a seryl-tRNA synthetase inhibitor, such as serine hydroxamate (SHX).

### Measuring SPA Promoter Activity

Activity of the SPA promoter may be activity for the production of transcripts from a nucleotide sequence that is under the control of the SPA promoter and/or the production of an expression protein (such as a reporter molecule) encoded by a nucleotide sequence (such as a reporter gene) that is under the control of the SPA promoter.

When tested under standard conditions and under stress conditions, the SPA promoter shows at least the same activity in the stress conditions compared to the standard conditions.

In one embodiment, when tested under standard conditions and under stress conditions, the SPA promoter shows an increase in activity in the stress conditions compared to the standard conditions. In one embodiment, when tested under standard conditions and under stress conditions, the SPA promoter shows a statistically significant increase in activity in the stress conditions compared to the standard conditions.

In one embodiment, when tested under standard conditions and under stress conditions, the SPA promoter shows at least a 2-fold increase in activity in the stress conditions compared to the standard conditions. The fold increase may be at least 3-fold, at least 4-fold, at least 5-fold. The fold increase may be a 10-fold increase. The fold increase may be a 20-fold increase. The fold increase may be a 50-fold increase.

In another embodiment, when tested under standard conditions and under stress conditions, the SPA promoter may show no activity in the standard conditions, but show activity in the stress conditions.

SPA promoter activity may be determined by measuring total mRNA levels and comparing these levels to the mRNA levels of the transcripts under the control of the SPA in both the standard conditions and the stress conditions. Thus, the SPA promoter activity in the standard conditions may be determined by:
(1) measuring the total mRNA levels in the reference cells,
(2) measuring the mRNA levels transcribed from the SPA promoter in the reference cells, and
(3) comparing the ratio of (2) to (1), and
   the promoter activity when tested in the stress conditions is determined by
(4) measuring the total mRNA levels in the reference cells,
(5) measuring the mRNA levels transcribed from the SPA promoter in the reference cells, and
(6) comparing the ratio of (5) to (4).

The mRNA levels of (1), (2), (4) and (5) may be measured by RNAseq. When the mRNA levels of (1), (2), (4) and (5) are measured by RNAseq, the mRNA levels of (1) and (4) are the number of reads for the total mRNA and the mRNA levels of (2) and (5) are the number of reads mapping to the transcript from the SPA promoter. The total mRNA reads of (1) and (4) may be normalised to the length of the reads mapping to the transcript from the SPA promoter.

Using this method, the SPA promoter activity may be considered the same when the ratio of (6) and the ratio of (3) are within statistical error of each other.

Using this method, the SPA promoter activity may considered an increase when the ratio of (6) is at least 2-fold the ratio of (3). The ratio of (6) may be at least 3-fold the ratio of (3). The ratio of (6) may be at least 4-fold the ratio of (3). The ratio of (6) may be at least 5-fold the ratio of (3).

The ratio of (6) may be at least 10-fold the ratio of (3). The ratio of (6) may be at least 20-fold the ratio of (3). The ratio of (6) may be at least 50-fold the ratio of (3).

Where it is said that “the SPA promoter activity in the standard conditions may be determined by”, it is meant that the activity of the SPA promoter is capable of being determined in the described manner, if and when tested, rather than the activity of the SPA promoter is actively being determined by the stated method.

Alternatively, the SPA promoter activity can be determined by fusing the promoter to a reporter gene encoding any of the reporter molecules described herein. In a particular embodiment, the reporter gene is selected from lacZ, a fluorescent protein (e.g. green fluorescent protein, GFP) and a luminescent protein (e.g. Lux proteins). Thus, the SPA promoter activity in the standard conditions and the stress conditions may be determined by:
(7) fusing the SPA promoter to a reporter gene,
(8) measuring output of the reporter gene under the control of the SPA promoter under the standard conditions,
(9) measuring output of the reporter gene under the control of the SPA promoter under the stress conditions, and
(10) determining the ratio of (9) to (8).

### Measuring Growth Rates

For a background on bacterial growth rates, see Todar's Online textbook of Bacteriology, www.textbookofbacteriology.net, Chapter entitled: “The Growth of Bacterial Populations”.

The growth rate and how to determine growth rate of cells, e.g. by determining the rate (such as doubling time) using a cell sample in vitro, will be readily apparent to the skilled person. For example, if the average doubling time for cells of the first species or strain (e.g. as determined to be the average doubling rate of said cells comprised by a cell sample in vitro) may be determined for cells under the standard conditions and separately for cells growing under stress conditions, such as cells comprising an alarmone (optionally one or both of alarmones ppGpp and pppGpp); or cells which are stationary phase cells, stressed cells, persister cells, biofilm cells or dormant cells. By comparing the determined growth rates of the reference cells under the standard conditions and the cells growing under stress conditions, one can determine that the stressed cells have a growth rate that is less than (e.g. either statistically significantly lower, or less than 50% of) the growth rate of the cells growing under standard conditions.

Thus, the growth rate in (a) and (b) herein can be determined by measuring the doubling time of the reference cells. The skilled person will be familiar with determining doubling times for cell populations. For example, the doubling time for the reference cells growing under stress conditions is at least 2× (e.g. at least 2, 2.5, 3, 3.5, 4, 4.5, 5, 5.5, 6, 6.5, 7, 7.5, 8, 8.5, 9, 9.5 or 10×) the doubling time for the reference cells growing under the standard conditions. For example, the doubling time for the reference cells under standard conditions may be 20 minutes (i.e. the number of cells in the sample has doubled in 20 minutes), whereas the doubling time for the reference cells growing under the stress conditions would be at least 40 minutes (e.g. ˜1 hour).

The growth rate can be considered reduced if the doubling time of the reference cells is statistically significantly lower in the stress conditions as compared to the standard conditions. The growth rate can be reduced by at least 50% in the stress conditions as compared to the standard conditions. The growth rate can be reduced by at least 20%, 30%, 40% or 45% in the stress conditions as compared to the standard conditions

The growth rate can be reduced by at least 55%, 60%, 65%, 70%, 75%, 80%, 85%, 90% or 95% in the stress conditions as compared to the standard conditions. In another embodiment; there is no observed doubling of the reference cells in the stress conditions.

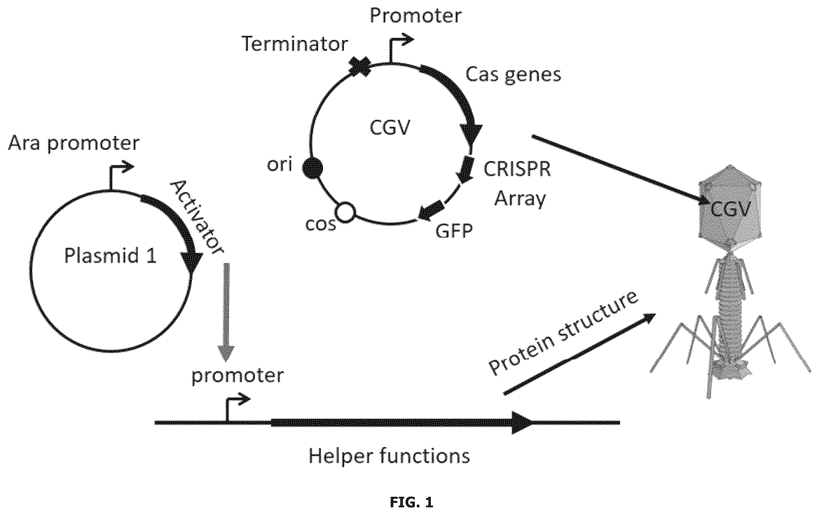

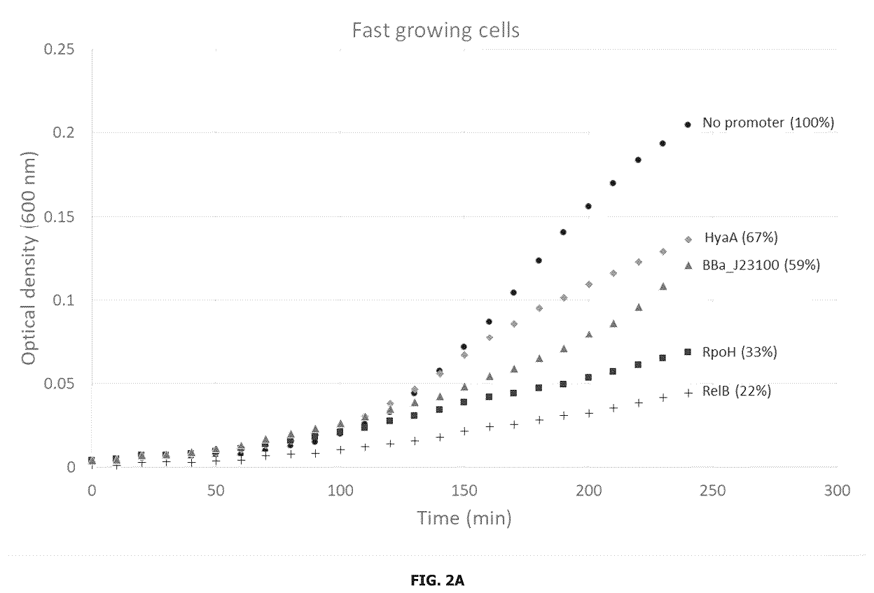

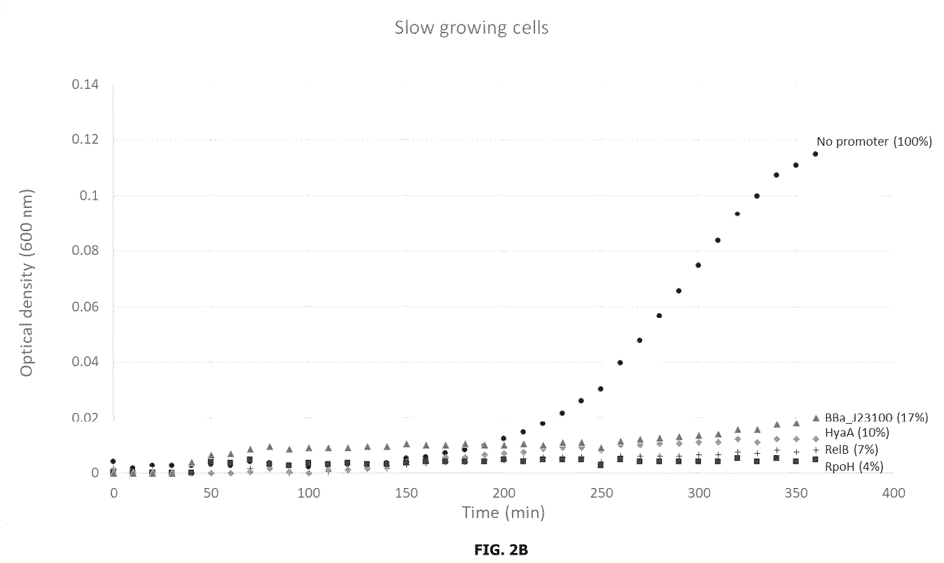

In an alternative method to observing the doubling time of the reference cells, for example in certain circumstances where a doubling time cannot be readily determined, the growth rate can be estimated by approximating the ribosomal protein fraction of cellular proteins. Because there is a linear conversion between the RNA weight/protein weight ratios and ribosomal protein weight/total protein weight ratios, comparing total RNA weight to total protein weight for a given number of cells under different conditions can provide information on relative growth rates of the cells, see Scott et al., “Interdependence of Cell Growth and Gene Expression: Origins and Consequences”, Science, Vol 330, 19 Nov. 2010, doi: 10.1126/science.1192588 (which is incorporated herein by reference), see in particular FIG. 1A. Also see Sauls et al., “Control of Bacillus subtilis Replication Initiation during Physiological Transitions and Perturbations”, 2019, mBio 10:e02205-19, https://doi.org/10.1128/mBio.02205-19 (which is incorporated herein by reference), in particular FIG. 2A which shows a strong correlation between cell size and growth rate in both gram positive and gram negative bacteria.

Thus, in one embodiment, the growth rate under standard conditions can be determined by:
(11) determining total protein weight for a given number of reference cells;
(12) determining total RNA weight for a given number of reference cells;
(13) providing a ratio of (12) to (11); and
the growth rate under stress conditions can be determined by:
(14) determining total protein weight for a given number of reference cells;
(15) determining total RNA weight for a given number of reference cells; and
(16) providing a ratio of (15) to (14); then
comparing the ratio of (13) to the ratio of (16), and if the ratio of (16) is lower than the ratio of (13), then the reference cells are growing slower under the stress conditions than the standard conditions.

The given number of cells in (11), (12), (14) and (15) can be determined by serially diluting the reference cells and determining CFU. The reference cells may be plated onto agar plates containing the growth nutrients to determine the CFU. If the given number of cells in each or any of (11), (12), (14) and (15) are not the same, then the number of given cells to the respective weights can be normalised for the same number of cells.

The total protein concentration in a known number of cells (e.g. in step (11) and/or step (14) can be measured for example by the Total Protein Kit (Sigma, TP0300).

The total RNA concentration in a known number of cells (e.g. in step (12) and/or step (15) can be measured for example by the TRIzol method (Invitrogen).

The ratio of (16) can be statistically significantly lower than the ratio of (13). The ratio of (16) can be at least 50% lower than the ratio of (13). The ratio of (16) can be at least 20%, 30%, 40% or 45% lower than the ratio of (13).

The ratio of (16) can be at least 55%, 60%, 65%, 70%, 75%, 80%, 85%, 90% or 95% lower than the ratio of (13).

### SPA Promoters

The inventors have realised that certain promoters that are still active whilst their host cells are experiencing high levels of stress which severely restricts their growth, or puts them into a dormant state can be harnessed to facilitate production of molecules in these stressed cells. Targeting such cells is known to be difficult, and many of them are resistant to antibiotics. These promoters are “stress-phase active” (SPA) promoters.

A person skilled in the art can determine the sequence of the promoters described herein as well as for newly-discovered promoters. For known promoters, the transcription start site (+1) that has been determined will be available to the skilled person. Typically, the RNA polymerase covers 40-70 nucleotides around the start site (roughly −36 to +12 relative to the start site). This sequence is the core promoter. In a less studied bacterium, or in less characterized stress conditions, the active transcription start sites can be mapped by methods such as differential RNA sequencing (see, for example, Sharma & Vogel, “Differential RNA-seq: the approach behind and the biological insight gained”, Current Opinion in Microbiology 2014, 19:97-105, http://dx.doi.org/10.1016/j.mib.2014.06.010, which is incorporated herein by reference).

Transcription of the nucleotide sequence expressing the toxic agent (or component thereof) or the NOI is under the control of the SPA promoter. The SPA promoter may be one which is operable for transcription of an endogenous gene of reference cells that are growing well under standard conditions, but is also active under stress conditions. The SPA promoter may be endogenous to the first cells (and reference cells) but is operably linked to transcribe a nucleotide sequence which is not transcribed by the SPA promoter in nature. In an alternative, the SPA promoter is not a promoter found in first cells, i.e. the SPA promoter is found in wild-type cells of a different species, or in a different strain (but same species). In another embodiment, the SPA is a synthetic sequence, or is a hybrid sequence, comprising sequences from two or more different endogenous promoters.

In some embodiments, the SPA promoter is a promoter which shows at least the same promoter activity in in vitro stress conditions compared to standard conditions for reference cells which are the same composition as the first cells, and wherein
(a) the standard conditions are a set of in vitro growing conditions which include growth nutrients and growing conditions, and in which the addition of further growth nutrients and change of growing conditions does not further improve the growth rate of the reference cells; and
(b) in the stress conditions,
   (I) one or more of the growth nutrients of (a) has been altered; or
   (II) one or more of the growing conditions of (a) has been altered; or
   (III) one or more stress-inducing parameter(s) has been added;
such that the growth rate of the reference cells is reduced, but otherwise the stress conditions are identical to the standard conditions of (a).

In other embodiments, the SPA promoter is active in cells which comprise an alarmone (optionally one or both of alarmones ppGpp and pppGpp). Alarmones are markers of stress which are discussed elsewhere herein.

In other embodiments, the SPA promoter is active in first cells which are selected from one or more of stationary phase cells, stressed cells, persister cells, biofilm cells and dormant cells. These cells are discussed elsewhere herein.

The SPA promoter may be a promoter of a gene that is upregulated in stationary phase cells of first cells as compared to exponential phase cells of said first cells.

The SPA promoter may be a promoter of a gene that is upregulated in stressed cells of said first cells as compared to exponential phase cells of said first cells.

The SPA promoter may be a promoter of a gene that is upregulated in dormant cells of said first cells as compared to exponential phase cells of said first cells.

The SPA promoter may be a promoter of a gene that is upregulated in persister cells of said first cells as compared to exponential phase cells of said first cells.

The SPA promoter may be a promoter of a gene that is upregulated in biofilm cells of said first cells as compared to exponential phase cells of said first cells.

By “upregulated” it is meant that activity is greater in the stressed cells compared to the cells growing in an exponential phase. Activity may be measured by any of the methods disclosed herein.

The SPA promoter may be a promoter of a stress response gene.

The SPA promoter may be a RpoS promoter. The SPA promoter may be a promoter of a RpoS-regulated gene. The RpoS-regulated gene may be a stress resistance, cell morphology, metabolism, virulence or lysis gene. The SPA promoter may be a RpoSp promoter, such as an E. coli RpoSp promoter, or an orthologue or homologue thereof. Consistent with its role as the master controller of the bacterial stress response, RpoS regulates the expression of stress-response genes that fall into various functional categories: stress resistance, cell morphology, metabolism, virulence and lysis, see for example, Schellhorn, “Function, Evolution, and Composition of the RpoS Regulon in Escherichia coli”, Front. Microbiol., 17 Sep. 2020, Sec. Microbial Physiology and Metabolism, Volume 11, https://doi.org/10.3389/fmicb.2020.560099, which is incorporated herein by reference. In these embodiments, the first cells may be E. coli cells. The σ38 (RpoS) gene encodes the starvation/stationary phase sigma factor.

The SPA promoter may be a σ24 (RpoE) promoter. The SPA promoter may be an E. coli RpoE promoter, or an orthologue or homologue thereof. The SPA promoter may be a promoter of a RpoE regulated gene. The SPA promoter may be a promoter of an E. coli RpoE regulated gene, or an orthologue or homologue thereof. The RpoE regulated gene may be a stress resistance, cell morphology, metabolism, virulence or lysis gene. In these embodiments, the first cells may be E. coli cells. The σ24 (RpoE) gene encodes a minor sigma factor specialized in extreme heat stress response and stresses on membrane and periplasmic proteins, see for example Barchinger & Ades, “Regulated proteolysis: control of the Escherichia coli σ(E)-dependent cell envelope stress response”, Subcell. Biochem., 2013, 66:129-60, doi: 10.1007/978-94-007-5940-4_6, which is incorporated herein by reference.

The SPA promoter may be a σ28 (RpoF or FliA) promoter. The SPA promoter may be an E. coli RpoF or FliA promoter, or an orthologue or homologue thereof. The SPA promoter may be a promoter of a RpoF- or F/44-regulated gene. The SPA promoter may be a promoter of an E. coli RpoF- or FliA-regulated gene, or an orthologue or homologue thereof. The RpoF or FliA-regulated gene may be a stress resistance, cell morphology, metabolism, virulence or lysis gene. In these embodiments, the first cells may be E. coli cells. The σ28 (RpoF/FliA) gene encodes the flagellar synthesis and chemotaxis sigma factor, see for example, Fitzgerald et al., “Comprehensive mapping of the Escherichia coli fiagellar regulatory network” PLoS Genet., 2014 Oct. 2, 10(10):e1004649, doi: 10.1371/journal.pgen.1004649, which is incorporated herein by reference.

The SPA promoter may be a σ32 (RpoH) promoter. The SPA promoter may be an E. coli RpoH promoter, or an orthologue or homologue thereof. The SPA promoter may be a promoter of a RpoH regulated gene. The SPA promoter may be a promoter of an E. coli RpoH-regulated gene, or an orthologue or homologue thereof. The RpoH-regulated gene may be a stress resistance, cell morphology, metabolism, virulence or lysis gene. In these embodiments, the first cells may be E. coli cells. The σ32 (RpoH) gene encodes the heat shock sigma factor, see for example, Yura, “Regulation of the heat shock response in Escherichia coli: history and perspectives”, Genes Genet. Syst., 2019 Jul. 27, 94(3):103-108, doi: 10.1266/ggs.19-00005, which is incorporated herein by reference.

The SPA promoter may be a σ54 (RpoN) promoter. The SPA promoter may be an E. coli RpoN promoter, or an orthologue or homologue thereof. The SPA promoter may be a promoter of a RpoN-regulated gene. The SPA promoter may be a promoter of an E. coli RpoN regulated gene, or an orthologue or homologue thereof. The RpoN regulated gene may be a stress resistance, cell morphology, metabolism, virulence or lysis gene. In these embodiments, the first cells may be E. coli cells. The σ54 (RpoN) gene encodes the nitrogen-limitation sigma factor, see for example, Wigneshweraraj et al., “Modus operandi of the bacterial RNA polymerase containing the sigma54 promoter-specificity factor”, Mol. Microbiol., 2008 May, 68(3):538-46, doi: 10.1111/j.1365-2958.2008.06181.x, which is incorporated herein by reference.

The SPA promoter may be a sigB promoter. The SPA promoter may be an S. aureus SigB promoter, or an orthologue or homologue thereof. The SPA promoter may be a promoter of a SigB-regulated gene. The SPA promoter may be a promoter of an S. aureus SigB-regulated gene, or an orthologue or homologue thereof. The sigB-regulated gene may be a stress resistance, cell morphology, metabolism, virulence or lysis gene. In these embodiments, the first cells may be gram positive bacteria cells. See M8der et al., “Staphylococcus aureus Transcriptome Architecture: From Laboratory to Infection-Mimicking Conditions”, PLoS Genet, 2016, 12(4): e1005962. https://doi.org/10.1371/journal.pgen.1005962, which is incorporated herein by reference.

The SPA promoter may be a sigS promoter. The SPA promoter may be an S. Aureus SigS promoter, or an orthologue or homologue thereof. The SPA promoter may be a promoter of a SigS-regulated gene. The SPA promoter may be a promoter of an S. Aureus SigS-regulated gene, or an orthologue or homologue thereof. The sigS-regulated gene may be a stress resistance, cell morphology, metabolism, virulence or lysis gene. In these embodiments, the first cells may be gram positive bacteria cells. See Shaw et al., “Identification and Characterization of σ^{s}, a Novel Component of the Staphylococcus aureus Stress and Virulence Responses”, PLoS ONE, 2008, 3(12): e3844. https://doi.org/10.1371/journal.pone.0003844, which is incorporated herein by reference.

The genes which are stress resistance, cell morphology, metabolism, virulence or lysis gene are genes whose gene product is used by the cells for a cell function selected from stress resistance, cell morphology, metabolism, virulence and lysis. A skilled person can determine the function of the genes using common general knowledge. For example, the gene products from the osm gene family (e.g. osmB or osmY), the bolA gene and the ftsQAZ operon genes are all used in cell morphology. The gene products from the poxB gene are used in cell metabolism. Some virulence genes are expressed by cells which are in the stationary phase (e.g. in Staphylococcus, such as S. aureus Streptococcus and C. dificile). Those virulence genes are usually Quorum Sensing (QS)-related. For a discussion on virulence genes in S. Aureus, see Jenul & Horswill, “Regulation of Staphylococcus aureus virulence”, Microbiol. Spectr., 2018 February; 6(1), doi: 10.1128/microbiolspec.GPP3-0031-2018. For a discussion on virulence genes in Group A Streptococcus, see Jimenez & Federle, “Quorum sensing in group A Streptococcus”, Front. Cell. Infect. Microbiol., 12 Sep. 2014, Sec. Molecular Bacterial Pathogenesis, Volume 4, 2014, https://doi.org/10.3389/fcimb.2014.00127. For a discussion on virulence genes in C. dificile Carter et al., “Quorum sensing in Clostridium dificile analysis of a luxS-type signalling system”, J. Med. Microbiol., 2005 February; 54(Pt 2):119-127, doi: 10.1099/jmm.0.45817-0, each of which is incorporated herein by reference. Thus, the SPA promoter may be a promoter which is controlled by QS. The SPA promoter may be a promoter controlled by QS by agr in Staphylococcus. The SPA promoter may be a promoter controlled by QS by GAS in Streptococcus. The SPA promoter may be a promoter controlled by QS by las and/or rhl in Pseudomonas.

Thus is one embodiment, the SPA promoter is a promoter of a gene of the awn family (e.g. osmB or osmY, such as an E. coli osm family gene or an orthologue or homologue from a different species. In another embodiment, the SPA promoter is a promoter of a bolA gene, such as an E. coli gene or an orthologue or homologue from a different species. In another embodiment, the SPA promoter is a promoter of a ftsQAZ operon gene, such as an E. coli gene or an orthologue or homologue from a different species. In another embodiment, the SPA promoter is a promoter of a poxB gene, such as an E. coli gene or an orthologue or homologue from a different species.

In another embodiment, the vector comprises one or more cAMP-CRP binding sites 5′ of the SPA promoter for activation of the promoter in the first cells under stress conditions. These cells under stress conditions will be growing slowly or not actively growing. They may comprise stationary phase cells, stressed cells, persister cells, biofilm cells and dormant cells.

The SPA promoter may be a promoter of a gene selected from gadA, gadB, dps, yiaG, hdeA, ycgZ, osmB, uxuA, ompC, rmf, gtgS, galE, cspE, yciF, hfq, otsB, ygaU, agnE, adhE, cspC, hns, yliH, dnaK, ompX, rob, osmY, osmC, sip, wrbA, glpD, bolA, galM, fxsA, sodA, ybgS, appY, stpA and clpP. The SPA promoter may be a promoter of an orthologue or homologue from a different species (e.g. a Proteobacteria, Gammaproteobacteria, Enterobacterales or Enterobacteriaceae species) of any of the genes in the previous sentence. In these embodiments, the first cells may comprise stationary phase cells.

The SPA promoter may be a promoter of a gene selected from yaiN, ylcB, ylcC, ybfA, cydA, cydB, ybgE, pfiB, hyaA, hyaB, hyaD, hyaE, hyaF, ndh, oppA, ydeV, ydeW, isrA, yneB, manX, manY, manZ, yecI, flu, yeeR, preT, yeiA, ompC, hycF, hycA, hypA, hybC, hybA, yhiU, yhiV, tnaL, rbsD, udp, malG, malE and treB. The SPA promoter may be a promoter of an orthologue or homologue from a different species (e.g. a Proteobacteria, Gammaproteobacteria, Enterobacterales or Enterobacteriaceae species) of any of the genes in the previous sentence. In these embodiments, the first cells may comprise biofilm cells.

The SPA promoter may be a promoter of a gene selected from pspA, mzE, dinJ, relE, yafQ, yelM, ygiU, yoeB, aroF, csdA, glpD, glpQ, gshA, lpxP, torZ, cpxP, marR, clpB, rmf and sulA. The SPA promoter may be a promoter of an orthologue or homologue from a different species (e.g. a Proteobacteria, Gammaproteobacteria, Enterobacterales or Enterobacteriaceae species) of any of the genes in the previous sentence. In these embodiments, the first cells may comprise persister phase cells.

The SPA promoter may be a promoter of a gene selected from dnaK, ileS, hepA, yadF, yafD, ribE, clpP, lon, htpG, ybeD, ybeZ, glnS, yceJ, yceP, phoP, topA, yciS, ycjX, mlc, ydhQ, gapA, sdaA, htpX, narP, clpB, grpE, yfjN, ygaD, ygbF, xerD, rdgB, rpoD, rrmW, yhdN, yrfH, gntY, prlC, mutM, ibpA, hslV, fxsA, groES, hflX, miaA, hfq, holC, creA, araB, sbcD, phaB; ybaO; ybbN; galT; macB; cpD; yljA; yccV; ycgU; ldhA; acpD; nohA; ydiH; ffh; sdaC; yidA; yheL and recF. The SPA promoter may be a promoter of an orthologue or homologue from a different species (e.g. a Proteobacteria, Gammaproteobacteria, Enterobacterales or Enterobacteriaceae species) of any of the genes in the previous sentence. In these embodiments, the first cells may comprise stressed cells.

The SPA promoter may be a promoter of a gene selected from asp23, budB, cap5A, cap5B, cap5C, cap5D, cap5E, cap5F, cap5G, cap5H, cap5I, cap5J, cap5L, cap5M, cap5N, cap5O, clpL, crtM, crtN, csbD, epiE, epiF, epiG, fabG, fabZ, hutG, lysP, mtlA, mtlD, murA, mvaK1, mvaD, mvaK2, opuD, rsbV, rsbW, sarA, sarS, sbtA, sigB, spoVG, truB and ydaD. The SPA promoter may be a promoter of an orthologue or homologue from a different species (e.g. a Firmicutes, Bacilli, Bacillales, Staphylococcaceae or Staphylococcus species) of any of the genes in the previous sentence. In these embodiments, the first cells may comprise stressed cells.

Each of the genes in the paragraphs above encode for genes having certain functionality.

Thus, the SPA promoter may be a promoter of a gene selected from a gene encoding a Glutamate decarboxylase, DNA-binding protein, Periplasmic protein, Osmotic stress-induced protein Mannonate dehydratase, Outer membrane protein, Ribosome modulation factor, Glycogen synthetase, UDP-galactose 4-epimerase, Cold shock protein; chromosome condensation protein, RNA-binding protein; RNA function control protein, Trehalose-6-phosphate phosphatase, Acetaldehyde dehydrogenase, Cold shock protein; mukB suppression protein, Heat shock chaperone, Replication right-origin-binding protein, Carbon starvation-induced outer membrane protein, Trp repressor-binding protein, sn-Glycerol-3-phosphate dehydrogenase, Morphogenesis protein; protein for control of PBP6 synthesis, Mutarotase; alpha-aldose to beta anomer conversion enzyme, F exclusion suppressor, Superoxide dismutase, Transcription factor for a stationary-phase gene, H-NS-like curved DNA-binding protein and ATP-dependent protease. The SPA promoter may be a promoter of an orthologue or homologue from a different species (e.g. a Proteobacteria, Gammaproteobacteria, Enterobacterales or Enterobacteriaceae species) of any of the genes in the previous sentence. In these embodiments, the first cells may comprise stationary phase cells.

The SPA promoter may be a promoter of a gene selected from a gene encoding an alpha helix chain, Cytochrome d terminal oxidase, polypeptide subunit, Formate acetyltransferase, Hydrogenase-1 subunit, enzyme for Processing of HyaA and/or HyaB proteins, Respiratory NADH dehydrogenase, protein for Oligopeptide transport; periplasmic-binding protein, kinase, transcriptional regulator, ATP-binding component of a transport system, PTS enzyme, Ferritin-like protein, Outer membrane fluffing protein, adhesin, oxidoreductase, iron-sulphur protein of hydrogenase, protein for transcriptional repression of hyc and/or hyp operons, regulator of 3 hydrogenase isozymes, Subunit of a hydrogenase, transport system permease protein, Tryptophanase, protein of D-ribose transport system, Uridine phosphorylase, Maltose permease and Periplasmic maltose-binding protein PTS system enzyme. The SPA promoter may be a promoter of an orthologue or homologue from a different species (e.g. a Proteobacteria, Gammaproteobacteria, Enterobacterales or Enterobacteriaceae species) of any of the genes in the previous sentence. In these embodiments, the first cells may comprise biofilm cells.

The SPA promoter may be a promoter of a gene selected from a gene encoding a Toxin-antitoxin system protein, DAHP synthase, Cysteine sulfinate desulphinase, Glycerol 3-phosphate dehydrogenase, Glycerophosphoryl diester phosphodiesterase, γ-Glutamate-cysteine ligase, Palmitoleoyl acyltransferase, Periplasmic oxidoreductase, Extracytoplasmic stress resistance protein, Chaperone protein, Ribosome modulation factor and Cell division inhibitor. The SPA promoter may be a promoter of an orthologue or homologue from a different species (e.g. a Proteobacteria, Gammaproteobacteria, Enterobacterales or Enterobacteriaceae species) of any of the genes in the previous sentence. In these embodiments, the first cells may comprise persister cells.

The SPA promoter may be a promoter of a gene selected from a gene encoding a Chaperone, tRNA ligase, RNAP recycling factor, Carbonic anhydrases, Lumazine synthase, protease (optionally Serine protease), PhoH-like protein, Regulator of biofilm formation, Transcriptional regulator, Topoisomerase, lipopolysaccharide assembly protein, Glycerakiehyde 3-phosphate dehydrogenase, L-serine deaminase, Nucleotide exchange factor, Toxin-antitoxin system protein, Cas2, Site specific recombinase, Nucleoside triphosphate pyrophosphatase, RNA polymerase, sigma 70, RNA methyltransferase, Heat shock protein, Iron-sulphur cluster carrier protein, oligopeptidase A, DNA glycosylase, F exclusion protein, Cochaperonine, Ribosome rescue factor, tRNA dimethylallyltransferase, RNA binding protein, DNA polymerase III subunit, Ribulokinase, Structure specific DNA nuclease, Chaperedoxin, Galactose 1-phosphate uridylyltransferase, ABC efflux pump, DNA replication inhibitor, Specificity adapter, NAD-linked lactate dehydrogenase, NADH:quinone oxidoreductase, Signal recognition particle protein component, L-serine:H+symporter, Sulfurtransferase complex subunit and DNA repair protein. The SPA promoter may be a promoter of an orthologue or homologue from a different species (e.g. a Proteobacteria, Gammaproteobacteria, Enterobacterales or Enterobacteriaceae species) of any of the genes in the previous sentence. In these embodiments, the first cells may comprise stressed cells.

The SPA promoter may be a promoter of a gene Selected from a gene encoding an Alkaline shock protein, acetolactate synthase, Capsular polysaccharide synthesis enzyme, ATP-dependent Cip proteinase chain, Squalene desaturase, Squalene synthase, Epidermin immunity protein, glucose I-dehydrogenase, (3R)-hydroxymyristoyl-[acyl carrier protein] dehydratase, ormiminoglutamase, Lysine-specific permease, PTS system protein, Mannitol-1-phosphate 5-dehydrogenase, UDP-N-acetylglucosamine 1-carboxyvinyl transferase, Mevalonate kinase, Mevalonate diphosphate decarboxylase, Phosphomevalonate kinase, Glycine betaine transporter opuD or homologue, Anti-B factor antagonist, Anti-B factor, Staphylococcal accessory regulator A, Staphylococcal accessory regulator A homologue and transmembrane efflux pump protein. The SPA promoter may be a promoter of an orthologue or homologue from a different species (e.g. a Firmicutes, Bacilli, Bacillales, Staphylococcaceae or Staphylococcus species) of any of the genes in the previous sentence. In these embodiments, the first cells may comprise stressed cells.

The SPA promoter may be a promoter of an S. aureus gene comprising a nucleotide sequence with an accession number selected from N315-SA1984, N315-SA2008, N315-SA0144, N315-SA0145, N315-SA0146, N315-SA0147, N315-SA0148, N315-SA0149, N315-SA0150, N315-SA0151, N315-SA0152, N315-SA0153, N315-SA0155, N315-SA0156, N315-SA0157, N315-SA0158, N315-SA2336, N315-SA2349, N315-SA2348, N315-SA1452, COL-SA1872, COL-SA1873, 315-SA1634, N315-SA2260, N315-SA1901, N315-SA2125, N315-SA1505, N315-SA1962, N315-SA1963, N315-SA1902, N315-SA0547, N315-SA0548, N315-SA0549, N315-SA1987, N315-SA1871, N315-SA1870, N315-SA0573, N315-SA0108, N315-SA0099, N315-SA1869, N315-SA0456, N315-SA1114, N315-SA2119, N315-SA0084, N315-SA0098, N315-SA0102, N315-SA0105, N315-SA0163, N315-SA0164, N315-SA0261, N315-SA0296, N315-SA0297, N315-SA0317, N315-SA0326, N315-SA0327, N315-SA0359, N315-SA0360, N315-SA0372, N315-SA0455, N315-SA0509, N315-SA0528, N315-SA0529, N315-SA0541, N315-SA0572, N315-SA0577, N315-SA0578, N315-SA0579, N315-SA0580, N315-SA0581, N315-SA0582, N315-SA0583, N315-SA0584, N315-SA0633, N315-SA0634, N315-SA0635, N315-SA0636, N315-SA0637, N315-SA0658, N315-SA0659, N315-SA0665, N315-SA0666, N315-SA0681, N315-SA0721, N315-SA0722, N315-SA0724, N315-SA0725, N315-SA0740, N315-SA0741, N315-SA0748, N315-SA0749, N315-SA0751, N315-SA0752, N315-SA0755, N315-SA0768, N315-SA0772, N315-SA0774, N315-SA0780, N315-SA0781, N315-SA0933, N315-SA1014, N315-SA1057, N315-SA1559, N315-SA1560, N315-SA1573, N315-SA1590, N315-SA1657, N315-SA1671, N315-SA1692, N315-SA1697, N315-SA1698, N315-SA1699, N315-SA1814, N315-SA1903, N315-SA1924, N315-SA1942, N315-SA1946, N315-SA1961, N315-SA1980, N315-SA1981, N315-SA1985, N315-SA1986, N315-SA2006, N315-SA2101, N315-SA2102, N315-SA2104, N315-SA2158, N315-SA2203, N315-SA2219, N315-SA2240, N315-SA2242, N315-SA2243, N315-SA2262, N315-SA2267, N315-SA2298, N315-SA2309, N315-SA2327, N315-SA2328, N315-SA2350, N315-SA2351, N315-SA2352, N315-SA2366, N315-SA2367, N315-SA2374, N315-SA2398, N315-SA2403, N315-SA2440, N315-SA2441, N315-SA2442, N315-SA2451, N315-SA2452, N315-SA2479, N315-SA2485, N315-SA2488, N315-SA2489, N315-SA2491, N315-SAS023, N315-SAS049, N315-SAS053, N315-SAS056, N315-SAS068, N315-SAS082, N315-SAS083, N315-SAS089, COL-SA0866, COL-SA1046, COL-SA2012, COL-SA2013, COL-SA2379, COL-SA2433, COL-SA2481, COL-SA2595 and COL-SA2631. The SPA promoter may be a promoter of an orthologue or homologue from a different species (e.g. a Firmicutes, Bacilli, Bacillales, Staphylococcaceae or Staphylococcus species) of any of the genes in the previous sentence.

The SPA promoter may be an E. coli hyaA promoter. The SPA promoter may be an E. coli hyaA promoter or an orthologue or homologue from a different species (e.g. a Firmicutes, Bacilli, Bacillales, Staphylococcaceae or Staphylococcus species). The SPA promoter may be an E. coli hyaA promoter and the first cells are E. coli cells. The SPA promoter may be an E. coli hyaA promoter or an orthologue or homologue from a different species (e.g. a Firmicutes, Bacilli, Bacillales, Staphylococcaceae or Staphylococcus species) and the first cells are E. coli cells. When the promoter is an hyaA promoter or an orthologue or homologue from a different species, the first cells may comprise biofilm cells.

In a particular embodiment, the SPA promoter may be an E. coli bolA promoter. The SPA promoter may be an E. coli bolA promoter or an orthologue or homologue from a different species (e.g. a Firmicutes, Bacilli, Bacillales, Staphylococcaceae or Staphylococcus species). The SPA promoter may be an E. coli bolA promoter and the first cells are E. coli cells. The SPA promoter may be an E. coli bolA promoter or an orthologue or homologue from a different species (e.g. a Firmicutes, Bacilli, Bacillales, Staphylococcaceae or Staphylococcus species) and the first cells are E. coli cells. When the promoter is an bolA promoter or an orthologue or homologue from a different species, the first cells may comprise planktonic cells. When the promoter is an bolA promoter or an orthologue or homologue from a different species, the first cells may comprise biofilm cells. When the promoter is an bolA promoter or an orthologue or homologue from a different species, the first cells may comprise planktonic cells and biofilm cells.

The SPA promoter may be an E. coli rpoH promoter. The SPA promoter may be an E. coli rpoH promoter or an orthologue or homologue from a different species (e.g. a Firmicutes, Bacilli, Bacillales, Staphylococcaceae or Staphylococcus species). The SPA promoter may be an E. coli rpoH promoter and the first cells are E. coli cells. The SPA promoter may be an E. coli rpoH promoter or an orthologue or homologue from a different species (e.g. a Firmicutes, Bacilli, Bacillales, Staphylococcaceae or Staphylococcus species) and the first cells are E. coli cells. When the promoter is an rpoH promoter or an orthologue or homologue from a different species, the first cells may comprise planktonic cells. When the promoter is an rpoH promoter or an orthologue or homologue from a different species, the first cells may comprise biofilm cells.

The SPA promoter may be an E. coli yiaG promoter. The SPA promoter may be an E. coli yiaG promoter or an orthologue or homologue from a different species (e.g. a Firmicutes, Bacilli, Bacillales, Staphylococcaceae or Staphylococcus species). The SPA promoter may be an E. coli yiaG promoter and the first cells are E. coli cells. The SPA promoter may be an E. coli yiaG promoter or an orthologue or homologue from a different species (e.g. a Firmicutes, Bacilli, Bacillales, Staphylococcaceae or Staphylococcus species) and the first cells are E. coli cells. When the promoter is an yiaG promoter or an orthologue or homologue from a different species, the first cells may comprise planktonic cells. When the promoter is an yiaG promoter or an orthologue or homologue from a different species, the first cells may comprise biofilm cells.

The SPA promoter may be an E. coli relB promoter. The SPA promoter may be an E. coli relB promoter or an orthologue or homologue from a different species (e.g. a Firmicutes, Bacilli, Bacillales, Staphylococcaceae or Staphylococcus species). The SPA promoter may be an E. coli relB promoter and the first cells are E. coli cells. The SPA promoter may be an E. coli relB promoter or an orthologue or homologue from a different species (e.g. a Firmicutes, Bacilli, Bacillales, Staphylococcaceae or Staphylococcus species) and the first cells are E. coli cells. When the promoter is an relB promoter or an orthologue or homologue from a different species, the first cells may comprise planktonic cells.

The SPA promoter may be any of the promoters listed in Table 2. Optionally, the SPA promoter selected from the promoters listed in Table 2 are used to target expression of the nucleotide sequence or POI in first cells which are gram-negative bacterial cells. Optionally, the SPA promoter selected from the promoters listed in Table 2 is used to are used to target expression of the nucleotide sequence or POI in first cells which are of the same species as the species from which the SPA promoter originates.

TABLE 2
Stationary phase promoters in selected Gram-negative bacteria

| | Name of | | | | Other | Length |
|---|---|---|---|---|---|---|
| Name of or | promoter | Gene product | −10 | −35 | motifs | of spacer |
| Escherichia coli | BolAp1 | BolA | CGGCTAGTA | CTGCAA | — | 15 |
| | [symbol] | [symbol] (Osmotically | ATGCAG | TAAGGT | — | 17 |
| | | inducible | | | | |
| | | periplasmic | | | | |
| | | trehalase) | | | | |
| | Cst-1 | Cst | — | — | — | — |
| | Fic | Fic (PABA or [symbol] ) | TATACT | — | — | — |
| | Hns | H-NS | TATTAT | TTGCAC | — | 17 |
| | PoxB | PoxB (pyrovate | TAAACT | — | [symbol] | — |
| | | oxidase) | | | [symbol] | |
| | Slp | Slp | TATTATG | GATGAAA | — | 16 |
| | AldB | AldB (Aldehyde | TACCCT | — | — | — |
| | | dehydrogenase) | | | | |
| | CsiD | CsiD ( [symbol] starvation | TATTTT | TGCGCA | — | 17 |
| | | inducible gene) | | | | |
| | OsmY | OsmY (Periplasmic | TATATT | CGAGCG | — | 15 |
| | (Csi-5) | protein of unknown | | | | |
| | | function) | | | | |
| Shigella fexneri | GadA | GadA (Glutamate | CTACTTT | — | — | — |
| | | decarboxylase) | | | | |
| vibrio anguillarum | EmpA | EmpA [symbol] | GATCCA | CCGTGCTAC | | 19 |

[symbol]

indicates data missing or illegible when filed

The SPA promoter may be any of the promoters listed in Table 3. Optionally, the SPA promoter selected from the promoters listed in Table 3 are used to target expression of the nucleotide sequence or POI in first cells which are gram-positive bacterial cells. Optionally, the SPA promoter selected from the promoters listed in Table 3 is used to are used to target expression of the nucleotide sequence or POI in first cells which are of the same species as the species from which the SPA promoter originates.

TABLE 3
Stationary phase promoters from selected Gram-positive bacteria

| | | Gene | | | Length of |
|---|---|---|---|---|---|
| Organism | Promoter | product | −10 | −35 | spacer (bp) |
| Bacillus subtilis | Pst | Phosphate | TTTACT | TTCAAA | 16 |
| | | specific | | | |
| | | transport | | | |
| Bacillus subtilis | Cry3a | Crystal proteins | TAAGCT | TTGCAA | 16 |
| Bacillus subtilis | [symbol] | — | TACAAT | TTGGA | 16 |
| Bacillus subtilis | [symbol] mutant | [symbol] | TTGACT | TATAAT | — |
| | | (lipopeptide | | | |
| | | antibiotic | | | |
| | | surfactant) | | | |
| Streptomyces coelicolor | [symbol] mutant | [symbol] | TAAAGT | TTGACA | 18 |
| Corynebacterium | Cg3141 mutant | Cg3141 | TGGGAT | TTAAGG | 17 |
| glutamicum | | ( [symbol] ) | | | |
| Gordonis sp. [symbol] | Stationary phase promoter | — | AATAAT | TTAACT | 22 |

[symbol]

indicates data missing or illegible when filed

In another embodiment, the SPA promoter may be a promoter from a phage, for example a T-even phage. It has been identified in Golec et al.., “Proteomic profiles and kinetics of development of bacteriophage T4 and its rI and rIII mutants in slowly growing Escherichia coli”, J. General Virol., 94(4), 896-905, 2013, doi:https://doi.org/10.1099/vir.0.048686-0 (incorporated herein by reference in its entirety) that the promoters of the genes listed in Table 2 (which table is reproduced below as Table 4) are SPA promoters.

Thus, in one embodiment, the SPA promoter is selected from a promoter of a gene found in T-even phage selected from alpha glucosyl transferase, (A-gf) (such as of the gene encoded by accession number NP_049673.1), protector from prophage-induced early lysis, (RIIB) (such as of the gene encoded by accession number NP_049889.1), conserved hypothetical protein, (E.6) (such as of the gene encoded by accession number NP_049742.1), DNMP kinase, (Gp1) (such as of the gene encoded by accession number NP_049752.1), major head protein, (Gp23) (such as of the gene encoded by accession number NP_049787.1), RecA-like recombination protein, (UrsX) (such as of the gene encoded by accession number NP_049656.2), single-stranded DNA binding protein, (Gp32) (such as of the gene encoded by accession number NP_049854.1), recombination endonuclease subunit, (Gp42) (such as of the gene encoded by accession number NP_049672.1), sliding clamp, DNA polymerase accessory protein, (Gp45) (such as of the gene encoded by accession number NP_049666.1), conserved hypothetical protein, (Vs.6) (such as of the gene encoded by accession number NP_049730.1), dsDNA binding protein, late transcription, (Dsb4) (such as of the gene encoded by accession number NP_049858.1), and protector from prophage-induced early lysis, (RIIA) (such as of the gene encoded by accession number NP_049616.1).

In a particular embodiment, the SPA promoter is a promoter of a T-even phage gene which is protector from prophage-induced early lysis, (RB). In another particular embodiment, the SPA promoter isa promoter of a T-even phage gene which is encoded by accession number NP_049889.1.

TABLE 4
SPA promoters found in T-even phage

| | | Accession | M [symbol] | | Sequence | Protein | Functional |
|---|---|---|---|---|---|---|---|
| Protein name, (short name) | Effect | number | (kDa) | pI | Coverage %^{a} | Score^{b} | category |
| Alpha glucosyl | [symbol] | NP_049673.1 | 46.7 | 6.11 | 71 | 581 | Host or phage |
| transferase, ( [symbol] ) | | | | | | | interactions |
| Protector from | [symbol] | NP_049889.1 | 35.5 | 6.04 | 57 | 477 | Host or phage |
| prophage-induced early | | | | | | | interactions |
| lysis, (RIIB) | | | | | | | |
| Conserved hypothetical | [symbol] | NP_049742.1 | 22 | 6.06 | 71 | [symbol] 4 | ND |
| protein, (E.6) | | | | | | | |
| DNMP kinase, (Gp1) | [symbol] | NP_049752.1 | 27.3 | 5.06 | 56 | 134 | Nucleotide |
| | | | | | | | metabolism |
| Major head protein, | [symbol] | NP_049787.1 | 55.9 | 5.34 | 52 | 625 | Virion protein |
| (Gp23) | | | | | | | |
| RecA-like | [symbol] | NP_649656.2 | 43.9 | 5.31 | 63 | 504 | DNA replication, |
| recombination | | | | | | | recombination, |
| protein, ( [symbol] ) | | | | | | | repair and |
| | | | | | | | processing |
| Single-stranded DNA | [symbol] | NP_649854.1 | 33.5 | 4.82 | 52 | 328 | DNA replication, |
| binding protein, (Gp32) | | | | | | | repair and |
| | | | | | | | recombination |
| Recombination | [symbol] | NP_049672.1 | 39.1 | 5.04 | 36 | 158 | DNA replication, |
| endonuclease subunit, | | | | | | | repair and |
| (Gp47) | | | | | | | recombination |
| Sliding clamp, DNA | [symbol] | NP_049666.1 | 24.8 | 4.89 | 41 | 261 | DNA replication, |
| polymerase accessory | | | | | | | repair and |
| protein, (Gp45) | | | | | | | recombination |
| Conserved hypothetical | [symbol] | NP_049730.1 | 13.8 | 5.71 | 88 | 314 | ND |
| protein, ( [symbol] ) | | | | | | | |
| DsDNA binding | [symbol] | NP_049858.1 | 16.4 | 5.04 | 93 | 249 | Transcription |
| protein, late | | | | | | | |
| transcription, (DsbA) | | | | | | | |
| Protector from | [symbol] | NP_049616.1 | 82.8 | 5.97 | 31 | 592 | Host or phage |
| prophage-induced early | | | | | | | interactions |
| lysis, (R [symbol] A) | | | | | | | |

^{a}The sequence coverage gives the percentage of the protein sequence covered by the peptides measured for the specific protein
^{b}The protein score is a measure of certainty for the identification of a protein calculated by Mascot. For this experiment, protein scores greater than 49 were significant (p < 0.05). [symbol]
indicates data missing or illegible when filed

### First Cells and Reference Cells

The first cells (and reference cells) may be any cell type, including, but not limited to eukaryotic and prokaryotic cells.

In some embodiments, the first cells (and reference cells) comprise a mixture of several (e.g. 2 to 5, such as two or three) different species. In some embodiments, the first cells (and reference cells) comprise a mixture of several (e.g. 2 to 5, such as two or three) different strains. In another embodiment, the first cells (and reference cells) are of a first species. In another embodiment, the first cells (and reference cells) are of a first strain.

For example, any of the first cells or reference cells may be cells which are stationary phase cells. Any of the first cells or reference cells may be stressed cells. Any of the first cells or reference cells may be persister cells. Any of the first cells or reference cells may be dormant cells. The dormant cell may be a bacterial spore, such as a C. difficile spore.

In particular, any of the first cells or reference cells may be biofilm cells, such as bacterial cells within a biofilm. In another embodiment the first cells are selected from one or more of stationary phase cells, stressed cells, persister cells, biofilm cells and dormant cells.

The first cells may be microbial cells, such as prokaryotic (e.g. bacterial or archaeal) or yeast cells. The first cells (and reference cells) may be prokaryotic cells. The first cells (and reference cells) may be selected from microorganism cells, bacterial cells, archaeal cells or fungal cells (e.g. yeast cells). The first cells (and reference cells) may be microorganism cells. The first cells (and reference cells) may be archaeal cells. The first cells (and reference cells) may be fungal cells (e.g. yeast cells). In a particular embodiment, the first cells (and reference cells) may be bacterial cells.

Bacterial cells may be of any species or strain disclosed herein. Thus the first cells (and reference cells) may be any of the species disclosed in Table 5 herein. The first cells (and reference cells) may be a species selected from an E. coli, Klebsiella pneumoniae, Clostridium dificile, Staphylococcus aureus, Helicobacter pylori, Fusobacterium nucleatum, Mycobacterium tuberculosis and an Enterococcus species.

Where the vector expresses an NOI which is not a toxic agent or component thereof, the first cells may be cells which are not pathogenic. The first cells may colonise an organ (e.g. microbiome) where delivery of the POI will be beneficial. For example, the first cells may be of a Lactococcus species. The first cells may be of a Lactobacillus species. The first cells may be of a Bifidobacterium species. The first cells may be of a Bacteroides species. The first cells may be of a Faecalibacterium species. The first cells may be of a Prevotella species.

The first cells may be comprised by a microbiome that also comprises cells of a different strain or species. The first cells may be selected from bacterial cells, archaeal cells or yeast cells comprised by a microbiome. The microbiome may be a mammalian, human, animal, plant, insect, protozoa or amoeba microbiome. The microbiome may be comprised within the gut, the lungs, the skin or the blood. In particular, the microbiome may be a human microbiome in the gut, the lungs, the skin or the blood, and more particularly a human gut microbiome. In one embodiment, the first cells are bacterial cells of a first strain comprised within a human gut microbiome.

The first cells (and reference cells) may comprise spores, such as microbial spores. The spores may be prokaryotic (e.g. bacterial or archaeal) spores or yeast spores, in particular bacterial spores.

The bacterial cells may be anaerobic bacterial cells (i.e. cells that do not require oxygen for growth). Anaerobic bacterial cells include facultative anaerobic cells such as, for example, E. coli, Shewanella oneidensis and Listeria. Anaerobic bacterial cells also include obligate anaerobic cells such as, for example, Bacteroides and Clostridium species. In humans, for example, anaerobic bacteria are most commonly found in the gastrointestinal tract. Thus, the first cells (and reference cells) may be anaerobic bacterial cells.

The first cells may comprise cells selected from stationary phase cells, stressed cells, persister cells, biofilm cells and dormant cells. The SPA promoter may be active for transcription in the first cells.

Additionally or alternatively, the first cells may comprise the alarmone ppGpp and/or the alarmone pppGpp. The SPA promoter may be active for transcription in the first cells.

First Cells that have Markers of being Under Stress Conditions

The present invention is based on the concept that certain promoters have activity in cells which are not actively growing, (i.e. in stationary phase cells, dormant cells, stressed cells and/or biofilm cells), and their activity can be harnessed to target production of toxic agents, therapeutic molecules and other products of interest in such cells. These cells have certain traits and characteristics (markers) which indicative of the state/stage that the cell is in, i.e. in stationary phase, dormant phase, under stressed conditions and/or in a biofilm. Thus, rather that testing promoters experimentally (as described for example in the first to sixth configurations and embodiments thereof) to see if they will be appropriate for delivering toxic agents (or components thereof) or POIs, the promoter can simply be determined to be active in the cells which have these certain traits and characteristics (markers). The first cells and reference cells may all express any of these markers or be cells of the types described herein.

Thus, there is provided the methods and vectors described in the seventh to twelfth configurations hereinabove.

In a particular embodiment, the first cells are selected from microorganism cells, bacterial cells, archaeal cells or fungal cells (e.g. yeast cells). The first cells may be bacterial cells. The first cells can be of the same strain or species. The first cells can be bacterial cells of the same strain. The first cells can be bacterial cells of the same species.

In a particular embodiment; the toxic agent (or component(s) thereof) or POI comprises one or more crRNA(s) one or more crRNA(s) or one or more RNA(s) (such as a guide RNA or crRNA), and
at least one Cas (e.g. a Cas) nuclease that is capable of recognising and modifying (e.g. cutting) at least one target sequence (e.g. protospacer sequence) comprised by the first cells,
wherein the or each crRNA or gRNA is operable in the first cells with a cognate Cas to guide the Cas to a target sequence (e.g. protospacer sequence) comprised by the first cells, wherein the Cas modifies (e.g. cuts) the target sequence (e.g. protospacer sequence) and the first cells are killed.

In another embodiment, the vector encodes component(s) of the toxic agent, or an NOI which encodes one or more (e.g. 1 to 5, such as 1 to 3) guided nuclease(s) or one or more (e.g. 1 to 5, such as 1 to 3) RNA(s) (such as a guide RNA or crRNA) for targeting a sequence comprised by the genome of the first cells.

For example, any of the first cells or reference cells may be cells which are stationary phase cells. Any of the first cells or reference cells may be stressed cells. Any of the first cells or reference cells may be persister cells. Any of the first cells or reference cells may be biofilm cells. Any of the first cells or reference cells may be dormant cells. In another embodiment the first cells are selected from one or more of stationary phase cells, stressed cells, persister cells, biofilm cells and dormant cells.

Thus, in an embodiment of the seventh configuration, there is provided a method of killing first cells comprised by a cell population, wherein the first cells comprise an alarmone (optionally one or both of alarmones ppGpp and pppGpp); and
the method comprises introducing a nucleic acid vector into the first cells, wherein the vector comprises a nucleotide sequence that encodes (i) an agent that is toxic to the first cells or (ii) encodes a component of the toxic agent,
wherein the nucleotide sequence is under the control of a stress-phase active (SPA) promoter in the vector for expression of the agent or component thereof and the sequence is expressed in the first cells to produce the toxic agent or component thereof, whereby the first cells are killed,
wherein the SPA promoter is a promoter that is active for transcription in the first cells.

There is also provided an equivalent embodiment of the tenth configuration for expressing a POI in first cells which comprise an alarmone (optionally one or both of alarmones ppGpp and pppGpp).

In another embodiment of the seventh configuration, there is provided a method of killing first cells comprised by a cell population, wherein the first cells are selected from one or more of stationary phase cells, stressed cells, persister cells, biofilm cells and dormant cells and the method comprises introducing a nucleic acid vector into the first cells, wherein the vector comprises a nucleotide sequence that encodes (i) an agent that is toxic to the first cells or (ii) encodes a component of the toxic agent,
wherein the nucleotide sequence is under the control of a stress-phase active (SPA) promoter in the vector for expression of the agent or component thereof and the sequence is expressed in the first cells to produce the toxic agent or component thereof, whereby the first cells are killed,
wherein the SPA promoter is a promoter that is active for transcription in the first cells.

There is also provided an equivalent embodiment of the tenth configuration for expressing a POI in first cells which are selected from one or more of stationary phase cells, stressed cells, persister cells, biofilm cells and dormant cells.

In an embodiment of the eighth configuration, there is provided a nucleic acid vector comprising a nucleotide sequence that encodes (i) an agent that is toxic to first cells comprised by a cell population or (ii) encodes a component of the toxic agent, wherein the nucleotide sequence is under the control of a stress-phase active (SPA) promoter in the vector, wherein the first cells comprise an alarmone (optionally one or both of alarmones ppGpp and pppGpp), and wherein the SPA promoter is a promoter that is active for transcription in the first cells.

There is also provided an equivalent embodiment of the eleventh configuration of a vector expressing a POI in first cells which comprise an alarmone (optionally one or both of alarmones ppGpp and pppGpp).

In another embodiment of the eighth configuration, there is provided a nucleic acid vector comprising a nucleotide sequence that encodes (i) an agent that is toxic to first cells comprised by a cell population or (ii) encodes a component of the toxic agent, wherein the nucleotide sequence is under the control of a stress-phase active (SPA) promoter in the vector, wherein the first cells are selected from one or more of stationary phase cells, stressed cells, persister cells, biofilm cells and dormant cells, and wherein the SPA promoter is a promoter that is active for transcription in the first cells.

There is also provided an equivalent embodiment of the eleventh configuration of a vector expressing a POI in first cells which are selected from one or more of stationary phase cells, stressed cells, persister cells, biofilm cells and dormant cells.

In an embodiment of the ninth configuration, there is provided a method of producing a nucleic acid vector comprising a nucleotide sequence a nucleotide sequence that encodes (i) an agent that is toxic to first cells comprised by a cell population or (ii) encodes a component of the toxic agent,
wherein the nucleotide sequence is under the control of a stress-phase active (SPA) promoter in the vector, wherein the first cells comprise an alarmone (optionally one or both of alarmones ppGpp and pppGpp),
and wherein the method comprises combining the nucleotide sequence and a promoter together in a deoxyribonucleic acid such that the nucleotide sequence is placed under the control of the promoter whereby a vector is produced,
and wherein the SPA promoter is a promoter that is active for transcription in the first cells, and
optionally formulating the vector in a pharmaceutical composition comprising a diluent, excipient or carrier.

There is also provided an equivalent embodiment of the twelfth configuration for making a vector expressing a POI in first cells which comprise an alarmone (optionally one or both of alarmones ppGpp and pppGpp).

In another embodiment of the ninth configuration, there is provided a method of producing a nucleic acid vector comprising a nucleotide sequence a nucleotide sequence that encodes (i) an agent that is toxic to first cells comprised by a cell population or (ii) encodes a component of the toxic agent,
wherein the nucleotide sequence is under the control of a stress-phase active (SPA) promoter in the vector, wherein the first cells are selected from one or more of stationary phase cells, stressed cells, persister cells, biofilm cells and dormant cells,
and wherein the method comprises combining the nucleotide sequence and a promoter together in a deoxyribonucleic acid such that the nucleotide sequence is placed under the control of the promoter whereby a vector is produced,
and wherein the SPA promoter is a promoter that is active for transcription in the first cells, and
optionally formulating the vector in a pharmaceutical composition comprising a diluent, excipient or carrier.

There is also provided an equivalent embodiment of the twelfth configuration for making a vector expressing a POI in first cells which are selected from one or more of stationary phase cells, stressed cells, persister cells, biofilm cells and dormant cells.

In E. coli; the BarA/UvrY system responds to the presence of acetate and other short-chain carboxylic acids by activating transcription of the noncoding RNAs, CsrB and CsrC, which sequester the RNA-binding protein CsrA, a global regulator of gene expression, see Alvarez et al., “The Escherichia coli two-component signal sensor BarA binds protonated acetate via a conserved hydrophobic-binding pocket”, J. Biol. Chem., 2021 December; 297(6): 101383, doi: 10.1016/j.jbc.2021.101383, incorporated herein by reference.

Thus, the first cells may comprise BarA (e.g. E. coli BarA) or an orthologue or homologue thereof. BarA, is a histidine sensor kinase which can activate OmpR and thereby promote porin synthesis.

Additionally or alternatively, the first cells may comprise UvrY (e.g. E. coli UvrY) or an orthologue or homologue thereof.

The biofilm may be comprised by an organ of a human or animal patient (e.g. comprised by a lung) or comprised by a joint or prosthesis (e.g. prosthetic joint or hip) of the patient. For example, the biofilm is comprised by a vagina, a urinary tract, a bladder, a kidney, a urethra, a catheter, an ear canal (e.g. a middle-ear), an oral cavity, a tooth, a gum, a contact lens, a heart, a devices implanted in a human or animal patient (e.g. such as a joint prosthesis, heart valve, or intervertebral disc), soft tissue of a human or animal, bone, skin, or a mucosa of a human or animal.

The first cells may comprise any genes which is upregulated in response to stress encountered by cells, see below for further examples (such as RpoS, RpoE, RpoF, FliA, RpoH, RpoN or genes which are regulated by a SigB and/or SigS promoter). Other examples of stress-related genes which may be upregulated in stressed cells include xthA, atG, katE, otsBA gor and sodC

A key regulator of stationary phase gene expression in E. coli is the transcription factor σ^{s} (a product of RpoS (katF) gene). The E. coli genome was found to contain two gene, katE and katG encoding for HPII and HP1w1-4× catalases. The expression of HPII was highest in stationary phase and has been shown to be completely dependent on katF gene product. The latter serves as sigma factor for RNA polymerase and therefore named as RpoS or σ^{s} or σ³⁸ or stationary phase sigma factor or starvation sigma factor. The amount of σ^{s} remains relatively low in the growing phase of cells but increases markedly when the cell encounters stress, starvation or enters stationary phase. The role of this protein is to aid in survival and improved resistance to stressful conditions. Induction of as is observed under conditions of low pH, heat or cold shock, UV-induced DNA damage, nutrient starvation, high cell density, high osmolarity, etc. The σ^{s}-dependent genes have been attributed to morphological changes, induction of starvation proteins, iron uptake, carbohydrate metabolism, amino acid transport, and so on, at the onset of stationary phase.

Thus, the first cells may comprise upregulation of a gene selected from xthA, atG, katE, otsBA, gor and sodC (e.g. wherein the gene is an E. coli gene or an orthologue or homologue from a different species).

When the cells are growing, the metabolism-linked genes are highly expressed, and get turned off when the cells enter stationary phase. The stationary phase is a period of no growth, however, genes essential for survival of organisms are still expressed at this stage. Around 20% of the genes of E. coli are found to express at higher level in the stationary phase. These genes are directly linked to many key events including DNA repair, glycogen production, thermotolerance, osmotolerance, etc. Transcriptome profiling/expression analysis of E. coli in stationary phase revealed upregulation of genes which are involved in survival during osmotic stress (ots, tre, osm), long-term survival (e.g. bolA, dps, cbpA, and glgS), periplasmic shock (RpoE and rseA), cold shock (csp genes), etc. Other genes include carbon storage regulator (csrA), trp repressor binding protein (wrbA) and universal stress protein (uspA). Moreover, several antibiotics including lactocin B of lactic acid bacteria, alfatoxin of Aspergillus species are produced mainly in stationary phase.

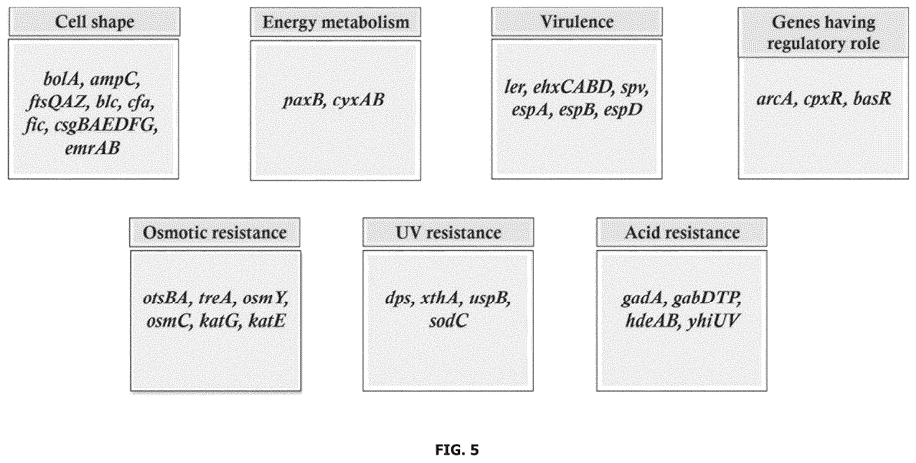

Persister cell formation has also been attributed to genes differentially expressed in stationary phase. These cells are recalcitrant to antibiotic treatments and often are the major cause of drug resistance. Several polyamines including putrescine, spermidine, and cadaverine direct persister formation through upregulation of genes such as rpoS, rmf, ygjD. This observation suggests that polyamine metabolism participates in the regulation of persister cells formation. To determine the genes upregulated at stationary phase microarray was done in Mycobacterium smegmatis grown under conditions of glycerol and glucose depletion. Different subset of genes were identified that were preferentially upregulated at stationary phase. The categories of genes included those involved in metabolism of sulfur, sigma factors including sigB, sigE, and sigH, fatty acid degradation, anaerobic respiration, etc. Also, of key interest in this study is the presence of stationary phase operons involving many gene clusters that are significantly upregulated in stationary phase. The pdh operon of Streptococcus mutans is expressed only in the stationary phase. This operon has been observed to be transcribed only by a subpopulation of bacteria in stationary phase and was vital for survival during long periods of sugar starvation. The dh operon consists of four genes that are transcribed as an operon: pdhD, pdhA, pdhB, pdhC, which encode the components of PDH (pyruvate dehydrogenase) complex, i.e., pyruvate dehydrogenase (two subunits encoded by pdhA and pdhB), dihydrolipoyl transacetylase (pdhC), and dihydrolipoyl dehydrogenase (pdhD). The inactivation of the first gene: pdhD results in impaired survival in both batch cultures and biofilms. Similarly, phage shock protein operon (pspABCE) of E. coli has been reported to be critical for survival under prolonged stationary phase at alkaline conditions. This operon was expressed strongly under extreme stressful conditions and remained significant for survival under nutrient-limited conditions. Categories of genes that are preferentially upregulated in stationary phase is shown in FIG. 5.

Thus the first cells may comprise upregulation of a gene selected from rmf, ygjD and pspABCE (e.g. wherein the gene is an E. coli gene or an orthologue or homologue from a different species). Thus the first cells may comprise upregulation of a gene selected from pdh, such as pdhD, pdhA, pdhB, pdhC (e.g. wherein the gene is an Streptococcus mutans gene or an orthologue or homologue from a different species).

These genes may be identified by expression and bioinformatics analysis. For examples, when the first cells are E. coli, any of the genes which are identified and differentially expressed genes in Bhatia et al., “Transcriptomic profiling of Escherichia coli K-12 in response to a compendium of stressors”, Nature, Sci Rep 12, 8788 (2022), https://doi.org/10.1038/s41598-022-12463-3, which is incorporated herein by reference.

The SPA promoters described herein may also comprise the native promoter of any gene which is mentioned herein as being upregulated in, or essential to survival of, stressed cells (e.g. stationary phase cells, stressed cells, persister cells, biofilm cells and dormant cells).

### Vectors

The person skilled in the art is aware of different types of vectors which may be used in the present disclosure.

The vector may be plasmid, for example, a conjugative plasmid. The conjugative plasmid may be introduced, or is capable of being introduced into the first cells.

The vector may be comprised within a virus, for example any of the viruses described herein.

The vector may be a phage (or comprised within a phage), for example any of the phages (e.g. tail phage or filamentous phage) described herein. The phage may be a lytic phage. The phage may be a lysogenic phage. The phage may be a phage which is able to infect the strain or species of the first cells.

The vector may be a phagemid, e.g. as described elsewhere herein. The vector may be a prophage.

Any of the vectors described herein may be comprised by transduction particles, for example a phage or a non-self-replicating transduction particle. Transduction particles and phages can infect, or may be capable of infecting the first cells and introducing the vector into the cells.

The vector may be DNA comprised by a nanoparticle or gold particle.

The vector may be comprised within a transposon which is capable of transfer into the first cells. The transposon may be a conjugative transposon. The transposon may be a Type I transposon. The transposon may be a Type II transposon.

The vectors herein, in addition to any POI or toxic agent (or component thereof) may express further products of interest (FPOIs) which are not native to the first cells or any adjacent second cells. The FPOIs may comprise any substance described herein as a POI, a toxic agent, or a component of a toxic agent. In some embodiments, FPOIs are part of an operon and are also under control of the SPA promoter. In other embodiments, any FPOIs are under the control of a second promoter which is not an SPA promoter. The second promoter may be a constitutive promoter. The second promoter may be an inducible promoter. The FPOIs expressed may interact with any POI, toxic agent, or component thereof. For example, the FPOI may be a component of a toxic agent (such as a CRISPR/Cas protein or cascade) that interacts with a different component of the toxic agent (such as a first CRISPR array, gRNA or the like) to product the full toxic agent. In such cases, for example, the FPOI component of a toxic agent may be expressed constitutively, and may be able to work with a third component (such as a third CRISPR array or gRNA or the like) expressed under a promoter which is not an SPA promoter to provide other toxic effects in other cells which are not growing under stress conditions nearby. Thus the FPOI component of a toxic agent may be able to interact with two alternative other toxic components to produce toxic effects in different cells and/or at different timepoints.

### Formulations and Compositions Comprising the Vectors

The vector may be formulated in a pharmaceutical composition comprising a diluent, excipient or carrier. The formulation may be comprised within a medical device (such as an ampoule, a syringe, or an inhaler) or is formulated in a tincture, a capsule or a slow-release formulation.

The formulation comprising the vector may be freeze dried prior to encapsulation. Sugars may be used to spray dry formulations comprising phage particles.

Acceptable carriers, excipients, or stabilizers are non-toxic to patients at the dosages and concentrations employed, and include buffers such as phosphate, citrate, and other organic acids; antioxidants including ascorbic acid and methionine; low molecular weight (less than about 10 residues) polypeptides; proteins, such as serum albumin, gelatine, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and metal complexes (e.g. Zn-protein complexes). In limited circumstances, due to stability of the vectors, the formulation may include preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol). A skilled formulator is aware of agents which are compatible with the different modes of delivery of the vectors described herein.

The vectors can also be formulated in liposomes. Liposomes containing the vectors are prepared by methods known in the art, such as described in Epstein et al. (1985) Proc. Natl. Acad. Sci. USA 82:3688; Hwang et al. (1980) Proc. Natl. Acad. Sci. USA 77:4030; and U.S. Pat. Nos. 4,485,045 and 4,544,545, incorporated herein by reference. Liposomes with enhanced circulation time are disclosed in U.S. Pat. No. 5,013,556, incorporated herein by reference.

Vectors described herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (1990) Mack Publishing Co., Easton, Pa.

Sustained-release preparations can also be prepared. Suitable examples of sustained-release preparations include semipermeable matrices of solid hydrophobic polymers containing the antagonist, which matrices are in the form of shaped articles, e.g. films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

Preferably, the pharmaceutical composition comprises a plurality of copies of the vector.

The formulation may be comprised within a medical device, such as an ampoule, a syringe, or an inhaler. The formulation may be an oral tablet, comprised within a blister pack.

### Uses of the Methods and Vectors Disclosed Herein

The methods described herein can be carried out ex vivo. The methods described herein can be carried out in vitro. The methods described herein can be carried out in vivo.

There is provided a nucleic acid vector for use in a method of treating or preventing a disease or condition in a patient that is mediated by the first cells, wherein the treating or preventing comprises administering a vector as described anywhere herein to the patient and killing the first cells. The first cells may be killed according to any method of killing cells described herein (for example by production of any toxic agent or component(s) thereof described herein). The first cells may be pathogenic cells and the treatment results in a reduction or elimination of the pathogenicity of the first cells.

There is also provided a nucleic acid vector for use in a method of treating or preventing a disease or condition in a patient, wherein the method comprises administering a vector expressing an NOI as described herein to the patient and delivering the POI to the first cells. The POI and NOI can be any described herein. The vector expressing the NOI can be produced according to any method described herein.

There is provided a method of killing first cells which method is any of the methods of killing first cells by expressing a toxic agent or component thereof described herein, wherein the method is carried out in a patient that comprises the first cells, to treat or prevent a disease or condition mediated in the patient by the first cells, and wherein the method comprises administering the vector to the patient, introducing the vector into the first cells and expressing the toxic agent or component thereof, whereby the first cells are killed, and the disease or condition is treated or prevented.

There is provided a method of killing first cells which method is any of the methods of killing first cells by expressing a toxic agent or component thereof described herein, wherein the method is carried out in a patient that comprises the first cells, to treat a pathogenic infection of the first cells in the patient, wherein the method comprises administering the vector to the patient, introducing the vector into the first cells and expressing the toxic agent or component thereof, whereby the first cells are killed by the agent or component thereof, and the infection is treated. The first cells may be killed by the toxic agent, or component thereof or by the POI.

There is provided a method of expressing an NOI to kill first cells, which method is any of the methods of expressing NOIs described herein, and wherein the NOI is a toxic agent or component thereof, wherein the method is carried out in a patient that comprises the first cells, to treat or prevent a disease or condition mediated in the patient by the first cells, wherein the method comprises administering the vector to the patient, introducing the vector into the first cells and expressing the POI, whereby the first cells are killed, and the disease or condition is treated or prevented. The first cells may be killed by the POI.

There is provided a method of expressing an NOI to kill first cells, which method may be any of the methods of expressing NOIs described herein, and wherein the NOI is a toxic agent or component thereof, wherein the method is carried out in a patient that comprises the first cells, to treat a pathogenic infection of the first cells in the patient, wherein the method comprises administering the vector to the patient, introducing the vector into the first cells and expressing the POI, whereby the first cells are killed, and the infection is treated. The first cells may be killed by the toxic agent, or component thereof or by the POI.

There is provided a method of expressing an NOI in first cells, which method is any of the methods of expressing NOIs herein, and wherein the NOI is a therapeutic molecule, wherein the method is carried out in a patient that comprises the first cells, to treat or prevent a disease or condition which can be treated or prevented by the NOI, wherein the method comprises administering the vector to the patient, introducing the vector into the first cells and expressing the POI, and the disease or condition is treated or prevented.

There is provided a method of expressing an NOI in first cells, which method is any of the methods of expressing NOIs herein, and wherein the NOI is a molecule that is beneficial to the local environment of the cell, wherein the method is carried out in a patient that comprises the first cells, to treat or prevent a disease or condition which can be treated or prevented by the NOI, wherein the method comprises administering the vector to the patient, introducing the vector into the first cells and expressing the POI, and the disease or condition is treated or prevented.

There is provided a vector as described herein for use in a method to treat or prevent a disease or condition mediated in a patient by the first cells. There is provided a vector as described herein for use in a method to treat a pathogenic infection of the first cells in the patient.

The patient can be a human or animal subject. The patient can be a mammal such as a non-primate (e.g. cows, pigs, horses, cats, dogs, rats, etc.) or a primate (e.g. monkey and human). The patient may be a rodent, mouse or rat. The patient may be a vertebrate, reptile, bird or fish. In particular, the patient is a human.

The first cells can be comprised within a microbiome. The microbiome may be a gut, a lung, a skin, or a blood microbiome. The microbiome may be a human gut, a human lung, a human skin, or a human blood microbiome, in particular a human gut microbiome. The first cells may be bacterial cells. The microbiome can be any microbiome described herein.

The vector can be administered to the patient in one or more doses. It is understood that the precise dosage and duration of treatment is a function of the disease being treated and can be determined empirically using known testing protocols or by extrapolation from in vivo or in vitro test data, and taking into account age, weight and sex of the patient. It is to be noted that concentrations and dosage values can also vary with the severity of the condition to be alleviated. It is to be further understood that for any particular patient, specific dosage regimens can be adjusted over time according to the individual need and the professional judgment of the person administering or supervising the administration of the compositions.

As used herein, “prevention” includes a reducing of the risk of contracting the disease. The “treatment or prevention” may be complete or partial treatment or prevention, i.e. a reduction, but not complete reduction of the disease/condition or symptoms thereof; or a reducing of the risk but not total prevention of the disease/condition or a symptom thereof. Similarly, the methods treat or prevent (i.e. reduces the risk of) an undesirable symptom of the disease or condition or the therapy.

In an example, bacterial or archaeal first cells are killed or growth thereof is inhibited in the patient described herein.

The disease or condition may be an autoimmune disease or condition (e.g. SLE), an inflammatory disease or condition (e.g. rheumatoid arthritis, IBD, Crohn's disease, colitis or psoriasis), a viral infection or mediated by a viral infection (e.g. HIV infection). In particular, the disease or condition is an inflammatory disease or condition of the gut (such as Crohn's disease, Ulcerative Colitis or IBD).

The disease or condition may be metabolic syndrome or cardiometabolic disease (e.g. selected from obesity, diabetes, insulin resistance and non-alcoholic fatty liver disease). The disease may be inflammatory bowel disease (e.g. selected from Crohn's disease and Ulcerative Colitis). The condition may be irritable bowel syndrome or leaky gut syndrome. The POI may be insulin. The POI may be GLP-1. The POI may be an indole derivative (e.g. indole-3-propionic acid or indole-3-propionic acid). The POI may be a short chain fatty acid (e.g. butyrate, acetate or propionate). The POI may be a bacterial sphingolipid. The POI may be an interleukin. The POI may be a molecule that produces an anti-inflammatory effect. The POI may be a tissue growth factor.

The disease or condition may be an autoimmune disease or condition (e.g. SLE) and the POI is a therapy is a treatment therefor, e.g. administration of a tumour necrosis factor ligand superfamily member antagonist, e.g. an anti-B-cell activating factor (BAFF) antibody, such as BENLYSTA™ or a generic version thereof. For example, the disease or condition is an inflammatory disease or condition (e.g. rheumatoid arthritis, IBD, Crohn's disease, colitis or psoriasis) and the POI is a therapy is a treatment therefor, e.g. administration of sarilumab, dupilumab, a tumour necrosis factor ligand superfamily member antagonist, e.g. an anti-TNFα antibody or trap, such as HUMIRA™, REMICADE™, SYMPONI™ or ENBREL™ or a generic version thereof. For example, the disease or condition is a viral infection or mediated by a viral infection (e.g. HIV infection) and the therapy is a treatment therefor, e.g. administration of an anti-retroviral medicament or an anti-HIV vaccine. For example, the disease or condition is a cancer (e.g. colon cancer, bowel cancer, melanoma, NSCLC, breast cancer or pancreatic cancer) and the therapy is a treatment therefor, e.g. administration of a chemotherapeutic agent, e.g. a checkpoint inhibitor or agonist antibody, in particular an antibody fragment, such as an anti-CTLA4, PD-1, PD-L1, PD-L2, LAG3, OX40, CD28, BTLA, CD137, CD27, HVEM, KIR, TIM-3, VISTA, ICOS, GITR, TIGIT or SIRPα antibody or fragment thereof. In an example, the antibody is a bispecific antibody, in particular a bispecific antibody fragment, that specifically binds first and second targets selected from CTLA4, PD-1, PD-L1, PD-L2, LAG3, OX40, CD28, BTLA, CD137, CD27, HVEM, KIR, TIM-3, VISTA, ICOS, G1TR, TIGIT and SIRPα, e.g. wherein the first target is CTLA4 and the second target is LAG3 or PD-1. Optionally, the POI is an antibody is a human gamma-1 antibody or fragment thereof and/or may be enhanced for ADCC or CDC. For example, the POI therapy is a vaccine therapy, e.g. a cancer vaccine therapy or a vaccine therapy for treating or preventing an infection or infectious disease, such as malaria, HIV infection, tuberculosis infection, cholera, Salmonella typhimurium infection, C. dificile infection, Bordetella pertussis infection or chlamydia infection.

The first cells may be tuberculosis cells, cholera cells, Salmonella typhimurium cells, C. dificile cells, Bordetella pertussis cells or Chlamydia cells which are killed by a toxic agent or component thereof.

The disease or condition may be mediated by immune cells (e.g. T-cells) in the patient, and the method comprises causing gut bacterial microbiota dysbiosis in the patient, whereby said dysbiosis modulates immune cells (e.g. TH17 cells) in the patient, thereby treating or reducing the risk of said disease or condition in the patient.

Cancers that may be treated include tumours that are not vascularized, or not substantially vascularized, as well as vascularized tumours. The cancers may comprise non-solid tumours (such as haematological tumours, for example, leukaemias and lymphomas) or may comprise solid tumours. Types of cancers to be treated with the invention include, but are not limited to, carcinoma, blastoma, and sarcoma, and certain leukaemia or lymphoid malignancies, benign and malignant tumours, and malignancies e.g. sarcomas, carcinomas, and melanomas. Adult tumours/cancers and paediatric tumours/cancers are also included.

Haematologic cancers are cancers of the blood or bone marrow. Examples of haematological (or haematogenous) cancers include leukaemias, including acute leukaemias (such as acute lymphocytic leukaemia, acute myelocytic leukaemia, acute myelogenous leukaemia and myeloblasts, promyeiocytic, myelomonocytic, monocytic and erythroleukaemia), chronic leukaemias (such as chronic myelocytic (granulocytic) leukaemia, chronic myelogenous leukaemia, and chronic lymphocytic leukaemia), polycythemia vera, lymphoma, Hodgkin's disease, non-Hodgkin's lymphoma (indolent and high grade forms), multiple myeloma, Waldenstrom's macroglobulinemia, heavy chain disease, myeiodysplastic syndrome, hairy cell leukaemia and myelodysplasia.

Solid tumours are abnormal masses of tissue that usually do not contain cysts or liquid areas. Solid tumours can be benign or malignant. Different types of solid tumours are named for the type of cells that form them (such as sarcomas, carcinomas, and lymphomas). Examples of solid tumours, such as sarcomas and carcinomas, include fibrosarcoma, myxosarcoma, liposarcoma, chondrosarcoma, osteosarcoma, and other sarcomas, synovioma, mesothelioma, Ewing's tumour, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous eel! carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumour, cervical cancer, testicular tumour, seminoma, bladder carcinoma, melanoma, and CNS tumours (such as a glioma (such as brainstem glioma and mixed gliomas), glioblastoma (also known as glioblastoma multiforme) astrocytoma, CNS lymphoma, germinoma, meduloblastoma, Schwannoma craniopharyogioma, ependymoma, pineaioma, hemangioblastoma, acoustic neuroma, oligodendroglioma, menangioma, neuroblastoma, retinoblastoma and brain metastases).

The methods described herein may be used to deliver POIs that treat, or alleviate the symptoms associated with cancer (e.g. gastrointestinal cancer), immune disorders, infections and/or other diseases (e.g. gastrointestinal disease). Thus, there is provided methods of delivering to a patient a recombinant bacteriophage (e.g. coliphage) and/or phagemid engineered to contain a vector that expresses an NOI as described herein. The POI may be a therapeutic molecule which is for example, an antibody, in particular antibody fragments, antibody-based drug, Fc fusion protein, anticoagulant, blood factor, bone morphogenetic protein, engineered protein scaffold, enzyme, growth factor, hormone, interferon, interleukin or thrombolytic as described elsewhere herein.

Examples of gastrointestinal cancers include, without limitation, cancers of the esophagus, gallbladder, liver, pancreas, stomach, small intestine, large intestine (colon) and rectum.

Examples of immune diseases include, without limitation, Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal & neuronal neuropathies, Balo disease, Behcet's disease, Bullous pemphigoid, Cardiomyopathy, Castieman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called thyroiditis, Hemolytic anemia, Henoch-Schonlein purpura, Herpes gestationis, Hypogammaglobulinemia, Idiopathic thrombocytopenic purpura (IP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo and Wegener's granulomatosis (also referred to as Granulomatosis with Polyangiitis (GPA)).

The methods of the present disclosure may be used to deliver POI molecules that treat, or alleviate the symptoms associated with, gastrointestinal diseases. Examples of gastrointestinal diseases include, without limitation, Crohn's disease, ulcerative colitis and colon cancer.

Crohn's disease is a condition of chronic inflammation potentially involving any location of the gastrointestinal tract, but it frequently affects the end of the small bowel and the beginning of the large bowel. In Crohn's disease, all layers of the intestine may be involved, and there can be normal healthy bowel in between patches of diseased bowel. Symptoms include persistent diarrhea (loose, watery, or frequent bowel movements), cramping abdominal pain, fever, and, at times, rectal bleeding. Loss of appetite and weight loss also may occur. However, the disease is not always limited to the gastrointestinal tract; it can also affect the joints, eyes, skin and liver. Fatigue is another common symptom. In some embodiments, the methods described herein are used to deliver to first cells an NOI that expresses gene(s) encoding a monoclonal antibody, or a fragment thereof, infliximab, Interleukin-4, Interleukin-6, Interleukin-10, Interleukin-11, Interleukin-13, or Interleukin-1 Receptor Antagonist. In some embodiments, the methods described here deliver to a patient having Crohn's disease a recombinant bacteriophage (e.g. coliphage) and/or phagemid engineered to contain at least one vector expressing an NOI as described herein.

Ulcerative colitis is a chronic gastrointestinal disorder that is limited to the large bowel (the colon). Ulcerative colitis does not affect all layers of the bowel, but only affects the top layers of the colon in an even and continuous distribution. The first symptom of ulcerative colitis is a progressive loosening of the stool. The stool is generally bloody and may be associated with cramping abdominal pain and severe urgency to have a bowel movement. The diarrhea may begin slowly or quite suddenly. Loss of appetite and subsequent weight loss are common, as is fatigue. In cases of severe bleeding, anemia may also occur. In addition, there may be skin lesions, joint pain, eye inflammation and liver disorders. Children with ulcerative colitis may fail to develop or grow property. In some embodiments, the methods of the present disclosure are used to deliver to first cells that expresses an NOI encoding an Interleukin-4, Interleukin-6, Interleukin-10, Interleukin-11, Interleukin-13, or Interleukin-1 Receptor Antagonist. In some embodiments, there is provided a method of delivering to a patient having ulcerative colitis a recombinant bacteriophage (e.g. coliphage) and/or phagemid engineered to contain at least one vector encoding an NOT selected from a monoclonal antibody (e.g. infliximab), Interleukin-4, Interleukin-6, Interleukin-10, Interleukin-11, Interleukin-13 and Interleukin-1 Receptor Antagonist.

TABLE 5
Example Bacteria
Optionally, the first cells and reference cells are selected from this Table.

Abiotrophia
Abiotrophia defectiva
Acaricomes
Acaricomes phytoseiuli
Acetitomaculum
Acetitomaculum ruminis
Acetivibrio
Acetivibrio cellulolyticus
Acetivibrio ethanolgignens
Acetivibrio multivorans
Acetoanaerobium
Acetoanaerobium noterae
Acetobacter
Acetobacter aceti
Acetobacter cerevisiae
Acetobacter cibinongensis
Acetobacter estunensis
Acetobacter fabarum
Acetobacter ghanensis
Acetobacter indonesiensis
Acetobacter lovaniensis
Acetobacter malorum
Acetobacter nitrogenifigens
Acetobacter oeni
Acetobacter orientalis
Acetobacter orleanensis
Acetobacter pasteurianus
Acetobacter pornorurn
Acetobacter senegalensis
Acetobacter xylinus
Acetobacterium
Acetobacterium bakii
Acetobacterium carbinolicum
Acetobacterium dehalogenans
Acetobacterium fimetarium
Acetobacterium malicum
Acetobacterium paludosum
Acetobacterium tundrae
Acetobacterium wieringae
Acetobacterium woodii
Acetofilamentum
Acetofilamentum rigidum
Acetohalobium
Acetohalobium arabaticum
Acetomicrobium
Acetomicrobium faecale
Acetomicrobium flavidum
Acetonema
Acetonema longum
Acetothermus
Acetothermus paucivorans
Acholeplasma
Acholeplasma axanthum
Acholeplasma brassicae
Acholeplasma cavigenitalium
Acholeplasma equifetale
Acholeplasma granularum
Acholeplasma hippikon
Acholeplasma laidlawii
Acholeplasma modicum
Acholeplasma morum
Acholeplasma multilocale
Acholeplasma oculi
Acholeplasma palmae
Acholeplasma parvum
Acholeplasma pleciae
Acholeplasma vituli
Achromobacter
Achromobacter denitrificans
Achromobacter insolitus
Achromobacter piechaudii
Achromobacter ruhlandii
Achromobacter spanius
Acidaminobacter
Acidaminobacter hydrogenoformans
Acidaminococcus
Acidaminococcus fermentans
Acidaminococcus intestini
Acidicaldus
Acidicaldus organivorans
Acidimicrobium
Acidimicrobium ferrooxidans
Acidiphilium
Acidiphilium acidophilum
Acidiphilium angustum
Acidiphilium cryptum
Acidiphilium multivorum
Acidiphilium organovorum
Acidiphilium rubrum
Acidisoma
Acidisoma sibiricum
Acidisoma tundrae
Acidisphaera
Acidisphaera rubrifaciens
Acidithiobacillus
Acidithiobacillus albertensis
Acidithiobacillus caldus
Acidithiobacillus ferrooxidans
Acidithiobacillus thiooxidans
Acidobacterium
Acidobacterium capsulatum
Acidocella
Acidocella aminolytica
Acidocella facilis
Acidomonas
Acidomonas methanolica
Acidothermus
Acidothermus cellulolyticus
Acidovorax
Acidovorax anthurii
Acidovorax caeni
Acidovorax cattleyae
Acidovorax citrulli
Acidovorax defluvii
Acidovorax delafieldii
Acidovorax facilis
Acidovorax konjaci
Acidovorax temperans
Acidovorax valerianellae
Acinetobacter
Acinetobacter baumannii
Acinetobacter baylyi
Acinetobacter bouvetii
Acinetobacter calcoaceticus
Acinetobacter gerneri
Acinetobacter haemolyticus
Acinetobacter johnsonii
Acinetobacter junii
Acinetobacter lwoffi
Acinetobacter parvus
Acinetobacter radioresistens
Acinetobacter schindleri
Acinetobacter soli
Acinetobacter tandoii
Acinetobacter tjernbergiae
Acinetobacter towneri
Acinetobacter ursingii
Acinetobacter venetianus
Acrocarpospora
Acrocarpospora corrugata
Acrocarpospora macrocephala
Acrocarpospora pleiomorpha
Actibacter
Actibacter sediminis
Actinoalloteichus
Actinoalloteichus cyanogriseus
Actinoalloteichus hymeniacidonis
Actinoalloteichus spitiensis
Actinobaccillus
Actinobacillus capsulatus
Actinobacillus delphinicola
Actinobacillus hominis
Actinobacillus indolicus
Actinobacillus lignieresii
Actinobacillus minor
Actinobacillus muris
Actinobacillus pleuropneumoniae
Actinobacillus porcinus
Actinobacillus rossii
Actinobacillus scotiae
Actinobacillus seminis
Actinobacillus succinogenes
Actinobaccillus suis
Actinobacillus ureae
Actinobaculum
Actinobaculum massiliense
Actinobaculum schaalii
Actinobaculum suis
Actinomyces urinale
Actinocatenispora
Actinocatenispora rupis
Actinocatenispora thailandica
Actinocatenispora sera
Actinocorallia
Actinocorallia aurantiaca
Actinocorallia aurea
Actinocorallia cavernae
Actinocorallia glomerata
Actinocorallia herbida
Actinocorallia libanotica
Actinocorallia longicatena
Actinomadura
Actinomadura alba
Actinomadura atramentaria
Actinomadura bangladeshensis
Actinomadura catellatispora
Actinomadura chibensis
Actinomadura chokoriensis
Actinomadura citrea
Actinomadura coerulea
Actinomadura echinospora
Actinomadura fibrosa
Actinomadura formosensis
Actinomadura hibisca
Actinomadura kijaniata
Actinomadura latina
Actinomadura livida
Actinomadura luteofluorescens
Actinomadura macra
Actinomadura madurae
Actinomadura oligospora
Actinomadura pelletieri
Actinomadura rubrobrunea
Actinomadura rugatobispora
Actinomadura umbrina
Actinomadura verrucosospora
Actinomadura vinacea
Actinomadura viridilutea
Actinomadura viridis
Actinomadura yumaensis
Actinomyces
Actinomyces bovis
Actinomyces denticolens
Actinomyces europaeus
Actinomyces georgiae
Actinomyces gerencseriae
Actinomyces hordeovulneris
Actinomyces howellii
Actinomyces hyovaginalis
Actinomyces israelii
Actinomyces johnsonii
Actinomyces meyeri
Actinomyces naeslundii
Actinomyces neuii
Actinomyces odontolyticus
Actinomyces oris
Actinomyces radingae
Actinomyces slackii
Actinomyces turicensis
Actinomyces viscosus
Actinoplanes
Actinoplanes auranticolor
Actinoplanes brasiliensis
Actinoplanes consettensis
Actinoplanes deccanensis
Actinoplanes derwentensis
Actinoplanes digitatis
Actinoplanes durhamensis
Actinoplanes ferrugineus
Actinoplanes globisporus
Actinoplanes humidus
Actinoplanes italicus
Actinoplanes liguriensis
Actinoplanes lobatus
Actinoplanes missouriensis
Actinoplanes palleronii
Actinoplanes philippinensis
Actinoplanes rectilineatus
Actinoplanes regularis
Actinoplanes teichomyceticus
Actinoplanes utahensis
Actinopolyspora
Actinopolyspora halophila
Actinopolyspora mortivallis
Actinosynnema
Actinosynnema mirum
Actinotalea
Actinotalea fermentans
Aerococcus
Aerococcus sanguinicola
Aerococcus urinae
Aerococcus urinaeequi
Aerococcus urinaehominis
Aerococcus viridans
Aeromicrobium
Aeromicrobium erythreum
Aeromonas
Aeromonas allosaccharophila
Aeromonas bestiarum
Aeromonas caviae
Aeromonas encheleia
Aeromonas enteropelogenes
Aeromonas eucrenophila
Aeromonas ichthiosmia
Aeromonas jandaei
Aeromonas media
Aeromonas popoffii
Aeromonas sobria
Aeromonas veronii
Agrobacterium
Agrobacterium
gelatinovorum
Agrococcus
Agrococcus citreus
Agrococcus jenensis
Agromonas
Agromonas oligotrophica
Agromyces
Agromyces fucosus
Agromyces hippuratus
Agromyces luteolus
Agromyces mediolanus
Agromyces ramosus
Agromyces rhizospherae
Akkermansia
Akkermansia muciniphila
Albidiferax
Albidiferax ferrireducens
Albidovulum
Albidovulum inexpectatum
Alcaligenes
Alcaligenes denitrificans
Alcaligenes faecalis
Alcanivorax
Alcanivorax borkumensis
Alcanivorax jadensis
Algicola
Algicola bacteriolytica
Alicyclobacillus
Alicyclobacillus disulfidooxidans
Alicyclobacillus sendaiensis
Alicyclobacillus vulcanalis
Alishewanella
Alishewanella fetalis
Alkalibacillus
Alkalibacillus haloalkaliphilus
Alkalilimnicola
Alkalilimnicola ehrlichii
Alkaliphilus
Alkaliphilus oremlandii
Alkaliphilus transvaalensis
Allochromatium
Allochromatium vinosum
Alloiococcus
Alloiococcus otitis
Allokutzneria
Allokutzneria albata
Altererythrobacter
Altererythrobacter
ishigakiensis
Altermonas
Altermonas haloplanktis
Altermonas macleodii
Alysiella
Alysiella crassa
Alysiella filiformis
Aminobacter
Aminobacter aganoensis
Aminobacter aminovorans
Aminobacter niigataensis
Aminobacterium
Aminobacterium mobile
Aminomonas
Aminomonas paucivorans
Ammoniphilus
Ammoniphilus oxalaticus
Ammoniphilus oxalivorans
Amphibacillus
Amphibacillus xylanus
Amphritea
Amphritea balenae
Amphritea japonica
Amycolatopsis
Amycolatopsis alba
Amycolatopsis albidoflavus
Amycolatopsis azurea
Amycolatopsis coloradensis
Amycolatopsis lurida
Amycolatopsis mediterranei
Amycolatopsis rifamycinica
Amycolatopsis rubida
Amycolatopsis sulphurea
Amycolatopsis tolypomycina
Anabaena
Anabaena cylindrica
Anabaena flos-aquae
Anabaena variabilis
Anaeroarcus
Anaeroarcus burkinensis
Anaerobaculum
Anaerobaculum mobile
Anaerobiospirillum
Anaerobiospirillum succiniciproducens
Anaerobiospirillum thomasii
Anaerococcus
Anaerococcus hydrogenalis
Anaerococcus lactolyticus
Anaerococcus prevotii
Anaerococcus tetradius
Anaerococcus vaginalis
Anaerofustis
Anaerofustis stercorihominis
Anaeromusa
Anaeromusa acidaminophila
Anaeromyxobacter
Anaeromyxobacter dehalogenans
Anaerorhabdus
Anaerorhabdus furcosa
Anaerosinus
Anaerosinus glycerini
Anaerovirgula
Anaerovirgula multivorans
Ancalomicrobium
Ancalomicrobium adetum
Ancylobacter
Ancylobacter aquaticus
Aneurinibacillus
Aneurinibacillus aneurinilyticus
Aneurinibacillus migulanus
Aneurinibacillus thermoaerophilus
Angiococcus
Angiococcus disciformis
Angulomicrobium
Angulomicrobium tetraedrale
Anoxybacillus
Anoxybacillus pushchinoensis
Aquabacterium
Aquabacterium commune
Aquabacterium parvum
Aquaspirillum
Aquaspirillum polymorphum
Aquaspirillum
putridiconchylium
Aquaspirillum serpens
Aquimarina
Aquimarina latercula
Arcanobacterium
Arcanobacterium
haemolyticum
Arcanobacterium pyogenes
Archangium
Archangium gephyra
Arcobacter
Arcobacter butzleri
Arcobacter cryaerophilus
Arcobacter halophilus
Arcobacter nitrofigilis
Arcobacter skirrowii
Arhodomonas
Arhodomonas aquaeolei
Arsenophonus
Arsenophonus nasoniae
Arthrobacter
Arthrobacter agilis
Arthrobacter albus
Arthrobacter aurescens
Arthrobacter chlorophenolicus
Arthrobacter citreus
Arthrobacter crystallopoietes
Arthrobacter cumminsii
Arthrobacter globiformis
Arthrobacter histidinolovorans
Arthrobacter ilicis
Arthrobacter luteus
Arthrobacter methylotrophus
Arthrobacter mysorens
Arthrobacter nicotianae
Arthrobacter nicotinovorans
Arthrobacter oxydans
Arthrobacter pascens
Arthrobacter phenanthrenivorans
Arthrobacter polychromogenes
Atrhrobacter protophormiae
Arthrobacter psychrolactophilus
Arthrobacter ramosus
Arthrobacter sulfonivorans
Arthrobacter sulfureus
Arthrobacter uratoxydans
Arthrobacter ureafaciens
Arthrobacter viscosus
Arthrobacter woluwensis
Asaia
Asaia bogorensis
Asanoa
Asanoa ferruginea
Asticcacaulis
Asticcacaulis biprosthecium
Asticcacaulis excentricus
Atopobacter
Atopobacter phocae
Atopobium
Atopobium fossor
Atopobium minutum
Atopobium parvulum
Atopobium rimae
Atopobium vaginae
Aureobacterium
Aureobacterium barkeri
Aurobacterium
Aurobacterium liquefaciens
Avibacterium
Avibacterium avium
Avibacterium gallinarum
Avibacterium paragallinarum
Avibacterium volantium
Azoarcus
Azoarcus indigens
Azoarcus tolulyticus
Azoarcus toluvorans
Azohydromonas
Azohydromonas australica
Azohydromonas lata
Azomonas
Azomonas agilis
Azomonas insignis
Azomonas macrocytogenes
Azorhizobium
Azorhizobium caulinodans
Azorhizophilus
Azorhizophilus paspali
Azospirillum
Azospirillum brasilense
Azospirillum halopraeferens
Azospirillum irakense
Azotobacter
Azotobacter beijerinckii
Azotobacter chroococcum
Azotobacter nigricans
Azotobacter salinestris
Azotobacter vinelandii
Bacillus
[see below]
Bacteriovorax
Bacteriovorax stolpii
Bacteroides
Bacteroides caccae
Bacteroides coagulans
Bacteroides eggerthii
Bacteroides fragilis
Bacteroides galacturonicus
Bacteroides helcogenes
Bacteroides ovatus
Bacteroides pectinophilus
Bacteroides pyogenes
Bacteroides salyersiae
Bacteroides stercoris
Bacteroides suis
Bacteroides tectus
Bacteroides thetaiotaomicron
Bacteroides uniformis
Bacteroides ureolyticus
Bacteroides vulgatus
Balnearium
Balnearium lithotrophicum
Balneatrix
Balneatrix alpica
Balneola
Balneola vulgaris
Barnesiella
Barnesiella viscericola
Bartonella
Bartonella alsatica
Bartonella bacilliformis
Bartonella clarridgeiae
Bartonella doshiae
Bartonella elizabethae
Bartonella grahamii
Bartonella henselae
Bartonella rochalimae
Bartonella vinsonii
Bavariicoccus
Bavariicoccus selleri
Bdellovibrio
Bdellovibrio bacteriovorus
Bdellovibrio exovorus
Beggiatoa
Beggiatoa alba
Beijerinckia
Beijerinckia derxii
Beijerinckia fluminensis
Beijerinckia indica
Beijerinckia mobilis
Belliella
Belliella baltica
Bellilinea
Bellilinea caldifistulae
Belnapia
Belnapia moabensis
Bergeriella
Bergeriella denitrificans
Beutenbergia
Beutenbergia cavernae
Bibersteinia
Bibersteinia trehalosi
Bifidobacterium
Bifidobacterium adolescentis
Bifidobacterium angulatum
Bifidobacterium animalis
Bifidobacterium asteroides
Bifidobacterium bifidum
Bifidobacterium boum
Bifidobacterium breve
Bifidobacterium catenulatum
Bifidobacterium choerinum
Bifidobacterium coryneforme
Bifidobacterium cuniculi
Bifidobacterium dentium
Bifidobacterium gallicum
Bifidobacterium gallinarum
Bifidobacterium indicum
Bifidobacterium longum
Bifidobacterium
magnumBifidobacterium
merycicum
Bifidobacterium minimum
Bifidobacterium pseudocatenulatum
Bifidobacterium pseudolongum
Bifidobacterium pullorum
Bifidobacterium ruminantium
Bifidobacterium saeculare
Bifidobacterium subtile
Bifidobacterium thermophilum
Bilophila
Bilophila wadsworthia
Biostraticola
Biostraticola tofi
Bizionia
Bizionia argentinensis
Blastobacter
Blastobacter capsulatus
Blastobacter denitrificans
Blastococcus
Blastococcus aggregatus
Blastococcus saxobsidens
Blastochloris
Blastochloris viridis
Blastomonas
Blastomonas natatoria
Blastopirellula
Blastopirellula marina
Blautia
Blautia coccoides
Blautia hansenii
Blautia producta
Blautia wexlerae
Bogoriella
Bogoriella caseilytica
Bordetella
Bordetella avium
Bordetella bronchiseptica
Bordetella hinzii
Bordetella holmesii
Bordetella parapertussis
Bordetella pertussis
Bordetella petrii
Bordetella trematum
Borrelia
Borrelia afzelii
Borrelia americana
Borrelia burgdorferi
Borrelia carolinensis
Borrelia coriaceae
Borrelia garinii
Borrelia japonica
Bosea
Bosea minatitlanensis
Bosea thiooxidans
Brachybacterium
Brachybacterium alimentarium
Brachybacterium faecium
Brachybacterium paraconglomeratum
Brachybacterium rhamnosum
Brachybacterium tyrofermentans
Brachyspira
Brachyspira alvinipulli
Brachyspira hyodysenteriae
Brachyspira innocens
Brachyspira murdochii
Brachyspira pilosicoli
Bradyrhizobium
Bradyrhizobium canariense
Bradyrhizobium elkanii
Bradyrhizobium japonicum
Bradyrhizobium liaoningense
Brenneria
Brenneria alni
Brenneria nigrifluens
Brenneria quercina
Brenneria quercina
Brenneria salicis
Brevibacillus
Brevibacillus agri
Brevibacillus borstelensis
Brevibacillus brevis
Brevibacillus centrosporus
Brevibacillus choshinensis
Brevibacillus invocatus
Brevibacillus laterosporus
Brevibacillus parabrevis
Brevibacillus reuszeri
Brevibacterium
Brevibacterium abidum
Brevibacterium album
Brevibacterium aurantiacum
Brevibacterium celere
Brevibacterium epidermidis
Brevibacterium frigoritolerans
Brevibacterium halotolerans
Brevibacterium iodinum
Brevibacterium linens
Brevibacterium lyticum
Brevibacterium mcbrellneri
Brevibacterium otitidis
Brevibacterium oxydans
Brevibacterium paucivorans
Brevibacterium stationis
Brevinema
Brevinema andersonii
Brevundimonas
Brevundimonas alba
Brevundimonas aurantiaca
Brevundimonas diminuta
Brevundimonas intermedia
Brevundimonas subvibrioides
Brevundimonas vancanneytii
Brevundimonas variabilis
Brevundimonas vesicularis
Brochothrix
Brochothrix campestris
Brochothrix thermosphacta
Brucella
Brucella canis
Brucella neotomae
Bryobacter
Bryobacter aggregatus
Burkholderia
Burkholderia ambifaria
Burkholderia andropogonis
Burkholderia anthina
Burkholderia caledonica
Burkholderia caryophylli
Burkholderia cenocepacia
Burkholderia cepacia
Burkholderia cocovenenans
Burkholderia dolosa
Burkholderia fungorum
Burkholderia glathei
Burkholderia glumae
Burkholderia graminis
Burkholderia kururiensis
Burkholderia multivorans
Burkholderia phenazinium
Burkholderia plantarii
Burkholderia pyrrocinia
Burkholderia silvatlantica
Burkholderia stabilis
Burkholderia thailandensis
Burkholderia tropica
Burkholderia unamae
Burkholderia vietnamiensis
Buttiauxella
Buttiauxella agrestis
Buttiauxella brennerae
Buttiauxella ferragutiae
Buttiauxella gaviniae
Buttiauxella izardii
Buttiauxella noackiae
Buttiauxella warmboldiae
Butyrivibrio
Butyrivibrio fibrisolvens
Butyrivibrio hungatei
Butyrivibrio proteoclasticus
Bacillus
B. acidiceler
B. acidicola
B. acidiproducens
B. acidocaldarius
B. acidoterrestris
B. aeolius
B. aerius
B. aerophilus
B. agaradhaerens
B. agri
B. aidingensis
B. akibai
B. alcalophilus
B. algicola
B. alginolyticus
B. alkalidiazotrophicus
B. alkalinitrilicus
B. alkalisediminis
B. alkalitelluris
B. altitudinis
B. alveayuensis
B. alvei
B. amyloliquefaciens
B. a. subsp.
amyloliquefaciens
B. a. subsp.
plantarum
B. dipsosauri
B. drentensis
B. edaphicus
B. ehimensis
B. eiseniae
B. enclensis
B. endophyticus
B. endoradicis
B. farraginis
B. fastidiosus
B. fengqiuensis
B. firmus
B. flexus
B. foraminis
B. fordii
B. formosus
B. fortis
B. fumarioli
B. funiculus
B. fusiformis
B. galactophilus
B. galactosidilyticus
B. galliciensis
B. gelatini
B. gibsonii
B. ginsengi
B. ginsengihumi
B. ginsengisoli
B. globisporus (eg, B. g.
subsp. Globisporus; or B. g.
subsp. Marinus)
B. aminovorans
B. amylolyticus
B. andreesenii
B. aneurinilyticus
B. anthracis
B. aquimaris
B. arenosi
B. arseniciselenatis
B. arsenicus
B. aurantiacus
B. arvi
B. aryabhattai
B. asahii
B. atrophaeus
B. axarquiensis
B. azotofixans
B. azotoformans
B. badius
B. barbaricus
B. bataviensis
B. beijingensis
B. benzoevorans
B. beringensis
B. berkeleyi
B. beveridgei
B. bogoriensis
B. boroniphilus
B. borstelensis
B. brevis Migula
B. butanolivorans
B. canaveralius
B. carboniphilus
B. cecembensis
B. cellulosilyticus
B. centrosporus
B. cereus
B. chagannorensis
B. chitinolyticus
B. chondroitinus
B. choshinensis
B. chungangensis
B. cibi
B. circulans
B. clarkii
B. clausii
B. coagulans
B. coahuilensis
B. cohnii
B. composti
B. curdlanolyticus
B. cycloheptanicus
B. cytotoxicus
B. daliensis
B. decisifrondis
B. decolorationis
B. deserti
B. glucanolyticus
B. gordonae
B. gottheilii
B. graminis
B. halmapalus
B. haloalkaliphilus
B. halochares
B. halodenitrificans
B. halodurans
B. halophilus
B. halosaccharovorans
B. hemicellulosilyticus
B. hemicentroti
B. herbersteinensis
B. horikoshii
B. horneckiae
B. horti
B. huizhouensis
B. humi
B. hwajinpoensis
B. idriensis
B. indicus
B. infantis
B. infernus
B. insolitus
B. invictae
B. iranensis
B. isabeliae
B. isronensis
B. jeotgali
B. kaustophilus
B. kobensis
B. kochii
B. kokeshiiformis
B. koreensis
B. korlensis
B. kribbensis
B. krulwichiae
B. laevolacticus
B. larvae
B. laterosporus
B. salexigens
B. saliphilus
B. schlegelii
B. sediminis
B. selenatarsenatis
B. selenitireducens
B. seohaeanensis
B. shacheensis
B. shackletonii
B. siamensis
B. silvestris
B. simplex
B. siralis
B. smithii
B. soli
B. solimangrovi
B. solisalsi
B. songklensis
B. sonorensis
B. sphaericus
B. sporothermodurans
B. stearothermophilus
B. stratosphericus
B. subterraneus
B. subtilis (eg, B. s. subsp.
Inaquosorum; or B. s. subsp.
Spizizeni; or B. s. subsp.
Subtilis)
B. taeanensis
B. tequilensis
B. thermantarcticus
B. thermoaerophilus
B. thermoamylovorans
B. thermocatenulatus
B. thermocloacae
B. thermocopriae
B. thermodenitrificans
B. thermoglucosidasius
B. thermolactis
B. thermoleovorans
B. thermophilus
B. thermoruber
B. thermosphaericus
B. thiaminolyticus
B. thioparans
B. thuringiensis
B. tianshenii
B. trypoxylicola
B. tusciae
B. validus
B. vallismortis
B. vedderi
B. velezensis
B. vietnamensis
B. vireti
B. vulcani
B. wakoensis
B. weihenstephanensis
B. xiamenensis
B. xiaoxiensis
B. zhanjiangensis
B. peoriae
B. persepolensis
B. persicus
B. pervagus
B. plakortidis
B. pocheonensis
B. polygoni
B. polymyxa
B. popilliae
B. pseudalcalophilus
B. pseudofirmus
B. pseudomycoides
B. psychrodurans
B. psychrophilus
B. psychrosaccharolyticus
B. psychrotolerans
B. pulvifaciens
B. pumilus
B. purgationiresistens
B. pycnus
B. qingdaonensis
B. qingshengii
B. reuszeri
B. rhizosphaerae
B. rigui
B. ruris
B. safensis
B. salarius
B. lautus
B. lehensis
B. lentimorbus
B. lentus
B. licheniformis
B. ligniniphilus
B. litoralis
B. locisalis
B. luciferensis
B. luteolus
B. luteus
B. macauensis
B. macerans
B. macquariensis
B. macyae
B. malacitensis
B. mannanilyticus
B. marisflavi
B. marismortui
B. marmarensis
B. massiliensis
B. megaterium
B. mesonae
B. methanolicus
B. methylotrophicus
B. migulanus
B. mojavensis
B. mucilaginosus
B. muralis
B. murimartini
B. mycoides
B. naganoensis
B. nanhaiensis
B. nanhalisediminis
B. nealsonii
B. neidei
B. neizhouensis
B. niabensis
B. niacini
B. novalis
B. oceanisediminis
B. odysseyi
B. okhensis
B. okuhidensis
B. oleronius
B. oryzaecorticis
B. oshimensis
B. pabuli
B. pakistanensis
B. pallidus
B. pallidus
B. panacisoli
B. panaciterrae
B. pantothenticus
B. parabrevis
B. paraflexus
B. pasteurii
B. patagoniensis
Caenimonas
Caenimonas koreensis
Caldalkalibacillus
Caldalkalibacillus uzonensis
Caldanaerobacter
Caldanaerobacter subterraneus
Caldanaerobius
Caldanaerobius fijiensis
Caldanaerobius
polysaccharolyticus
Caldanaerobius zeae
Caldanaerovirga
Caldanaerovirga acetigignens
Caldicellulosiruptor
Caldicellulosiruptor bescii
Caldicellulosiruptor kristjanssonii
Caldicellulosiruptor owensensis
Campylobacter
Campylobacter coli
Campylobacter concisus
Campylobacter curvus
Campylobacter fetus
Campylobacter gracilis
Campylobacter helveticus
Campylobacter hominis
Campylobacter hyointestinalis
Campylobacter jejuni
Campylobacter lari
Campylobacter mucosalis
Campylobacter rectus
Campylobacter showae
Campylobacter sputorum
Campylobacter upsaliensis
Capnocytophaga
Capnocytophaga canimorsus
Capnocytophaga cynodegmi
Capnocytophaga gingivalis
Capnocytophaga granulosa
Capnocytophaga haemolytica
Capnocytophaga ochracea
Capnocytophaga sputigena
Cardiobacterium
Cardiobacterium hominis
Carnimonas
Carnimonas nigrificans
Carnobacterium
Carnobacterium alterfunditum
Carnobacterium divergens
Carnobacterium funditum
Carnobacterium gallinarum
Carnobacterium maltaromaticum
Carnobacterium mobile
Carnobacterium viridans
Caryophanon
Caryophanon latum
Caryophanon tenue
Catellatospora
Catellatospora citrea
Catellatospora methionotrophica
Catenococcus
Catenococcus thiocycli
Catenuloplanes
Catenuloplanes atrovinosus
Catenuloplanes castaneus
Catenuloplanes crispus
Catenuloplanes indicus
Catenuloplanes japonicus
Catenuloplanes nepalensis
Catenuloplanes niger
Chryseobacterium
Chryseobacterium
balustinum
Citrobacter
C. amalonaticus
C. braakii
C. diversus
C. farmeri
C. freundii
C. gillenii
C. koseri
C. murliniae
C. pasteurii[1]
C. rodentium
C. sedlakii
C. werkmanii
C. youngae
Clostridium
(see below)
Coccochloris
Coccochloris elabens
Corynebacterium
Corynebacterium flavescens
Corynebacterium variabile
Curtobacterium
Curtobacterium albidum
Curtobacterium citreus
Clostridium
Clostridium absonum,
Clostridium aceticum,
Clostridium acetireducens,
Clostridium acetobutylicum,
Clostridium acidisoli,
Clostridium aciditolerans,
Clostridium acidurici,
Clostridium aerotolerans,
Clostridium aestuarii,
Clostridium akagii,
Clostridium aldenense,
Clostridium aldrichii,
Clostridium algidicarni,
Clostridium algidixylanolyticum,
Clostridium algifaecis,
Clostridium algoriphilum,
Clostridium alkalicellulosi,
Clostridium aminophilum,
Clostridium aminovalericum,
Clostridium amygdalinum,
Clostridium amylolyticum,
Clostridium arbusti,
Clostridium arcticum,
Clostridium argentinense,
Clostridium asparagiforme,
Clostridium aurantibutyricum,
Clostridium autoethanogenum,
Clostridium baratii,
Clostridium barkeri,
Clostridium bartlettii,
Clostridium beijerinckii,
Clostridium bifermentans,
Clostridium bolteae,
Clostridium bornimense,
Clostridium botulinum,
Clostridium bowmanii,
Clostridium bryantii,
Clostridium butyricum,
Clostridium cadaveris,
Clostridium caenicola,
Clostridium caminithermale,
Clostridium carboxidivorans,
Clostridium carnis,
Clostridium cavendishii,
Clostridium celatum,
Clostridium celerecrescens,
Clostridium cellobioparum,
Clostridium cellulofermentans,
Clostridium cellulolyticum,
Clostridium cellulosi,
Clostridium cellulovorans,
Clostridium chartatabidum,
Clostridium chauvoei,
Clostridium chromiireducens,
Clostridium citroniae,
Clostridium clariflavum,
Clostridium clostridioforme,
Clostridium coccoides,
Clostridium cochlearium,
Clostridium colletant,
Clostridium colicanis,
Clostridium colinum,
Clostridium collagenovorans,
Clostridium cylindrosporum,
Clostridium difficile,
Clostridium diolis,
Clostridium disporicum,
Clostridium drakei,
Clostridium durum,
Clostridium estertheticum,
Clostridium estertheticum
estertheticum,
Clostridium estertheticum
laramiense,
Clostridium fallax,
Clostridium felsineum,
Clostridium fervidum,
Clostridium fimetarium,
Clostridium formicaceticum,
Clostridium frigidicarnis,
Clostridium frigoris,
Clostridium ganghwense,
Clostridium gasigenes,
Clostridium ghonii,
Clostridium glycolicum,
Clostridium glycyrrhizinilyticum,
Clostridium grantii,
Clostridium haemolyticum,
Clostridium halophilum,
Clostridium hastiforme,
Clostridium hathewayi,
Clostridium herbivorans,
Clostridium hiranonis,
Clostridium histolyticum,
Clostridium homopropionicum,
Clostridium huakuii,
Clostridium hungatei,
Clostridium hydrogeniformans,
Clostridium hydroxybenzoicum,
Clostridium hylemonae,
Clostridium jejuense,
Clostridium indolis,
Clostridium innocuum,
Clostridium intestinale,
Clostridium irregulare,
Clostridium isatidis,
Clostridium josui,
Clostridium kluyveri,
Clostridium lactatifermentans,
Clostridium lacusfryxellense,
Clostridium laramiense,
Clostridium lavalense,
Clostridium lentocellum,
Clostridium lentoputrescens,
Clostridium leptum,
Clostridium limosum,
Clostridium litorale,
Clostridium lituseburense,
Clostridium ljungdahlii,
Clostridium lortetii,
Clostridium lundense,
Clostridium magnum,
Clostridium malenominatum,
Clostridium mangenotii,
Clostridium mayombei,
Clostridium methoxybenzovorans,
Clostridium methylpentosum,
Clostridium neopropionicum,
Clostridium nexile,
Clostridium nitrophenolicum,
Clostridium novyi,
Clostridium oceanicum,
Clostridium orbiscindens,
Clostridium oroticum,
Clostridium oxalicum,
Clostridium papyrosolvens,
Clostridium paradoxum,
Clostridium paraperfringens
(Alias: C. welchii),
Clostridium paraputrificum,
Clostridium pascui,
Clostridium pasteurianum,
Clostridium peptidivorans,
Clostridium perenne,
Clostridium perfringens,
Clostridium pfennigii,
Clostridium phytofermentans,
Clostridium piliforme,
Clostridium polysaccharolyticum,
Clostridium populeti,
Clostridium propionicum,
Clostridium proteoclasticum,
Clostridium proteolyticum,
Clostridium psychrophilum,
Clostridium puniceum,
Clostridium purinilyticum,
Clostridium putrefaciens,
Clostridium putrificum,
Clostridium quercicolum,
Clostridium quinii,
Clostridium ramosum,
Clostridium rectum,
Clostridium roseum,
Clostridium saccharobutylicum,
Clostridium saccharogumia,
Clostridium saccharolyticum,
Clostridium saccharoperbutylacetonicum,
Clostridium sardiniense,
Clostridium sartagoforme,
Clostridium scatologenes,
Clostridium schirmacherense,
Clostridium scindens,
Clostridium septicum,
Clostridium sordellii,
Clostridium sphenoides,
Clostridium spiroforme,
Clostridium sporogenes,
Clostridium sporosphaeroides,
Clostridium stercorarium,
Clostridium stercorarium
leptospartum,
Clostridium stercorarium,
Clostridium stercorarium
thermolacticum,
Clostridium sticklandii,
Clostridium straminisolvens,
Clostridium subterminale,
Clostridium sufflavum,
Clostridium sulfidigenes,
Clostridium symbiosum,
Clostridium tagluense,
Clostridium tepidiprofundi,
Clostridium termitidis,
Clostridium tertium,
Clostridium tetani,
Clostridium tetanomorphum,
Clostridium thermaceticum,
Clostridium thermautotrophicum,
Clostridium thermoalcaliphilum,
Clostridium thermobutyricum,
Clostridium thermocellum,
Clostridium thermocopriae,
Clostridium thermohydrosulfuricum,
Clostridium thermolacticum,
Clostridium thermopalmarium,
Clostridium thermopapyrolyticum,
Clostridium thermosaccharolyticum,
Clostridium thermosuccinogenes,
Clostridium thermosulfurigenes,
Clostridium thiosulfatireducens,
Clostridium tyrobutyricum,
Clostridium uliginosum,
Clostridium ultunense,
Clostridium villosum,
Clostridium vincentii,
Clostridium viride,
Clostridium xylanolyticum,
Clostridium xylanovorans
Dactylosporangium
Dactylosporangium aurantiacum
Dactylosporangium fulvum
Dactylosporangium
matsuzakiense
Dactylosporangium roseum
Dactylosporangium thailandense
Dactylosporangium vinaceum
Deinococcus
Deinococcus aerius
Deinococcus apachensis
Deinococcus aquaticus
Deinococcus aquatilis
Deinococcus caeni
Deinococcus radiodurans
Deinococcus radiophilus
Delftia
Delftia acidovorans
Desulfovibrio
Desulfovibrio desulfuricans
Diplococcus
Diplococcus pneumoniae
Echinicola
Echinicola pacifica
Echinicola vietnamensis
Enterobacter
E. aerogenes
E. amnigenus
E. agglomerans
E. arachidis
E. asburiae
E. cancerogenous
E. cloacae
E. cowanii
E. dissolvens
E. gergoviae
E. helveticus
E. hormaechei
E. intermedius
Enterobacter kobei
E. ludwigii
E. mori
E. nimipressuralis
E. oryzae
E. pulveris
E. pyrinus
E. radicincitans
E. taylorae
E. turicensis
E. sakazakii Enterobacter soli
Enterococcus
Enterococcus durans
Enterococcus faecalis
Enterococcus faecium
Erwinia
Erwinia hapontici
Escherichia
Escherichia coli
Faecalibacterium
Faecalibacterium prausnitzii
Fangia
Fangia hongkongensis
Fastidiosipila
Fastidiosipila sanguinis
Fusobacterium
Fusobacterium nucleatum
Flavobacterium
Flavobacterium antarcticum
Flavobacterium aquatile
Flavobacterium aquidurense
Flavobacterium balustinum
Flavobacterium croceum
Flavobacterium cucumis
Flavobacterium daejeonense
Flavobacterium defluvii
Flavobacterium degerlachei
Flavobacterium denitrificans
Flavobacterium filum
Flavobacterium flevense
Flavobacterium frigidarium
Flavobacterium mizutaii
Flavobacterium okeanokoites
Gaetbulibacter
Gaetbulibacter saemankumensis
Gallibacterium
Gallibacterium anatis
Gallicola
Gallicola barnesae
Garciella
Garciella nitratireducens
Geobacillus
Geobacillus thermoglucosidasius
Geobacillus stearothermophilus
Geobacter
Geobacter bemidjiensis
Geobacter bremensis
Geobacter chapellei
Geobacter grbiciae
Geobacter hydrogenophilus
Geobacter lovleyi
Geobacter metallireducens
Geobacter pelophilus
Geobacter pickeringii
Geobacter sulfurreducens
Geodermatophilus
Geodermatophilus obscurus
Gluconacetobacter
Gluconacetobacter xylinus
Gordonia
Gordonia rubripertincta
Haemophilus
Haemophilus aegyptius
Haemophilus aphrophilus
Haemophilus felis
Haemophilus gallinarum
Haemophilus haemolyticus
Haemophilus influenzae
Haemophilus paracuniculus
Haemophilus parahaemolyticus
Haemophilus parainfluenzae
Haemophilus paraphrohaemolyticus
Haemophilus parasuis
Haemophilus pittmaniae
Hafnia
Hafnia alvei
Hahella
Hahella ganghwensis
Halalkalibacillus
Halalkalibacillus halophilus
Helicobacter
Helicobacter pylori
Ideonella
Ideonella azotifigens
Idiomarina
Idiomarina abyssalis
Idiomarina baltica
Idiomarina fontislapidosi
Idiomarina loihiensis
Idiomarina ramblicola
Idiomarina seosinensis
Idiomarina zobellii
Ignatzschineria
Ignatzschineria larvae
Ignavigranum
Ignavigranum ruoffiae
Ilumatobacter
Ilumatobacter fluminis
Ilyobacter
Ilyobacter delafieldii
Ilyobacter insuetus
Ilyobacter polytropus
Ilyobacter tartaricus
Janibacter
Janibacter anophelis
Janibacter corallicola
Janibacter limosus
Janibacter melonis
Janibacter terrae
Jannaschia
Jannaschia cystaugens
Jannaschia helgolandensis
Jannaschia pohangensis
Jannaschia rubra
Janthinobacterium
Janthinobacterium
agaricidamnosum
Janthinobacterium lividum
Jejuia
Jejuia pallidilutea
Jeotgalibacillus
Jeotgalibacillus alimentarius
Jeotgalicoccus
Jeotgalicoccus halotolerans
Kaistia
Kaistia adipata
Kaistia soli
Kangiella
Kangiella aquimarina
Kangiella koreensis
Kerstersia
Kerstersia gyiorum
Kiloniella
Kiloniella laminariae
Klebsiella
K. granulomatis
K. oxytoca
K. pneumoniae
K. terrigena
K. variicola
Kluyvera
Kluyvera ascorbata
Kocuria
Kocuria roasea
Kocuria varians
Kurthia
Kurthia zopfii
Labedella
Labedella gwakjiensis
Labrenzia
Labrenzia aggregata
Labrenzia alba
Labrenzia alexandrii
Labrenzia marina
Labrys
Labrys methylaminiphilus
Labrys miyagiensis
Labrys monachus
Labrys okinawensis
Labrys portucalensis
Lactobacillus
[see below]
Laceyella
Laceyella putida
Lechevalieria
Lechevalieria
aerocolonigenes
Legionella
[see below]
Listeria
L. aquatica
L. booriae
L. cornellensis
L. fleischmannii
L. floridensis
L. grandensis
L. grayi
L. innocua
Listeria ivanovii
L. marthii
L. monocytogenes
L. newyorkensis
L. riparia
L. rocourtiae
L. seeligeri
L. weihenstephanensis
L. welshimeri
Listonella
Listonella anguillarum
Macrococcus
Macrococcus bovicus
Marinobacter
Marinobacter algicola
Marinobacter bryozoorum
Marinobacter flavimaris
Meiothermus
Meiothermus ruber
Methylophilus
Methylophilus methylotrophus
Microbacterium
Microbacterium ammoniaphilum
Microbacterium arborescens
Microbacterium liquefaciens
Microbacterium oxydans
Micrococcus
Micrococcus luteus
Micrococcus lylae
Moraxella
Moraxella bovis
Moraxella nonliquefaciens
Moraxella osloensis
Nakamurella
Nakamurella multipartita
Nannocystis
Nannocystis pusilla
Natranaerobius
Natranaerobius thermophilus
Natranaerobius trueperi
Naxibacter
Naxibacter alkalitolerans
Neisseria
Neisseria cinerea
Neisseria denitrificans
Neisseria gonorrhoeae
Neisseria lactamica
Neisseria mucosa
Neisseria sicca
Neisseria subflava
Neptunomonas
Neptunomonas japonica
Nesterenkonia
Nesterenkonia holobia
Nocardia
Nocardia argentinensis
Nocardia corallina
Nocardia otitidiscaviarum
Lactobacillus
L. acetotolerans
L. acidifarinae
L. acidipiscis
L. acidophilus
Lactobacillus agilis
L. algidus
L. alimentarius
L. amylolyticus
L. amylophilus
L. amylotrophicus
L. amylovorus
L. animalis
L. antri
L. apodemi
L. aviarius
L. bifermentans
L. brevis
L. buchneri
L. camelliae
L. casei
L. kitasatonis
L. kunkeei
L. leichmannii
L. lindneri
L. malefermentans
L. catenaformis
L. ceti
L. coleohominis
L. collinoides
L. composti
L. concavus
L. coryniformis
L. crispatus
L. crustorum
L. curvatus
L. delbrueckii subsp. bulgaricus
L. delbrueckii subsp. delbrueckii
L. delbrueckii subsp. lactis
L. dextrinicus
L. diolivorans
L. equi
L. equigenerosi
L. farraginis
L. farciminis
L. fermentum
L. fornicalis
L. fructivorans
L. frumenti
L. mali
L. manihotivorans
L. mindensis
L. mucosae
L. murinus
L. nagelii
L. namurensis
L. nantensis
L. oligofermentans
L. oris
L. panis
L. pantheris
L. parabrevis
L. parabuchneri
L. paracasei
L. paracollinoides
L. parafarraginis
L. homohiochii
L. iners
L. ingluviei
L. intestinalis
L. fuchuensis
L. gallinarum
L. gasseri
L. parakefiri
L. paralimentarius
L. paraplantarum
L. pentosus
L. perolens
L. plantarum
L. pontis
L. protectus
L. psittaci
L. rennini
L. reuteri
L. rhamnosus
L. rimae
L. rogosae
L. rossiae
L. ruminis
L. saerimneri
L. jensenii
L. johnsonii
L. kalixensis
L. kefiranofaciens
L. kefiri
L. kimchii
L. helveticus
L. hilgardii
L. sakei
L. salivarius
L. sanfranciscensis
L. satsumensis
L. secaliphilus
L. sharpeae
L. siliginis
L. spicheri
L. suebicus
L. thailandensis
L. ultunensis
L. vaccinostercus
L. vaginalis
L. versmoldensis
L. vini
L. vitulinus
L. zeae
L. zymae
L. gastricus
L. ghanensis
L. graminis
L. hammesii
L. hamsteri
L. harbinensis
L. hayakitensis
Legionella
Legionella adelaidensis
Legionella anisa
Legionella beliardensis
Legionella birminghamensis
Legionella bozemanae
Legionella brunensis
Legionella busanensis
Legionella cardiaca
Legionella cherrii
Legionella cincinnatiensis
Legionella clemsonensis
Legionella donaldsonii
Legionella drancourtii
Legionella dresdenensis
Legionella drozanskii
Legionella dumoffii
Legionella erythra
Legionella fairfieldensis
Legionella fallonii
Legionella feeleii
Legionella geestiana
Legionella genomospecies
Legionella gormanii
Legionella gratiana
Legionella gresilensis
Legionella hackeliae
Legionella impletisoli
Legionella israelensis
Legionella jamestowniensis
Candidatus Legionella jeonii
Legionella jordanis
Legionella lansingensis
Legionella londiniensis
Legionella longbeachae
Legionella lytica
Legionella maceachernii
Legionella massiliensis
Legionella micdadei
Legionella monrovica
Legionella moravica
Legionella nagasakiensis
Legionella nautarum
Legionella norrlandica
Legionella oakridgensis
Legionella parisiensis
Legionella pittsburghensis
Legionella pneumophila
Legionella quateirensis
Legionella quinlivanii
Legionella rowbothamii
Legionella rubrilucens
Legionella sainthelensi
Legionella santicrucis
Legionella shakespearei
Legionella spiritensis
Legionella steelei
Legionella steigerwaltii
Legionella taurinensis
Legionella tucsonensis
Legionella tunisiensis
Legionella wadsworthii
Legionella waltersii
Legionella worsleiensis
Legionella yabuuchiae
Oceanibulbus
Oceanibulbus indolifex
Oceanicaulis
Oceanicaulis alexandrii
Oceanicola
Oceanicola batsensis
Oceanicola granulosus
Oceanicola nanhaiensis
Oceanimonas
Oceanimonas baumannii
Oceaniserpentilla
Oceaniserpentilla haliotis
Oceanisphaera
Oceanisphaera donghaensis
Oceanisphaera litoralis
Oceanithermus
Oceanithermus desulfurans
Oceanithermus profundus
Oceanobacillus
Oceanobacillus caeni
Oceanospirillum
Oceanospirillum linum
Paenibacillus
Paenibacillus thiaminolyticus
Pantoea
Pantoea agglomerans
Paracoccus
Paracoccus alcaliphilus
Paucimonas
Paucimonas lemoignei
Pectobacterium
Pectobacterium aroidearum
Pectobacterium atrosepticum
Pectobacterium betavasculorum
Pectobacterium cacticida
Pectobacterium carnegieana
Pectobacterium carotovorum
Pectobacterium chrysanthemi
Pectobacterium cypripedii
Pectobacterium rhapontici
Pectobacterium wasabiae
Planococcus
Planococcus citreus
Planomicrobium
Planomicrobium okeanokoites
Plesiomonas
Plesiomonas shigelloides
Proteus
Proteus vulgaris
Prevotella
Prevotella albensis
Prevotella amnii
Prevotella bergensis
Prevotella bivia
Prevotella brevis
Prevotella bryantii
Prevotella buccae
Prevotella buccalis
Prevotella copri
Prevotella dentalis
Prevotella denticola
Prevotella disiens
Prevotella histicola
Prevotella intermedia
Prevotella maculosa
Prevotella marshii
Prevotella melaninogenica
Prevotella micans
Prevotella multiformis
Prevotella nigrescens
Prevotella oralis
Prevotella oris
Prevotella oulorum
Prevotella pallens
Prevotella salivae
Prevotella stercorea
Prevotella tannerae
Prevotella timonensis
Prevotella veroralis
Providencia
Providencia stuartii
Pseudomonas
Pseudomonas aeruginosa
Pseudomonas alcaligenes
Pseudomonas anguillispetica
Pseudomonas fluorescens
Pseudoalteromonas haloplanktis
Pseudomonas mendocina
Pseudomonas pseudoalcaligenes
Pseudomonas putida
Pseudomonas tutzeri
Pseudomonas syringae
Psychrobacter
Psychrobacter faecalis
Psychrobacter phenylpyruvicus
Quadrisphaera
Quadrisphaera granulorum
Quatrionicoccus
Quatrionicoccus australiensis
Quinella
Quinella ovalis
Ralstonia
Ralstonia eutropha
Ralstonia insidiosa
Ralstonia mannitolilytica
Ralstonia pickettii
Ralstonia pseudosolanacearum
Ralstonia syzygii
Ralstonia solanacearum
Ramlibacter
Ramlibacter henchirensis
Ramlibacter tataouinensis
Raoultella
Raoultella ornithinolytica
Raoultella planticola
Raoultella terrigena
Rathayibacter
Rathayibacter caricis
Rathayibacter festucae
Rathayibacter iranicus
Rathayibacter rathayi
Rathayibacter toxicus
Rathayibacter tritici
Rhodobacter
Rhodobacter sphaeroides
Ruegeria
Ruegeria gelatinovorans
Saccharococcus
Saccharococcus thermophilus
Saccharomonospora
Saccharomonospora azurea
Saccharomonospora cyanea
Saccharomonospora viridis
Saccharophagus
Saccharophagus degradans
Saccharopolyspora
Saccharopolyspora erythraea
Saccharopolyspora gregorii
Saccharopolyspora hirsuta
Saccharopolyspora hordei
Saccharopolyspora rectivirgula
Saccharopolyspora spinosa
Saccharopolyspora taberi
Saccharothrix
Saccharothrix australiensis
Saccharothrix coeruleofusca
Saccharothrix espanaensis
Saccharothrix longispora
Saccharothrix mutabilis
Saccharothrix syringae
Saccharothrix tangerinus
Saccharothrix texasensis
Sagittula
Sagittula stellata
Salegentibacter
Salegentibacter salegens
Salimicrobium
Salimicrobium album
Salinibacter
Salinibacter ruber
Salinicoccus
Salinicoccus alkaliphilus
Salinicoccus hispanicus
Salinicoccus roseus
Salinispora
Salinispora arenicola
Salinispora tropica
Salinivibrio
Salinivibrio costicola
Salmonella
Salmonella bongori
Salmonella enterica
Salmonella subterranea
Salmonella typhi
Sanguibacter
Sanguibacter keddieii
Sanguibacter suarezii
Saprospira
Saprospira grandis
Sarcina
Sarcina maxima
Sarcina ventriculi
Sebaldella
Sebaldella termitidis
Serratia
Serratia fonticola
Serratia marcescens
Sphaerotilus
Sphaerotilus natans
Sphingobacterium
Sphingobacterium multivorum
Staphylococcus
[see below]
Stenotrophomonas
Stenotrophomonas maltophilia
Streptococcus
[also see below]
Streptomyces
Streptomyces achromogenes
Streptomyces cesalbus
Streptomyces cescaepitosus
Streptomyces cesdiastaticus
Streptomyces cesexfoliatus
Streptomyces fimbriatus
Streptomyces fradiae
Streptomyces fulvissimus
Streptomyces griseoruber
Streptomyces griseus
Streptomyces lavendulae
Streptomyces phaeochromogenes
Streptomyces thermodiastaticus
Streptomyces tubercidicus
Tatlockia
Tatlockia maceachernii
Tatlockia micdadei
Tenacibaculum
Tenacibaculum amylolyticum
Tenacibaculum discolor
Tenacibaculum gallaicum
Tenacibaculum lutimaris
Tenacibaculum mesophilum
Tenacibaculum skagerrakense
Tepidanaerobacter
Tepidanaerobacter syntrophicus
Tepidibacter
Tepidibacter formicigenes
Tepidibacter thalassicus
Thermus
Thermus aquaticus
Thermus filiformis
Thermus thermophilus
Staphylococcus
S. arlettae
S. agnetis
S. aureus
S. auricularis
S. capitis
S. caprae
S. carnosus
S. caseolyticus
S. chromogenes
S. cohnii
S. condimenti
S. delphini
S. devriesei
S. epidermidis
S. equorum
S. felis
S. fleurettii
S. gallinarum
S. haemolyticus
S. hominis
S. hyicus
S. intermedius
S. kloosii
S. leei
S. lentus
S. lugdunensis
S. lutrae
S. lyticans
S. massiliensis
S. microti
S. muscae
S. nepalensis
S. pasteuri
S. petrasii
S. pettenkoferi
S. piscifermentans
S. pseudintermedius
S. pseudolugdunensis
S. pulvereri
S. rostri
S. saccharolyticus
S. saprophyticus
S. schleiferi
S. sciuri
S. simiae
S. simulans
S. stepanovicii
S. succinus
S. vitulinus
S. warneri
S. xylosus
Streptococcus
Streptococcus agalactiae
Streptococcus anginosus
Streptococcus bovis
Streptococcus canis
Streptococcus constellatus
Streptococcus downei
Streptococcus dysgalactiae
Streptococcus equines
Streptococcus faecalis
Streptococcus ferus
Streptococcus infantarius
Streptococcus iniae
Streptococcus intermedius
Streptococcus lactarius
Streptococcus milleri
Streptococcus mitis
Streptococcus mutans
Streptococcus oralis
Streptococcus tigurinus
Streptococcus orisratti
Streptococcus parasanguinis
Streptococcus peroris
Streptococcus pneumoniae
Streptococcus pseudopneumoniae
Streptococcus pyogenes
Streptococcus ratti
Streptococcus salivariu
Streptococcus thermophilus
Streptococcus sanguinis
Streptococcus sobrinus
Streptococcus suis
Streptococcus uberis
Streptococcus vestibularis
Streptococcus viridans
Streptococcus zooepidemicus
Uliginosibacterium
Uliginosibacterium gangwonense
Ulvibacter
Ulvibacter litoralis
Umezawaea
Umezawaea tangerina
Undibacterium
Undibacterium pigrum
Ureaplasma
Ureaplasma urealyticum
Ureibacillus
Ureibacillus composti
Ureibacillus suwonensis
Ureibacillus terrenus
Ureibacillus thermophilus
Ureibacillus thermosphaericus
Vagococcus
Vagococcus carniphilus
Vagococcus elongatus
Vagococcus fessus
Vagococcus fluvialis
Vagococcus lutrae
Vagococcus salmoninarum
Variovorax
Variovorax boronicumulans
Variovorax dokdonensis
Variovorax paradoxus
Variovorax soli
Veillonella
Veillonella atypica
Veillonella caviae
Veillonella criceti
Veillonella dispar
Veillonella montpellierensis
Veillonella parvula
Veillonella ratti
Veillonella rodentium
Venenivibrio
Venenivibrio stagnispumantis
Verminephrobacter
Verminephrobacter eiseniae
Verrucomicrobium
Verrucomicrobium spinosum
Vibrio
Vibrio aerogenes
Vibrio aestuarianus
Vibrio albensis
Vibrio alginolyticus
Vibrio campbellii
Vibrio cholerae
Vibrio cincinnatiensis
Vibrio coralliilyticus
Vibrio cyclitrophicus
Vibrio diazotrophicus
Vibrio fluvialis
Vibrio furnissii
Vibrio gazogenes
Vibrio halioticoli
Vibrio harveyi
Vibrio ichthyoenteri
Vibrio mediterranei
Vibrio metschnikovii
Vibrio mytili
Vibrio natriegens
Vibrio navarrensis
Vibrio nereis
Vibrio nigripulchritudo
Vibrio ordalii
Vibrio orientalis
Vibrio parahaemolyticus
Vibrio pectenicida
Vibrio penaeicida
Vibrio proteolyticus
Vibrio shilonii
Vibrio splendidus
Vibrio tubiashii
Vibrio vulnificus
Virgibacillus
Virgibacillus halodenitrificans
Virgibacillus pantothenticus
Weissella
Weissella cibaria
Weissella confusa
Weissella halotolerans
Weissella hellenica
Weissella kandleri
Weissella koreensis
Weissella minor
Weissella paramesenteroides
Weissella soli
Weissella thailandensis
Weissella viridescens
Williamsia
Williamsia marianensis
Williamsia maris
Williamsia serinedens
Winogradskyella
Winogradskyella thalassocola
Wolbachia
Wolbachia persica
Wolinella
Wolinella succinogenes
Zobellia
Zobellia galactanivorans
Zobellia uliginosa
Zoogloea
Zoogloea ramigera
Zoogloea resiniphila
Xanthobacter
Xanthobacter agilis
Xanthobacter aminoxidans
Xanthobacter autotrophicus
Xanthobacter flavus
Xanthobacter tagetidis
Xanthobacter viscosus
Xanthomonas
Xanthomonas albilineans
Xanthomonas alfalfae
Xanthomonas arboricola
Xanthomonas axonopodis
Xanthomonas campestris
Xanthomonas citri
Xanthomonas codiaei
Xanthomonas cucurbitae
Xanthomonas euvesicatoria
Xanthomonas fragariae
Xanthomonas fuscans
Xanthomonas gardneri
Xanthomonas hortorum
Xanthomonas hyacinthi
Xanthomonas perforans
Xanthomonas phaseoli
Xanthomonas pisi
Xanthomonas populi
Xanthomonas theicola
Xanthomonas translucens
Xanthomonas vesicatoria
Xylella
Xylella fastidiosa
Xylophilus
Xylophilus ampelinus
Xenophilus
Xenophilus azovorans
Xenorhabdus
Xenorhabdus beddingii
Xenorhabdus bovienii
Xenorhabdus cabanillasii
Xenorhabdus doucetiae
Xenorhabdus griffiniae
Xenorhabdus hominickii
Xenorhabdus koppenhoeferi
Xenorhabdus nematophila
Xenorhabdus poinarii
Xylanibacter
Xylanibacter oryzae
Yangia
Yangia pacifica
Yaniella
Yaniella flava
Yaniella halotolerans
Yeosuana
Yeosuana aromativorans
Yersinia
Yersinia aldovae
Yersinia bercovieri
Yersinia enterocolitica
Yersinia entomophaga
Yersinia frederiksenii
Yersinia intermedia
Yersinia kristensenii
Yersinia mollaretii
Xylanibacterium ulmi
Yersinia philomiragia
Yersinia pestis
Yersinia pseudotuberculosis
Yersinia rohdei
Yersinia ruckeri
Yokenella
Yokenella regensburgei
Yonghaparkia
Yonghaparkia alkaliphila
Zavarzinia
Zavarzinia compransoris
Zooshikella
Zooshikella ganghwensis
Zunongwangia
Zunongwangia profunda
Zymobacter
Zymobacter palmae
Zymomonas
Zymomonas mobilis
Zymophilus
Zymophilus paucivorans
Zymophilus raffinosivorans
Zobellella
Zobellella denitrificans
Zobellella taiwanensis
Zeaxanthinibacter
Zeaxanthinibacter enoshimensis
Zhihengliuella
Zhihengliuella halotolerans
Xylanibacterium

The present invention is described in more detail in the following non-limiting Examples.

### EXAMPLES

### Example 1. Determining Fast Growth Rate of E. coli Under Standard Conditions

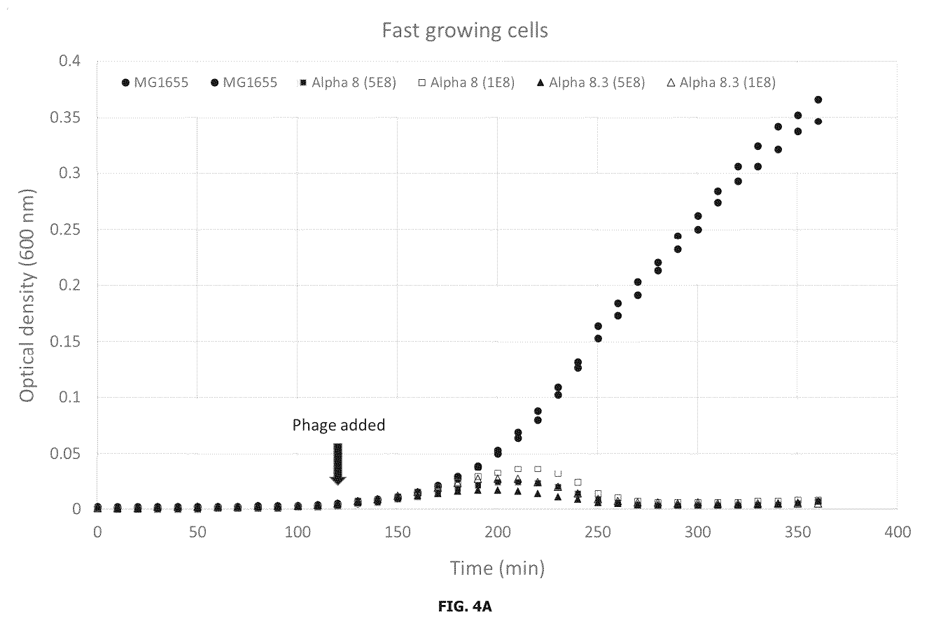

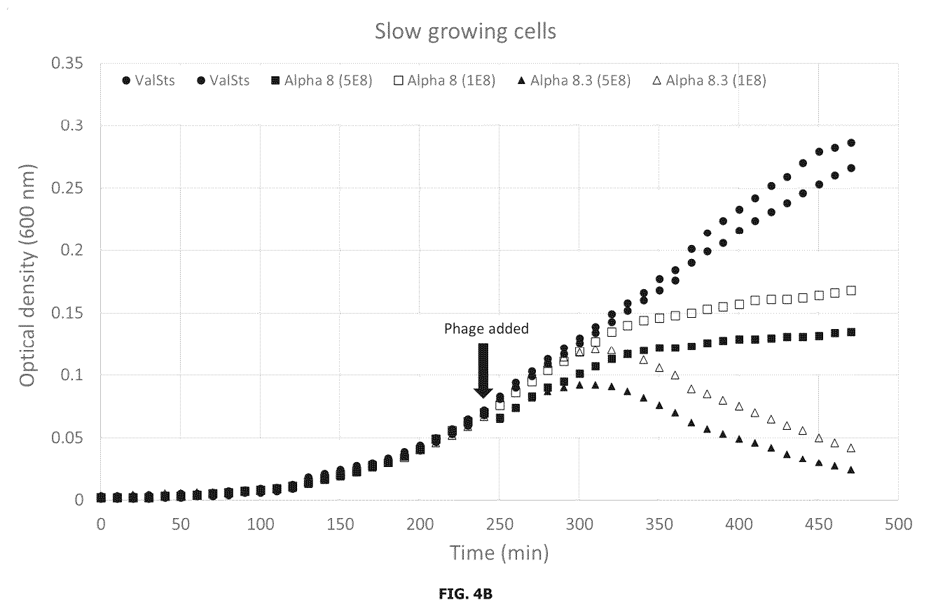

For this experiment, we used the standard laboratory E. coli K-12 strain MG1655 (ATCC 700926). The strain was streaked on LB (Sigma-Aldrich) agar plates and incubated at 37° C. to obtain single colonies. One colony was inoculated in 5 ml 2×YT (Sigma-Aldrich) in a 14 mL culture tube and incubated with orbital shaking (250 rpm) for 12-16 h at 37° C. (overnight culture). Traditionally, such culture would be used to inoculate a new culture which was grown with orbital shaking (250 rpm) at 37° C. and the optical density (OD600) of the culture was recorded at every 10 minutes. However, this method is labour intensive, therefore we used an automated system instead. The overnight culture was diluted 1:1000 in 2×YT in 96-well microtiter plates (˜3×10⁶ cells/300 μL medium) and the plate was incubated at 37° C. in a microplate reader (Biotek Synergy H1) with shaking and reading of optical density (OD600) every 10 minutes (example curves shown in FIG. 4A, dots).

Using Equation 1, the generation time of our fast-growing E. coli cultures (i.e., growth under optimal laboratory conditions which are standard conditions) was determined to be 21±2 minutes.

### Example 2. Determining Fast Growth Rate of Other Gram Negative Enterobacteriaceae Under Standard Conditions

We used the same growth conditions (i.e. standard conditions) as for E. coli (Example 1) to determine the exponential phase generation time for Pseudomonas aeruginosa strain PA01 (ATCC 15692) and Klebsiella pneumoniae strain KpGe (DBS0351098), except that for K. pneumonaie, the growth medium used was TSB (Tryptic Soy Broth, Sigma-Aldrich) and for P. aeruginosa, it was BHI (Brain-Heart Infusion broth Sigma-Aldrich).

The exponential phase generation time for fast-growing P. aeruginosa PA01 (i.e., under optimal laboratory conditions which are standard conditions) was determined to be 20 t 3 minutes.

The exponential phase generation time for fast-growing K pneumoniae KpGe (i.e., under optimal laboratory conditions which are standard conditions) was determined to be 26±5 minutes.

### Example 3. Determining Fast Growth Rate of Staphylococcus aures Under Standard Conditions

The exponential phase generation time for Staphylococcus aureus strain RN4220 (NCBI Taxonomy ID: 561307) was determined in TSB (tryptic Soy Broth, Sigma Aldrich). The exponential phase generation time for fast-growing S. aureus RN4220 (i.e., under optimal laboratory conditions which are standard conditions) was determined to be 39±1.8 minutes.

### Example 4. Determining Fast Growth Rate of Clostridium dificile Under Standard Conditions

C. difficile is strictly anaerobic and all culturing (solid and liquid) is thus performed in an anaerobic chamber using pre-reduced media. Brain Heart Infusion Salt (BHIS) broth and agar (Sigma Aldrich) was used for growth in liquid and on solid agar conditions, respectively.

The exponential phase generation time for fast-growing C. difficile strain 630 (NCBI Taxonomy ID: 272563), under optimal laboratory conditions (which are standard conditions) was not determined but expected to be 40 minutes (see Curry, “Clostridium difficile”, Clin Lab Med. 2010 March; 30(1): 329-342, doi: 10.1016/j.cll.2010.04.001, incorporated herein by reference).

### Example 5. E. coli Model Systems for Sub-Optimal Growth Conditions (i.e. Stress Conditions)

For the experiment we used E. coli MG1655 valS^{ts} or gltX^{ts} (temperature-sensitive allele of valS or gltX is introduced into the E. coli/K12 reference strain MG1655 (ATCC 700926)). Briefly, we induce synthesis of the alarmones (p)ppGpp by limiting the charging of valyl- or glutamyl-tRNAs. Strain construction and stress associated physiological changes were previously analysed in these strains (Svenningsen M S et al., (2019). “Birth and Resuscitation of (p)ppGpp Induced Antibiotic Tolerant Persister Cells”. Scientific Reports volume 9: 6056) i.e. the activity of the enzyme decreases as the temperature is increased. Due to the decreased enzyme activity at elevated temperatures, the level of uncharged tRNAs is increased and leads to Rel-mediated (p)ppGpp synthesis. High levels of (p)ppGpp activates the general stress response, which leads to switching from fast growth to slow growth, increased tolerance to antimicrobials, and a high rate of persister cell formation. To induce stress (alarmone synthesis), we grew the culture at semi-permissive (partially inhibiting ValS/GltX function) temperature (36° C.) but otherwise same growth conditions defined in Example 1.

Using this experimental setup and Equation 1 (example curves are shown in FIG. 48, dots), we were able to determine the generation time during the exponential growth phase to be 112 k 6 minutes for MG1655 valS^{ts} and 162±8 minutes for MG1655 gltX^{ts}, which was in both cases more than 1.5 times the generation time obtained for the cell culture in example 1.

A similar stress can be induced by the addition of the seryl-tRNA synthetase inhibitor, serine hydroxamate (SHX). To determine the growth inhibitory concentration of SHX for E. coli bSNP52, we used the experimental setup of Example 1, and added different concentrations of SHX to the media. In the presence of 4.8 mM SHX (i.e. under stress conditions), the generation time of cells was more than 1.5 times longer compared to the untreated cells (example curve is shown in FIG. 2B, dots).

### Example 6. Promoters that are Active Under Stress Conditions Vs Standard Conditions (in Fast-Growing. Vs Slow-Growing Cells)

In bacterial cells gene products, such as enzymes, structural proteins, and RNA molecules, are synthesised when they are needed and in appropriate amounts. Consequently, fast growing cells (under standard conditions) and slow growing cells (under stress conditions) show different gene expression patterns. The first step of gene expression is initiation of transcription, which occurs at promoter sequences. The relative promoter activities in cells in different states and in transitions between states are typically routinely studied globally by transcriptomics and transcription start site mapping, and individually, using reporter genes. These methods allow the identification of promoters that are required for the establishment and/or maintenance of slow growing states.

Promoters required for fast growth are generally recognised by RNA polymerase associated with the vegetative (primary) sigma factor (e.g. σ70, SigA, MysA, HrdB). Promoters that are activated in certain stress conditions are recognised by RNA polymerase associated with a stress related sigma factor, e.g.:

In E. coli and relatives:
σ24 (RpoE)—extreme heat stress response and the extracellular proteins sigma factor
σ28 (RpoF/FliA)—the flagellar synthesis and chemotaxis sigma factor
σ32 (RpoH)—the heat shock sigma factor
σ38 (RpoS)—the starvation/stationary phase sigma factor
σ54 (RpoN)—the nitrogen-limitation sigma factor

In gram positive bacteria:
sigB—induced under various stress conditions, including heat shock, cold shock, low aeration, and stationary phase
sigS—induced upon exposure to a variety of chemical stressors that elicit DNA damage, and highly induced during growth in serum

### Example 7. Constructing CRISPR-Guided Vectors with Promoters Active in Slow Growing Cells of E. coli Under Stress Conditions

We engineered phagemid-type CRISPR-guided vectors (CGV), which are nucleic acid vectors that can be packaged into phage-like transducing particles comprising sequences encoding CRISPR arrays and Cas3 type nucleases. We constructed a series of CGVs that differed only in the promoter transcribing the CRISPR/Cas kill circuits, which target chromosomal sequences of E. coli (FIG. 1). CGVs were based on the low copy number CloDF13 replicon, and contained a terminator sequence upstream of the promoter that transcribed the E. coli cas genes (cas3 and casABCDE in a single operon), the CRISPR array targeting the chromosomal lptA and mu/A genes, and the gfp reporter gene. Each CGV contained one of the promoters active in stressed cells (relB, hyaA, rpoH, and the synthetic promoter iGEM BBa_J23100) or contained no promoter transcribing the kill circuit. The CGV could be maintained in cells in which the target sequences of the kill circuit used was altered. The CGV DNA also contained the packaging site (cos) of the helper phage, and it was packaged into a phage-based delivery vector using the system shown in FIG. 1. E. coli cells were engineered to carry a defective P2 prophage. This defective prophage carried all the genes required for the production and assembly of the phage structure, for specific packaging of the CGV into the phage head, and for host cell lysis (helper functions). Expression of these genes was enabled by the activator (P4 delta) carried by Plasmid 1), which was induced by addition of arabinose to the cell culture. Using this system we were able to produce phage-like transducing particles carrying only our CGV DNA. This method can be readily applied to other bacteria.

### Example 8: CRISPR/Cas Killing of Slow Growing Cells Using Promoters Active in Slow Growing Cells

Using E. coli bSNP52 and conditions outlined in those examples we obtained cultures of fast growing (2×YT medium) and slow growing (2×YT medium+4.8 mM SHX) cells.

Cells were grown in microtiter plates as in Example 1. At T=120 minutes, cultures were diluted times in fresh medium (fast growers (under standard conditions) in 2×YT, slow growers (under stressed conditions) in 2×YT+4.8 mM SHX) and infected with CGV constructs containing nucleotide sequences encoding a CRISPR/Cas system under the control of promoters listed in Table 5.

TABLE 5
Promoters tested in Example 8

| | Transcribed | |
|---|---|---|
| Promoter | genes | Regulation |
| BBa_J23100 | | Constitutive synthetic promoter |
| | | showing high reporter levels in |
| | | stationary phase cells |
| HyaA | hyaABCDEF | RpoS dependent promoter |
| RelB | relBEF | Low level of expression in the |
| | | absence of stress, induced by |
| | | amino acid starvation |
| RpoH | rpoH | Transcribed by σ24, σ38, σ54, and |
| | | σ70, Induced by various stress. |

Growth and survival of the cells was followed by measuring the optical density of the cultures (FIG. 2). The generation time of the cells that received the CGV without a promoter (No promoter) was ˜26 min in the fast growing condition and ˜40 min in the stress conditions. As expected, all the promoter carrying CGVs inhibited cell growth more than the CGV without a promoter. However, we observed a higher level of growth inhibition relative to the no promoter CGV in the slow growing cells under stressed conditions, indicating that these promoters are indeed more active in the slow growing cells (and are thus SPA promoters).

### Example 9: CRISPR/Cas Killing of Biofilms Using Promoters Active in Slow Growing Cells Under Stress Conditions

To compare the effect of expression of kill circuits on bacteria in biofilms, we first incubated cells under conditions that promote biofilm formation, then infected them with CGV carrying phage particles, and finally, assayed the metabolic activity of biofilms by measuring conversion of the AlamarBlue dye to a fluorescent product (ThermoFisher). Biofilms were developed on peg lids in 96-well microtiter plates (ThermoFisher). In each well, cells were grown in 180 μL M9 media (SigmaAldrich), supplemented with 20 mM Glucose, 2 mM MgSO₄, 0.1 mM CaCl2, 0.1% amicase and 0.1% Mannitol. Biofilms were allowed to develop on the pegs of the lid for 24 hours at 37° C.

Next, the lid was transferred to a new microtiter plate containing fresh media. After 24 hours of incubation at 37° C., the pegs on the lid were washed three times by soaking for 10 seconds in microtiter plates containing sterile water. After washing, the lid was transferred to a new microtiter plate, containing 100 μL media and 100 μL lysate in each well, and incubated for 5 hours. Next the pegs were washed in sterile water three times as described above, and moved to a black microtiter plate containing 180 μL media and 20 μL AlamarBlue HS dye (ThermoFisher) in each well. After 1.5 hours of incubation at 37° C., fluorescence (excitation: 560 nm; emission 590 nm) was measured using a microplate reader (Biotek Synergy H1).

In the CRISPR-armed vectors, we aimed to achieve CRISPR/Cas function in diverse growth conditions. Table 6 summarizes the performance of the five evaluated promoters in our assays, and their previously reported characteristics. Four of the five tested promoters were active in biofilms, and hyaA showed the highest activity. However, our results showed that the activity of this promoter is not sufficient to support CRISPR/Cas mediated killing in planktonic cells growing in standard growth conditions. The other three promoters, bolA, rpoH, and yiaG, showed similar activities in the biofilms. However, the bolA promoter outperformed the others in planktonic cells.

TABLE 6
Relevant characteristics of the tested promoters

| | | | Killing of | Killing of |
|---|---|---|---|---|
| | | | planktonic | cells in |
| Promoter | Structure | Reported activity | cells | biofilms |
| relB | simple | Normal growth | +++ | − |
| | | Nutrient starvation | | |
| bolA | simple | Normal growth | +++ | + |
| | | Stationary phase | | |
| hyaA | simple | Biofilms | − | ++ |
| rpoH | complex | Always active | ++ | + |
| yiaG | simple | Stationary phase | + | + |

### Example 10: CRISPR/Cas Killing Complements Lytic Function of Lytic Phage Targeting Slow-Growing Cells Under Stress Conditions

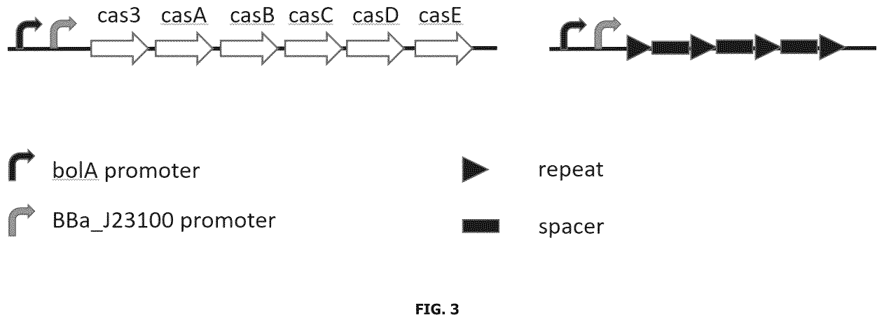

To further exemplify the use of CRISPR/Cas kill circuits expressed from promoters that are active in slow-growing cells, we engineered CRISPR/Cas-armed lytic phages carrying nucleotide sequences encoding a CRISPR/Cas system (from E. coli) expressed from the bolA and BBa_J23100 promoters and specifically targeting the chromosome of E. coli. The cas genes and the arrays were inserted into the phage genome as separate transcription units (FIG. 3).

These phages were designed to usefully leverage the lytic kill function of the phage under fast growth conditions/standard conditions (i.e., where lysis is dependent on active target cell growth), but the killing mechanism is complemented by a CRISPR/Cas mode of killing in slow growth conditions/stress conditions, made possible by use of promoter which are active in slow growing cells/under stress conditions. In FIG. 4A, we compare SNIPR Biome's phage α8 with a CRISPR/Cas armed version (08.3). Cells were grown at 36° C. in a plate reader as described in Example 5. Phages were added to the culture at the indicated time points, at the indicated amounts. The optical density of the cultures, which corresponds to the number of cells, was recorded every 10 minutes for 4 hours. We observed a more extensive killing of the cell population with the CRISPR/Cas armed phage (08.3) in the slow growing cells under stress conditions (valS^{ts}), but not in the fast growing cells under standard conditions (MG1655), as both fast and slow growing cells can be targeted for killing by α8.3. At the time of phage addition, the generation time was about 22 minutes for the fast growing cells under standard conditions, and about 52 minutes for the slow growing cells under stress conditions.

### Example 11: Staphylococcus Model System of Stressed, Slow Growing Cells

The general stress response of S. aureus is regulated by the alternative sigma factor B (σB) and it is activated by multiple stresses including oxidative stress, acidic stress, and heat. Therefore, all σ8 promoters can be considered to be SPA promoters. To study promoters responding to nutrient starvation, starvation can be induced by limiting a nutrient (e.g. in a chemically defined medium Lee et al., “Comparative genome-scale metabolic reconstruction and flux balance analysis of multiple Staphylococcus aureus genomes identify novel antimicrobial drug targets”, J. Bacteriol., 2009 June; 191(12):4015-24. doi: 10.1128/JB.01743-08), or by applying a small molecule that interferes with a specific process in the cell. For example, mupirocin is a structural analogue of isoleucyl-adenylate (Ile-AMP) and competes with Ile-AMP for overlapping binding sites of isoleucyl-tRNA synthetases (IleRSs). As a consequence, addition of mupirocin blocks the charging of isoleucyl-tRNA with isoleucine, leading to depletion of the aminoacetylated isoleucyl-tRNA pool and hence accumulation of uncharged isoleucyl-tRNAs. The increase in uncharged tRNAs relative to charged tRNAs is a signal of amino acid starvation for the cell. Proteins that are synthesized in response to amino acid starvation were identified by the method disclosed in Reiβ et al., “Global Analysis of the Staphylococcus aureus Response to Mupirocin”, Antimicrobial Agents and Chemotherapy, 2012, Vol. 56, No. 2, 787-804, which methods are incorporated herein by reference. Promoters that are active in stress conditions can be identified by using the method described in Example 6.

The increase in uncharged tRNAs relative to charged tRNAs is a signal of amino acid starvation for the cell, which triggers the stringent stress response.

### REFERENCES

Escherichia coli:
Shimada T, Makinoshima H, Ogawa Y, Miki T, Maeda M, Ishihama A., “Classification and strength measurement of stationary-phase promoters by use of a newly developed promoter cloning vector”, J Bacteriol., 2004 November; 186(21):7112-22. doi: 10.1128/JB.186.21.7112-7122.2004. PMID: 15489422; PMCID: PMC523215.
Schembri et al., “Global gene expression in Escherichia coli biofilms”, Mol. Microbiol. 2003, 48, 1, 253-267, https://doi.org/10.1046/j.1365-2958.2003.03432.x
Shah D, Zhang Z, Khodursky A, Kaldalu N, Kurg K, Lewis K. Persisters: a distinct physiological state of E. coli. BMC Microbiol. 2006 Jun. 12; 6:53. doi: 10.1186/1471-2180-6-53. PMID: 16768798; PMCID: PMC1557402.
Keren I, Shah D, Spoering A, Kaldalu N, Lewis K., “Specialised persister cells and the mechanism of multidrug tolerance in Escherichia coli”, J Bacteriol., 2004 December; 186(24):8172-80. doi: 10.1128/JB.186.24.8172-8180.2004. PMID: 15576765; PMCID: PMC532439.
Nonaka G, Blankschien M, Herman C, Gross C A, Rhodius V A., “Regulon and promoter analysis of the E. coli heat-shock factor, sigma32, reveals a multifaceted cellular response to heat stress”; Genes Dev., 2006 Jul. 1; 20(13):1776-89. doi: 10.1101/gad.1428206. PMID: 16818608; PMCID: PMC1522074.

### Staphylococcus Aureus:

Bischoff M, Dunman P, Kormanec J, Macapagal D, Murphy E, Mounts W, Berger-Bächi B, Projan S., “Microarray-based analysis of the Staphylococcus aureus sigmaB regulon”, J Bacteriol., 2004 July; 186(13):4085-99. doi: 10.1128/JB.186.13.4085-4099.2004. PMID: 15205410; PMCID: PMC421609.

Each of these references is incorporated herein by reference.

TABLE 7
Sequence listing

| Sequence | | |
|---|---|---|
| number | Description | Sequence |
| Seq ID | RelB promoter | cgactactttctgacttccttcgtgacttgccctaagcatgttgtagtgcgatacttgtaAt |
| No: 1 | sequence, σ70 | gacatttgtaattacaagaggtg |
| Seq ID | BolA promoter | aacctaaatatttgttgttaagctgcaatggaaacggtaaaagcggctagtatttaaagg |
| No: 2 | sequence, σS, σ70 | Gatggatgacatctcagcgttgtcg |
| Seq ID | Hya promoter | aaaagataaatccacacagtttgtattgttttgtgcaaaagtttcactacgctttattaaCa |
| No: 3 | sequence, σS, σ70 | atactttctggcgacgtgcgcca |
| Seq ID | YiaG promoter | agccagagcatgccctgacttcaccccgctgtgtctgcttttcccgactattcttaatgaGc |
| No: 4 | sequence, σS | ttcgatgcaattcacgatcccgc |
| Seq ID | RpoH promoter | acggtacaacatttacgccactttacgcctgaataataaaagcgtgttatactctttcccTg |
| No: 5 | sequence P1, σ70 | caatgggttccgtagcagggaaa |
| Seq ID | RpoH promoter | acggtacaacatttacgccactttacgcctgaataataaaagcgtgttatactctttcccTg |
| No: 6 | sequence P2, σS | acatgggttccgtagcagggaaa |
| Seq ID | RpoH promoter | tgcgtaatttattcacaagcttgcattgaacttgtggataaaatcacggtctgataaaacAg |
| No: 7 | sequence P3, σ24 | tgaatgataacctcgttgctctt |
| Seq ID | RpoH promoter | tttattcacaagcttgcattgaacttgtggataaaatcacggtctgataaaacagtgaatGa |
| No: 8 | sequence P4, σ70 | taacctcgttgctcttaagctct |
| Seq ID | RpoH promoter | tgcattgaacttgtggataaaatcacggtctgataaaacagtgaatgataacctcgttgcT |
| No: 9 | sequence P5, σ70 | citaagctctggcacagttgttgc |
| Seq ID | RpoH promoter | aacagtgaatgataacctcgttgctcttaagctctggcacagttgttgctaccactgaagC |
| No: 10 | sequence P6, σ54 | gccagaagatatcgattgagagga |

Core promoter sequences used (from www.ecocyc.org). Conserved promoter elements are marked: −35 or −24 boldface, −10 or −12 underlined. Transcription start sites are capitalized. The rpoH promoter region contains six transcription initiation sites (P1 to P6). Specific Q factors that recognize the promoters are indicated. The vegetative a factor is 670, that is responsible for transcription in normal growth conditions; σS is specific to stationary phase cells; σ54 is specific to nitrogen starvation; and σ24 is specific to heat shock and membrane/periplasmic stress.

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

In one embodiment, the invention comprises a combination of any one of independent claims 1 to 6 with dependent claims 10, 17, 33, 42, 43, 64 and 72.

In one embodiment, the invention comprises a combination of any one of independent claims 1 to 6 with dependent claims 15, 17, 33, 42, 43, 64 and 72.

In one embodiment, the invention comprises a combination of any one of independent claims 1 to 6 with dependent claims 16, 17, 33, 42, 43, 64 and 72.

In one embodiment, the invention comprises a combination of any one of independent claims 1 to 6 with dependent claims 10, 18, 33, 42, 43, 64 and 72.

In one embodiment, the invention comprises a combination of any one of independent claims 1 to 6 with dependent claims 15, 18, 33, 42, 43, 64 and 72.

In one embodiment, the invention comprises a combination of any one of independent claims 1 to 6 with dependent claims 16, 18, 33, 42, 43, 64 and 72.

In one embodiment, the invention comprises a combination of any one of independent claims 1 to 6 with dependent claims 10, 27, 33, 42, 43, 64 and 72.

In one embodiment, the invention comprises a combination of any one of independent claims 1 to 6 with dependent claims 15, 27, 33, 42, 43, 64 and 72.

In one embodiment, the invention comprises a combination of any one of independent claims 1 to 6 with dependent claims 16, 27, 33, 42, 43, 64 and 72.

In one embodiment, the invention comprises a combination of any one of independent claims 1 to 6 with dependent claims 10, 17, 34, 35, 36, 37, 38, 42, 43, 64 and 72.

In one embodiment, the invention comprises a combination of any one of independent claims 1 to 6 with dependent claims 15, 17, 34, 35, 36, 37, 38, 42, 43, 64 and 72.

In one embodiment, the invention comprises a combination of any one of independent claims 1 to 6 with dependent claims 16, 17, 34, 35, 36, 37, 38, 42, 43, 64 and 72.

In one embodiment, the invention comprises a combination of any one of independent claims 1 to 6 with dependent claims 10, 18, 34, 35, 36, 37, 38, 42, 43, 64 and 72.

In one embodiment, the invention comprises a combination of any one of independent claims 1 to 6 with dependent claims 15, 18, 34, 35, 36, 37, 38, 42, 43, 64 and 72.

In one embodiment, the invention comprises a combination of any one of independent claims 1 to 6 with dependent claims 16, 18, 34, 35, 36, 37, 38, 42, 43, 64 and 72.

In one embodiment, the invention comprises a combination of any one of independent claims 1 to 6 with dependent claims 10, 27, 34, 35, 36, 37, 38, 42, 43, 64 and 72.

In one embodiment, the invention comprises a combination of any one of independent claims 1 to 6 with dependent claims 15, 27, 34, 35, 36, 37, 38, 42, 43, 64 and 72.

In one embodiment, the invention comprises a combination of any one of independent claims 1 to 6 with dependent claims 16, 27, 34, 35, 36, 37, 38, 42, 43, 64 and 72.

In one embodiment, the invention comprises a combination of any one of independent claims 1 to 6 with dependent claims 10, 17, 33, 42, 44, 64 and 72.

In one embodiment, the invention comprises a combination of any one of independent claims 1 to 6 with dependent claims 15, 17, 33, 42, 44, 64 and 72.

In one embodiment, the invention comprises a combination of any one of independent claims 1 to 6 with dependent claims 16, 17, 33, 42, 44, 64 and 72.

In one embodiment, the invention comprises a combination of any one of independent claims 1 to 6 with dependent claims 10, 18, 33, 42, 44, 64 and 72.

In one embodiment, the invention comprises a combination of any one of independent claims 1 to 6 with dependent claims 15, 18, 33, 42, 44, 64 and 72.

In one embodiment, the invention comprises a combination of any one of independent claims 1 to 6 with dependent claims 16, 18, 33, 42, 44, 64 and 72.

In one embodiment, the invention comprises a combination of any one of independent claims 1 to 6 with dependent claims 10, 27, 33, 42, 44, 64 and 72.

In one embodiment, the invention comprises a combination of any one of independent claims 1 to 6 with dependent claims 15, 27, 33, 42, 44, 64 and 72.

In one embodiment, the invention comprises a combination of any one of independent claims 1 to 6 with dependent claims 16, 27, 33, 42, 44, 64 and 72.

In one embodiment, the invention comprises a combination of any one of independent claims 1 to 6 with dependent claims 10, 17, 34, 35, 36, 37, 38, 42, 44, 64 and 72.

In one embodiment, the invention comprises a combination of any one of independent claims 1 to 6 with dependent claims 15, 17, 34, 35, 36, 37, 38, 42, 44, 64 and 72.

In one embodiment, the invention comprises a combination of any one of independent claims 1 to 6 with dependent claims 16, 17, 34, 35, 36, 37, 38, 42, 44, 64 and 72.

In one embodiment, the invention comprises a combination of any one of independent claims 1 to 6 with dependent claims 10, 18, 34, 35, 36, 37, 38, 42, 44, 64 and 72.

In one embodiment, the invention comprises a combination of any one of independent claims 1 to 6 with dependent claims 15, 18, 34, 35, 36, 37, 38, 42, 44, 64 and 72.

In one embodiment, the invention comprises a combination of any one of independent claims 1 to 6 with dependent claims 16, 18, 34, 35, 36, 37, 38, 42, 44, 64 and 72.

In one embodiment, the invention comprises a combination of any one of independent claims 1 to 6 with dependent claims 10, 27, 34, 35, 36, 37, 38, 42, 44, 64 and 72.

In one embodiment, the invention comprises a combination of any one of independent claims 1 to 6 with dependent claims 15, 27, 34, 35, 36, 37, 38, 42, 44, 64 and 72.

In one embodiment, the invention comprises a combination of any one of independent claims 1 to 6 with dependent claims 16, 27, 34, 35, 36, 37, 38, 42, 44, 64 and 72.

In one embodiment, the invention comprises a combination of any one of independent claims 1 to 6 with dependent claims 10, 17, 33, 42, 45, 64 and 72.

In one embodiment, the invention comprises a combination of any one of independent claims 1 to 6 with dependent claims 15, 17, 33, 42, 45, 64 and 72.

In one embodiment, the invention comprises a combination of any one of independent claims 1 to 6 with dependent claims 16, 17, 33, 42, 45, 64 and 72.

In one embodiment, the invention comprises a combination of any one of independent claims 1 to 6 with dependent claims 10, 18, 33, 42, 45, 64 and 72.

In one embodiment, the invention comprises a combination of any one of independent claims 1 to 6 with dependent claims 15, 18, 33, 42, 45, 64 and 72.

In one embodiment, the invention comprises a combination of any one of independent claims 1 to 6 with dependent claims 16, 18, 33, 42, 45, 64 and 72.

In one embodiment, the invention comprises a combination of any one of independent claims 1 to 6 with dependent claims 10, 27, 33, 42, 45, 64 and 72.

In one embodiment, the invention comprises a combination of any one of independent claims 1 to 6 with dependent claims 15, 27, 33, 42, 45, 64 and 72.

In one embodiment, the invention comprises a combination of any one of independent claims 1 to 6 with dependent claims 16, 27, 33, 42, 45, 64 and 72.

In one embodiment, the invention comprises a combination of any one of independent claims 1 to 6 with dependent claims 10, 17, 34, 35, 36, 37, 38, 42, 45, 64 and 72.

In one embodiment, the invention comprises a combination of any one of independent claims 1 to 6 with dependent claims 15, 17, 34, 35, 36, 37, 38, 42, 45, 64 and 72.

In one embodiment, the invention comprises a combination of any one of independent claims 1 to 6 with dependent claims 16, 17, 34, 35, 36, 37, 38, 42, 45, 64 and 72.

In one embodiment, the invention comprises a combination of any one of independent claims 1 to 6 with dependent claims 10, 18, 34, 35, 36, 37, 38, 42, 45, 64 and 72.

In one embodiment, the invention comprises a combination of any one of independent claims 1 to 6 with dependent claims 15, 18, 34, 35, 36, 37, 38, 42, 45, 64 and 72.

In one embodiment, the invention comprises a combination of any one of independent claims 1 to 6 with dependent claims 16, 18, 34, 35, 36, 37, 38, 42, 45, 64 and 72.

In one embodiment, the invention comprises a combination of any one of independent claims 1 to 6 with dependent claims 10, 27, 34, 35, 36, 37, 38, 42, 45, 64 and 72.

In one embodiment, the invention comprises a combination of any one of independent claims 1 to 6 with dependent claims 15, 27, 34, 35, 36, 37, 38, 42, 45, 64 and 72.

In one embodiment, the invention comprises a combination of any one of independent claims 1 to 6 with dependent claims 16, 27, 34, 35, 36, 37, 38, 42, 45, 64 and 72.

In one embodiment, the invention comprises a combination of any one of independent claims 1 to 6 with dependent claims 10, 17, 33, 42, 43, 64, 67 and 72.

In one embodiment, the invention comprises a combination of any one of independent claims 1 to 6 with dependent claims 15, 17, 33, 42, 43, 64, 67 and 72.

In one embodiment, the invention comprises a combination of any one of independent claims 1 to 6 with dependent claims 16, 17, 33, 42, 43, 64, 67 and 72.

In one embodiment, the invention comprises a combination of any one of independent claims 1 to 6 with dependent claims 10, 18, 33, 42, 43, 64, 67 and 72.

In one embodiment, the invention comprises a combination of any one of independent claims 1 to 6 with dependent claims 15, 18, 33, 42, 43, 64, 67 and 72.

In one embodiment, the invention comprises a combination of any one of independent claims 1 to 6 with dependent claims 16, 18, 33, 42, 43, 64, 67 and 72.

In one embodiment, the invention comprises a combination of any one of independent claims 1 to 6 with dependent claims 10, 27, 33, 42, 43, 64, 67 and 72.

In one embodiment, the invention comprises a combination of any one of independent claims 1 to 6 with dependent claims 15, 27, 33, 42, 43, 64, 67 and 72.

In one embodiment, the invention comprises a combination of any one of independent claims 1 to 6 with dependent claims 16, 27, 33, 42, 43, 64, 67 and 72.

In one embodiment, the invention comprises a combination of any one of independent claims 1 to 6 with dependent claims 10, 17, 34, 35, 36, 37, 38, 42, 43, 64, 67 and 72.

In one embodiment, the invention comprises a combination of any one of independent claims 1 to 6 with dependent claims 15, 17, 34, 35, 36, 37, 38, 42, 43, 64, 67 and 72.

In one embodiment, the invention comprises a combination of any one of independent claims 1 to 6 with dependent claims 16, 17, 34, 35, 36, 37, 38, 42, 43, 64, 67 and 72.

In one embodiment, the invention comprises a combination of any one of independent claims 1 to 6 with dependent claims 10, 18, 34, 35, 36, 37, 38, 42, 43, 64, 67 and 72.

In one embodiment, the invention comprises a combination of any one of independent claims 1 to 6 with dependent claims 15, 18, 34, 35, 36, 37, 38, 42, 43, 64, 67 and 72.

In one embodiment, the invention comprises a combination of any one of independent claims 1 to 6 with dependent claims 16, 18, 34, 35, 36, 37, 38, 42, 43, 64, 67 and 72.

In one embodiment, the invention comprises a combination of any one of independent claims 1 to 6 with dependent claims 10, 27, 34, 35, 36, 37, 38, 42, 43, 64, 67 and 72.

In one embodiment, the invention comprises a combination of any one of independent claims 1 to 6 with dependent claims 15, 27, 34, 35, 36, 37, 38, 42, 43, 64, 67 and 72.

In one embodiment, the invention comprises a combination of any one of independent claims 1 to 6 with dependent claims 16, 27, 34, 35, 36, 37, 38, 42, 43, 64, 67 and 72.

In one embodiment, the invention comprises a combination of any one of independent claims 1 to 6 with dependent claims 10, 17, 33, 42, 44, 64, 67 and 72.

In one embodiment, the invention comprises a combination of any one of independent claims 1 to 6 with dependent claims 15, 17, 33, 42, 44, 64, 67 and 72.

In one embodiment, the invention comprises a combination of any one of independent claims 1 to 6 with dependent claims 16, 17, 33, 42, 44, 64, 67 and 72.

In one embodiment, the invention comprises a combination of any one of independent claims 1 to 6 with dependent claims 10, 18, 33, 42, 44, 64, 67 and 72.

In one embodiment, the invention comprises a combination of any one of independent claims 1 to 6 with dependent claims 15, 18, 33, 42, 44, 64, 67 and 72.

In one embodiment, the invention comprises a combination of any one of independent claims 1 to 6 with dependent claims 16, 18, 33, 42, 44, 64, 67 and 72.

In one embodiment, the invention comprises a combination of any one of independent claims 1 to 6 with dependent claims 10, 27, 33, 42, 44, 64, 67 and 72.

In one embodiment, the invention comprises a combination of any one of independent claims 1 to 6 with dependent claims 15, 27, 33, 42, 44, 64, 67 and 72.

In one embodiment, the invention comprises a combination of any one of independent claims 1 to 6 with dependent claims 16, 27, 33, 42, 44, 64, 67 and 72.

In one embodiment, the invention comprises a combination of any one of independent claims 1 to 6 with dependent claims 10, 17, 34, 35, 36, 37, 38, 42, 44, 64, 67 and 72.

In one embodiment, the invention comprises a combination of any one of independent claims 1 to 6 with dependent claims 15, 17, 34, 35, 36, 37, 38, 42, 44, 64, 67 and 72.

In one embodiment, the invention comprises a combination of any one of independent claims 1 to 6 with dependent claims 16, 17, 34, 35, 36, 37, 38, 42, 44, 64, 67 and 72.

In one embodiment, the invention comprises a combination of any one of independent claims 1 to 6 with dependent claims 10, 18, 34, 35, 36, 37, 38, 42, 44, 64, 67 and 72.

In one embodiment, the invention comprises a combination of any one of independent claims 1 to 6 with dependent claims 15, 18, 34, 35, 36, 37, 38, 42, 44, 64, 67 and 72.

In one embodiment, the invention comprises a combination of any one of independent claims 1 to 6 with dependent claims 16, 18, 34, 35, 36, 37, 38, 42, 44, 64, 67 and 72.

In one embodiment, the invention comprises a combination of any one of independent claims 1 to 6 with dependent claims 10, 27, 34, 35, 36, 37, 38, 42, 44, 64, 67 and 72.

In one embodiment, the invention comprises a combination of any one of independent claims 1 to 6 with dependent claims 15, 27, 34, 35, 36, 37, 38, 42, 44, 64, 67 and 72.

In one embodiment, the invention comprises a combination of any one of independent claims 1 to 6 with dependent claims 16, 27, 34, 35, 36, 37, 38, 42, 44, 64, 67 and 72.

In one embodiment, the invention comprises a combination of any one of independent claims 1 to 6 with dependent claims 10, 17, 33, 42, 45, 64, 67 and 72.

In one embodiment, the invention comprises a combination of any one of independent claims 1 to 6 with dependent claims 15, 17, 33, 42, 45, 64, 67 and 72.

In one embodiment, the invention comprises a combination of any one of independent claims 1 to 6 with dependent claims 16, 17, 33, 42, 45, 64, 67 and 72.

In one embodiment, the invention comprises a combination of any one of independent claims 1 to 6 with dependent claims 10, 18, 33, 42, 45, 64, 67 and 72.

In one embodiment, the invention comprises a combination of any one of independent claims 1 to 6 with dependent claims 15, 18, 33, 42, 45, 64, 67 and 72.

In one embodiment, the invention comprises a combination of any one of independent claims 1 to 6 with dependent claims 16, 18, 33, 42, 45, 64, 67 and 72.

In one embodiment, the invention comprises a combination of any one of independent claims 1 to 6 with dependent claims 10, 27, 33, 42, 45, 64, 67 and 72.

In one embodiment, the invention comprises a combination of any one of independent claims 1 to 6 with dependent claims 15, 27, 33, 42, 45, 64, 67 and 72.

In one embodiment, the invention comprises a combination of any one of independent claims 1 to 6 with dependent claims 16, 27, 33, 42, 45, 64, 67 and 72.

In one embodiment, the invention comprises a combination of any one of independent claims 1 to 6 with dependent claims 10, 17, 34, 35, 36, 37, 38, 42, 45, 64, 67 and 72.

In one embodiment, the invention comprises a combination of any one of independent claims 1 to 6 with dependent claims 15, 17, 34, 35, 36, 37, 38, 42, 45, 64, 67 and 72.

In one embodiment, the invention comprises a combination of any one of independent claims 1 to 6 with dependent claims 16, 17, 34, 35, 36, 37, 38, 42, 45, 64, 67 and 72.

In one embodiment, the invention comprises a combination of any one of independent claims 1 to 6 with dependent claims 10, 18, 34, 35, 36, 37, 38, 42, 45, 64, 67 and 72.

In one embodiment, the invention comprises a combination of any one of independent claims 1 to 6 with dependent claims 15, 18, 34, 35, 36, 37, 38, 42, 45, 64, 67 and 72.

In one embodiment, the invention comprises a combination of any one of independent claims 1 to 6 with dependent claims 16, 18, 34, 35, 36, 37, 38, 42, 45, 64, 67 and 72.

In one embodiment, the invention comprises a combination of any one of independent claims 1 to 6 with dependent claims 10, 27, 34, 35, 36, 37, 38, 42, 45, 64, 67 and 72.

In one embodiment, the invention comprises a combination of any one of independent claims 1 to 6 with dependent claims 15, 27, 34, 35, 36, 37, 38, 42, 45, 64, 67 and 72.

In one embodiment, the invention comprises a combination of any one of independent claims 1 to 6 with dependent claims 16, 27, 34, 35, 36, 37, 38, 42, 45, 64, 67 and 72.

In one embodiment, the invention comprises a combination of any one of independent claims 1 to 6 with dependent claims 10, 17, 33, 42, 43, 64 and 74.

In one embodiment, the invention comprises a combination of any one of independent claims 1 to 6 with dependent claims 15, 17, 33, 42, 43, 64, and 74.

In one embodiment, the invention comprises a combination of any one of independent claims 1 to 6 with dependent claims 16, 17, 33, 42, 43, 64 and 74.

In one embodiment, the invention comprises a combination of any one of independent claims 1 to 6 with dependent claims 10, 18, 33, 42, 43, 64 and 74.

In one embodiment, the invention comprises a combination of any one of independent claims 1 to 6 with dependent claims 15, 18, 33, 42, 43, 64 and 74.

In one embodiment, the invention comprises a combination of any one of independent claims 1 to 6 with dependent claims 16, 18, 33, 42, 43, 64 and 74.

In one embodiment, the invention comprises a combination of any one of independent claims 1 to 6 with dependent claims 10, 27, 33, 42, 43, 64 and 74.

In one embodiment, the invention comprises a combination of any one of independent claims 1 to 6 with dependent claims 15, 27, 33, 42, 43, 64 and 74.

In one embodiment, the invention comprises a combination of any one of independent claims 1 to 6 with dependent claims 16, 27, 33, 42, 43, 64 and 74.

In one embodiment, the invention comprises a combination of any one of independent claims 1 to 6 with dependent claims 10, 17, 34, 35, 36, 37, 38, 42, 43, 64 and 74.

In one embodiment, the invention comprises a combination of any one of independent claims 1 to 6 with dependent claims 15, 17, 34, 35, 36, 37, 38, 42, 43, 64 and 74.

In one embodiment, the invention comprises a combination of any one of independent claims 1 to 6 with dependent claims 16, 17, 34, 35, 36, 37, 38, 42, 43, 64 and 74.

In one embodiment, the invention comprises a combination of any one of independent claims 1 to 6 with dependent claims 10, 18, 34, 35, 36, 37, 38, 42, 43, 64 and 74.

In one embodiment, the invention comprises a combination of any one of independent claims 1 to 6 with dependent claims 15, 18, 34, 35, 36, 37, 38, 42, 43, 64 and 74.

In one embodiment, the invention comprises a combination of any one of independent claims 1 to 6 with dependent claims 16, 18, 34, 35, 36, 37, 38, 42, 43, 64 and 74.

In one embodiment, the invention comprises a combination of any one of independent claims 1 to 6 with dependent claims 10, 27, 34, 35, 36, 37, 38, 42, 43, 64 and 74.

In one embodiment, the invention comprises a combination of any one of independent claims 1 to 6 with dependent claims 15, 27, 34, 35, 36, 37, 38, 42, 43, 64 and 74.

In one embodiment, the invention comprises a combination of any one of independent claims 1 to 6 with dependent claims 16, 27, 34, 35, 36, 37, 38, 42, 43, 64 and 74.

In one embodiment, the invention comprises a combination of any one of independent claims 1 to 6 with dependent claims 10, 17, 33, 42, 44, 64 and 74.

In one embodiment, the invention comprises a combination of any one of independent claims 1 to 6 with dependent claims 15, 17, 33, 42, 44, 64 and 74.

In one embodiment, the invention comprises a combination of any one of independent claims 1 to 6 with dependent claims 16, 17, 33, 42, 44, 64 and 74.

In one embodiment, the invention comprises a combination of any one of independent claims 1 to 6 with dependent claims 10, 18, 33, 42, 44, 64 and 74.

In one embodiment, the invention comprises a combination of any one of independent claims 1 to 6 with dependent claims 15, 18, 33, 42, 44, 64 and 74.

In one embodiment, the invention comprises a combination of any one of independent claims 1 to 6 with dependent claims 16, 18, 33, 42, 44, 64 and 74.

In one embodiment, the invention comprises a combination of any one of independent claims 1 to 6 with dependent claims 10, 27, 33, 42, 44, 64 and 74.

In one embodiment, the invention comprises a combination of any one of independent claims 1 to 6 with dependent claims 15, 27, 33, 42, 44, 64 and 74.

In one embodiment, the invention comprises a combination of any one of independent claims 1 to 6 with dependent claims 16, 27, 33, 42, 44, 64 and 74.

In one embodiment, the invention comprises a combination of any one of independent claims 1 to 6 with dependent claims 10, 17, 34, 35, 36, 37, 38, 42, 44, 64 and 74.

In one embodiment, the invention comprises a combination of any one of independent claims 1 to 6 with dependent claims 15, 17, 34, 35, 36, 37, 38, 42, 44, 64 and 74.

In one embodiment, the invention comprises a combination of any one of independent claims 1 to 6 with dependent claims 16, 17, 34, 35, 36, 37, 38, 42, 44, 64 and 74.

In one embodiment, the invention comprises a combination of any one of independent claims 1 to 6 with dependent claims 10, 18, 34, 35, 36, 37, 38, 42, 44, 64 and 74.

In one embodiment, the invention comprises a combination of any one of independent claims 1 to 6 with dependent claims 15, 18, 34, 35, 36, 37, 38, 42, 44, 64 and 74.

In one embodiment, the invention comprises a combination of any one of independent claims 1 to 6 with dependent claims 16, 18, 34, 35, 36, 37, 38, 42, 44, 64 and 74.

In one embodiment, the invention comprises a combination of any one of independent claims 1 to 6 with dependent claims 10, 27, 34, 35, 36, 37, 38, 42, 44, 64 and 74.

In one embodiment, the invention comprises a combination of any one of independent claims 1 to 6 with dependent claims 15, 27, 34, 35, 36, 37, 38, 42, 44, 64 and 74.

In one embodiment, the invention comprises a combination of any one of independent claims 1 to 6 with dependent claims 16, 27, 34, 35, 36, 37, 38, 42, 44, 64 and 74.

In one embodiment, the invention comprises a combination of any one of independent claims 1 to 6 with dependent claims 10, 17, 33, 42, 45, 64 and 74.

In one embodiment, the invention comprises a combination of any one of independent claims 1 to 6 with dependent claims 15, 17, 33, 42, 45, 64 and 74.

In one embodiment, the invention comprises a combination of any one of independent claims 1 to 6 with dependent claims 16, 17, 33, 42, 45, 64 and 74.

In one embodiment, the invention comprises a combination of any one of independent claims 1 to 6 with dependent claims 10, 18, 33, 42, 45, 64 and 74.

In one embodiment, the invention comprises a combination of any one of independent claims 1 to 6 with dependent claims 15, 18, 33, 42, 45, 64 and 74.

In one embodiment, the invention comprises a combination of any one of independent claims 1 to 6 with dependent claims 16, 18, 33, 42, 45, 64 and 74.

In one embodiment, the invention comprises a combination of any one of independent claims 1 to 6 with dependent claims 10, 27, 33, 42, 45, 64 and 74.

In one embodiment, the invention comprises a combination of any one of independent claims 1 to 6 with dependent claims 15, 27, 33, 42, 45, 64 and 74.

In one embodiment, the invention comprises a combination of any one of independent claims 1 to 6 with dependent claims 16, 27, 33, 42, 45, 64 and 74.

In one embodiment, the invention comprises a combination of any one of independent claims 1 to 6 with dependent claims 10, 17, 34, 35, 36, 37, 38, 42, 45, 64 and 74.

In one embodiment, the invention comprises a combination of any one of independent claims 1 to 6 with dependent claims 15, 17, 34, 35, 36, 37, 38, 42, 45, 64 and 74.

In one embodiment, the invention comprises a combination of any one of independent claims 1 to 6 with dependent claims 16, 17, 34, 35, 36, 37, 38, 42, 45, 64 and 74.

In one embodiment, the invention comprises a combination of any one of independent claims 1 to 6 with dependent claims 10, 18, 34, 35, 36, 37, 38, 42, 45, 64 and 74.

In one embodiment, the invention comprises a combination of any one of independent claims 1 to 6 with dependent claims 15, 18, 34, 35, 36, 37, 38, 42, 45, 64 and 74.

In one embodiment, the invention comprises a combination of any one of independent claims 1 to 6 with dependent claims 16, 18, 34, 35, 36, 37, 38, 42, 45, 64 and 74.

In one embodiment, the invention comprises a combination of any one of independent claims 1 to 6 with dependent claims 10, 27, 34, 35, 36, 37, 38, 42, 45, 64 and 74.

In one embodiment, the invention comprises a combination of any one of independent claims 1 to 6 with dependent claims 15, 27, 34, 35, 36, 37, 38, 42, 45, 64 and 74.

In one embodiment, the invention comprises a combination of any one of independent claims 1 to 6 with dependent claims 16, 27, 34, 35, 36, 37, 38, 42, 45, 64 and 74.

In one embodiment, the invention comprises a combination of any one of independent claims 1 to 6 with dependent claims 10, 17, 33, 42, 43, 64, 67 and 74.

In one embodiment, the invention comprises a combination of any one of independent claims 1 to 6 with dependent claims 15, 17, 33, 42, 43, 64, 67 and 74.

In one embodiment, the invention comprises a combination of any one of independent claims 1 to 6 with dependent claims 16, 17, 33, 42, 43, 64, 67 and 74.

In one embodiment, the invention comprises a combination of any one of independent claims 1 to 6 with dependent claims 10, 18, 33, 42, 43, 64, 67 and 74.

In one embodiment, the invention comprises a combination of any one of independent claims 1 to 6 with dependent claims 15, 18, 33, 42, 43, 64, 67 and 74.

In one embodiment, the invention comprises a combination of any one of independent claims 1 to 6 with dependent claims 16, 18, 33, 42, 43, 64, 67 and 74.

In one embodiment, the invention comprises a combination of any one of independent claims 1 to 6 with dependent claims 10, 27, 33, 42, 43, 64, 67 and 74.

In one embodiment, the invention comprises a combination of any one of independent claims 1 to 6 with dependent claims 15, 27, 33, 42, 43, 64, 67 and 74.

In one embodiment, the invention comprises a combination of any one of independent claims 1 to 6 with dependent claims 16, 27, 33, 42, 43, 64, 67 and 74.

In one embodiment, the invention comprises a combination of any one of independent claims 1 to 6 with dependent claims 10, 17, 34, 35, 36, 37, 38, 42, 43, 64, 67 and 74.

In one embodiment, the invention comprises a combination of any one of independent claims 1 to 6 with dependent claims 15, 17, 34, 35, 36, 37, 38, 42, 43, 64, 67 and 74.

In one embodiment, the invention comprises a combination of any one of independent claims 1 to 6 with dependent claims 16, 17, 34, 35, 36, 37, 38, 42, 43, 64, 67 and 74.

In one embodiment, the invention comprises a combination of any one of independent claims 1 to 6 with dependent claims 10, 18, 34, 35, 36, 37, 38, 42, 43, 64, 67 and 74.

In one embodiment, the invention comprises a combination of any one of independent claims 1 to 6 with dependent claims 15, 18, 34, 35, 36, 37, 38, 42, 43, 64, 67 and 74.

In one embodiment, the invention comprises a combination of any one of independent claims 1 to 6 with dependent claims 16, 18, 34, 35, 36, 37, 38, 42, 43, 64, 67 and 74.

In one embodiment, the invention comprises a combination of any one of independent claims 1 to 6 with dependent claims 10, 27, 34, 35, 36, 37, 38, 42, 43, 64, 67 and 74.

In one embodiment, the invention comprises a combination of any one of independent claims 1 to 6 with dependent claims 15, 27, 34, 35, 36, 37, 38, 42, 43, 64, 67 and 74.

In one embodiment, the invention comprises a combination of any one of independent claims 1 to 6 with dependent claims 16, 27, 34, 35, 36, 37, 38, 42, 43, 64, 67 and 74.

In one embodiment, the invention comprises a combination of any one of independent claims 1 to 6 with dependent claims 10, 17, 33, 42, 44, 64, 67 and 74.

In one embodiment, the invention comprises a combination of any one of independent claims 1 to 6 with dependent claims 15, 17, 33, 42, 44, 64, 67 and 74.

In one embodiment, the invention comprises a combination of any one of independent claims 1 to 6 with dependent claims 16, 17, 33, 42, 44, 64, 67 and 74.

In one embodiment, the invention comprises a combination of any one of independent claims 1 to 6 with dependent claims 10, 18, 33, 42, 44, 64, 67 and 74.

In one embodiment, the invention comprises a combination of any one of independent claims 1 to 6 with dependent claims 15, 18, 33, 42, 44, 64, 67 and 74.

In one embodiment, the invention comprises a combination of any one of independent claims 1 to 6 with dependent claims 16, 18, 33, 42, 44, 64, 67 and 74.

In one embodiment, the invention comprises a combination of any one of independent claims 1 to 6 with dependent claims 10, 27, 33, 42, 44, 64, 67 and 74.

In one embodiment, the invention comprises a combination of any one of independent claims 1 to 6 with dependent claims 15, 27, 33, 42, 44, 64, 67 and 74.

In one embodiment, the invention comprises a combination of any one of independent claims 1 to 6 with dependent claims 16, 27, 33, 42, 44, 64, 67 and 74.

In one embodiment, the invention comprises a combination of any one of independent claims 1 to 6 with dependent claims 10, 17, 34, 35, 36, 37, 38, 42, 44, 64, 67 and 74.

In one embodiment, the invention comprises a combination of any one of independent claims 1 to 6 with dependent claims 15, 17, 34, 35, 36, 37, 38, 42, 44, 64, 67 and 74.

In one embodiment, the invention comprises a combination of any one of independent claims 1 to 6 with dependent claims 16, 17, 34, 35, 36, 37, 38, 42, 44, 64, 67 and 74.

In one embodiment, the invention comprises a combination of any one of independent claims 1 to 6 with dependent claims 10, 18, 34, 35, 36, 37, 38, 42, 44, 64, 67 and 74.

In one embodiment, the invention comprises a combination of any one of independent claims 1 to 6 with dependent claims 15, 18, 34, 35, 36, 37, 38, 42, 44, 64, 67 and 74.

In one embodiment, the invention comprises a combination of any one of independent claims 1 to 6 with dependent claims 16, 18, 34, 35, 36, 37, 38, 42, 44, 64, 67 and 74.

In one embodiment, the invention comprises a combination of any one of independent claims 1 to 6 with dependent claims 10, 27, 34, 35, 36, 37, 38, 42, 44, 64, 67 and 74.

In one embodiment, the invention comprises a combination of any one of independent claims 1 to 6 with dependent claims 15, 27, 34, 35, 36, 37, 38, 42, 44, 64, 67 and 74.

In one embodiment, the invention comprises a combination of any one of independent claims 1 to 6 with dependent claims 16, 27, 34, 35, 36, 37, 38, 42, 44, 64, 67 and 74.

In one embodiment, the invention comprises a combination of any one of independent claims 1 to 6 with dependent claims 10, 17, 33, 42, 45, 64, 67 and 74.

In one embodiment, the invention comprises a combination of any one of independent claims 1 to 6 with dependent claims 15, 17, 33, 42, 45, 64, 67 and 74.

In one embodiment, the invention comprises a combination of any one of independent claims 1 to 6 with dependent claims 16, 17, 33, 42, 45, 64, 67 and 74.

In one embodiment, the invention comprises a combination of any one of independent claims 1 to 6 with dependent claims 10, 18, 33, 42, 45, 64, 67 and 74.

In one embodiment, the invention comprises a combination of any one of independent claims 1 to 6 with dependent claims 15, 18, 33, 42, 45, 64, 67 and 74.

In one embodiment, the invention comprises a combination of any one of independent claims 1 to 6 with dependent claims 16, 18, 33, 42, 45, 64, 67 and 74.

In one embodiment, the invention comprises a combination of any one of independent claims 1 to 6 with dependent claims 10, 27, 33, 42, 45, 64, 67 and 74.

In one embodiment, the invention comprises a combination of any one of independent claims 1 to 6 with dependent claims 15, 27, 33, 42, 45, 64, 67 and 74.

In one embodiment, the invention comprises a combination of any one of independent claims 1 to 6 with dependent claims 16, 27, 33, 42, 45, 64, 67 and 74.

In one embodiment, the invention comprises a combination of any one of independent claims 1 to 6 with dependent claims 10, 17, 34, 35, 36, 37, 38, 42, 45, 64, 67 and 74.

In one embodiment, the invention comprises a combination of any one of independent claims 1 to 6 with dependent claims 15, 17, 34, 35, 36, 37, 38, 42, 45, 64, 67 and 74.

In one embodiment, the invention comprises a combination of any one of independent claims 1 to 6 with dependent claims 16, 17, 34, 35, 36, 37, 38, 42, 45, 64, 67 and 74.

In one embodiment, the invention comprises a combination of any one of independent claims 1 to 6 with dependent claims 10, 18, 34, 35, 36, 37, 38, 42, 45, 64, 67 and 74.

In one embodiment, the invention comprises a combination of any one of independent claims 1 to 6 with dependent claims 15, 18, 34, 35, 36, 37, 38, 42, 45, 64, 67 and 74.

In one embodiment, the invention comprises a combination of any one of independent claims 1 to 6 with dependent claims 16, 18, 34, 35, 36, 37, 38, 42, 45, 64, 67 and 74.

In one embodiment, the invention comprises a combination of any one of independent claims 1 to 6 with dependent claims 10, 27, 34, 35, 36, 37, 38, 42, 45, 64, 67 and 74.

In one embodiment, the invention comprises a combination of any one of independent claims 1 to 6 with dependent claims 15, 27, 34, 35, 36, 37, 38, 42, 45, 64, 67 and 74.

In one embodiment, the invention comprises a combination of any one of independent claims 1 to 6 with dependent claims 16, 27, 34, 35, 36, 37, 38, 42, 45, 64, 67 and 74.

In one embodiment, the invention comprises a combination of any one of independent claims 1 to 6 with dependent claims 10, 17, 33, 42, 43, 64, 72 and 86.

In one embodiment, the invention comprises a combination of any one of independent claims 1 to 6 with dependent claims 15, 17, 33, 42, 43, 64, 72 and 86.

In one embodiment, the invention comprises a combination of any one of independent claims 1 to 6 with dependent claims 16, 17, 33, 42, 43, 64, 72 and 86.

In one embodiment, the invention comprises a combination of any one of independent claims 1 to 6 with dependent claims 10, 18, 33, 42, 43, 64, 72 and 86.

In one embodiment, the invention comprises a combination of any one of independent claims 1 to 6 with dependent claims 15, 18, 33, 42, 43, 64, 72 and 86.

In one embodiment, the invention comprises a combination of any one of independent claims 1 to 6 with dependent claims 16, 18, 33, 42, 43, 64, 72 and 86.

In one embodiment, the invention comprises a combination of any one of independent claims 1 to 6 with dependent claims 10, 27, 33, 42, 43, 64, 72 and 86.

In one embodiment, the invention comprises a combination of any one of independent claims 1 to 6 with dependent claims 15, 27, 33, 42, 43, 64, 72 and 86.

In one embodiment, the invention comprises a combination of any one of independent claims 1 to 6 with dependent claims 16, 27, 33, 42, 43, 64, 72 and 86.

In one embodiment, the invention comprises a combination of any one of independent claims 1 to 6 with dependent claims 10, 17, 34, 35, 36, 37, 38, 42, 43, 64, 72 and 86.

In one embodiment, the invention comprises a combination of any one of independent claims 1 to 6 with dependent claims 15, 17, 34, 35, 36, 37, 38, 42, 43, 64, 72 and 86.

In one embodiment, the invention comprises a combination of any one of independent claims 1 to 6 with dependent claims 16, 17, 34, 35, 36, 37, 38, 42, 43, 64, 72 and 86.

In one embodiment, the invention comprises a combination of any one of independent claims 1 to 6 with dependent claims 10, 18, 34, 35, 36, 37, 38, 42, 43, 64, 72 and 86.

In one embodiment, the invention comprises a combination of any one of independent claims 1 to 6 with dependent claims 15, 18, 34, 35, 36, 37, 38, 42, 43, 64, 72 and 86.

In one embodiment, the invention comprises a combination of any one of independent claims 1 to 6 with dependent claims 16, 18, 34, 35, 36, 37, 38, 42, 43, 64, 72 and 86.

In one embodiment, the invention comprises a combination of any one of independent claims 1 to 6 with dependent claims 10, 27, 34, 35, 36, 37, 38, 42, 43, 64, 72 and 86.

In one embodiment, the invention comprises a combination of any one of independent claims 1 to 6 with dependent claims 15, 27, 34, 35, 36, 37, 38, 42, 43, 64, 72 and 86.

In one embodiment, the invention comprises a combination of any one of independent claims 1 to 6 with dependent claims 16, 27, 34, 35, 36, 37, 38, 42, 43, 64, 72 and 86.

In one embodiment, the invention comprises a combination of any one of independent claims 1 to 6 with dependent claims 10, 17, 33, 42, 44, 64, 72 and 86.

In one embodiment, the invention comprises a combination of any one of independent claims 1 to 6 with dependent claims 15, 17, 33, 42, 44, 64, 72 and 86.

In one embodiment, the invention comprises a combination of any one of independent claims 1 to 6 with dependent claims 16, 17, 33, 42, 44, 64, 72 and 86.

In one embodiment, the invention comprises a combination of any one of independent claims 1 to 6 with dependent claims 10, 18, 33, 42, 44, 64, 72 and 86.

In one embodiment, the invention comprises a combination of any one of independent claims 1 to 6 with dependent claims 15, 18, 33, 42, 44, 64, 72 and 86.

In one embodiment, the invention comprises a combination of any one of independent claims 1 to 6 with dependent claims 16, 18, 33, 42, 44, 64, 72 and 86.

In one embodiment, the invention comprises a combination of any one of independent claims 1 to 6 with dependent claims 10, 27, 33, 42, 44, 64, 72 and 86.

In one embodiment, the invention comprises a combination of any one of independent claims 1 to 6 with dependent claims 15, 27, 33, 42, 44, 64, 72 and 86.

In one embodiment, the invention comprises a combination of any one of independent claims 1 to 6 with dependent claims 16, 27, 33, 42, 44, 64, 72 and 86.

In one embodiment, the invention comprises a combination of any one of independent claims 1 to 6 with dependent claims 10, 17, 34, 35, 36, 37, 38, 42, 44, 64, 72 and 86.

In one embodiment, the invention comprises a combination of any one of independent claims 1 to 6 with dependent claims 15, 17, 34, 35, 36, 37, 38, 42, 44, 64, 72 and 86.

In one embodiment, the invention comprises a combination of any one of independent claims 1 to 6 with dependent claims 16, 17, 34, 35, 36, 37, 38, 42, 44, 64, 72 and 86.

In one embodiment, the invention comprises a combination of any one of independent claims 1 to 6 with dependent claims 10, 18, 34, 35, 36, 37, 38, 42, 44, 64, 72 and 86.

In one embodiment, the invention comprises a combination of any one of independent claims 1 to 6 with dependent claims 15, 18, 34, 35, 36, 37, 38, 42, 44, 64, 72 and 86.

In one embodiment, the invention comprises a combination of any one of independent claims 1 to 6 with dependent claims 16, 18, 34, 35, 36, 37, 38, 42, 44, 64, 72 and 86.

In one embodiment, the invention comprises a combination of any one of independent claims 1 to 6 with dependent claims 10, 27, 34, 35, 36, 37, 38, 42, 44, 64, 72 and 86.

In one embodiment, the invention comprises a combination of any one of independent claims 1 to 6 with dependent claims 15, 27, 34, 35, 36, 37, 38, 42, 44, 64, 72 and 86.

In one embodiment, the invention comprises a combination of any one of independent claims 1 to 6 with dependent claims 16, 27, 34, 35, 36, 37, 38, 42, 44, 64, 72 and 86.

In one embodiment, the invention comprises a combination of any one of independent claims 1 to 6 with dependent claims 10, 17, 33, 42, 45, 64, 72 and 86.

In one embodiment, the invention comprises a combination of any one of independent claims 1 to 6 with dependent claims 15, 17, 33, 42, 45, 64, 72 and 86.

In one embodiment, the invention comprises a combination of any one of independent claims 1 to 6 with dependent claims 16, 17, 33, 42, 45, 64, 72 and 86.

In one embodiment, the invention comprises a combination of any one of independent claims 1 to 6 with dependent claims 10, 18, 33, 42, 45, 64, 72 and 86.

In one embodiment, the invention comprises a combination of any one of independent claims 1 to 6 with dependent claims 15, 18, 33, 42, 45, 64, 72 and 86.

In one embodiment, the invention comprises a combination of any one of independent claims 1 to 6 with dependent claims 16, 18, 33, 42, 45, 64, 72 and 86.

In one embodiment, the invention comprises a combination of any one of independent claims 1 to 6 with dependent claims 10, 27, 33, 42, 45, 64, 72 and 86.

In one embodiment, the invention comprises a combination of any one of independent claims 1 to 6 with dependent claims 15, 27, 33, 42, 45, 64, 72 and 86.

In one embodiment, the invention comprises a combination of any one of independent claims 1 to 6 with dependent claims 16, 27, 33, 42, 45, 64, 72 and 86.

In one embodiment, the invention comprises a combination of any one of independent claims 1 to 6 with dependent claims 10, 17, 34, 35, 36, 37, 38, 42, 45, 64, 72 and 86.

In one embodiment, the invention comprises a combination of any one of independent claims 1 to 6 with dependent claims 15, 17, 34, 35, 36, 37, 38, 42, 45, 64, 72 and 86.

In one embodiment, the invention comprises a combination of any one of independent claims 1 to 6 with dependent claims 16, 17, 34, 35, 36, 37, 38, 42, 45, 64, 72 and 86.

In one embodiment, the invention comprises a combination of any one of independent claims 1 to 6 with dependent claims 10, 18, 34, 35, 36, 37, 38, 42, 45, 64, 72 and 86.

In one embodiment, the invention comprises a combination of any one of independent claims 1 to 6 with dependent claims 15, 18, 34, 35, 36, 37, 38, 42, 45, 64, 72 and 86.

In one embodiment, the invention comprises a combination of any one of independent claims 1 to 6 with dependent claims 16, 18, 34, 35, 36, 37, 38, 42, 45, 64, 72 and 86.

In one embodiment, the invention comprises a combination of any one of independent claims 1 to 6 with dependent claims 10, 27, 34, 35, 36, 37, 38, 42, 45, 64, 72 and 86.

In one embodiment, the invention comprises a combination of any one of independent claims 1 to 6 with dependent claims 15, 27, 34, 35, 36, 37, 38, 42, 45, 64, 72 and 86.

In one embodiment, the invention comprises a combination of any one of independent claims 1 to 6 with dependent claims 16, 27, 34, 35, 36, 37, 38, 42, 45, 64, 72 and 86.

In one embodiment, the invention comprises a combination of any one of independent claims 1 to 6 with dependent claims 10, 17, 33, 42, 43, 64, 67, 72 and 86.

In one embodiment, the invention comprises a combination of any one of independent claims 1 to 6 with dependent claims 15, 17, 33, 42, 43, 64, 67, 72 and 86.

In one embodiment, the invention comprises a combination of any one of independent claims 1 to 6 with dependent claims 16, 17, 33, 42, 43, 64, 67, 72 and 86.

In one embodiment, the invention comprises a combination of any one of independent claims 1 to 6 with dependent claims 10, 18, 33, 42, 43, 64, 67, 72 and 86.

In one embodiment, the invention comprises a combination of any one of independent claims 1 to 6 with dependent claims 15, 18, 33, 42, 43, 64, 67, 72 and 86.

In one embodiment, the invention comprises a combination of any one of independent claims 1 to 6 with dependent claims 16, 18, 33, 42, 43, 64, 67, 72 and 86.

In one embodiment, the invention comprises a combination of any one of independent claims 1 to 6 with dependent claims 10, 27, 33, 42, 43, 64, 67, 72 and 86.

In one embodiment, the invention comprises a combination of any one of independent claims 1 to 6 with dependent claims 15, 27, 33, 42, 43, 64, 67, 72 and 86.

In one embodiment, the invention comprises a combination of any one of independent claims 1 to 6 with dependent claims 16, 27, 33, 42, 43, 64, 67, 72 and 86.

In one embodiment, the invention comprises a combination of any one of independent claims 1 to 6 with dependent claims 10, 17, 34, 35, 36, 37, 38, 42, 43, 64, 67, 72 and 86.

In one embodiment, the invention comprises a combination of any one of independent claims 1 to 6 with dependent claims 15, 17, 34, 35, 36, 37, 38, 42, 43, 64, 67, 72 and 86.

In one embodiment, the invention comprises a combination of any one of independent claims 1 to 6 with dependent claims 16, 17, 34, 35, 36, 37, 38, 42, 43, 64, 67, 72 and 86.

In one embodiment, the invention comprises a combination of any one of independent claims 1 to 6 with dependent claims 10, 18, 34, 35, 36, 37, 38, 42, 43, 64, 67, 72 and 86.

In one embodiment, the invention comprises a combination of any one of independent claims 1 to 6 with dependent claims 15, 18, 34, 35, 36, 37, 38, 42, 43, 64, 67, 72 and 86.

In one embodiment, the invention comprises a combination of any one of independent claims 1 to 6 with dependent claims 16, 18, 34, 35, 36, 37, 38, 42, 43, 64, 67, 72 and 86.

In one embodiment, the invention comprises a combination of any one of independent claims 1 to 6 with dependent claims 10, 27, 34, 35, 36, 37, 38, 42, 43, 64, 67, 72 and 86.

In one embodiment, the invention comprises a combination of any one of independent claims 1 to 6 with dependent claims 15, 27, 34, 35, 36, 37, 38, 42, 43, 64, 67, 72 and 86.

In one embodiment, the invention comprises a combination of any one of independent claims 1 to 6 with dependent claims 16, 27, 34, 35, 36, 37, 38, 42, 43, 64, 67, 72 and 86.

In one embodiment, the invention comprises a combination of any one of independent claims 1 to 6 with dependent claims 10, 17, 33, 42, 44, 64, 67, 72 and 86.

In one embodiment, the invention comprises a combination of any one of independent claims 1 to 6 with dependent claims 15, 17, 33, 42, 44, 64, 67, 72 and 86.

In one embodiment, the invention comprises a combination of any one of independent claims 1 to 6 with dependent claims 16, 17, 33, 42, 44, 64, 67, 72 and 86.

In one embodiment, the invention comprises a combination of any one of independent claims 1 to 6 with dependent claims 10, 18, 33, 42, 44, 64, 67, 72 and 86.

In one embodiment, the invention comprises a combination of any one of independent claims 1 to 6 with dependent claims 15, 18, 33, 42, 44, 64, 67, 72 and 86.

In one embodiment, the invention comprises a combination of any one of independent claims 1 to 6 with dependent claims 16, 18, 33, 42, 44, 64, 67, 72 and 86.

In one embodiment, the invention comprises a combination of any one of independent claims 1 to 6 with dependent claims 10, 27, 33, 42, 44, 64, 67, 72 and 86.

In one embodiment, the invention comprises a combination of any one of independent claims 1 to 6 with dependent claims 15, 27, 33, 42, 44, 64, 67, 72 and 86.

In one embodiment, the invention comprises a combination of any one of independent claims 1 to 6 with dependent claims 16, 27, 33, 42, 44, 64, 67, 72 and 86.

In one embodiment, the invention comprises a combination of any one of independent claims 1 to 6 with dependent claims 10, 17, 34, 35, 36, 37, 38, 42, 44, 64, 67, 72 and 86.

In one embodiment, the invention comprises a combination of any one of independent claims 1 to 6 with dependent claims 15, 17, 34, 35, 36, 37, 38, 42, 44, 64, 67, 72 and 86.

In one embodiment, the invention comprises a combination of any one of independent claims 1 to 6 with dependent claims 16, 17, 34, 35, 36, 37, 38, 42, 44, 64, 67, 72 and 86.

In one embodiment, the invention comprises a combination of any one of independent claims 1 to 6 with dependent claims 10, 18, 34, 35, 36, 37, 38, 42, 44, 64, 67, 72 and 86.

In one embodiment, the invention comprises a combination of any one of independent claims 1 to 6 with dependent claims 15, 18, 34, 35, 36, 37, 38, 42, 44, 64, 67, 72 and 86.

In one embodiment, the invention comprises a combination of any one of independent claims 1 to 6 with dependent claims 16, 18, 34, 35, 36, 37, 38, 42, 44, 64, 67, 72 and 86.

In one embodiment, the invention comprises a combination of any one of independent claims 1 to 6 with dependent claims 10, 27, 34, 35, 36, 37, 38, 42, 44, 64, 67, 72 and 86.

In one embodiment, the invention comprises a combination of any one of independent claims 1 to 6 with dependent claims 15, 27, 34, 35, 36, 37, 38, 42, 44, 64, 67, 72 and 86.

In one embodiment, the invention comprises a combination of any one of independent claims 1 to 6 with dependent claims 16, 27, 34, 35, 36, 37, 38, 42, 44, 64, 67, 72 and 86.

In one embodiment, the invention comprises a combination of any one of independent claims 1 to 6 with dependent claims 10, 17, 33, 42, 45, 64, 67, 72 and 86.

In one embodiment, the invention comprises a combination of any one of independent claims 1 to 6 with dependent claims 15, 17, 33, 42, 45, 64, 67, 72 and 86.

In one embodiment, the invention comprises a combination of any one of independent claims 1 to 6 with dependent claims 16, 17, 33, 42, 45, 64, 67, 72 and 86.

In one embodiment, the invention comprises a combination of any one of independent claims 1 to 6 with dependent claims 10, 18, 33, 42, 45, 64, 67, 72 and 86.

In one embodiment, the invention comprises a combination of any one of independent claims 1 to 6 with dependent claims 15, 18, 33, 42, 45, 64, 67, 72 and 86.

In one embodiment, the invention comprises a combination of any one of independent claims 1 to 6 with dependent claims 16, 18, 33, 42, 45, 64, 67, 72 and 86.

In one embodiment, the invention comprises a combination of any one of independent claims 1 to 6 with dependent claims 10, 27, 33, 42, 45, 64, 67, 72 and 86.

In one embodiment, the invention comprises a combination of any one of independent claims 1 to 6 with dependent claims 15, 27, 33, 42, 45, 64, 67, 72 and 86.

In one embodiment, the invention comprises a combination of any one of independent claims 1 to 6 with dependent claims 16, 27, 33, 42, 45, 64, 67, 72 and 86.

In one embodiment, the invention comprises a combination of any one of independent claims 1 to 6 with dependent claims 10, 17, 34, 35, 36, 37, 38, 42, 45, 64, 67, 72 and 86.

In one embodiment, the invention comprises a combination of any one of independent claims 1 to 6 with dependent claims 15, 17, 34, 35, 36, 37, 38, 42, 45, 64, 67, 72 and 86.

In one embodiment, the invention comprises a combination of any one of independent claims 1 to 6 with dependent claims 16, 17, 34, 35, 36, 37, 38, 42, 45, 64, 67, 72 and 86.

In one embodiment, the invention comprises a combination of any one of independent claims 1 to 6 with dependent claims 10, 18, 34, 35, 36, 37, 38, 42, 45, 64, 67, 72 and 86.

In one embodiment, the invention comprises a combination of any one of independent claims 1 to 6 with dependent claims 15, 18, 34, 35, 36, 37, 38, 42, 45, 64, 67, 72 and 86.

In one embodiment, the invention comprises a combination of any one of independent claims 1 to 6 with dependent claims 16, 18, 34, 35, 36, 37, 38, 42, 45, 64, 67, 72 and 86.

In one embodiment, the invention comprises a combination of any one of independent claims 1 to 6 with dependent claims 10, 27, 34, 35, 36, 37, 38, 42, 45, 64, 67, 72 and 86.

In one embodiment, the invention comprises a combination of any one of independent claims 1 to 6 with dependent claims 15, 27, 34, 35, 36, 37, 38, 42, 45, 64, 67, 72 and 86.

In one embodiment, the invention comprises a combination of any one of independent claims 1 to 6 with dependent claims 16, 27, 34, 35, 36, 37, 38, 42, 45, 64, 67, 72 and 86.

In one embodiment, the invention comprises a combination of any one of independent claims 1 to 6 with dependent claims 10, 17, 33, 42, 43, 64, 74 and 86.

In one embodiment, the invention comprises a combination of any one of independent claims 1 to 6 with dependent claims 15, 17, 33, 42, 43, 64, 74 and 86.

In one embodiment, the invention comprises a combination of any one of independent claims 1 to 6 with dependent claims 16, 17, 33, 42, 43, 64, 74 and 86.

In one embodiment, the invention comprises a combination of any one of independent claims 1 to 6 with dependent claims 10, 18, 33, 42, 43, 64, 74 and 86.

In one embodiment, the invention comprises a combination of any one of independent claims 1 to 6 with dependent claims 15, 18, 33, 42, 43, 64, 74 and 86.

In one embodiment, the invention comprises a combination of any one of independent claims 1 to 6 with dependent claims 16, 18, 33, 42, 43, 64, 74 and 86.

In one embodiment, the invention comprises a combination of any one of independent claims 1 to 6 with dependent claims 10, 27, 33, 42, 43, 64, 74 and 86.

In one embodiment, the invention comprises a combination of any one of independent claims 1 to 6 with dependent claims 15, 27, 33, 42, 43, 64, 74 and 86.

In one embodiment, the invention comprises a combination of any one of independent claims 1 to 6 with dependent claims 16, 27, 33, 42, 43, 64, 74 and 86.

In one embodiment, the invention comprises a combination of any one of independent claims 1 to 6 with dependent claims 10, 17, 34, 35, 36, 37, 38, 42, 43, 64, 74 and 86.

In one embodiment, the invention comprises a combination of any one of independent claims 1 to 6 with dependent claims 15, 17, 34, 35, 36, 37, 38, 42, 43, 64, 74 and 86.

In one embodiment, the invention comprises a combination of any one of independent claims 1 to 6 with dependent claims 16, 17, 34, 35, 36, 37, 38, 42, 43, 64, 74 and 86.

In one embodiment, the invention comprises a combination of any one of independent claims 1 to 6 with dependent claims 10, 18, 34, 35, 36, 37, 38, 42, 43, 64, 74 and 86.

In one embodiment, the invention comprises a combination of any one of independent claims 1 to 6 with dependent claims 15, 18, 34, 35, 36, 37, 38, 42, 43, 64, 74 and 86.

In one embodiment, the invention comprises a combination of any one of independent claims 1 to 6 with dependent claims 16, 18, 34, 35, 36, 37, 38, 42, 43, 64, 74 and 86.

In one embodiment, the invention comprises a combination of any one of independent claims 1 to 6 with dependent claims 10, 27, 34, 35, 36, 37, 38, 42, 43, 64, 74 and 86.

In one embodiment, the invention comprises a combination of any one of independent claims 1 to 6 with dependent claims 15, 27, 34, 35, 36, 37, 38, 42, 43, 64, 74 and 86.

In one embodiment, the invention comprises a combination of any one of independent claims 1 to 6 with dependent claims 16, 27, 34, 35, 36, 37, 38, 42, 43, 64, 74 and 86.

In one embodiment, the invention comprises a combination of any one of independent claims 1 to 6 with dependent claims 10, 17, 33, 42, 44, 64, 74 and 86.

In one embodiment, the invention comprises a combination of any one of independent claims 1 to 6 with dependent claims 15, 17, 33, 42, 44, 64, 74 and 86.

In one embodiment, the invention comprises a combination of any one of independent claims 1 to 6 with dependent claims 16, 17, 33, 42, 44, 64, 74 and 86.

In one embodiment, the invention comprises a combination of any one of independent claims 1 to 6 with dependent claims 10, 18, 33, 42, 44, 64, 74 and 86.

In one embodiment, the invention comprises a combination of any one of independent claims 1 to 6 with dependent claims 15, 18, 33, 42, 44, 64, 74 and 86.

In one embodiment, the invention comprises a combination of any one of independent claims 1 to 6 with dependent claims 16, 18, 33, 42, 44, 64, 74 and 86.

In one embodiment, the invention comprises a combination of any one of independent claims 1 to 6 with dependent claims 10, 27, 33, 42, 44, 64, 74 and 86.

In one embodiment, the invention comprises a combination of any one of independent claims 1 to 6 with dependent claims 15, 27, 33, 42, 44, 64, 74 and 86.

In one embodiment, the invention comprises a combination of any one of independent claims 1 to 6 with dependent claims 16, 27, 33, 42, 44, 64, 74 and 86.

In one embodiment, the invention comprises a combination of any one of independent claims 1 to 6 with dependent claims 10, 17, 34, 35, 36, 37, 38, 42, 44, 64, 74 and 86.

In one embodiment, the invention comprises a combination of any one of independent claims 1 to 6 with dependent claims 15, 17, 34, 35, 36, 37, 38, 42, 44, 64, 74 and 86.

In one embodiment, the invention comprises a combination of any one of independent claims 1 to 6 with dependent claims 16, 17, 34, 35, 36, 37, 38, 42, 44, 64, 74 and 86.

In one embodiment, the invention comprises a combination of any one of independent claims 1 to 6 with dependent claims 10, 18, 34, 35, 36, 37, 38, 42, 44, 64, 74 and 86.

In one embodiment, the invention comprises a combination of any one of independent claims 1 to 6 with dependent claims 15, 18, 34, 35, 36, 37, 38, 42, 44, 64, 74 and 86.

In one embodiment, the invention comprises a combination of any one of independent claims 1 to 6 with dependent claims 16, 18, 34, 35, 36, 37, 38, 42, 44, 64, 74 and 86.

In one embodiment, the invention comprises a combination of any one of independent claims 1 to 6 with dependent claims 10, 27, 34, 35, 36, 37, 38, 42, 44, 64, 74 and 86.

In one embodiment, the invention comprises a combination of any one of independent claims 1 to 6 with dependent claims 15, 27, 34, 35, 36, 37, 38, 42, 44, 64, 74 and 86.

In one embodiment, the invention comprises a combination of any one of independent claims 1 to 6 with dependent claims 16, 27, 34, 35, 36, 37, 38, 42, 44, 64, 74 and 86.

In one embodiment, the invention comprises a combination of any one of independent claims 1 to 6 with dependent claims 10, 17, 33, 42, 45, 64, 74 and 86.

In one embodiment, the invention comprises a combination of any one of independent claims 1 to 6 with dependent claims 15, 17, 33, 42, 45, 64, 74 and 86.

In one embodiment, the invention comprises a combination of any one of independent claims 1 to 6 with dependent claims 16, 17, 33, 42, 45, 64, 74 and 86.

In one embodiment, the invention comprises a combination of any one of independent claims 1 to 6 with dependent claims 10, 18, 33, 42, 45, 64, 74 and 86.

In one embodiment, the invention comprises a combination of any one of independent claims 1 to 6 with dependent claims 15, 18, 33, 42, 45, 64, 74 and 86.

In one embodiment, the invention comprises a combination of any one of independent claims 1 to 6 with dependent claims 16, 18, 33, 42, 45, 64, 74 and 86.

In one embodiment, the invention comprises a combination of any one of independent claims 1 to 6 with dependent claims 10, 27, 33, 42, 45, 64, 74 and 86.

In one embodiment, the invention comprises a combination of any one of independent claims 1 to 6 with dependent claims 15, 27, 33, 42, 45, 64, 74 and 86.

In one embodiment, the invention comprises a combination of any one of independent claims 1 to 6 with dependent claims 16, 27, 33, 42, 45, 64, 74 and 86.

In one embodiment, the invention comprises a combination of any one of independent claims 1 to 6 with dependent claims 10, 17, 34, 35, 36, 37, 38, 42, 45, 64, 74 and 86.

In one embodiment, the invention comprises a combination of any one of independent claims 1 to 6 with dependent claims 15, 17, 34, 35, 36, 37, 38, 42, 45, 64, 74 and 86.

In one embodiment, the invention comprises a combination of any one of independent claims 1 to 6 with dependent claims 16, 17, 34, 35, 36, 37, 38, 42, 45, 64, 74 and 86.

In one embodiment, the invention comprises a combination of any one of independent claims 1 to 6 with dependent claims 10, 18, 34, 35, 36, 37, 38, 42, 45, 64, 74 and 86.

In one embodiment, the invention comprises a combination of any one of independent claims 1 to 6 with dependent claims 15, 18, 34, 35, 36, 37, 38, 42, 45, 64, 74 and 86.

In one embodiment, the invention comprises a combination of any one of independent claims 1 to 6 with dependent claims 16, 18, 34, 35, 36, 37, 38, 42, 45, 64, 74 and 86.

In one embodiment, the invention comprises a combination of any one of independent claims 1 to 6 with dependent claims 10, 27, 34, 35, 36, 37, 38, 42, 45, 64, 74 and 86.

In one embodiment, the invention comprises a combination of any one of independent claims 1 to 6 with dependent claims 15, 27, 34, 35, 36, 37, 38, 42, 45, 64, 74 and 86.

In one embodiment, the invention comprises a combination of any one of independent claims 1 to 6 with dependent claims 16, 27, 34, 35, 36, 37, 38, 42, 45, 64, 74 and 86.

In one embodiment, the invention comprises a combination of any one of independent claims 1 to 6 with dependent claims 10, 17, 33, 42, 43, 64, 67, 74 and 86.

In one embodiment, the invention comprises a combination of any one of independent claims 1 to 6 with dependent claims 15, 17, 33, 42, 43, 64, 67, 74 and 86.

In one embodiment, the invention comprises a combination of any one of independent claims 1 to 6 with dependent claims 16, 17, 33, 42, 43, 64, 67, 74 and 86.

In one embodiment, the invention comprises a combination of any one of independent claims 1 to 6 with dependent claims 10, 18, 33, 42, 43, 64, 67, 74 and 86.

In one embodiment, the invention comprises a combination of any one of independent claims 1 to 6 with dependent claims 15, 18, 33, 42, 43, 64, 67, 74 and 86.

In one embodiment, the invention comprises a combination of any one of independent claims 1 to 6 with dependent claims 16, 18, 33, 42, 43, 64, 67, 74 and 86.

In one embodiment, the invention comprises a combination of any one of independent claims 1 to 6 with dependent claims 10, 27, 33, 42, 43, 64, 67, 74 and 86.

In one embodiment, the invention comprises a combination of any one of independent claims 1 to 6 with dependent claims 15, 27, 33, 42, 43, 64, 67, 74 and 86.

In one embodiment, the invention comprises a combination of any one of independent claims 1 to 6 with dependent claims 16, 27, 33, 42, 43, 64, 67, 74 and 86.

In one embodiment, the invention comprises a combination of any one of independent claims 1 to 6 with dependent claims 10, 17, 34, 35, 36, 37, 38, 42, 43, 64, 67, 74 and 86.

In one embodiment, the invention comprises a combination of any one of independent claims 1 to 6 with dependent claims 15, 17, 34, 35, 36, 37, 38, 42, 43, 64, 67, 74 and 86.

In one embodiment, the invention comprises a combination of any one of independent claims 1 to 6 with dependent claims 16, 17, 34, 35, 36, 37, 38, 42, 43, 64, 67, 74 and 86.

In one embodiment, the invention comprises a combination of any one of independent claims 1 to 6 with dependent claims 10, 18, 34, 35, 36, 37, 38, 42, 43, 64, 67, 74 and 86.

In one embodiment, the invention comprises a combination of any one of independent claims 1 to 6 with dependent claims 15, 18, 34, 35, 36, 37, 38, 42, 43, 64, 67, 74 and 86.

In one embodiment, the invention comprises a combination of any one of independent claims 1 to 6 with dependent claims 16, 18, 34, 35, 36, 37, 38, 42, 43, 64, 67, 74 and 86.

In one embodiment, the invention comprises a combination of any one of independent claims 1 to 6 with dependent claims 10, 27, 34, 35, 36, 37, 38, 42, 43, 64, 67, 74 and 86.

In one embodiment, the invention comprises a combination of any one of independent claims 1 to 6 with dependent claims 15, 27, 34, 35, 36, 37, 38, 42, 43, 64, 67, 74 and 86.

In one embodiment, the invention comprises a combination of any one of independent claims 1 to 6 with dependent claims 16, 27, 34, 35, 36, 37, 38, 42, 43, 64, 67, 74 and 86.

In one embodiment, the invention comprises a combination of any one of independent claims 1 to 6 with dependent claims 10, 17, 33, 42, 44, 64, 67, 74 and 86.

In one embodiment, the invention comprises a combination of any one of independent claims 1 to 6 with dependent claims 15, 17, 33, 42, 44, 64, 67, 74 and 86.

In one embodiment, the invention comprises a combination of any one of independent claims 1 to 6 with dependent claims 16, 17, 33, 42, 44, 64, 67, 74 and 86.

In one embodiment, the invention comprises a combination of any one of independent claims 1 to 6 with dependent claims 10, 18, 33, 42, 44, 64, 67, 74 and 86.

In one embodiment, the invention comprises a combination of any one of independent claims 1 to 6 with dependent claims 15, 18, 33, 42, 44, 64, 67, 74 and 86.

In one embodiment, the invention comprises a combination of any one of independent claims 1 to 6 with dependent claims 16, 18, 33, 42, 44, 64, 67, 74 and 86.

In one embodiment, the invention comprises a combination of any one of independent claims 1 to 6 with dependent claims 10, 27, 33, 42, 44, 64, 67, 74 and 86.

In one embodiment, the invention comprises a combination of any one of independent claims 1 to 6 with dependent claims 15, 27, 33, 42, 44, 64, 67, 74 and 86.

In one embodiment, the invention comprises a combination of any one of independent claims 1 to 6 with dependent claims 16, 27, 33, 42, 44, 64, 67, 74 and 86.

In one embodiment, the invention comprises a combination of any one of independent claims 1 to 6 with dependent claims 10, 17, 34, 35, 36, 37, 38, 42, 44, 64, 67, 74 and 86.

In one embodiment, the invention comprises a combination of any one of independent claims 1 to 6 with dependent claims 15, 17, 34, 35, 36, 37, 38, 42, 44, 64, 67, 74 and 86.

In one embodiment, the invention comprises a combination of any one of independent claims 1 to 6 with dependent claims 16, 17, 34, 35, 36, 37, 38, 42, 44, 64, 67, 74 and 86.

In one embodiment, the invention comprises a combination of any one of independent claims 1 to 6 with dependent claims 10, 18, 34, 35, 36, 37, 38, 42, 44, 64, 67, 74 and 86.

In one embodiment, the invention comprises a combination of any one of independent claims 1 to 6 with dependent claims 15, 18, 34, 35, 36, 37, 38, 42, 44, 64, 67, 74 and 86.

In one embodiment, the invention comprises a combination of any one of independent claims 1 to 6 with dependent claims 16, 18, 34, 35, 36, 37, 38, 42, 44, 64, 67, 74 and 86.

In one embodiment, the invention comprises a combination of any one of independent claims 1 to 6 with dependent claims 10, 27, 34, 35, 36, 37, 38, 42, 44, 64, 67, 74 and 86.

In one embodiment, the invention comprises a combination of any one of independent claims 1 to 6 with dependent claims 15, 27, 34, 35, 36, 37, 38, 42, 44, 64, 67, 74 and 86.

In one embodiment, the invention comprises a combination of any one of independent claims 1 to 6 with dependent claims 16, 27, 34, 35, 36, 37, 38, 42, 44, 64, 67, 74 and 86.

In one embodiment, the invention comprises a combination of any one of independent claims 1 to 6 with dependent claims 10, 17, 33, 42, 45, 64, 67, 74 and 86.

In one embodiment, the invention comprises a combination of any one of independent claims 1 to 6 with dependent claims 15, 17, 33, 42, 45, 64, 67, 74 and 86.

In one embodiment, the invention comprises a combination of any one of independent claims 1 to 6 with dependent claims 16, 17, 33, 42, 45, 64, 67, 74 and 86.

In one embodiment, the invention comprises a combination of any one of independent claims 1 to 6 with dependent claims 10, 18, 33, 42, 45, 64, 67, 74 and 86.

In one embodiment, the invention comprises a combination of any one of independent claims 1 to 6 with dependent claims 15, 18, 33, 42, 45, 64, 67, 74 and 86.

In one embodiment, the invention comprises a combination of any one of independent claims 1 to 6 with dependent claims 16, 18, 33, 42, 45, 64, 67, 74 and 86.

In one embodiment, the invention comprises a combination of any one of independent claims 1 to 6 with dependent claims 10, 27, 33, 42, 45, 64, 67, 74 and 86.

In one embodiment, the invention comprises a combination of any one of independent claims 1 to 6 with dependent claims 15, 27, 33, 42, 45, 64, 67, 74 and 86.

In one embodiment, the invention comprises a combination of any one of independent claims 1 to 6 with dependent claims 16, 27, 33, 42, 45, 64, 67, 74 and 86.

In one embodiment, the invention comprises a combination of any one of independent claims 1 to 6 with dependent claims 10, 17, 34, 35, 36, 37, 38, 42, 45, 64, 67, 74 and 86.

In one embodiment, the invention comprises a combination of any one of independent claims 1 to 6 with dependent claims 15, 17, 34, 35, 36, 37, 38, 42, 45, 64, 67, 74 and 86.

In one embodiment, the invention comprises a combination of any one of independent claims 1 to 6 with dependent claims 16, 17, 34, 35, 36, 37, 38, 42, 45, 64, 67, 74 and 86.

In one embodiment, the invention comprises a combination of any one of independent claims 1 to 6 with dependent claims 10, 18, 34, 35, 36, 37, 38, 42, 45, 64, 67, 74 and 86.

In one embodiment, the invention comprises a combination of any one of independent claims 1 to 6 with dependent claims 15, 18, 34, 35, 36, 37, 38, 42, 45, 64, 67, 74 and 86.

In one embodiment, the invention comprises a combination of any one of independent claims 1 to 6 with dependent claims 16, 18, 34, 35, 36, 37, 38, 42, 45, 64, 67, 74 and 86.

In one embodiment, the invention comprises a combination of any one of independent claims 1 to 6 with dependent claims 10, 27, 34, 35, 36, 37, 38, 42, 45, 64, 67, 74 and 86.

In one embodiment, the invention comprises a combination of any one of independent claims 1 to 6 with dependent claims 15, 27, 34, 35, 36, 37, 38, 42, 45, 64, 67, 74 and 86.

In one embodiment, the invention comprises a combination of any one of independent claims 1 to 6 with dependent claims 16, 27, 34, 35, 36, 37, 38, 42, 45, 64, 67, 74 and 86.

1: A method of killing planktonic cells and biofilm cells comprised by a cell population, the method comprising introducing a nucleic acid vector into the planktonic cells and biofilm cells,
wherein the nucleic acid vector comprises a nucleotide sequence that encodes a CRISPR/Cas system or a component thereof that is capable of recognizing and cutting at least one target sequence comprised by the planktonic cells and biofilm cells, whereby the planktonic cells and biofilm cells are killed, and
wherein the nucleotide sequence of the vector is under the control of a stress-phase active (SPA) promoter which is a BolA promoter or an orthologue or homologue promoter from a different species for expression of the CRISPR/Cas system or component thereof and the sequence is expressed in the planktonic cells and biofilm cells to produce the CRISPR/Cas system or component thereof.

2: A nucleic acid vector comprising a nucleotide sequence that encodes a CRISPR/Cas system or a component thereof that is capable of recognizing and cutting at least one target sequence comprised by planktonic cells and biofilm cells, whereby the planktonic cells and biofilm cells are killed,
wherein the nucleotide sequence of the vector is under the control of a stress-phase active (SPA) which is a BolA promoter or an orthologue or homologue promoter from a different species for expression of the CRISPR/Cas system or component thereof, and
wherein the nucleotide sequence of the vector is expressed in the planktonic cells and biofilm cells to produce the CRISPR/Cas system or component thereof.

3: The method according to claim 1, wherein the nucleic acid vector is formulated in a pharmaceutical composition comprising a diluent, excipient or carrier, optionally wherein the formulation is comprised within a medical device (such as an ampoule, a syringe, or an inhaler) or is formulated in a tincture, a capsule or a slow-release formulation.

4: A method of treating or preventing a disease or condition in a patient that is mediated by planktonic cells and biofilm cells, the method comprising administering a nucleic acid vector as defined in claim 2 to the patient thereby killing the planktonic cells and biofilm cells.

5: A method of treating or preventing a disease or condition in a patient, the method comprising administering a nucleic acid vector as defined in claim 2 to the patient to deliver the CRISPR/Cas system to the planktonic cells and biofilm cells.

6: The method according to claim 1, wherein the nucleic acid vector is a plasmid, phage DNA or phagemid.

7: The method according to claim 6, wherein the nucleic acid vector is comprised by transduction particles (optionally phage or a non-self-replicative transduction particle) that infect, or is capable of infecting, the planktonic cells and biofilm cells to introduce the vector into the planktonic cells and biofilm cells.

8: The method according to claim 1, wherein the nucleic acid vector is a plasmid, optionally a conjugative plasmid that is introduced, or is capable of being introduced, into the planktonic cells and biofilm cells.

9: The method according to claim 1, wherein the nucleic acid vector is comprised by a lytic phage.

10: The method according to claim 1, wherein the planktonic cells and biofilm cells are of a first species or strain.

11: The method according to claim 1, wherein the planktonic cells and biofilm cells are prokaryotic cells.

12: The method according to claim 1, wherein the planktonic cells and biofilm cells are selected from the group consisting of microorganism cells, bacterial cells, archaeal cells and fungal cells (e.g. yeast cells).

13: The method according to claim 12, wherein the planktonic cells and biofilm cells are selected from the group consisting of bacterial cells, archaeal cells and yeast cells comprised by a microbiome.

14: The method according to claim 12, wherein the planktonic cells and biofilm cells are bacterial cells.

15: The method according to claim 14, wherein the planktonic cells and biofilm cells are selected from any species disclosed in Table 5, optionally E. coli, Klebsiella pneumoniae, Clostridium difficile, Staphylococcus aureus, Helicobacter pylori, Fusobacterium nucleatum, Mycobacterium tuberculosis or an Enterococcus species.

16: The method according to claim 1, wherein the CRISPR/Cas system is one or more guided nuclease(s) or one or more RNA(s) (such as a guide RNA or crRNA) for targeting a sequence of the at least one target sequence comprised by the genome of the planktonic cells and biofilm cells, optionally one to three guided nuclease(s) or one to three RNA(s), e.g. one or two guided nuclease(s) or one or two RNA(s).

17: The method according to claim 1, wherein the target sequence is comprised by a chromosomal sequence.

18: The method according to claim 1, wherein the CRISPR/Cas system comprises one or more crRNA(s) or one or more RNA(s) (such as a guide RNA or crRNA), and at least one Cas (e.g. a Cas) nuclease,
wherein each crRNA or gRNA is operable in the planktonic cells and biofilm cells with a cognate Cas to guide the Cas to the target sequence (e.g. protospacer sequence) comprised by the planktonic cells and biofilm cells.

19: The method according to claim 18, wherein the CRISPR/Cas system encodes a Cas nuclease, such as a Cas9, Cas3, Cas12 or Cas13, that is operable in the planktonic cells and biofilm cells with the crRNA(s) or the RNA(s) to guide the Cas to a the target sequence (e.g. protospacer sequence) comprised by the planktonic cells and biofilm cells.

20: The method according to claim 1, wherein the method is carried out ex vivo or in vitro.


SEQUENCE LISTING (10 sequences)

SEQ ID NO: 1 - DNA, 85 bp, Escherichia coli
  source 1..85: mol_type = other DNA
cgactactttctgacttccttcgtgacttgccctaagcatgttgtagtgcgatacttgtaatgacatttgtaattacaagaggtg

SEQ ID NO: 2 - DNA, 85 bp, Escherichia coli
  source 1..85: mol_type = other DNA
aacctaaatatttgttgttaagctgcaatggaaacggtaaaagcggctagtatttaaagggatggatgacatctcagcgttgtcg

SEQ ID NO: 3 - DNA, 85 bp, Escherichia coli
  source 1..85: mol_type = other DNA
aaaagataaatccacacagtttgtattgttttgtgcaaaagtttcactacgctttattaacaatactttctggcgacgtgcgcca

SEQ ID NO: 4 - DNA, 85 bp, Escherichia coli
  source 1..85: mol_type = other DNA
agccagagcatgccctgacttcaccccgctgtgtctgcttttcccgactattcttaatgagcttcgatgcaattcacgatcccgc

SEQ ID NO: 5 - DNA, 85 bp, Escherichia coli
  source 1..85: mol_type = other DNA
acggtacaacatttacgccactttacgcctgaataataaaagcgtgttatactctttccctgcaatgggttccgtagcagggaaa

SEQ ID NO: 6 - DNA, 85 bp, Escherichia coli; identical to SEQ ID NO: 5
  source 1..85: mol_type = other DNA

SEQ ID NO: 7 - DNA, 85 bp, Escherichia coli
  source 1..85: mol_type = other DNA
tgcgtaatttattcacaagcttgcattgaacttgtggataaaatcacggtctgataaaacagtgaatgataacctcgttgctctt

SEQ ID NO: 8 - DNA, 85 bp, Escherichia coli
  source 1..85: mol_type = other DNA
tttattcacaagcttgcattgaacttgtggataaaatcacggtctgataaaacagtgaatgataacctcgttgctcttaagctct

SEQ ID NO: 9 - DNA, 85 bp, Escherichia coli
  source 1..85: mol_type = other DNA
tgcattgaacttgtggataaaatcacggtctgataaaacagtgaatgataacctcgttgctcttaagctctggcacagttgttgc

SEQ ID NO: 10 - DNA, 85 bp, Escherichia coli
  source 1..85: mol_type = other DNA
aacagtgaatgataacctcgttgctcttaagctctggcacagttgttgctaccactgaagcgccagaagatatcgattgagagga